(19) United States
(12) Patent Application Publication
(10) Pub. No.: US 2025/0295814 A1
(43) Pub. Date: Sep. 25, 2025

(54) COMPOSITIONS AND METHODS FOR MODIFYING DUX4

(71) Applicant: Mammoth Biosciences, Inc., Brisbane, CA (US)

(72) Inventors: Renan B. SPER, San Francisco, CA (US); Pei-Qi LIU, Oakland, CA (US); Wiputra Jaya HARTONO, San Francisco, CA (US); Yuchen GAO, South San Francisco, CA (US); Jason Chen LIN, Oakland, CA (US); Ning CHAI, Dublin, CA (US); Aaron DELOUGHERY, San Francisco, CA (US); Benjamin Julius RAUCH, San Francisco, CA (US); Stepan TYMOSHENKO, Sacramento, CA (US); William Douglass WRIGHT, Fairfield, CA (US)

(73) Assignee: Mammoth Biosciences, Inc., Brisbane, CA (US)

(21) Appl. No.: 19/230,223

(22) Filed: Jun. 6, 2025

Related U.S. Application Data
(63) Continuation of application No. PCT/US2023/085044, filed on Dec. 20, 2023.
(60) Provisional application No. 63/476,829, filed on Dec. 22, 2022.
(60) Provisional application No. 63/476,850, filed on Dec. 22, 2022.
(60) Provisional application No. 63/486,704, filed on Feb. 24, 2023.
(60) Provisional application No. 63/486,708, filed on Feb. 24, 2023.
(60) Provisional application No. 63/514,815, filed on Jul. 21, 2023.
(60) Provisional application No. 63/586,111, filed on Sep. 28, 2023.

(51) Int. Cl. A61K 48/00; C12N 9/22; C12N 15/11; C12N 15/86

(52) U.S. Cl. CPC A61K 48/0058; C12N 9/226; C12N 15/11; C12N 15/86; C12N 2310/20; C12N 2750/14143

(57) ABSTRACT

Provided herein are compositions, systems, and methods comprising effector proteins for treating DUX4 mutations. These effector proteins may be characterized as CRISPR-associated (Cas) proteins. Various compositions, systems, and methods of the present disclosure may leverage the activities of these effector proteins for the modification, detection, and engineering the DUX4 gene.

### CROSS-REFERENCE TO RELATED APPLICATIONS

The present application is a continuation of International PCT Application No. PCT/US2023/085044, filed Dec. 20, 2023, which claims priority to U.S. Provisional Application 63/476,829, filed Dec. 22, 2022; U.S. Provisional Application 63/476,850, filed Dec. 22, 2022; U.S. Provisional Application 63/486,704, filed Feb. 24, 2023; U.S. Provisional Application 63/486,708, filed Feb. 24, 2023; U.S. Provisional Application 63/514,815, filed Jul. 21, 2023; and U.S. Provisional Application 63/586,111, filed Sep. 28, 2023, the contents each of which are incorporated herein by reference in their entireties.

### SEQUENCE LISTING

The contents of the electronic sequence listing (MABI_030_04US_SeqList_ST26.xml; Size: 690,771 bytes; and Date of Creation: May 6, 2025) are herein incorporated by reference in its entirety.

### BACKGROUND

The DUX4 protein is expressed in the testes and thymus during early embryonic development. However, aberrant expression of the DUX4 protein causes aberrant cell signaling and is, in some embodiments, the cause of facioscapulohumeral muscular dystrophy (FSHD). FSHD is characterized by the degradation of myofibers in the face, scapula, and humerus among other muscles.

The DUX4 gene is located within a D4Z4 repeat array in the subtelomeric region of chromosome 4q. Each D4Z4 repeat unit has an open reading frame (named DUX4) that encodes two homeoboxes. The two homeodomains allow DUX4 protein to bind to DNA. The encoded protein has been reported to function as a transcriptional activator of paired-like homeodomain transcription factor 1 (PITX1). DUX4 is normally expressed in the testes, thymus, and cleavage-stage embryos; however, inappropriate expression of DUX4 in muscle cells is the cause of facioscapulohumeral muscular dystrophy (FSHD).

FSHD is the third most common form of muscular dystrophy, affecting about 1 in 15,000 live births. FSHD is characterized in the degradation of myofibers in the face, scapula, and humerus among other muscles. An autosomal dominant disease, adult-onset FSHD consists of appearance of symptoms in the late twenties or thirties, with subsequent progressive degeneration of muscles of the face, shoulder blades, and upper arms. With roughly one-fifth of patients being confined to a wheelchair by age 50, this is an extremely debilitating condition involving expensive palliative care, and currently does not have a cure or effective therapy.

Additionally, overexpression of DUX4 due to translocations can also cause B-cell leukemia (see, e.g., Lee et al. (December 2018). “Crystal Structure of the Double Homeodomain of DUX4 in Complex with DNA”. Cell Reports. 25 (11): 2955-2962), and a translocation that merges DUX4 with CIC can cause an aggressive type of sarcoma (see, e.g., Wong D, Yip S (April 2020). “Making heads or tails—the emergence of capicua (CIC) as an important multifunctional tumour suppressor”. The Journal of Pathology. 250 (5): 532-540).

### SUMMARY

Preventing or reducing expression of the DUX4 protein may have therapeutic potential for muscular dystrophies such as FSHD. Disclosed herein, in some aspects, are compositions and systems comprising a guide ribonucleic acid (RNA) or a polynucleotide encoding the same, wherein the guide RNA comprises: a first region comprising a protein binding sequence, and a second region comprising a targeting sequence that is complementary to a target sequence that is within a DUX4 gene, wherein the target sequence is adjacent to a protospacer adjacent motif (PAM) selected from 5′-NTTN-3′ and 5′-NNTN-3′. In some embodiments, the targeting sequence comprises a nucleotide sequence that is at least 70%, at least 75%, at least 80%, at least 85%, at least 90%, at least 95% or 100% identical to any one of SEQ ID NOs: 1-114, 275-349, 456-460, and 481-596. In some embodiments, wherein the PAM is 5′-NTTN-3′ and the targeting sequence comprises a nucleotide sequence that is at least 70%, at least 75%, at least 80%, at least 85%, at least 90%, at least 95% or 100% identical to any one of SEQ ID NOs: 1-114, 456, and 481-596, and the protein binding sequence comprises a nucleotide sequence that is at least 70%, at least 75%, at least 80%, at least 85%, at least 90%, at least 95% or 100% identical to any one of SEQ ID NOs: 115, and 237-242. In some embodiments, the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NO: 230. In some embodiments, the guide RNA comprises a nucleotide sequence that is at least 70%, at least 75%, at least 80%, at least 85%, at least 90%, at least 95% or 100% identical to any one of SEQ ID NOS: 116-229, 461, and 602-717.

In some embodiments, the PAM is 5′-NNTN-3′, and wherein the targeting sequence comprises a nucleotide sequence that is at least 70%, at least 75%, at least 80%, at least 85%, at least 90%, at least 95% or 100% identical to any one of SEQ ID NOs: 275-349, 457-460, and 476-480, and the protein binding sequence comprises a nucleotide sequence that is at least 70%, at least 75%, at least 80%, at least 85%, at least 90%, at least 95% or 100% identical to SEQ ID NO: 350. In some embodiments, the protein binding sequence further comprises a nucleotide sequence that is at least 70%, at least 75%, at least 80%, at least 85%, at least 90%, at least 95% or 100% identical to SEQ ID NOs: 351 or 352. In some embodiments, the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NO: 428. In some embodiments, the guide RNA comprises a nucleotide sequence that is at least 70%, at least 75%, at least 80%, at least 85%, at least 90%, at least 95% or 100% identical to any one of SEQ ID NOs: 353-427, 462-465, and 597-601. In some embodiments, the effector protein is fused to an effector partner protein, optionally wherein the effector partner protein is selected from a deaminase, a reverse transcriptase, a recombinase, and a methyltransferase. In some embodiments, the targeting sequence is at least 80%, at least 85%, at least 90%, at least 95%, or 100% identical to a sequence selected from SEQ ID NOs: 481-485, and wherein the effector protein is at least 80%, at least 85%, at least 90%, at least 95%, or 100% identical to SEQ ID NO: 230, and wherein the effector protein is fused to a base editing enzyme. In some embodiments, the targeting sequence is at least 80%, at least 85%, at least 90%, at least 95%, or 100% identical to a sequence selected from SEQ ID NOs: 476-480, wherein the effector protein is at least 80%, at least 85%, at least 90%, at least 95%, or 100% identical to SEQ ID NO: 428, and wherein the effector protein is fused to a base editing enzyme. In some embodiments, the targeting sequence is at least 80%, at least 85%, at least 90%, at least 95%, or 100% identical to a sequence selected from SEQ ID NOs: 486-596, wherein the effector protein is at least 80%, at least 85%, at least 90%, at least 95%, or 100% identical to SEQ ID NO: 230 and wherein the effector protein is fused to a KRAB domain, a methyltransferase, or a combination thereof.

Also, disclosed herein, in some aspects, are expression cassettes comprising, from 5′ to 3′: a first inverted terminal repeat (ITR); a first promoter sequence operably linked to a nucleic acid sequence encoding a guide RNA wherein the guide RNA comprises: a first region comprising a protein binding sequence; and a second region comprising a spacer sequence that is complementary to a target sequence of a DUX4 gene, wherein the spacer sequence is at least 70%, at least 75%, at least 80%, at least 85%, at least 90%, at least 95% or 100% identical to any one of SEQ ID NOs: 1-114, 275-349, 456-460, and 481-596; a second promoter sequence operably linked to a nucleic acid sequence encoding an effector protein; a poly(A) signal; and a second ITR. In some embodiments, the expression cassette further comprises a WPRE sequence located between the nucleic acid sequence encoding an effector protein and the poly(A) signal. In some embodiments, the first promoter is a U6 promoter, the second promoter is a CK8E promoter or a SPC5 promoter or a combination thereof. In some embodiments, the poly(A) signal is a bGH or an hGH poly(A) signal. In some embodiments, the targeting sequence comprises a nucleotide sequence that is at least 70%, at least 75%, at least 80%, at least 85%, at least 90%, at least 95% or 100% identical to any one of SEQ ID NOs: 1-114, 456, and 481-596, and the effector protein comprises an amino acid sequence that is at least 70%, at least 75%, at least 80%, at least 85%, at least 90%, at least 95% or 100% identical to SEQ ID NO: 230, optionally wherein the protein binding sequence comprises a nucleotide sequence that is at least 70%, at least 75%, at least 80%, at least 85%, at least 90%, at least 95% or 100% identical to any one of SEQ ID NOs: 115 and 237-242. In some embodiments, the guide RNA comprises a nucleotide sequence that is at least 70%, at least 75%, at least 80%, at least 85%, at least 90%, at least 95% or 100% identical to any one of SEQ ID NOs: 116-229, 461, and 602-717. In some embodiments, the targeting sequence comprises a nucleotide sequence that is at least 70%, at least 75%, at least 80%, at least 85%, at least 90%, at least 95% or 100% identical to any one of SEQ ID NOs: 275-349, 457-460, and 476-480, and the effector protein comprises an amino acid sequence that is at least 70%, at least 75%, at least 80%, at least 85%, at least 90%, at least 95% or 100% identical to SEQ ID NO: 428, optionally wherein the protein binding sequence comprises a nucleotide sequence that is at least 70%, at least 75%, at least 80%, at least 85%, at least 90%, at least 95% or 100% identical to SEQ ID NOs: 350, 351, or 352, or a combination thereof. In some embodiments, the guide RNA comprises a nucleotide sequence that is at least herein and throughout. Also disclosed herein are cells, populations of cells, comprising or modified by any of the compositions 70%, at least 75%, at least 80%, at least 85%, at least 90%, at least 95% or 100% identical to any one of SEQ ID NOs: 353-427, 462-465, and 597-601. Also disclosed herein, in some aspects, are adeno-associated virus (AAV) vectors that comprise any of the aforementioned expression cassettes.

Also disclosed herein are pharmaceutical compositions comprising any of the compositions, systems (and components thereof), expression cassettes, or AAV vectors described, systems (and components thereof), expression cassettes, or AAV vectors described herein and throughout.

Also disclosed herein are methods of modifying a DUX4 gene, comprising contacting the DUX4 gene with any of the compositions, systems (and components thereof), expression cassettes, or AAV vectors described herein and throughout. In some embodiments, modifying the DUX4 gene comprises inserting, deleting, or substituting one or more nucleotides in the DUX4 gene. In some embodiments, modifying the DUX4 gene reduces the expression of the DUX4 gene. In some embodiments, the reduced expression of the DUX4 gene is transient. In some embodiments, the reduced expression of the DUX4 gene is permanent. In some embodiments, methods comprise modifying the DUX4 gene in a muscle cell, optionally wherein the muscle cell is selected from a skeletal muscle cell, a myoblast, and a myotube muscle cell. In some embodiments, the muscle cell is in vivo. In some embodiments, the muscle cell is within a subject having facioscapulohumeral muscular dystrophy (FSHD).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1. shows possible locations along the DUX4 gene where therapeutic interventions can reduce expression of the DUX4 gene or reduce the expression of the Dux4 protein.

FIG. 2A illustrates locations within the DUX4 gene that can be targeted with guide RNAs disclosed herein. FIG. 2B shows the results of editing DUX4 with a CasPhi.12 effector protein and the associated guide RNAs disclosed herein.

FIG. 3A illustrates locations within the DUX4 gene that can be targeted with guide RNAs disclosed herein. FIG. 3B shows the results of editing DUX4 with a CasM.265466 effector protein and the associated guide RNAs disclosed herein.

FIG. 4 depicts in vivo gene editing in muscle tissues using AAV9-A4 delivery of CasPhi.12 and CasM.265466 variants.

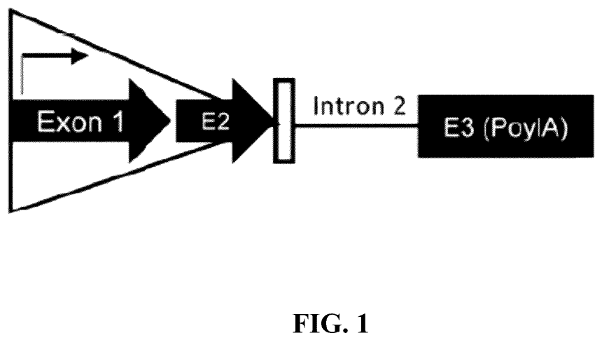

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### I. Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, include plural references unless the context clearly dictates otherwise.

The terms, “or” and “and/or,” as used herein, include any, and all, combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is understood to mean the stated number and numbers +/−10% thereof, or 10% below the lower listed limit and 10% above the higher listed limit for the values listed for a range.

The terms, “% identical,” “% identity,” and “percent identity,” or grammatical equivalents thereof, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4 (1): 11-7), FASTA (Pearson and Lipman, Proc Natl Acad Sci USA. 1988 April; 85 (8): 2444-8; Pearson, Methods Enzymol. 1990; 183:63-98) and gapped BLAST (Altschul et al., Nucleic Acids Res. 1997 Sep. 1; 25 (17): 3389-40), BLASTP, BLASTN, or GCG.

The term “base editing enzyme,” as used herein, refers to a protein, polypeptide, or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term “base editor,” as used herein, refers to a fusion protein comprising a base editing enzyme linked to an effector protein. The base editing enzyme may be referred to as a fusion partner. The base editing enzyme can differ from a naturally occurring base editing enzyme. It is understood that any reference to a base editing enzyme herein also refers to a base editing enzyme variant. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

The term “catalytically inactive effector protein,” also referred to as a “dCas” protein, as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein. In some embodiments, the catalytically inactive effector protein is referred to as a dead Cas protein or a dCas protein.

The term “cis cleavage,” as used herein, refers to cleavage (hydrolysis of a phosphodiester bond) of a target nucleic acid by an effector protein complexed with a guide nucleic acid (e.g., an RNP complex), wherein at least a portion of the guide nucleic acid is hybridized to at least a portion of the target nucleic acid. Cleavage may occur within or directly adjacent to the region of the target nucleic acid that is hybridized to the guide nucleic acid.

The terms “complementary” and “complementarity,” as used herein, with reference to a nucleic acid molecule or nucleotide sequence, refer to the characteristic of a polynucleotide having nucleotides that base pair with their Watson-Crick counterparts (C with G; or A with T or U) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its-3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term “cleavage assay,” as used herein, refers to an assay designed to visualize, quantitate, or identify cleavage of a nucleic acid. In some cases, the cleavage activity may be cis-cleavage activity. In some cases, the cleavage activity may be trans-cleavage activity.

The terms “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The terms “CRISPR RNA” or “crRNA,” as used herein, refer to a type of guide nucleic acid, wherein the nucleic acid is RNA comprising a first sequence that is capable of interacting with an effector protein either directly (by being bound by an effector protein) or indirectly (e.g., by hybridization with a second nucleic acid molecule that can be bound by an effector, such as a tracrRNA); and a second sequence that hybridizes to a target sequence of a target nucleic acid. In some embodiments, the first sequence is referred to as a repeat sequence and the second sequence is referred to as a spacer sequence. The first sequence and the second sequence are directly connected to each other or by a linker.

The term, “disrupt,” as used herein, refers to reducing or abolishing a function of a gene regulatory element by altering or modifying the nucleotide sequence of the gene regulatory element or the nucleotide sequence located in proximity (e.g., less than 200 linked nucleotides) to the gene regulatory element. In some embodiments, the gene regulatory element is a splicing-regulatory element. In some embodiments, the original function of the gene regulatory element is repressing exonic splicing. In some embodiments, there is an increased inclusion of an exon region in a mature mRNA after the disruption.

The term, “donor nucleic acid,” as used herein, refers to a nucleic acid that is (designed or intended to be) incorporated into a target nucleic acid or target sequence.

The term “dual nucleic acid system” as used herein refers to a system that uses a transactivated or transactivating RNA-crRNA duplex complexed with one or more polypeptides described herein, wherein the complex is capable of interacting with a target nucleic acid in a sequence selective manner.

The term “effector protein,” as used herein, refers to a protein, polypeptide, or peptide that is capable of interacting with a guide nucleic acid to form a complex (e.g., a RNP complex), wherein the complex interacts with a target nucleic acid. A complex between an effector protein and a guide nucleic acid can include multiple effector proteins or a single effector protein. In some embodiments, the effector protein modifies the target nucleic acid when the complex contacts the target nucleic acid. In some embodiments, the effector protein does not modify the target nucleic acid, but it is linked to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout. Herein, reference to an effector protein includes reference to a nucleic acid encoding the effector protein, unless indicated otherwise.

The term, “engineered modification,” as used herein, refers to a structural change of one or more nucleic acid residues of a nucleotide sequence or one or more amino acid residue of an amino acid sequence, such as chemical modification of one or more nucleobases; or a chemical change to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition, or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some embodiments, the nucleic acids provided herein are not uniformly modified along the entire length of the molecule. Different nucleotide modifications and/or backbone structures can exist at various positions within the nucleic acid.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression.

The terms “fusion protein,” or “fusion effector protein,” as used herein, refer to a protein comprising at least two heterologous polypeptides. The fusion protein may comprise one or more effector proteins and fusion partners. In some embodiments, an effector protein and fusion partner are not found connected to one another as a native protein or complex that occurs together in nature.

The term “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term, “genetic disease,” as used herein, refers to a disease, disorder, condition, or syndrome associated with or caused by one or more mutations in the DNA of an organism having the genetic disease.

The term “guide nucleic acid,” as used herein, refers to a nucleic acid comprising: a first nucleotide sequence that is capable of being non-covalently bound by an effector protein; and a second nucleotide sequence that hybridizes to a target nucleic acid. When in a complex with one or more polypeptides described herein (e.g., an RNP complex), a guide nucleic acid can impart sequence selectivity to the complex when the complex interacts with a target nucleic acid. The first sequence may be referred to herein as a repeat sequence. The second sequence may be referred to herein as a spacer sequence. The term, “guide nucleic acid,” may be used interchangeably herein with the term “guide RNA” (gRNA) however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “handle sequence,” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid. In general, the handle sequence comprises an intermediary RNA sequence, that is capable of being non-covalently bound by an effector protein. In some embodiments, the handle sequence further comprises a repeat sequence. In such embodiments, the intermediary RNA sequence or a combination of the intermediary RNA and the repeat sequence is capable of being non-covalently bound by an effector protein.

The term “heterologous,” as used herein, means a nucleotide or polypeptide sequence that is not found in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked via an amide bond to the effector protein in nature. In some embodiments, a heterologous protein is not encoded by a species that encodes the effector protein. In some embodiments, the heterologous protein exhibits an activity (e.g., enzymatic activity) when it is linked to the effector protein. In some embodiments, the heterologous protein exhibits increased or reduced activity (e.g., enzymatic activity) when it is linked to the effector protein, relative to when it is not linked to the effector protein. In some embodiments, the heterologous protein exhibits an activity (e.g., enzymatic activity) that it does not exhibit when it is linked to the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The terms, “intermediary RNA,” “intermediary RNA sequence,” and “intermediary sequence” as used herein, in a context of a single nucleic acid system, refers to a nucleotide sequence in a handle sequence, wherein the intermediary RNA sequence is capable of, at least partially, being non-covalently bound to an effector protein to form a complex (e.g., an RNP complex). An intermediary RNA sequence is not a transactivating nucleic acid in systems, methods, and compositions described herein.

The term “linked” when used in reference to biopolymers (e.g., nucleic acids, polypeptides) refers to being covalently connected. In some embodiments, two polymers are linked by at least a covalent bond. In some embodiments, two nucleic acids are linked by at least one nucleotide. In some embodiments, two nucleic acids are linked by at least one amino acid. The terms “fused” and “linked” are used interchangeably herein.

The term “linker,” as used herein, refers to a covalent bond or molecule that links a first polypeptide to a second polypeptide (e.g., by an amide bond, or one or more amino acids) or a first nucleic acid to a second nucleic acid (e.g., by a phosphodiester bond, or one or more nucleotides).

The term “modified target nucleic acid,” as used herein, refers to a target nucleic acid, wherein the target nucleic acid has undergone a modification, for example, after contact with an effector protein. In some cases, the modification is an alteration in the sequence of the target nucleic acid. In some cases, the modified target nucleic acid comprises an insertion, deletion, or replacement of one or more nucleotides compared to the unmodified target nucleic acid.

The terms “non-naturally occurring” and “engineered,” as used herein, are used interchangeably and indicate the involvement of the hand of man. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term “nucleic acid expression vector,” as used herein, refers to a nucleic acid that can be used to express a nucleic acid of interest.

The term “nuclear localization signal (NLS),” as used herein, refers to an entity (e.g., peptide) that facilitates localization of a nucleic acid, protein, or small molecule to the nucleus, when present in a cell that contains a nuclear compartment.

The term “nuclease activity,” as used herein, refers to the catalytic activity that results in nucleic acid cleavage (e.g., ribonuclease activity (ribonucleic acid cleavage), or deoxyribonuclease activity (deoxyribonucleic acid cleavage), etc.).

The terms “partner protein,” “fusion partner,” or “fusion partner protein” as used herein, refer to a protein, polypeptide or peptide that is linked to an effector protein or capable of being proximal to an effector protein. In some embodiments, a fusion partner that is capable of being proximal to an effector protein is a fusion partner that is capable of binding a guide nucleic acid, wherein the effector protein is also capable of binding the guide nucleic acid. In some embodiments, a fusion partner directly interacts with (e.g., binds to/by) an effector protein. In some embodiments, a fusion partner indirectly interacts with an effector protein (e.g., through another protein or moiety).

The term “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington, The Science and Practice of Pharmacy 23^{rd} edition, A. Adejare, ed., Elsevier Publishing Co., 2020).

The terms, “promoter” and “promoter sequence,” as used herein, refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The term “protospacer adjacent motif” and “PAM,” as used herein, refers to a nucleotide sequence found in a target nucleic acid that directs an effector protein to modify the target nucleic acid at a specific location. In some embodiments, a PAM sequence is required for a complex of an effector protein and a guide nucleic acid (e.g., an RNP complex) to hybridize to and edit the target nucleic acid. In some embodiments, the complex does not require a PAM to edit the target nucleic acid.

In some embodiments, the term “region” as used herein may be used to describe a portion of, or all of, a corresponding sequence, for example, a spacer region is understood to comprise a portion of or all of a spacer sequence.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The term, “repeat sequence,” as used herein, refers to a sequence of nucleotides in a guide nucleic acid that is capable of, at least partially, interacting with an effector protein.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The terms, “RuvC” and “RuvC domain,” as used herein, refer to a region of an effector protein that is capable of cleaving a target nucleic acid, and in certain embodiments, of processing a pre-crRNA. In some embodiments, the RuvC domain is located near the C-terminus of the effector protein. A single RuvC domain may comprise RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as InterPro (ebi.ac.uk/interpro/). For example, a RuvC-like domain may be a domain which shares homology with a region of TnpB proteins of the IS605 and other related families of transposons

The term “sample,” as used herein, generally refers to something comprising a target nucleic acid. In some embodiments, the sample is a biological sample, such as a biological fluid or tissue sample. In some embodiments, the sample is an environmental sample. The sample may be a biological sample or environmental sample that is modified or manipulated. By way of non-limiting example, samples may be modified or manipulated with purification techniques, heat, nucleic acid amplification, salts and buffers.

The terms, “single guide nucleic acid”, “single guide RNA” and “sgRNA,” as used herein, in the context of a single nucleic acid system, refers to a guide nucleic acid, wherein the guide nucleic acid is a single polynucleotide chain having all the required sequence for a functional complex with an effector protein (e.g., being bound by an effector protein, including in some embodiments activating the effector protein, and hybridizing to a target nucleic acid, without the need for a second nucleic acid molecule). For example, an sgRNA can have two or more linked guide nucleic acid components (e.g., an intermediary RNA sequence, a repeat sequence, a spacer sequence and optionally a linker). In some embodiments, a sgRNA comprises a handle sequence, wherein the handle sequence comprises an intermediary sequence, a repeat sequence, and optionally a linker sequence.

The term, “single guide nucleic acid system,” as used herein, refers to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein.

The term, “spacer sequence,” as used herein, refers to a nucleotide sequence in a guide nucleic acid that is capable of, at least partially, hybridizing to an equal length portion of a sequence (e.g., a target sequence) of a target nucleic acid.

The term “subject,” as used herein, refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a non-human primate. The mammal can be a cynomolgus monkey. The mammal can be a mouse, rat, or other rodent. The mammal can be a human. The subject may be diagnosed or suspected of being at high risk for a disease. In some embodiments, the subject is not necessarily diagnosed or suspected of being at high risk for the disease.

The term “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The terms “target nucleic acid sequence” and “target sequence,” as used herein, when used in reference to a target nucleic acid, refers to a sequence of nucleotides found within a target nucleic acid. Such a sequence of nucleotides can, for example, hybridize to an equal length portion of a guide nucleic acid. Hybridization of the guide nucleic acid to the target sequence may bring an effector protein into contact with the target nucleic acid.

The term, “trans cleavage,” as used herein, in the context of cleavage (e.g., hydrolysis of a phosphodiester bond) of one or more target nucleic acids or non-target nucleic acids, or both, by an effector protein that is complexed with a guide nucleic acid and the target nucleic acid. Trans cleavage activity may be triggered by the hybridization of a guide nucleic acid to a target nucleic acid. The effector may cleave a target strand as well as non-target strand, wherein the target nucleic is a double stranded nucleic acid. Trans cleavage of the target nucleic acid may occur away from (e.g., not within or directly adjacent to) the portion of the target nucleic acid that is hybridized to the portion of the guide nucleic acid.

The terms, “trans-activating RNA”, “transactivating RNA” and “tracrRNA,” refer to a transactivating or transactivated nucleic acid in a dual nucleic acid system that is capable of hybridizing, at least partially, to a crRNA to form a tracrRNA-crRNA duplex, and of interacting with an effector protein to form a complex (e.g., an RNP complex).

The terms, “transactivating”, “trans-activating”, “trans-activated”, “transactivated” and grammatical equivalents thereof, as used herein, in the context of a dual nucleic acid system refers to an outcome of the system, wherein a polypeptide is enabled to have a binding and/or nuclease activity on a target nucleic acid, by a tracrRNA or a tracrRNA-crRNA duplex.

The term, “transcriptional activator,” as used herein, refers to a polypeptide or a fragment thereof that can activate or increase transcription of a target nucleic acid molecule.

The term “transcriptional repressor,” as used herein, refers to a polypeptide or a fragment thereof that is capable of arresting, preventing, or reducing transcription of a target nucleic acid.

The term, “transgene,” as used herein, refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell.

The terms “treatment” and “treating,” as used herein, are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “viral vector,” as used herein, refers to a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

### II. Introduction

In some embodiments, the present disclosure provides compositions and methods for modification of the double homeobox 4 (DUX4) gene. Modifications include epigenetic modifications.

In some embodiments, the present disclosure provides guide nucleic acids that are capable of binding to a target sequence in the DUX4 gene. In some embodiments, the present disclosure provides guide nucleic acids that are capable of binding to a target sequence of the DUX4 gene and an effector protein. In some embodiments, the effector protein is a CRISPR-associated (Cas) protein. In general, Cas proteins bind and/or modify nucleic acids in a sequence-specific manner. Cas proteins with guide nucleic acids my modify DNA at a precise target location in the genome of a wide variety of cells and organisms, allowing for precise and efficient editing of DNA sequences of interest (e.g., DUX4). In some embodiments, the present disclosure provides methods for treating a genetic disease (e.g., FSHD) by modifying one or more target genes (e.g., DUX4).

Disclosed herein are non-naturally occurring compositions and systems comprising an effector protein and/or a guide nucleic acid. In general, an effector protein and a guide nucleic acid refer to an effector protein and a guide nucleic acid, respectively, that are not found in nature. In some embodiments, systems and compositions herein comprise at least one non-naturally occurring component. For example, compositions and systems may comprise a guide nucleic acid, wherein the sequence of the guide nucleic acid is different or modified from that of a naturally-occurring guide nucleic acid. In some embodiments, compositions and systems comprise at least two components that do not naturally occur together. For example, compositions and systems may comprise a guide nucleic acid comprising a repeat sequence and a spacer sequence which do not naturally occur together. Also, by way of example, composition and systems may comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

### III. Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid.

In general guide nucleic acids comprises a nucleotide sequence. Such a nucleotide sequence may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid. Similarly, disclosure of the nucleotide sequences described herein also discloses a complementary nucleotide sequence, a reverse nucleotide sequence, and the reverse complement nucleotide sequence, any one of which can be a nucleotide sequence for use in a guide nucleic acid. In some embodiments, a guide nucleic acid sequence(s) comprises one or more nucleotide alterations at one or more positions in any one of the sequences described herein. Alternative nucleotides can be any one or more of A, C, G, T or U, or a deletion, or an insertion.

A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring guide nucleic acid. A guide nucleic acid of the present disclosure comprises one or more of the following: a) a single guide nucleic acid molecule; b) a DNA base; c) an RNA base; d) a modified base; e) a modified sugar; f) a modified backbone; and the like. Modifications are described herein and throughout the present disclosure. A guide nucleic acid may be chemically synthesized or recombinantly produced by any suitable methods. Guide nucleic acids and portions thereof may be found in or identified from a CRISPR array present in the genome of a host organism or cell.

In some embodiments, the guide nucleic acid comprises a non-natural nucleobase sequence. In some embodiments, the non-natural sequence is a nucleobase sequence that is not found in nature. The non-natural sequence may comprise a portion of a naturally-occurring sequence, wherein the portion of the naturally-occurring sequence is not present in nature absent the remainder of the naturally-occurring sequence. In some embodiments, the nucleotide sequence of the guide nucleic acid is not found in nature. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. Engineered guide nucleic acids may comprise a first sequence and a second sequence that do not occur naturally together. For example, a guide nucleic acid may comprise a sequence of a naturally-occurring repeat region and a spacer region that is complementary to a naturally-occurring eukaryotic sequence. The guide nucleic acid may comprise a sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, a guide nucleic acid is a crRNA, wherein the crRNA comprises a repeat sequence and a spacer sequence that is complementary to a eukaryotic target sequence. In some embodiments a guide nucleic acid may comprise a repeat sequence, an intermediary sequence, and a spacer sequence coupled by one or more linker sequences. In some embodiments, the guide nucleic acid comprises two heterologous sequences arranged in an order or proximity that is not observed in nature. Therefore, guide nucleic acid compositions described herein are not naturally occurring.

In general, a guide nucleic acid comprises a first nucleotide sequence that is capable of being non-covalently bound by an effector protein and a second nucleotide sequence that hybridizes to a target nucleic acid. In some embodiments, the first nucleotide sequence is located 5′ to second nucleotide sequence. In some embodiments, the second nucleotide sequence is located 5′ to first nucleotide sequence. In some embodiments, the first nucleotide sequence comprises a repeat sequence. In some embodiments, the first nucleotide sequence comprises an intermediary sequence. In some embodiments, an effector protein binds to at least a portion of the first nucleotide sequence. In some embodiments, the second nucleotide sequence comprises a spacer sequence, wherein the spacer sequence can interact in a sequence-specific manner with (e.g., has complementarity with, or can hybridize to a target sequence in) a target nucleic acid (e.g., the DUX4 gene). Although the term may imply that a gRNA consists of RNA, in some embodiments a gRNA may comprise one or more deoxyribonucleotides and/or a deoxyribonucleotide nucleobase (e.g., thymine). However, the majority of the nucleotides in a guide nucleic acid (at least 50%) are ribonucleotides.

In some embodiments, uridines can be exchanged for pseudouridines (e.g., 1N-Methyl-Pseudouridine). In some embodiments, all uridines can be exchanged for 1N-Methyl-Pseudouridine. In this application, U can represent uracil or 1N-Methyl-Pseudouridine. Modifications can further include changing of nucleic acids described herein (e.g., engineered guide nucleic acids) to provide the nucleic acid with a new or enhanced feature, such as improved stability. Such modifications of a nucleic acid include a nucleobase base modification, a backbone modification, a sugar modification, or combinations thereof. In some embodiments, the modifications can be of one or more nucleotides, nucleosides, or nucleobases in a nucleic acid. In some embodiments, uridines can be exchanged for pseudouridines (e.g., 1N-Methyl-Pseudouridine). In some embodiments, all uridines can be exchanged for 1N-Methyl-Pseudouridine. In this application, U can represent uracil or 1N-Methyl-Pseudouridine.

The guide nucleic acid may also form complexes as described through herein. For example, a guide nucleic acid may hybridize to another nucleic acid, such as target nucleic acid, or a portion thereof. In another example, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid (e.g., a target sequence in the DUX4 gene). For example, when a guide nucleic acid and an effector protein are complexed to form an RNP, at least a portion of the guide nucleic acid hybridizes to a target sequence in a target nucleic acid (e.g., the DUX4 gene). Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different loci in the target nucleic acid by hybridizing to different target sequences. In some embodiments, a first guide nucleic acid may hybridize within a location of the target nucleic acid that is different from where a second guide nucleic acid may hybridize the target nucleic acid. In some embodiments, the first loci and the second loci of the target nucleic acid may be located at least 1, at least 10, at least 20, at least 30, at least 40, at least 50, at least 60, at least 70, at least 80, at least 90, or at least 100 nucleotides apart. In some embodiments, the first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart.

In some embodiments, the first loci and/or the second loci of the target nucleic acid are located in an intron of a gene (e.g., an intron of the DUX4 gene). In some embodiments, the first loci and/or the second loci of the target nucleic acid are located in an exon of a gene (e.g., an exon of the DUX4 gene). In some embodiments, the first portion and/or the second portion of the target nucleic acid are located on either side of an exon and cutting at both sites results in deletion of the exon. In some embodiments, composition, systems, and methods comprise a donor nucleic acid that may be inserted in replacement of a deleted or cleaved sequence of the target nucleic acid. In some embodiments, compositions, systems, and methods comprising multiple guide nucleic acids or uses thereof comprise multiple effector proteins, wherein the effector proteins may be identical, non-identical, or combinations thereof.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides. In some embodiments, a guide nucleic acid comprises at least 25 linked nucleotides.

A guide nucleic acid may comprise 10 to 50 linked nucleotides. In some embodiments, the guide nucleic acid comprises or consists essentially of about 12 to about 80 linked nucleotides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleotides. In some embodiments, the guide nucleic acid comprises about 10 to about 60, about 20 to about 50, or about 30 to about 40 linked nucleotides.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

Guide nucleic acids may comprise deoxyribonucleotides, ribonucleotides or a combination thereof. In some embodiments, a guide nucleic acid comprises a ribonucleotide with a thymine nucleobase. Guide nucleic acids may comprise a chemically modified nucleobase or phosphate backbone. Guide nucleic acids may be referred to herein as a guide RNA (gRNA). However, a guide RNA is not limited to ribonucleotides, but may comprise deoxyribonucleotides and other chemically modified nucleotides. A guide nucleic acid may comprise a non-naturally occurring guide nucleic acid, including a guide nucleic acid that is designed to contain a chemical or biochemical modification.

In some embodiments, effector proteins are targeted by a guide nucleic acid (e.g., a guide RNA) to a specific location in the target nucleic acid where they exert locus-specific nucleotide modification or gene regulation. Non-limiting examples of gene regulation include blocking RNA polymerase binding to a promoter (which selectively inhibits transcription activator function), and/or modifying local chromatin (e.g., modifying the target nucleic acid or modifying a protein associated with the target nucleic acid). The guide RNA may bind to a target nucleic acid (e.g., a single strand of a target nucleic acid) or a portion thereof, an amplicon thereof, or a portion thereof. By way of non-limiting example, a guide nucleic acid may bind to a portion of a gene associated with a genetic disorder, or an amplicon thereof, as described herein.

In some embodiments, the compositions, systems, and methods of the present disclosure may comprise an additional guide nucleic acid or a use thereof. An additional guide nucleic acid can target an effector protein to a different location in the target nucleic acid by binding to a different portion of the target nucleic acid from the first guide nucleic acid. A system in which two different guide nucleic acids are used to target two different locations in the target nucleic acid may be referred to as a dual guided system. In certain embodiments, upon removal of a sequence between two guide nucleic acids, the wild-type reading frame may be restored, e.g., by a polymerase, resulting in at least a partially functional protein.

### Single Guide Nucleic Acid Systems

In some embodiments, compositions, systems and methods described herein comprise a single guide nucleic acid. In the single guide nucleic acid system, the effector protein is not transactivated by a guide nucleic acid. By way of non-limiting example, a single guide nucleic acid system does not require a tracrRNA. In other words, activity of the effector protein does not require binding to a second or intermediary guide nucleic acid molecule. An exemplary guide nucleic acid for a single guide nucleic acid system is a crRNA or a sgRNA.

crRNA

In some embodiments, the single guide nucleic acid comprises a crRNA. In general, a crRNA comprises a first region (FR) and a second region (SR), wherein the FR of the crRNA comprises a repeat sequence, and the SR of the crRNA comprises a spacer sequence. In some embodiments, the spacer sequence follows the repeat sequence in a 5′ to 3′ direction. In some embodiments, the spacer sequence precedes the repeat sequence in a 5′ to 3′ direction. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single guide nucleic acid system for compositions, methods, and systems described herein or as part of a single guide nucleic acid system for compositions, methods, and systems described herein. In such embodiments, a single guide nucleic acid system comprises a guide nucleic acid comprising a crRNA wherein, a repeat sequence of a crRNA is capable of causing a crRNA to interact with an effector protein. In some embodiments, a single guide nucleic acid system comprises a guide nucleic acid comprising a crRNA linked to another nucleotide sequence that is capable of being non-covalently bound by an effector protein. In some embodiments, a crRNA is sufficient to form complex with an effector protein (e.g., to form an RNP) through the repeat sequence and direct the effector protein to a target nucleic acid sequence through the spacer sequence.

In some embodiments, compositions and systems described herein comprise an effector protein or a nucleic acid encoding the effector protein, wherein the effector protein comprises an amino acid sequence that is at least 75%, at least 80%, at least 85%, at least 90%, at least 95%, at least 97%, at least 98%, at least 99% or 100% identical to SEQ ID NOs: 230-233, 243-244, 262-274, and 449-451; and a guide nucleic acid that consists essentially of a crRNA. In some embodiments, the crRNA comprises a sequence that is at least 70%, at least 75%, at least 80%, at least 85%, at least 90%, at least 95%, or 100% identical to a sequence selected from SEQ ID NOs: 116-229, 461, and SEQ ID NO: 602-717. In some embodiments, the crRNA consists of a sequence that is at least 70%, at least 75%, at least 80%, at least 85%, at least 90%, at least 95%, or 100% identical to a sequence selected from SEQ ID NOS: 116-229, 461, and SEQ ID NO: 602-717.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

sgRNA

In some embodiments, a guide nucleic acid comprises a single guide RNA (sgRNA). In some embodiments, an sgRNA can have two or more linked guide nucleic acid components (e.g., an intermediary RNA sequence, a repeat sequence, a spacer sequence and optionally a linker). In some embodiments, a sgRNA comprises a handle sequence, wherein the handle sequence comprises an intermediary sequence, a repeat sequence, and optionally a linker sequence. In some embodiments, the guide nucleic acid is a sgRNA. The combination of a spacer sequence (e.g., a nucleotide sequence that hybridizes to a target sequence in a target nucleic acid) with a handle sequence may be referred to herein as a single guide RNA (sgRNA), wherein the spacer sequence and the handle sequence are covalently linked. In some embodiments, the spacer sequence and handle sequence are linked by a phosphodiester bond. In some embodiments, the spacer sequence and handle sequence are linked by one or more linked nucleotides. In some embodiments, a guide nucleic acid may comprise a spacer sequence, a repeat sequence, or handle sequence, or a combination thereof. In some embodiments, the handle sequence may comprise a portion of, or all of, a repeat sequence. In general, a sgRNA comprises a first region (FR) and a second region (SR), wherein the FR comprises a handle sequence and the SR comprises a spacer sequence.

In some embodiments, the compositions comprising a guide RNA and an effector protein without a traceRNA (e.g., a single nucleic acid system), wherein the guide RNA is a sgRNA. A sgRNA may include deoxyribonucleosides, ribonucleosides, chemically modified nucleosides, or any combination thereof. A sgRNA may also include a nucleotide sequence that forms a secondary structure (e.g., one or more hairpin loops) that facilitates the binding of an effector protein to the sgRNA and/or modification activity of an effector protein on a target nucleic acid (e.g., a hairpin region). Such a sequence can be contained within a handle sequence as described herein.

In some embodiments, a sgRNA comprises one or more of one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence, and a spacer sequence; and the like.

In some embodiments, sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g., covalently linked, such as through a phosphodiester bond) In some embodiments, a repeat sequence and a spacer sequence are linked in an sgRNA by any suitable linker, examples of which are provided herein.

An exemplary handle sequence in a sgRNA may comprise, from 5′ to 3′, a 5′ region, a hairpin region, and a 3′ region. In some embodiments, the 5′ region may hybridize to the 3′ region. In some embodiments, the 5′ region does not hybridize to the 3′ region. In some embodiments, the 3′ region is covalently linked to a spacer sequence (e.g., through a phosphodiester bond). In some embodiments, the 5′ region is covalently linked to a spacer sequence (e.g., through a phosphodiester bond).

In some embodiments, compositions and systems described herein comprise an effector protein or a nucleic acid encoding the effector protein, wherein the effector protein comprises an amino acid sequence that is at least 75%, at least 80%, at least 85%, at least 90%, at least 95%, at least 97%, at least 98%, at least 99% or 100% identical to any one of SEQ ID NOs: 428-446 or 452; and a guide nucleic acid that comprises a sgRNA. In some embodiments, the sgRNA comprises a sequence that is at least 70%, at least 75%, at least 80%, at least 85%, at least 90%, at least 95%, or 100% identical to a sequence selected from SEQ ID NOs: 353-427, 462-465, and 597-601. In some embodiments, the sgRNA consists of a sequence that is at least 70%, at least 75%, at least 80%, at least 85%, at least 90%, at least 95%, or 100% identical to a sequence selected from SEQ ID NOs: 353-427 and 597-601.

### Dual Nucleic Acid Systems

In some embodiments, compositions, systems and methods described herein comprise a dual nucleic acid system comprising a crRNA or a nucleotide sequence encoding the crRNA, a tracrRNA, or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, in a dual nucleic acid system, activity of the effector protein requires binding to a tracrRNA molecule.

In some embodiments, a repeat hybridization sequence is at the 3′ end of a tracrRNA sequence. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid. In some embodiments, the secondary structure comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the secondary structure comprises a pseudoknot (e.g., a secondary structure comprising a stem at least partially hybridized to a second stem or half-stem secondary structure). An effector protein may recognize a secondary structure comprising multiple stem regions. In some embodiments, nucleotide sequences of the multiple stem regions are identical to one another. In some embodiments, the nucleotide sequences of at least one of the multiple stem regions is not identical to those of the others. In some embodiments, the secondary structure comprises at least two, at least three, at least four, or at least five stem regions. In some embodiments, the secondary structure comprises one or more loops. In some embodiments, the secondary structure comprises at least one, at least two, at least three, at least four, or at least five loops.

### Spacer Sequences

Guide nucleic acids described herein may comprise one or more spacer sequences. In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

The spacer sequence of a guide nucleic acid is complementary to a target sequence of a target nucleic acid. The spacer sequence of a guide nucleic acid may be at least 70%, at least 75%, at least 80%, at least 85%, at least 90%, at least 95%, or 100% complementary to a target sequence of a target nucleic acid. In general, the spacer sequence is capable of hybridizing to a target sequence of a target nucleic acid. It is understood that the spacer sequence need not be 100% complementary to that of a target sequence of a target nucleic acid to hybridize or hybridize specifically to the target sequence.

In some embodiments, the spacer region is 5-50 linked nucleotides in length. In some embodiments, the spacer region is 15-28 linked nucleotides in length. In some embodiments, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer region is 18-24 linked nucleotides in length. In some embodiments, the spacer region is at least 15 linked nucleotides in length. In some embodiments, the spacer region is at least 16, 18, 20, or 22 linked nucleotides in length. In some embodiments, the spacer region comprises at least 10, 11, 12, 13, 14, 15, 16, 17, 18, 19, 20, 21, 22, 23, 24, 25, 26, 27, 28, 29, or 30 nucleotides. In some embodiments, the spacer region is at least 17 linked nucleotides in length. In some embodiments, the spacer region is at least 18 linked nucleotides in length. In some embodiments, the spacer region is at least 20 linked nucleotides in length. In some embodiments, the spacer region is at least 80%, at least 85%, at least 90%, at least 95% or 100% complementary to a target sequence of the target nucleic acid. In some embodiments, the spacer region is 100% complementary to the target sequence of the target nucleic acid. In some embodiments, the spacer region comprises at least 15 contiguous nucleobases that are complementary to the target nucleic acid.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid (e.g., the DUX4 gene). A spacer sequence is capable of hybridizing to an equal length portion of a target nucleic acid (e.g., a target sequence). In some embodiments, a spacer sequence comprises a sequence that is at least 65%, at least 70%, at least 75%, at least 80%, at least 85%, at least 90%, at least 95% or 100% complementary to a target sequence of a DUX4 gene. In some embodiments, the spacer sequence comprises at least 10, at least 11, at least 12, at least 13, at least 14, at least 15, at least 16, at least 17, at least 18, at least 19, or at least 20 contiguous nucleotides that are capable of hybridizing to the target sequence. In some embodiments, the spacer sequence comprises at least 10, at least 11, at least 12, at least 13, at least 14, at least 15, at least 16, at least 17, at least 18, at least 19, or at least 20 contiguous nucleotides that are complementary to the target sequence.

TABLE 1 provides illustrative spacer sequences for use with the compositions, systems, and methods of the disclosure. In particular, TABLE 1 provides spacer sequences suitable for use in combination with an effector protein of SEQ ID NO: 230 or variants thereof. In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to a sequence as set forth in TABLE 1. In some embodiments, spacer sequences comprise at least 10, at least 11, at least 12, at least 13, at least 14, at least 15, at least 16, at least 17, at least 18, at least 19, or at least 20, contiguous nucleotides of a sequence selected from any one of SEQ ID NOS: 1-114, 456, and 481-596. In some embodiments, guide nucleic acids comprising a spacer sequence in TABLE 1 are used with an effector protein that is at least 90%, at least 95%, at least 98%, at least 99% or 100% identical to SEQ ID NO: 230.

TABLE 1
Exemplary Spacer Sequences for CasPhi.12 Effector
Proteins

| | Spacer sequence | SEQ |
|---|---|---|
| Target Region | (5′ t′ 3′), | ID |
| of DUX4 | shown as RNA | NO: |
| Exon #1 | AGCGGAACCCGUACCCGGGC | 1 |
| Exon #1 | AGAAGGAUCGCUUUCCAGGC | 2 |
| Exon #1 | GCCUACGCCGCCCCGGCUCC | 3 |
| Exon #1 | AGAUCUGGUUUCAGAAUCGA | 4 |
| Exon #1 | CGCCUACGCCGCCCCGGCUC | 5 |
| Exon #1 | CGUGAGCCAGGCAGCGAGGG | 6 |
| Exon #1 | CAGGCAUCGCCGCCCGGGAG | 7 |
| Exon #1 | GACCCCGAGCCAAAGCGAGG | 8 |
| Exon #1 | CAGAAUGAGAGGUCACGCCA | 9 |
| Exon #1 | GUGAGCCAGGCAGCGAGGGC | 10 |
| Exon #1 | CCAGGCAUCGCCGCCCGGGA | 11 |
| Exon #1 | GAGCGGAACCCGUACCCGGG | 12 |
| Exon #1 | GGACCCCGAGCCAAAGCGAG | 13 |
| Exon #1 | GGUUUCAGAAUGAGAGGUCA | 14 |
| Exon #1 | UGCAGCAGGCGCAACCUCUC | 15 |
| Exon #1 | GUUUCAGAAUGAGAGGUCAC | 16 |
| Exon #1 | AGAAUGAGAGGUCACGCCAG | 17 |
| Exon #1 | CCGCACCCCACGUGCCCUGC | 18 |
| Exon #1 | CGGAGCCCAGGGUCCAGAUU | 19 |
| Exon #1 | AGAAUCGAAGGGCCAGGCAC | 20 |
| Exon #1 | GCCCACACCGGCGCGUGGGG | 21 |
| Exon #1 | CAGAAUCGAAGGGCCAGGCA | 22 |
| Exon #1 | CUGCAGCAGGCGCAACCUCU | 23 |
| Exon #1 | CGCCACCCACGUCCCAGGGG | 24 |
| Exon #1 | GAGAAGGAUCGCUUUCCAGG | 25 |
| Exon #1 | UGAAACCAGAUCUGAAUCCU | 26 |
| Exon #1 | GCCCGGGUGCGGAGGCCACC | 27 |
| Exon #1 | UCAAAGGCUCGGAGGAGCAG | 28 |
| Exon #1 | CGCGGGGAGGGUGCUGUCCG | 29 |
| Exon #1 | CCGCCGGUGCUGCCUCAGCU | 30 |
| Exon #1 | CCCGGGUGCGGAGGCCACCG | 31 |
| Exon #1 | CAUCUGCCCCUGCCGCGCGG | 32 |
| Exon #1 | CGCCUGCUGCAGAAACUCCG | 33 |
| Exon #1 | CCACGCCGCCCCGGCGACCU | 34 |
| Exon #1 | GCCCUGCGGCCCCGCUUGAG | 35 |
| Exon #1 | CUAGGAGAGGUUGCGCCUGC | 36 |
| Exon #1 | AGCGGGCCCAGGCUGUGCCA | 37 |
| Exon #1 | CUCGCUGAGGGGUGCUUCCA | 38 |
| Exon #1 | CGCUCAAAGCAGGCUCGCAG | 39 |
| Exon #1 | GGCUCGGGGUCCAAACGAGU | 40 |
| Exon #1 | GAUUCUGAAACCAGAUCUGA | 41 |
| Exon #1 | UGCCCGGGUGCGGAGGCCAC | 42 |
| Exon #1 | UGAAACCAAAUCUGGACCCU | 43 |
| Exon #1 | CGAGGCCUCCAGCUCCCCCG | 44 |
| Exon #1 | UCUGGUGGCGAUGCCCGGGU | 45 |
| Exon #1 | GGAGAUCCCCUCUGCCGGCG | 46 |
| Exon #1 | UAGGAGAGGUUGCGCCUGCU | 47 |
| Exon #1 | GGGUUCCCACGCCGCCCCGG | 48 |
| Exon #1 | CGCCGGCCUUCUGGCGGGCC | 49 |
| Exon #1 | CCCACGCGCCGGUGUGGGCG | 50 |
| Exon #1 | CAGCGAGGCGGCCUCUUCCG | 51 |
| Exon #1 | UUCCUCGCUGAGGGGUGCUU | 52 |
| Exon #1 | GCUCGGGGUCCAAACGAGUC | 53 |
| Exon #1 | UGGCGGGCCGCGUCUCCCGG | 54 |
| Exon #1 | GCCGGCCUUCUGGCGGGCCG | 55 |
| Exon #2 | CGACGCUGUCUAGGCAAACC | 56 |
| Exon #2 | AGAGAUAUAUUAAAAUGCCC | 57 |
| Exon #2 | CGUGAAAUUCUGGCUGAAUG | 58 |
| Exon #2 | UUCCGUGAAAUUCUGGCUGA | 59 |
| Exon #2 | GAGUUACAUCUCCUGGAUGA | 60 |
| Exon #2 | UUCUUCCGUGAAAUUCUGGC | 61 |
| Exon #2 | UGGCUGAAUGUCUCCCCCCA | 62 |
| Exon #2 | CAUCUCCUGGAUGAUUAGUU | 63 |
| Exon #2 | AAAUGCCCCCUCCCUGUGGA | 64 |
| Exon #2 | CACGUCAGCCGGGGUGCGCA | 65 |
| Exon #2 | AUAUAUCUCUGAACUAAUCA | 66 |
| Exon #2 | ACGGAAGAACAAGGGCACAG | 67 |
| Exon #2 | CCUAGACAGCGUCGGAAGGU | 68 |
| Exon #2 | CACGGAAGAACAAGGGCACA | 69 |
| Exon #2 | GCCUAGACAGCGUCGGAAGG | 70 |
| Exon #2 | AAUAUAUCUCUGAACUAAUC | 71 |
| Exon #2 | AGCCAGAAUUUCACGGAAGA | 72 |
| Exon #2 | CCCGCUUCCUGGCUAGACCU | 73 |
| Exon #2 | CUGGCUAGACCUGCGCGCAG | 74 |
| Exon #2 | UAUAGGAUCCACAGGGAGGG | 75 |
| Intron #2 | CGGGCAGCCGCCUGGGCUGU | 76 |
| Intron #2 | GCGGGCAGCCGCCUGGGCUG | 77 |
| Intron #2 | CGGGGGUGGGGGGUGGGGGU | 78 |
| Intron #2 | GCGGGACGGGGGUCUCCACC | 79 |
| Intron #2 | GACCGCCAGGCGCUCCGUGC | 80 |
| Intron #2 | UCCGGGGGUGGGGGGUGGGG | 81 |
| Intron #2 | CCGGGGGUGGGGGGUGGGGG | 82 |
| Intron #2 | CGCGGGACGGGGGUCUCCAC | 83 |
| Intron #2 | UGACCGCCAGGCGCUCCGUG | 84 |
| Intron #2 | ACCGCCAGGCGCUCCGUGCU | 85 |
| Intron #2-Exon #3 | GCCCGCUUCCUGGCUAGACC | 86 |
| Downstream 3′ UTR | GGUGAUCAGUGCAGAUGUGU | 87 |
| Downstream 3′ UTR | CAGAACUCCAUAGUAGACUG | 88 |
| Downstream 3′ UTR | UGUGUGAUGAGUGCAGAGAU | 89 |
| Downstream 3′ UTR | CAUCUUUUGUGUGAUGAGUG | 90 |
| Downstream 3′ UTR | UGUGAUGAGUGCAGAGAUAU | 91 |
| Downstream 3′ UTR | ACAGAACUUCGGUGAUCAGU | 92 |
| Downstream 3′ UTR | GCAUCUUUUGUGUGAUGAGU | 93 |
| Downstream 3′ UTR | CAGAACUUCGGUGAUCAGUG | 94 |
| Downstream 3′ UTR | AGAACUCCAUAGUAGACUGA | 95 |
| Downstream 3′ UTR | GUGUGAUGAGUGCAGAGAUA | 96 |
| Downstream 3′ UTR | CAUCACUUAGGUGAUCAGUG | 97 |
| Downstream 3′ UTR | GGUGAUCAGUGUAGAGAUAU | 98 |
| Downstream 3′ UTR | AAAUUCUCGUGUAGACAGAG | 99 |
| Downstream 3′ UTR | AUUUACAGAACUUCGGUGAU | 100 |
| Downstream 3′ UTR | UGAAACACAUCUGCACUGAU | 101 |
| Downstream 3′ UTR | UUCUACAGGGGAUAUUGUGA | 102 |
| Downstream 3′ UTR | AGUCUACUAUGGAGUUCUGA | 103 |
| Downstream 3′ UTR | CAGGCUUUUUCUACAGGGGA | 104 |
| Downstream 3′ UTR | UCUACAGGGGAUAUUGUGAC | 105 |
| Downstream 3′ UTR | CUACAGGGGAUAUUGUGACA | 106 |
| Downstream 3′ UTR | UAACAUAUCUCUACACUGAU | 107 |
| Downstream 3′ UTR | ACAUAUCUCUACACUGAUCA | 108 |
| Downstream 3′ UTR | UGACAUAUCUCUGCACUCAU | 109 |
| Downstream 3′ UTR | AACAUAUCUCUACACUGAUC | 110 |
| Downstream 3′ UTR | UGUAAAUCAAUUUCAGGCUU | 111 |
| Downstream 3′ UTR | UACAGGGGAUAUUGUGACAU | 112 |
| Downstream 3′ UTR | UCUAGGUUCAGUCUACUAUG | 113 |
| Downstream 3′ UTR | AGGCUUUUUCUACAGGGGAU | 114 |
| | AGAGAUAUAUCAAAAUGCCC | 456 |
| Exon #3 | AGAGAUAUAUUAAAAUGCCC | 481 |
| Exon #3 | AAAUGCCCCCUCCCUGUGGA | 482 |
| Exon #3 | AUAUAUCUCUGAACUAAUCA | 483 |
| Exon #3 | AAUAUAUCUCUGAACUAAUC | 484 |
| Exon #3 | AAAUGCCCCCUCCCUGU | 485 |
| | CUCUUCGUCUCUCCGGC | 486 |
| | CAAGGGCGGCUGGCUGG | 487 |
| | CGGGGUGGGGCGGGCUG | 488 |
| | GUCUCUCCGGCCCCACC | 489 |
| | CACACUCCCCUCCACCC | 490 |
| | CCGUUCCCGCGGGAUCC | 491 |
| | AGUUCCACACUCCCCUC | 492 |
| | ACGGAGAGAGGGCCUGG | 493 |
| | UCCCUGCUGCCGACGCG | 494 |
| | CCGCGGGAUCCCUGGAG | 495 |
| | AUGAAGGGGUGGAGCCU | 496 |
| | ACAAGGGCGGCUGGCUG | 497 |
| | CCUCCUUCACGGAGAGA | 498 |
| | CGGCCGGGGCUCACCGC | 499 |
| | CUCCCUGCUGCCGACGC | 500 |
| | CGGGGGCCGGCUCUCCG | 501 |
| | GGGGGCCGGCUCUCCGG | 502 |
| | AGUGUGCCAGGCCCUCU | 503 |
| | AUGAAUGGCGGUGAGCC | 504 |
| | CACGGACGGACGCGGGC | 505 |
| | ACGGACGGACGCGGGCA | 506 |
| | UAAAGGCCCACAGGCAG | 507 |
| Exon #1 | GAGCGGAACCCGUACCCGGG | 508 |
| Exon #1 | AGAAUCGAAGGGCCAGGCAC | 509 |
| Exon #1 | CCGCACCCCACGUGCCCUGC | 510 |
| Exon #1 | GCCCACACCGGCGCGUGGGG | 511 |
| Exon #1 | GAGAAGGAUCGCUUUCCAGG | 512 |
| Exon #1 | AGAAUGAGAGGUCACGCCAG | 513 |
| Exon #1 | AGAAGGAUCGCUUUCCAGGC | 514 |
| Exon #1 | GGUUUCAGAAUGAGAGGUCA | 515 |
| Exon #1 | AGCGGAACCCGUACCCGGGC | 516 |
| Exon #1 | CCAGGCAUCGCCGCCCGGGA | 517 |
| Exon #1 | CGGAGCCCAGGGUCCAGAUU | 518 |
| Exon #1 | AGAUCUGGUUUCAGAAUCGA | 519 |
| Exon #1 | GUUUCAGAAUGAGAGGUCAC | 520 |
| Exon #1 | CAGGCAUCGCCGCCCGGGAG | 521 |
| Exon #1 | CAGAAUCGAAGGGCCAGGCA | 522 |
| Exon #1 | CAGAAUGAGAGGUCACGCCA | 523 |
| Exon #1 | GUGAGCCAGGCAGCGAGGGC | 524 |
| Exon #1 | GGACCCCGAGCCAAAGCGAG | 525 |
| Exon #1 | GACCCCGAGCCAAAGCGAGG | 526 |
| Exon #1 | CGUGAGCCAGGCAGCGAGGG | 527 |
| Exon #1 | CGCGGGGAGGGUGCUGUCCG | 528 |
| Exon #1 | GGCUCGGGGUCCAAACGAGU | 529 |
| Exon #1 | CCGCCGGUGCUGCCUCAGCU | 530 |
| Exon #1 | CGCCGGCCUUCUGGCGGGCC | 531 |
| Exon #1 | UGAAACCAGAUCUGAAUCCU | 532 |
| Exon #1 | CGCUCAAAGCAGGCUCGCAG | 533 |
| Exon #1 | UGGCGGGCCGCGUCUCCCGG | 534 |
| Exon #1 | GCCGGCCUUCUGGCGGGCCG | 535 |
| Exon #1 | GCUCGGGGUCCAAACGAGUC | 536 |
| Exon #1 | UCAAAGGCUCGGAGGAGCAG | 537 |
| Exon #1 | CCCACGCGCCGGUGUGGGCG | 538 |
| Exon #1 | UCUGGUGGCGAUGCCCGGGU | 539 |
| Exon #1 | UGAAACCAAAUCUGGACCCU | 540 |
| Exon #1 | GAUUCUGAAACCAGAUCUGA | 541 |
| Exon #1 | GACCCCGAGCCAAAGCG | 542 |
| Exon #1 | CGCCACCCACGUCCCAG | 543 |
| Exon #1 | GAGAAGGAUCGCUUUCC | 544 |
| Exon #1 | CAGAAUCGAAGGGCCAG | 545 |
| Exon #1 | CCGCACCCCACGUGCCC | 546 |
| Exon #1 | CGGAGCCCAGGGUCCAG | 547 |
| Exon #1 | CCAGGCAUCGCCGCCCG | 548 |
| Exon #1 | CAGAAUGAGAGGUCACG | 549 |
| Exon #1 | AGAAGGAUCGCUUUCCA | 550 |
| Exon #1 | GAGCGGAACCCGUACCC | 551 |
| Exon #1 | GUUUCAGAAUGAGAGGU | 552 |
| Exon #1 | GCCUACGCCGCCCCGGC | 553 |
| Exon #1 | AGAAUGAGAGGUCACGC | 554 |
| Exon #1 | AGCGGAACCCGUACCCG | 555 |
| Exon #1 | CGUGAGCCAGGCAGCGA | 556 |
| Exon #1 | AGAUCUGGUUUCAGAAU | 557 |
| Exon #1 | AGAAUCGAAGGGCCAGG | 558 |
| Exon #1 | CGCCUACGCCGCCCCGG | 559 |
| Exon #1 | CUGCAGCAGGCGCAACC | 560 |
| Exon #1 | UGCAGCAGGCGCAACCU | 561 |
| Exon #1 | GGACCCCGAGCCAAAGC | 562 |
| Exon #1 | CAGGCAUCGCCGCCCGG | 563 |
| Exon #1 | GUGAGCCAGGCAGCGAG | 564 |
| Exon #1 | GCCCACACCGGCGCGUG | 565 |
| Exon #1 | GGUUUCAGAAUGAGAGG | 566 |
| Exon #1 | UUCCUCGCUGAGGGGUG | 567 |
| Exon #1 | CGCCUGCUGCAGAAACU | 568 |
| Exon #1 | UGCCCGGGUGCGGAGGC | 569 |
| Exon #1 | CAGCGAGGCGGCCUCUU | 570 |
| Exon #1 | UGAAACCAAAUCUGGAC | 571 |
| Exon #1 | CGAGGCCUCCAGCUCCC | 572 |
| Exon #1 | UAGGAGAGGUUGCGCCU | 573 |
| Exon #1 | AGCGGGCCCAGGCUGUG | 574 |
| Exon #1 | GGGUUCCCACGCCGCCC | 575 |
| Exon #1 | UGAAACCAGAUCUGAAU | 576 |
| Exon #1 | GCUCGGGGUCCAAACGA | 577 |
| Exon #1 | GGAGAUCCCCUCUGCCG | 578 |
| Exon #1 | CCCGGGUGCGGAGGCCA | 579 |
| Exon #1 | CCGCCGGUGCUGCCUCA | 580 |
| Exon #1 | CCACGCCGCCCCGGCGA | 581 |
| Exon #1 | GCCCUGCGGCCCCGCUU | 582 |
| Exon #1 | CGCUCAAAGCAGGCUCG | 583 |
| Exon #1 | CUAGGAGAGGUUGCGCC | 584 |
| Exon #1 | CUCGCUGAGGGGUGCUU | 585 |
| Exon #1 | CAUCUGCCCCUGCCGCG | 586 |
| Exon #1 | GCCCGGGUGCGGAGGCC | 587 |
| Exon #1 | CGCGGGGAGGGUGCUGU | 588 |
| Exon #1 | CGCCGGCCUUCUGGCGG | 589 |
| Exon #1 | GGCUCGGGGUCCAAACG | 590 |
| Exon #1 | UCAAAGGCUCGGAGGAG | 591 |
| Exon #1 | GCCGGCCUUCUGGCGGG | 592 |
| Exon #1 | GAUUCUGAAACCAGAUC | 593 |
| Exon #1 | CCCACGCGCCGGUGUGG | 594 |
| Exon #1 | UCUGGUGGCGAUGCCCG | 595 |
| Exon #1 | UGGCGGGCCGCGUCUCC | 596 |

TABLE 2 provides illustrative spacer sequences for use with the compositions, systems, and methods of the disclosure. In particular, TABLE 2 provides spacer sequences suitable for use in combination with an effector protein of SEQ ID NO: 428. In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to a sequence as set forth in TABLE 2. In some embodiments, spacer sequences comprise at least 10, at least 11, at least 12, at least 13, at least 14, at least 15, at least 16, at least 17, at least 18, at least 19, or at least 20, contiguous nucleotides of a sequence selected from any one of SEQ ID NOs: 275-349, 457-460, and 476-480. In some embodiments, guide nucleic acids comprising a spacer sequence in TABLE 2 are used with an effector protein that is at least 90%, at least 95%, at least 98%, at least 99% or 100% identical to SEQ ID NO: 428.

TABLE 2
Exemplary Spacer Sequences for CasM.265466
Effector Proteins

| | Spacer sequence | SEQ |
|---|---|---|
| Target Region | (5′ to 3′), | ID |
| of DUX4 | shown as RNA | NO: |
| Exon #1 | GACCCCGAGCCAAAGCGAGG | 275 |
| Exon #1 | AGCGGAACCCGUACCCGGGC | 276 |
| Exon #1 | CUGGAGGAGCUUUAGGACGC | 277 |
| Exon #1 | AGAAGGAUCGCUUUCCAGGC | 278 |
| Exon #1 | CAGCGCGGCCCCCGGCGGGG | 279 |
| Exon #1 | CAGCAGGCGCAACCUCUCCU | 280 |
| Exon #1 | GUUUCAGAAUGAGAGGUCAC | 281 |
| Exon #1 | CACUCCCCUGCGGCCUGCUG | 282 |
| Exon #1 | GUUUCAGAAUCGAAGGGCCA | 283 |
| Exon #1 | GGAUCCGGUGACGGCGGUCC | 284 |
| Exon #1 | CCCCUGCCGCGCGGAGGCGG | 285 |
| Exon #1 | CCGGCGCGGCCUGGCUGGGC | 286 |
| Exon #1 | GGAGAGGUUGCGCCUGCUGC | 287 |
| Exon #1 | GGCGAAGGCGACCCACGAGG | 288 |
| Exon #1 | AAUCCUGGACUCCGGGAGGC | 289 |
| Exon #1 | GCCAGCUCCUCCCGGGCGGC | 290 |
| Exon #1 | AAACCAAAUCUGGACCCUGG | 291 |
| Exon #1 | CCCGGGUGCGGAGGCCACCG | 292 |
| Exon #1 | GCGGGCCGCGUCUCCCGGGC | 293 |
| Exon #1 | GCUCGGGGUCCAAACGAGUC | 294 |
| Exon #1 | AAACCAGAUCUGAAUCCUGG | 295 |
| Exon #1 | GUGGCGAUGCCCGGGUACGG | 296 |
| Exon #1 | GGAGAGCCCCAGGCGCGCAG | 297 |
| Exon #1 | CCACCGCGCAGGGGCCCGGC | 298 |
| Exon #1 | GACCCUGGGCUCCGGAAUGC | 299 |
| Exon #3 | CCCUUGUUCUUCCGUGAAAU | 300 |
| Exon #3 | UUAAAAUGCCCCCUCCCUGU | 301 |
| Exon #3 | GCUGAAUGUCUCCCCCCACC | 302 |
| Exon #3 | UGCCCUUGUUCUUCCGUGAA | 303 |
| Exon #3 | GGCAAACCUGGAUUAGAGUU | 304 |
| Exon #3 | AUAUAUCUCUGAACUAAUCA | 305 |
| Exon #3 | UCUCUGAACUAAUCAUCCAG | 306 |
| Exon #3 | AACUAAUCAUCCAGGAGAUG | 307 |
| Exon #3 | CCUAGACAGCGUCGGAAGGU | 308 |
| Exon #3 | GGAUCCACAGGGAGGGGGCA | 309 |
| Exon #3 | AUCCAGGUUUGCCUAGACAG | 310 |
| Exon #3 | ACUCUAAUCCAGGUUUGCCU | 311 |
| Exon #3 | CCCGCUUCCUGGCUAGACCU | 312 |
| Exon #3 | UAGGAUCCACAGGGAGGGGG | 313 |
| Intron #2 | GGAGCAGCCCGGGCAGAGCU | 314 |
| Intron #2 | UCUGUCUUUGCCCGCUUCCU | 315 |
| Intron #2 | UCUUUGCCCGCUUCCUGGCU | 316 |
| Intron #2 | CGGGCAGCCGCCUGGGCUGU | 317 |
| Intron #2 | CGCCCCCGCGCCACCGUCGC | 318 |
| Intron #2 | CCCGGGCUGCUCCCACAGCC | 319 |
| Intron #2 | CUUUUGACCGCCAGGCGCUC | 320 |
| Intron #2 | ACCGCCAGGCGCUCCGUGCU | 321 |
| Intron #2 | GCCAGGAAGCGGGCAAAGAC | 322 |
| Downstream 3′ UTR | UUUCAGAACUCCAUAGUAGA | 323 |
| Downstream 3′ UTR | AUGAGUGCAGAGAUAUGUCA | 324 |
| Downstream 3′ UTR | UGAUGAGUGCAGAGAUAUGU | 325 |
| Downstream 3′ UTR | UUAAAAUUCUCGUGUAGACA | 326 |
| Downstream 3′ UTR | GAGAUAUGUUAAAAUUCUCG | 327 |
| Downstream 3′ UTR | GAUCCUAUAGAAGAUUUGCA | 328 |
| Downstream 3′ UTR | CAGAACUUCGGUGAUCAGUG | 329 |
| Downstream 3′ UTR | GAAAAAGCCUGAAAUUGAUU | 330 |
| Downstream 3′ UTR | UGUGAUGAGUGCAGAGAUAU | 331 |
| Downstream 3′ UTR | CAUCUUUUGUGUGAUGAGUG | 332 |
| Downstream 3′ UTR | GAAGAUUUGCAUCUUUUGUG | 333 |
| Downstream 3′ UTR | UCACAAUAUCCCCUGUAGAA | 334 |
| Downstream 3′ UTR | CACUGAUCACCGAAGUUCUG | 335 |
| Downstream 3′ UTR | CACUGAUCACCUAAGUGAUG | 336 |
| Downstream 3′ UTR | ACAUAUCUCUACACUGAUCA | 337 |
| Downstream 3′ UTR | ACAUAUCUCUGCACUCAUCA | 338 |
| Downstream 3′ UTR | CACUCAUCACACAAAAGAUG | 339 |
| Downstream 3′ UTR | GGUUCAGUCUACUAUGGAGU | 340 |
| Downstream 3′ UTR | AAUCAAUUUCAGGCUUUUUC | 341 |
| Downstream 3′ UTR | UAAAUCAAUUUCAGGCUUUU | 342 |
| Downstream 3′ UTR | ACCAUUCUCUAGGUUCAGUC | 343 |
| Downstream 3′ UTR | CACGAGAAUUUUAACAUAUC | 344 |
| Downstream 3′ UTR | CAGGGGAUAUUGUGACAUAU | 345 |
| Downstream 3′ UTR | AAACACAUCUGCACUGAUCA | 346 |
| Downstream 3′ UTR | UCUACACGAGAAUUUUAACA | 347 |
| Downstream 3′ UTR | CUAUGGAGUUCUGAAACACA | 348 |
| Downstream 3′ UTR | GAGUUCUGAAACACAUCUGC | 349 |
| | GUUCAGAGAUAUAUCAAAAU | 457 |
| | UCAAAAUGCCCCCUCCCUGU | 458 |
| | AUUAGUUCAGAGAUAUAUCA | 459 |
| | GAUGAUUAGUUCAGAGAUAU | 460 |
| Exon #3 | UUAAAAUGCCCCCUCCCUGU | 476 |
| Exon #3 | UAUUAAAAUGCCCCCUCCCU | 477 |
| Exon #3 | AAAUGCCCCCUCCCUGUGGA | 478 |
| Exon #3 | AAAAUGCCCCCUCCCUGUGG | 479 |
| Exon #3 | AAUAUAUCUCUGAACUAAUC | 480 |

In some embodiments, the spacer sequence comprises one or more nucleobase alterations at one or more positions in any one of the sequences of TABLE 1 or TABLE 2. Alternative nucleobases can be any one or more of A, C, G, T or U, or a deletion, or an insertion. In some embodiments, the U is pseudouracil. By way of non-limiting example, a guanine nucleobase could be replaced with the nucleobase of any one of a cytosine, adenosine, thymine, and uracil. In some instance, the spacer sequence comprises only one nucleobase alterations relative to a sequence of TABLE 1 or TABLE 2. In some instance, the spacer sequence comprises not more than 1, not more than 2, nor more than 3, or not more than 4 nucleobase alterations relative to a sequence of TABLE 1 or TABLE 2. Categories based on target as delineated in TABLE 1 or TABLE 2 should be construed as suggestions and not limitations. A sequence that is in the exon 1 category for example, should not be construed as limited to a target sequence in exon 1 and no other location in the DUX4 gene.

### Repeat Sequences

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence includes a nucleotide sequence that is capable of forming a guide nucleic acid-effector protein complex (e.g., a RNP complex). In some embodiments, the repeat sequence may also be referred to as a “protein-binding segment.”

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a guide nucleic acid comprises a repeat sequence linked to a spacer sequence, which may be a direct link or by any suitable linker, examples of which are described herein. In some embodiments, the repeat sequence is adjacent to an intermediary RNA sequence. In some embodiments, a repeat sequence is 3′ to an intermediary RNA sequence. In some embodiments, an intermediary RNA sequence is followed by a repeat sequence, which is followed by a spacer sequence in the 5′ to 3′ direction. In some embodiments, a repeat sequence is linked to a spacer sequence and/or an intermediary RNA sequence. In some embodiments, a guide nucleic acid comprises a repeat sequence that is at least 80%, at least 90%, at least 92%, at least 95%, at least 97%, or at least 99%, or 100% identical to a sequence that is provided in TABLE 3. In some embodiments, guide nucleic acids comprise a repeat sequence, wherein the repeat sequence comprises at least 10, at least 12, at least 14, at least 16, at least 18 or at least 20 contiguous nucleotides of a sequence provided in TABLE 3.

TABLE 3
Exemplary Repeat Sequences

| Repeat sequence (shown | | SEQ |
|---|---|---|
| as RNA), | | ID |
| 5′- 3′ | Cas protein | NO: |
| GUAGAUUGCUCCUUACGAGGAGAC | CasPhi.12 | 115 |
| CUUUCAAGACUAAUAGAUUGCUCC | CasPhi.12 | 237 |
| UUACGAGGAGAC | | |
| AUAGAUUGCUCCUUACGAGGAGAC | CasPhi.12 | 238 |
| UAGAUUGCUCCUUACGAGGAGAC | CasPhi.12 | 239 |
| AGAUUGCUCCUUACGAGGAGAC | CasPhi.12 | 240 |
| GAUUGCUCCUUACGAGGAGAC | CasPhi.12 | 241 |
| AUUGCUCCUUACGAGGAGAC | CasPhi.12 | 242 |
| AAGGAUGCCAAAC | CasM.265466 | 350 |

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is at least partially, complementary. In some embodiments, such sequences may have 65% to 100% complementarity (e.g., at least 65%, at least 70%, at least 75%, at least 80%, at least 85%, at least 90%, at least 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, guide nucleic acids comprise a sequence that is at least 80%, at least 90%, at least 92%, at least 95%, at least 97%, or at least 99%, or 100% identical to SEQ ID NOs: 115 or 237-242.

In some embodiments, guide nucleic acids comprise a spacer sequence that is at least 80%, at least 85%, at least 90%, at least 95%, or 100% identical to any one of the sequences as set forth in TABLE 1; and a repeat sequence that is at least 80%, at least 85%, at least 90%, at least 95%, or 100% identical to a sequence selected from SEQ ID NOs: 115 and 237-242. In some embodiments, guide nucleic acids comprise a spacer sequence that is at least 80%, at least 85%, at least 90%, at least 95%, or 100% identical to any one of the sequences as set forth in TABLE 1; and a repeat sequence that is at least 80%, at least 85%, at least 90%, at least 95%, or 100% identical to SEQ ID NO: 242.

In some embodiments, guide nucleic acids comprise a sequence that is at least 80%, at least 90%, at least 92%, at least 95%, at least 97%, or at least 99%, or 100% identical to AAGGAUGCCAAAC (SEQ ID NO: 350).

In some embodiments, guide nucleic acids comprise a spacer sequence that is at least 80%, at least 85%, at least 90%, at least 95%, or 100% identical to any one of the sequences as set forth in TABLE 2; and a repeat sequence that is at least 80%, at least 85%, at least 90%, at least 95%, or 100% identical to SEQ ID NO: 350.

### Intermediary Sequences

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

In some embodiments, an intermediary sequence comprises a nucleotide sequence that is at least 65%, at least 70%, at least 75%, at least 80%, at least 85%, at least 90%, at least 95%, at least 97%, or at least 98%, at least 99%, or 100% identical to the sequence: ACAGCUUAUUUGGAAGCUGAAAUGUGAGGUUUAUAACACUCACAAGAAUCCU (SEQ ID NO: 351). In some embodiments, an intermediary sequence comprises at least 5, at least 6, at least 7, at least 8, at least 9, at least 10, at least 20, at least 30, at least 40, at least 45, or at least 50 contiguous nucleotides of any one of SEQ ID NO: 351. Such an intermediary sequence may be useful in a guide nucleic acid that is to be used with an effector protein that is at least 80%, at least 85%, at least 90%, at least 95%, at least 98%, at least 99% or 100% identical to any of SEQ ID NOs: 428-446 and 452.

### Handle Sequence

In some embodiments, compositions, systems and methods described herein comprise the nucleic acid, wherein the nucleic acid comprises a handle sequence. In some embodiments, the handle sequence comprises an intermediary sequence. In some embodiments, the intermediary sequence is at the 3′-end of the handle sequence. In some embodiments, the intermediary sequence is at the 5′-end of the handle sequence. In some embodiments, the handle sequence further comprises one or more of linkers and repeat sequences. In some embodiments, the linker comprises a sequence of 5′-GAAA-3′ (SEQ ID NO: 236). In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker.

In some embodiments, an sgRNA may include a handle sequence having a hairpin region, as well as a linker and a repeat sequence. The sgRNA having a handle sequence can have a hairpin region positioned 3′ of the linker and/or repeat sequence. The sgRNA having a handle sequence can have a hairpin region positioned 5′ of the linker and/or repeat sequence. The hairpin region may include a first sequence, a second sequence that is reverse complementary to the first sequence, and a stem-loop linking the first sequence and the second sequence.

In some embodiments, an effector protein may recognize a secondary structure of a handle sequence. In some embodiments, at least a portion of the handle sequence interacts with an effector protein described herein. Accordingly, in some embodiments, at least a portion of the intermediary sequence interacts with the effector protein described herein. In some embodiments, both, at least a portion of the intermediary sequence and at least a portion of the repeat sequence, interacts with the effector protein. In general, the handle sequence is capable of interacting (e.g., non-covalent binding) with any one of the effector proteins described herein.

In some embodiments, the handle sequence of a sgRNA comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the sgRNA comprises a pseudoknot (e.g., a secondary structure comprising a stem at least partially hybridized to a second stem or half-stem secondary structure). An effector protein may recognize a sgRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the sgRNA comprises at least 2, at least 3, at least 4, or at least 5 stem regions.

A handle sequence may include deoxyribonucleosides, ribonucleosides, chemically modified nucleosides, or any combination thereof. In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

In some embodiments, the length of a handle sequence in a sgRNA is not greater than 50, 56, 66, 67, 68, 69, 70, 71, 72, 73, 95, or 105 linked nucleotides. In some embodiments, the length of a handle sequence in a sgRNA is about 30 to about 120 linked nucleotides. In some embodiments, the length of a handle sequence in a sgRNA is about 50 to about 105, about 50 to about 95, about 50 to about 73, about 50 to about 71, about 50 to about 70, or about 50 to about 69 linked nucleotides. In some embodiments, the length of a handle sequence in a sgRNA is 56 to 105 linked nucleotides, from 56 to 105 linked nucleotides, 66 to 105 linked nucleotides, 67 to 105 linked nucleotides, 68 to 105 linked nucleotides, 69 to 105 linked nucleotides, 70 to 105 linked nucleotides, 71 to 105 linked nucleotides, 72 to 105 linked nucleotides, 73 to 105 linked nucleotides, or 95 to 105 linked nucleotides. In some embodiments, the length of a handle sequence in a sgRNA is 40 to 70 nucleotides. In some embodiments, the length of a handle sequence in a sgRNA is 50, 56, 66, 67, 68, 69, 70, 71, 72, 73, 95, or 105 linked nucleotides.

In some embodiments, a handle sequence comprises a nucleotide sequence that is at least 65%, at least 70%, at least 75%, at least 80%, at least 85%, at least 90%, at least 95%, at least 97%, at least 98%, at least 99%, or 100% identical to the sequence:

(SEQ ID NO: 352)
ACAGCUUAUUUGGAAGCUGAAAUGUGAGGUUUAUAACACUCACAAGAAUC
CUGAAAAAGGAUGCCAAAC.

### Exemplary Guide Nucleic Acids

In some embodiments, the guide nucleic acids disclosed herein comprise a spacer sequence that is at least 70%, at least 75%, at least 80%, at least 85%, at least 90%, at least 95%, at least 96%, at least 97%, at least 98%, at least 99%, or 100% identical to any one of the sequences as set forth in TABLE 1 and a repeat sequence that is at least 70%, at least 75%, at least 80%, at least 85%, at least 90%, at least 95%, at least 96%, at least 97%, at least 98%, at least 99%, or 100% identical to any one of the sequences of TABLE 3.

Exemplary guide nucleic acid sequences useful for systems, compositions and methods described herein are presented in TABLE 4. In some embodiments, the guide nucleic acid comprises a sequence that is at least 70%, at least 75%, at least 80%, at least 85%, at least 90%, at least 95%, or 100% identical to any one of the sequences of TABLE 4. In some embodiments, the guide nucleic acid consists of a sequence that is at least 70%, at least 75%, at least 80%, at least 85%, at least 90%, at least 95%, or 100% identical to any one of the sequences of TABLE 4. In some embodiments, the guide nucleic acids provided in TABLE 4 comprise an additional “G” at the 5′ end of the sequence.

The exemplary guide nucleic acids shown in TABLE 4 comprise a 24nt repeat sequence (SEQ ID: 238) or a 20nt repeat sequence (SEQ ID NO: 242). However, it should be understood that these guides can comprise any of the repeat sequences disclosed herein (e.g., any one of SEQ ID NOs: 115, and 237-242). For example, in some embodiments, the guide sequence comprises a spacer sequence of any one of SEQ ID NOs: 1-114, 456, and 481-596 with the repeat sequence of SEQ ID NO: 242.

TABLE 4
Exemplary Guide Nucleic Acids for CasPhi.12 Effector Proteins

| Target | | |
|---|---|---|
| Region of | | SEQ |
| DUX4 | Guide sequence (shown as RNA), (5′ to 3′) | ID: |
| Exon #1 | UAGAUUGCUCCUUACGAGGAGACAGCGGAACCCGUACCCGGGC | 116 |
| Exon #1 | AUAGAUUGCUCCUUACGAGGAGACAGAAGGAUCGCUUUCCAGGC | 117 |
| Exon #1 | AUAGAUUGCUCCUUACGAGGAGACGCCUACGCCGCCCCGGCUCC | 118 |
| Exon #1 | AUAGAUUGCUCCUUACGAGGAGACAGAUCUGGUUUCAGAAUCGA | 119 |
| Exon #1 | AUAGAUUGCUCCUUACGAGGAGACCGCCUACGCCGCCCCGGCUC | 120 |
| Exon #1 | AUAGAUUGCUCCUUACGAGGAGACCGUGAGCCAGGCAGCGAGGG | 121 |
| Exon #1 | AUAGAUUGCUCCUUACGAGGAGACCAGGCAUCGCCGCCCGGGAG | 122 |
| Exon #1 | AUAGAUUGCUCCUUACGAGGAGACGACCCCGAGCCAAAGCGAGG | 123 |
| Exon #1 | AUAGAUUGCUCCUUACGAGGAGACCAGAAUGAGAGGUCACGCCA | 124 |
| Exon #1 | AUAGAUUGCUCCUUACGAGGAGACGUGAGCCAGGCAGCGAGGGC | 125 |
| Exon #1 | AUAGAUUGCUCCUUACGAGGAGACCCAGGCAUCGCCGCCCGGGA | 126 |
| Exon #1 | AUAGAUUGCUCCUUACGAGGAGACGAGCGGAACCCGUACCCGGG | 127 |
| Exon #1 | AUAGAUUGCUCCUUACGAGGAGACGGACCCCGAGCCAAAGCGAG | 128 |
| Exon #1 | AUAGAUUGCUCCUUACGAGGAGACGGUUUCAGAAUGAGAGGUC | 129 |
| | A | |
| Exon #1 | AUAGAUUGCUCCUUACGAGGAGACUGCAGCAGGCGCAACCUCUC | 130 |
| Exon #1 | AUAGAUUGCUCCUUACGAGGAGACGUUUCAGAAUGAGAGGUCAC | 131 |
| Exon #1 | AUAGAUUGCUCCUUACGAGGAGACAGAAUGAGAGGUCACGCCAG | 132 |
| Exon #1 | AUAGAUUGCUCCUUACGAGGAGACCCGCACCCCACGUGCCCUGC | 133 |
| Exon #1 | AUAGAUUGCUCCUUACGAGGAGACCGGAGCCCAGGGUCCAGAUU | 134 |
| Exon #1 | AUAGAUUGCUCCUUACGAGGAGACAGAAUCGAAGGGCCAGGCAC | 135 |
| Exon #1 | AUAGAUUGCUCCUUACGAGGAGACGCCCACACCGGCGCGUGGGG | 136 |
| Exon #1 | AUAGAUUGCUCCUUACGAGGAGACCAGAAUCGAAGGGCCAGGCA | 137 |
| Exon #1 | AUAGAUUGCUCCUUACGAGGAGACCUGCAGCAGGCGCAACCUCU | 138 |
| Exon #1 | AUAGAUUGCUCCUUACGAGGAGACCGCCACCCACGUCCCAGGGG | 139 |
| Exon #1 | AUAGAUUGCUCCUUACGAGGAGACGAGAAGGAUCGCUUUCCAGG | 140 |
| Exon #1 | AUAGAUUGCUCCUUACGAGGAGACUGAAACCAGAUCUGAAUCCU | 141 |
| Exon #1 | AUAGAUUGCUCCUUACGAGGAGACGCCCGGGUGCGGAGGCCACC | 142 |
| Exon #1 | AUAGAUUGCUCCUUACGAGGAGACUCAAAGGCUCGGAGGAGCAG | 143 |
| Exon #1 | AUAGAUUGCUCCUUACGAGGAGACCGCGGGGAGGGUGCUGUCCG | 144 |
| Exon #1 | AUAGAUUGCUCCUUACGAGGAGACCCGCCGGUGCUGCCUCAGCU | 145 |
| Exon #1 | AUAGAUUGCUCCUUACGAGGAGACCCCGGGUGCGGAGGCCACCG | 146 |
| Exon #1 | AUAGAUUGCUCCUUACGAGGAGACCAUCUGCCCCUGCCGCGCGG | 147 |
| Exon #1 | AUAGAUUGCUCCUUACGAGGAGACCGCCUGCUGCAGAAACUCCG | 148 |
| Exon #1 | AUAGAUUGCUCCUUACGAGGAGACCCACGCCGCCCCGGCGACCU | 149 |
| Exon #1 | AUAGAUUGCUCCUUACGAGGAGACGCCCUGCGGCCCCGCUUGAG | 150 |
| Exon #1 | AUAGAUUGCUCCUUACGAGGAGACCUAGGAGAGGUUGCGCCUGC | 151 |
| Exon #1 | AUAGAUUGCUCCUUACGAGGAGACAGCGGGCCCAGGCUGUGCCA | 152 |
| Exon #1 | AUAGAUUGCUCCUUACGAGGAGACCUCGCUGAGGGGUGCUUCCA | 153 |
| Exon #1 | AUAGAUUGCUCCUUACGAGGAGACCGCUCAAAGCAGGCUCGCAG | 154 |
| Exon #1 | AUAGAUUGCUCCUUACGAGGAGACGGCUCGGGGUCCAAACGAGU | 155 |
| Exon #1 | AUAGAUUGCUCCUUACGAGGAGACGAUUCUGAAACCAGAUCUGA | 156 |
| Exon #1 | AUAGAUUGCUCCUUACGAGGAGACUGCCCGGGUGCGGAGGCCAC | 157 |
| Exon #1 | AUAGAUUGCUCCUUACGAGGAGACUGAAACCAAAUCUGGACCCU | 158 |
| Exon #1 | AUAGAUUGCUCCUUACGAGGAGACCGAGGCCUCCAGCUCCCCCG | 159 |
| Exon #1 | AUAGAUUGCUCCUUACGAGGAGACUCUGGUGGCGAUGCCCGGGU | 160 |
| Exon #1 | AUAGAUUGCUCCUUACGAGGAGACGGAGAUCCCCUCUGCCGGCG | 161 |
| Exon #1 | AUAGAUUGCUCCUUACGAGGAGACUAGGAGAGGUUGCGCCUGCU | 162 |
| Exon #1 | AUAGAUUGCUCCUUACGAGGAGACGGGUUCCCACGCCGCCCCGG | 163 |
| Exon #1 | AUAGAUUGCUCCUUACGAGGAGACCGCCGGCCUUCUGGCGGGCC | 164 |
| Exon #1 | AUAGAUUGCUCCUUACGAGGAGACCCCACGCGCCGGUGUGGGCG | 165 |
| Exon #1 | AUAGAUUGCUCCUUACGAGGAGACCAGCGAGGCGGCCUCUUCCG | 166 |
| Exon #1 | AUAGAUUGCUCCUUACGAGGAGACUUCCUCGCUGAGGGGUGCUU | 167 |
| Exon #1 | AUAGAUUGCUCCUUACGAGGAGACGCUCGGGGUCCAAACGAGUC | 168 |
| Exon #1 | AUAGAUUGCUCCUUACGAGGAGACUGGCGGGCCGCGUCUCCCGG | 169 |
| Exon #2 | AUAGAUUGCUCCUUACGAGGAGACGCCGGCCUUCUGGCGGGCCG | 170 |
| Exon #2 | AUAGAUUGCUCCUUACGAGGAGACCGACGCUGUCUAGGCAAACC | 171 |
| Exon #2 | AUAGAUUGCUCCUUACGAGGAGACAGAGAUAUAUUAAAAUGCCC | 172 |
| Exon #2 | AUAGAUUGCUCCUUACGAGGAGACCGUGAAAUUCUGGCUGAAUG | 173 |
| Exon #2 | AUAGAUUGCUCCUUACGAGGAGACUUCCGUGAAAUUCUGGCUGA | 174 |
| Exon #2 | AUAGAUUGCUCCUUACGAGGAGACGAGUUACAUCUCCUGGAUGA | 175 |
| Exon #2 | AUAGAUUGCUCCUUACGAGGAGACUUCUUCCGUGAAAUUCUGGC | 176 |
| Exon #2 | AUAGAUUGCUCCUUACGAGGAGACUGGCUGAAUGUCUCCCCCCA | 177 |
| Exon #2 | AUAGAUUGCUCCUUACGAGGAGACCAUCUCCUGGAUGAUUAGUU | 178 |
| Exon #2 | AUAGAUUGCUCCUUACGAGGAGACAAAUGCCCCCUCCCUGUGGA | 179 |
| Exon #2 | AUAGAUUGCUCCUUACGAGGAGACCACGUCAGCCGGGGUGCGCA | 180 |
| Exon #2 | AUAGAUUGCUCCUUACGAGGAGACAUAUAUCUCUGAACUAAUCA | 181 |
| Exon #2 | AUAGAUUGCUCCUUACGAGGAGACACGGAAGAACAAGGGCACAG | 182 |
| Exon #2 | AUAGAUUGCUCCUUACGAGGAGACCCUAGACAGCGUCGGAAGGU | 183 |
| Exon #2 | AUAGAUUGCUCCUUACGAGGAGACCACGGAAGAACAAGGGCACA | 184 |
| Exon #2 | AUAGAUUGCUCCUUACGAGGAGACGCCUAGACAGCGUCGGAAGG | 185 |
| Exon #2 | AUAGAUUGCUCCUUACGAGGAGACAAUAUAUCUCUGAACUAAUC | 186 |
| Exon #2 | AUAGAUUGCUCCUUACGAGGAGACAGCCAGAAUUUCACGGAAGA | 187 |
| Exon #2 | AUAGAUUGCUCCUUACGAGGAGACCCCGCUUCCUGGCUAGACCU | 188 |
| Intron #2 | AUAGAUUGCUCCUUACGAGGAGACCUGGCUAGACCUGCGCGCAG | 189 |
| Intron #2 | AUAGAUUGCUCCUUACGAGGAGACUAUAGGAUCCACAGGGAGGG | 190 |
| Intron #2 | AUAGAUUGCUCCUUACGAGGAGACCGGGCAGCCGCCUGGGCUGU | 191 |
| Intron #2 | AUAGAUUGCUCCUUACGAGGAGACGCGGGCAGCCGCCUGGGCUG | 192 |
| Intron #2 | AUAGAUUGCUCCUUACGAGGAGACCGGGGGUGGGGGGUGGGGG | 193 |
| | U | |
| Intron #2 | AUAGAUUGCUCCUUACGAGGAGACGCGGGACGGGGGUCUCCACC | 194 |
| Intron #2 | AUAGAUUGCUCCUUACGAGGAGACGACCGCCAGGCGCUCCGUGC | 195 |
| Intron #2 | AUAGAUUGCUCCUUACGAGGAGACUCCGGGGGUGGGGGGUGGG | 196 |
| | G | |
| Intron #2 | AUAGAUUGCUCCUUACGAGGAGACCCGGGGGUGGGGGGUGGGG | 197 |
| | G | |
| Intron #2- | AUAGAUUGCUCCUUACGAGGAGACCGCGGGACGGGGGUCUCCAC | 198 |
| Exon #3 | | |
| Downstream | AUAGAUUGCUCCUUACGAGGAGACUGACCGCCAGGCGCUCCGUG | 199 |
| 3′ UTR | | |
| Downstream | AUAGAUUGCUCCUUACGAGGAGACACCGCCAGGCGCUCCGUGCU | 200 |
| 3′ UTR | | |
| Downstream | AUAGAUUGCUCCUUACGAGGAGACGCCCGCUUCCUGGCUAGACC | 201 |
| 3′ UTR | | |
| Downstream | AUAGAUUGCUCCUUACGAGGAGACGGUGAUCAGUGCAGAUGUG | 202 |
| 3′ UTR | U | |
| Downstream | AUAGAUUGCUCCUUACGAGGAGACCAGAACUCCAUAGUAGACUG | 203 |
| 3′ UTR | | |
| Downstream | AUAGAUUGCUCCUUACGAGGAGACUGUGUGAUGAGUGCAGAGA | 204 |
| 3′ UTR | U | |
| Downstream | AUAGAUUGCUCCUUACGAGGAGACCAUCUUUUGUGUGAUGAGU | 20 |
| 3′ UTR | G | |
| Downstream | AUAGAUUGCUCCUUACGAGGAGACUGUGAUGAGUGCAGAGAUA | 206 |
| 3′ UTR | U | |
| Downstream | AUAGAUUGCUCCUUACGAGGAGACACAGAACUUCGGUGAUCAGU | 207 |
| 3′ UTR | | |
| Downstream | AUAGAUUGCUCCUUACGAGGAGACGCAUCUUUUGUGUGAUGAG | 208 |
| 3′ UTR | U | |
| Downstream | AUAGAUUGCUCCUUACGAGGAGACCAGAACUUCGGUGAUCAGUG | 209 |
| 3′ UTR | | |
| Downstream | AUAGAUUGCUCCUUACGAGGAGACAGAACUCCAUAGUAGACUGA | 210 |
| 3′ UTR | | |
| Downstream | AUAGAUUGCUCCUUACGAGGAGACGUGUGAUGAGUGCAGAGAU | 211 |
| 3′ UTR | A | |
| Downstream | AUAGAUUGCUCCUUACGAGGAGACCAUCACUUAGGUGAUCAGUG | 212 |
| 3′ UTR | | |
| Downstream | AUAGAUUGCUCCUUACGAGGAGACGGUGAUCAGUGUAGAGAUA | 213 |
| 3′ UTR | U | |
| Downstream | AUAGAUUGCUCCUUACGAGGAGACAAAUUCUCGUGUAGACAGAG | 214 |
| 3′ UTR | | |
| Downstream | AUAGAUUGCUCCUUACGAGGAGACAUUUACAGAACUUCGGUGAU | 215 |
| 3′ UTR | | |
| Downstream | AUAGAUUGCUCCUUACGAGGAGACUGAAACACAUCUGCACUGAU | 216 |
| 3′ UTR | | |
| Downstream | AUAGAUUGCUCCUUACGAGGAGACUUCUACAGGGGAUAUUGUG | 217 |
| 3′ UTR | A | |
| Downstream | AUAGAUUGCUCCUUACGAGGAGACAGUCUACUAUGGAGUUCUGA | 218 |
| 3′ UTR | | |
| Downstream | AUAGAUUGCUCCUUACGAGGAGACCAGGCUUUUUCUACAGGGGA | 219 |
| 3′ UTR | | |
| Downstream | AUAGAUUGCUCCUUACGAGGAGACUCUACAGGGGAUAUUGUGAC | 220 |
| 3′ UTR | | |
| Downstream | AUAGAUUGCUCCUUACGAGGAGACCUACAGGGGAUAUUGUGACA | 22 |
| 3′ UTR | | |
| Downstream | AUAGAUUGCUCCUUACGAGGAGACUAACAUAUCUCUACACUGAU | 222 |
| 3′ UTR | | |
| Downstream | AUAGAUUGCUCCUUACGAGGAGACACAUAUCUCUACACUGAUCA | 223 |
| 3′ UTR | | |
| Downstream | AUAGAUUGCUCCUUACGAGGAGACUGACAUAUCUCUGCACUCAU | 224 |
| 3′ UTR | | |
| Downstream | AUAGAUUGCUCCUUACGAGGAGACAACAUAUCUCUACACUGAUC | 225 |
| 3′ UTR | | |
| Downstream | AUAGAUUGCUCCUUACGAGGAGACUGUAAAUCAAUUUCAGGCUU | 226 |
| 3′ UTR | | |
| Downstream | AUAGAUUGCUCCUUACGAGGAGACUACAGGGGAUAUUGUGACA | 227 |
| 3′ UTR | U | |
| Downstream | AUAGAUUGCUCCUUACGAGGAGACUCUAGGUUCAGUCUACUAUG | 228 |
| 3′ UTR | | |
| Downstream | AUAGAUUGCUCCUUACGAGGAGACAGGCUUUUUCUACAGGGGAU | 229 |
| 3′ UTR | | |
| | AUUGCUCCUUACGAGGAGACAGAGAUAUAUCAAAAUGCCC | 461 |
| Exon #3 | AUUGCUCCUUACGAGGAGACAGAGAUAUAUUAAAAUGCCC | 602 |
| Exon #3 | AUUGCUCCUUACGAGGAGACAAAUGCCCCCUCCCUGUGGA | 603 |
| Exon #3 | AUUGCUCCUUACGAGGAGACAUAUAUCUCUGAACUAAUCA | 604 |
| Exon #3 | AUUGCUCCUUACGAGGAGACAAUAUAUCUCUGAACUAAUC | 605 |
| Exon #3 | AUUGCUCCUUACGAGGAGACAAAUGCCCCCUCCCUGU | 606 |
| | AUUGCUCCUUACGAGGAGACCUCUUCGUCUCUCCGGC | 607 |
| | AUUGCUCCUUACGAGGAGACCAAGGGCGGCUGGCUGG | 608 |
| | AUUGCUCCUUACGAGGAGACCGGGGUGGGGGGGGCUG | 609 |
| | AUUGCUCCUUACGAGGAGACGUCUCUCCGGCCCCACC | 610 |
| | AUUGCUCCUUACGAGGAGACCACACUCCCCUCCACCC | 611 |
| | AUUGCUCCUUACGAGGAGACCCGUUCCCGCGGGAUCC | 612 |
| | AUUGCUCCUUACGAGGAGACAGUUCCACACUCCCCUC | 613 |
| | AUUGCUCCUUACGAGGAGACACGGAGAGAGGGCCUGG | 614 |
| | AUUGCUCCUUACGAGGAGACUCCCUGCUGCCGACGCG | 615 |
| | AUUGCUCCUUACGAGGAGACCCGCGGGAUCCCUGGAG | 616 |
| | AUUGCUCCUUACGAGGAGACAUGAAGGGGUGGAGCCU | 617 |
| | AUUGCUCCUUACGAGGAGACACAAGGGCGGCUGGCUG | 618 |
| | AUUGCUCCUUACGAGGAGACCCUCCUUCACGGAGAGA | 619 |
| | AUUGCUCCUUACGAGGAGACCGGCCGGGGCUCACCGC | 620 |
| | AUUGCUCCUUACGAGGAGACCUCCCUGCUGCCGACGC | 621 |
| | AUUGCUCCUUACGAGGAGACCGGGGGCCGGCUCUCCG | 622 |
| | AUUGCUCCUUACGAGGAGACGGGGGCCGGCUCUCCGG | 623 |
| | AUUGCUCCUUACGAGGAGACAGUGUGCCAGGCCCUCU | 624 |
| | AUUGCUCCUUACGAGGAGACAUGAAUGGCGGUGAGCC | 625 |
| | AUUGCUCCUUACGAGGAGACCACGGACGGACGCGGGC | 626 |
| | AUUGCUCCUUACGAGGAGACACGGACGGACGCGGGCA | 627 |
| | AUUGCUCCUUACGAGGAGACUAAAGGCCCACAGGCAG | 628 |
| Exon #1 | AUUGCUCCUUACGAGGAGACGAGCGGAACCCGUACCCGGG | 629 |
| Exon #1 | AUUGCUCCUUACGAGGAGACAGAAUCGAAGGGCCAGGCAC | 630 |
| Exon #1 | AUUGCUCCUUACGAGGAGACCCGCACCCCACGUGCCCUGC | 631 |
| Exon #1 | AUUGCUCCUUACGAGGAGACGCCCACACCGGCGCGUGGGG | 632 |
| Exon #1 | AUUGCUCCUUACGAGGAGACGAGAAGGAUCGCUUUCCAGG | 633 |
| Exon #1 | AUUGCUCCUUACGAGGAGACAGAAUGAGAGGUCACGCCAG | 634 |
| Exon #1 | AUUGCUCCUUACGAGGAGACAGAAGGAUCGCUUUCCAGGC | 635 |
| Exon #1 | AUUGCUCCUUACGAGGAGACGGUUUCAGAAUGAGAGGUCA | 636 |
| Exon #1 | AUUGCUCCUUACGAGGAGACAGCGGAACCCGUACCCGGGC | 637 |
| Exon #1 | AUUGCUCCUUACGAGGAGACCCAGGCAUCGCCGCCCGGGA | 638 |
| Exon #1 | AUUGCUCCUUACGAGGAGACCGGAGCCCAGGGUCCAGAUU | 639 |
| Exon #1 | AUUGCUCCUUACGAGGAGACAGAUCUGGUUUCAGAAUCGA | 640 |
| Exon #1 | AUUGCUCCUUACGAGGAGACGUUUCAGAAUGAGAGGUCAC | 641 |
| Exon #1 | AUUGCUCCUUACGAGGAGACCAGGCAUCGCCGCCCGGGAG | 642 |
| Exon #1 | AUUGCUCCUUACGAGGAGACCAGAAUCGAAGGGCCAGGCA | 643 |
| Exon #1 | AUUGCUCCUUACGAGGAGACCAGAAUGAGAGGUCACGCCA | 644 |
| Exon #1 | AUUGCUCCUUACGAGGAGACGUGAGCCAGGCAGCGAGGGC | 645 |
| Exon #1 | AUUGCUCCUUACGAGGAGACGGACCCCGAGCCAAAGCGAG | 646 |
| Exon #1 | AUUGCUCCUUACGAGGAGACGACCCCGAGCCAAAGCGAGG | 647 |
| Exon #1 | AUUGCUCCUUACGAGGAGACCGUGAGCCAGGCAGCGAGGG | 648 |
| Exon #1 | AUUGCUCCUUACGAGGAGACCGCGGGGAGGGUGCUGUCCG | 649 |
| Exon #1 | AUUGCUCCUUACGAGGAGACGGCUCGGGGUCCAAACGAGU | 650 |
| Exon #1 | AUUGCUCCUUACGAGGAGACCCGCCGGUGCUGCCUCAGCU | 651 |
| Exon #1 | AUUGCUCCUUACGAGGAGACCGCCGGCCUUCUGGCGGGCC | 652 |
| Exon #1 | AUUGCUCCUUACGAGGAGACUGAAACCAGAUCUGAAUCCU | 653 |
| Exon #1 | AUUGCUCCUUACGAGGAGACCGCUCAAAGCAGGCUCGCAG | 654 |
| Exon #1 | AUUGCUCCUUACGAGGAGACUGGCGGGCCGCGUCUCCCGG | 655 |
| Exon #1 | AUUGCUCCUUACGAGGAGACGCCGGCCUUCUGGCGGGCCG | 656 |
| Exon #1 | AUUGCUCCUUACGAGGAGACGCUCGGGGUCCAAACGAGUC | 657 |
| Exon #1 | AUUGCUCCUUACGAGGAGACUCAAAGGCUCGGAGGAGCAG | 658 |
| Exon #1 | AUUGCUCCUUACGAGGAGACCCCACGCGCCGGUGUGGGCG | 659 |
| Exon #1 | AUUGCUCCUUACGAGGAGACUCUGGUGGCGAUGCCCGGGU | 660 |
| Exon #1 | AUUGCUCCUUACGAGGAGACUGAAACCAAAUCUGGACCCU | 661 |
| Exon #1 | AUUGCUCCUUACGAGGAGACGAUUCUGAAACCAGAUCUGA | 662 |
| Exon #1 | AUUGCUCCUUACGAGGAGACGACCCCGAGCCAAAGCG | 663 |
| Exon #1 | AUUGCUCCUUACGAGGAGACCGCCACCCACGUCCCAG | 664 |
| Exon #1 | AUUGCUCCUUACGAGGAGACGAGAAGGAUCGCUUUCC | 665 |
| Exon #1 | AUUGCUCCUUACGAGGAGACCAGAAUCGAAGGGCCAG | 666 |
| Exon #1 | AUUGCUCCUUACGAGGAGACCCGCACCCCACGUGCCC | 667 |
| Exon #1 | AUUGCUCCUUACGAGGAGACCGGAGCCCAGGGUCCAG | 668 |
| Exon #1 | AUUGCUCCUUACGAGGAGACCCAGGCAUCGCCGCCCG | 669 |
| Exon #1 | AUUGCUCCUUACGAGGAGACCAGAAUGAGAGGUCACG | 670 |
| Exon #1 | AUUGCUCCUUACGAGGAGACAGAAGGAUCGCUUUCCA | 671 |
| Exon #1 | AUUGCUCCUUACGAGGAGACGAGCGGAACCCGUACCC | 672 |
| Exon #1 | AUUGCUCCUUACGAGGAGACGUUUCAGAAUGAGAGGU | 673 |
| Exon #1 | AUUGCUCCUUACGAGGAGACGCCUACGCCGCCCCGGC | 674 |
| Exon #1 | AUUGCUCCUUACGAGGAGACAGAAUGAGAGGUCACGC | 675 |
| Exon #1 | AUUGCUCCUUACGAGGAGACAGCGGAACCCGUACCCG | 676 |
| Exon #1 | AUUGCUCCUUACGAGGAGACCGUGAGCCAGGCAGCGA | 677 |
| Exon #1 | AUUGCUCCUUACGAGGAGACAGAUCUGGUUUCAGAAU | 678 |
| Exon #1 | AUUGCUCCUUACGAGGAGACAGAAUCGAAGGGCCAGG | 679 |
| Exon #1 | AUUGCUCCUUACGAGGAGACCGCCUACGCCGCCCCGG | 680 |
| Exon #1 | AUUGCUCCUUACGAGGAGACCUGCAGCAGGCGCAACC | 681 |
| Exon #1 | AUUGCUCCUUACGAGGAGACUGCAGCAGGCGCAACCU | 682 |
| Exon #1 | AUUGCUCCUUACGAGGAGACGGACCCCGAGCCAAAGC | 683 |
| Exon #1 | AUUGCUCCUUACGAGGAGACCAGGCAUCGCCGCCCGG | 684 |
| Exon #1 | AUUGCUCCUUACGAGGAGACGUGAGCCAGGCAGCGAG | 685 |
| Exon #1 | AUUGCUCCUUACGAGGAGACGCCCACACCGGCGCGUG | 686 |
| Exon #1 | AUUGCUCCUUACGAGGAGACGGUUUCAGAAUGAGAGG | 687 |
| Exon #1 | AUUGCUCCUUACGAGGAGACUUCCUCGCUGAGGGGUG | 688 |
| Exon #1 | AUUGCUCCUUACGAGGAGACCGCCUGCUGCAGAAACU | 689 |
| Exon #1 | AUUGCUCCUUACGAGGAGACUGCCCGGGUGCGGAGGC | 690 |
| Exon #1 | AUUGCUCCUUACGAGGAGACCAGCGAGGCGGCCUCUU | 691 |
| Exon #1 | AUUGCUCCUUACGAGGAGACUGAAACCAAAUCUGGAC | 692 |
| Exon #1 | AUUGCUCCUUACGAGGAGACCGAGGCCUCCAGCUCCC | 693 |
| Exon #1 | AUUGCUCCUUACGAGGAGACUAGGAGAGGUUGCGCCU | 694 |
| Exon #1 | AUUGCUCCUUACGAGGAGACAGCGGGCCCAGGCUGUG | 695 |
| Exon #1 | AUUGCUCCUUACGAGGAGACGGGUUCCCACGCCGCCC | 696 |
| Exon #1 | AUUGCUCCUUACGAGGAGACUGAAACCAGAUCUGAAU | 697 |
| Exon #1 | AUUGCUCCUUACGAGGAGACGCUCGGGGUCCAAACGA | 698 |
| Exon #1 | AUUGCUCCUUACGAGGAGACGGAGAUCCCCUCUGCCG | 699 |
| Exon #1 | AUUGCUCCUUACGAGGAGACCCCGGGUGCGGAGGCCA | 700 |
| Exon #1 | AUUGCUCCUUACGAGGAGACCCGCCGGUGCUGCCUCA | 701 |
| Exon #1 | AUUGCUCCUUACGAGGAGACCCACGCCGCCCCGGCGA | 702 |
| Exon #1 | AUUGCUCCUUACGAGGAGACGCCCUGCGGCCCCGCUU | 703 |
| Exon #1 | AUUGCUCCUUACGAGGAGACCGCUCAAAGCAGGCUCG | 704 |
| Exon #1 | AUUGCUCCUUACGAGGAGACCUAGGAGAGGUUGCGCC | 705 |
| Exon #1 | AUUGCUCCUUACGAGGAGACCUCGCUGAGGGGUGCUU | 706 |
| Exon #1 | AUUGCUCCUUACGAGGAGACCAUCUGCCCCUGCCGCG | 707 |
| Exon #1 | AUUGCUCCUUACGAGGAGACGCCCGGGUGCGGAGGCC | 708 |
| Exon #1 | AUUGCUCCUUACGAGGAGACCGCGGGGAGGGUGCUGU | 709 |
| Exon #1 | AUUGCUCCUUACGAGGAGACCGCCGGCCUUCUGGCGG | 710 |
| Exon #1 | AUUGCUCCUUACGAGGAGACGGCUCGGGGUCCAAACG | 711 |
| Exon #1 | AUUGCUCCUUACGAGGAGACUCAAAGGCUCGGAGGAG | 712 |
| Exon #1 | AUUGCUCCUUACGAGGAGACGCCGGCCUUCUGGCGGG | 713 |
| Exon #1 | AUUGCUCCUUACGAGGAGACGAUUCUGAAACCAGAUC | 714 |
| Exon #1 | AUUGCUCCUUACGAGGAGACCCCACGCGCCGGUGUGG | 715 |
| Exon #1 | AUUGCUCCUUACGAGGAGACUCUGGUGGCGAUGCCCG | 716 |
| Exon #1 | AUUGCUCCUUACGAGGAGACUGGCGGGCCGCGUCUCC | 717 |

In some embodiments, the guide nucleic acids disclosed herein comprise a spacer sequence that is at least 70%, at least 75%, at least 80%, at least 85%, at least 90%, at least 95%, or 100% identical to any one of the sequences as set forth in TABLE 2, a repeat sequence that is at least 70%, at least 75%, at least 80%, at least 85%, at least 90%, at least 95%, or 100% identical to SEQ ID NO: 350, and an intermediary sequence that is at least 70%, at least 75%, at least 80%, at least 85%, at least 90%, at least 95%, or 100% identical to SEQ ID NO: 351.

Exemplary guide nucleic acid sequences useful for systems, compositions and methods described herein are presented in TABLE 5. In some embodiments, the guide nucleic acid comprises a sequence that is at least 70%, at least 75%, at least 80%, at least 85%, at least 90%, at least 95%, at least 96%, at least 97%, at least 98%, at least 99%, or 100% identical to any one of the sequences of TABLE 5. In some embodiments, the guide nucleic acid consists of a sequence that is at least 70%, at least 75%, at least 80%, at least 85%, at least 90%, at least 95%, at least 96%, at least 97%, at least 98%, at least 99%, or 100% identical to any one of the sequences of TABLE 5. In some embodiments, the guide nucleic acids provided in TABLE 5 comprise an additional “G” at the 5′ end of the sequence.

TABLE 5
Exemplary Guide Nucleic Acids for CasM.265466 Effector Proteins

| Target | | |
|---|---|---|
| Region of | | SEQ ID |
| DUX4 | Sequence (5′ to 3′) | NO: |
| Exon #1 | ACAGCUUAUUUGGAAGCUGAAAUGUGAGGUUUAUAACACUC | 353 |
| | ACAAGAAUCCUGAAAAAGGAUGCCAAACGACCCCGAGCCAA | |
| | AGCGAGG | |
| Exon #1 | ACAGCUUAUUUGGAAGCUGAAAUGUGAGGUUUAUAACACUC | 354 |
| | ACAAGAAUCCUGAAAAAGGAUGCCAAACAGCGGAACCCGUA | |
| | CCCGGGC | |
| Exon #1 | ACAGCUUAUUUGGAAGCUGAAAUGUGAGGUUUAUAACACUC | 355 |
| | ACAAGAAUCCUGAAAAAGGAUGCCAAACCUGGAGGAGCUUU | |
| | AGGACGC | |
| Exon #1 | ACAGCUUAUUUGGAAGCUGAAAUGUGAGGUUUAUAACACUC | 356 |
| | ACAAGAAUCCUGAAAAAGGAUGCCAAACAGAAGGAUCGCUU | |
| | UCCAGGC | |
| Exon #1 | ACAGCUUAUUUGGAAGCUGAAAUGUGAGGUUUAUAACACUC | 357 |
| | ACAAGAAUCCUGAAAAAGGAUGCCAAACCAGCGCGGCCCCC | |
| | GGCGGGG | |
| Exon #1 | ACAGCUUAUUUGGAAGCUGAAAUGUGAGGUUUAUAACACUC | 358 |
| | ACAAGAAUCCUGAAAAAGGAUGCCAAACCAGCAGGCGCAAC | |
| | CUCUCCU | |
| Exon #1 | ACAGCUUAUUUGGAAGCUGAAAUGUGAGGUUUAUAACACUC | 359 |
| | ACAAGAAUCCUGAAAAAGGAUGCCAAACGUUUCAGAAUGAG | |
| | AGGUCAC | |
| Exon #1 | ACAGCUUAUUUGGAAGCUGAAAUGUGAGGUUUAUAACACUC | 360 |
| | ACAAGAAUCCUGAAAAAGGAUGCCAAACCACUCCCCUGCGG | |
| | CCUGCUG | |
| Exon #1 | ACAGCUUAUUUGGAAGCUGAAAUGUGAGGUUUAUAACACUC | 361 |
| | ACAAGAAUCCUGAAAAAGGAUGCCAAACGUUUCAGAAUCGA | |
| | AGGGCCA | |
| Exon #1 | ACAGCUUAUUUGGAAGCUGAAAUGUGAGGUUUAUAACACUC | 362 |
| | ACAAGAAUCCUGAAAAAGGAUGCCAAACGGAUCCGGUGACG | |
| | GCGGUCC | |
| Exon #1 | ACAGCUUAUUUGGAAGCUGAAAUGUGAGGUUUAUAACACUC | 363 |
| | ACAAGAAUCCUGAAAAAGGAUGCCAAACCCCCUGCCGCGCG | |
| | GAGGCGG | |
| Exon #1 | ACAGCUUAUUUGGAAGCUGAAAUGUGAGGUUUAUAACACUC | 364 |
| | ACAAGAAUCCUGAAAAAGGAUGCCAAACCCGGCGCGGCCUG | |
| | GCUGGGC | |
| Exon #1 | ACAGCUUAUUUGGAAGCUGAAAUGUGAGGUUUAUAACACUC | 365 |
| | ACAAGAAUCCUGAAAAAGGAUGCCAAACGGAGAGGUUGCGC | |
| | CUGCUGC | |
| Exon #1 | ACAGCUUAUUUGGAAGCUGAAAUGUGAGGUUUAUAACACUC | 366 |
| | ACAAGAAUCCUGAAAAAGGAUGCCAAACGGCGAAGGCGACC | |
| | CACGAGG | |
| Exon #1 | ACAGCUUAUUUGGAAGCUGAAAUGUGAGGUUUAUAACACUC | 367 |
| | ACAAGAAUCCUGAAAAAGGAUGCCAAACAAUCCUGGACUCC | |
| | GGGAGGC | |
| Exon #1 | ACAGCUUAUUUGGAAGCUGAAAUGUGAGGUUUAUAACACUC | 368 |
| | ACAAGAAUCCUGAAAAAGGAUGCCAAACGCCAGCUCCUCCC | |
| | GGGCGGC | |
| Exon #1 | ACAGCUUAUUUGGAAGCUGAAAUGUGAGGUUUAUAACACUC | 369 |
| | ACAAGAAUCCUGAAAAAGGAUGCCAAACAAACCAAAUCUGG | |
| | ACCCUGG | |
| Exon #1 | ACAGCUUAUUUGGAAGCUGAAAUGUGAGGUUUAUAACACUC | 370 |
| | ACAAGAAUCCUGAAAAAGGAUGCCAAACCCCGGGUGCGGAG | |
| | GCCACCG | |
| Exon #1 | ACAGCUUAUUUGGAAGCUGAAAUGUGAGGUUUAUAACACUC | 371 |
| | ACAAGAAUCCUGAAAAAGGAUGCCAAACGCGGGCCGCGUCU | |
| | CCCGGGC | |
| Exon #1 | ACAGCUUAUUUGGAAGCUGAAAUGUGAGGUUUAUAACACUC | 372 |
| | ACAAGAAUCCUGAAAAAGGAUGCCAAACGCUCGGGGUCCAA | |
| | ACGAGUC | |
| Exon #1 | ACAGCUUAUUUGGAAGCUGAAAUGUGAGGUUUAUAACACUC | 373 |
| | ACAAGAAUCCUGAAAAAGGAUGCCAAACAAACCAGAUCUGA | |
| | AUCCUGG | |
| Exon #1 | ACAGCUUAUUUGGAAGCUGAAAUGUGAGGUUUAUAACACUC | 374 |
| | ACAAGAAUCCUGAAAAAGGAUGCCAAACGUGGCGAUGCCCG | |
| | GGUACGG | |
| Exon #1 | ACAGCUUAUUUGGAAGCUGAAAUGUGAGGUUUAUAACACUC | 375 |
| | ACAAGAAUCCUGAAAAAGGAUGCCAAACGGAGAGCCCCAGG | |
| | CGCGCAG | |
| Exon #1 | ACAGCUUAUUUGGAAGCUGAAAUGUGAGGUUUAUAACACUC | 376 |
| | ACAAGAAUCCUGAAAAAGGAUGCCAAACCCACCGCGCAGGG | |
| | GCCCGGC | |
| Exon #1 | ACAGCUUAUUUGGAAGCUGAAAUGUGAGGUUUAUAACACUC | 377 |
| | ACAAGAAUCCUGAAAAAGGAUGCCAAACGACCCUGGGCUCC | |
| | GGAAUGC | |
| Exon #3 | ACAGCUUAUUUGGAAGCUGAAAUGUGAGGUUUAUAACACUC | 378 |
| | ACAAGAAUCCUGAAAAAGGAUGCCAAACCCCUUGUUCUUCC | |
| | GUGAAAU | |
| Exon #3 | ACAGCUUAUUUGGAAGCUGAAAUGUGAGGUUUAUAACACUC | 379 |
| | ACAAGAAUCCUGAAAAAGGAUGCCAAACUUAAAAUGCCCCC | |
| | UCCCUGU | |
| Exon #3 | ACAGCUUAUUUGGAAGCUGAAAUGUGAGGUUUAUAACACUC | 380 |
| | ACAAGAAUCCUGAAAAAGGAUGCCAAACGCUGAAUGUCUCC | |
| | CCCCACC | |
| Exon #3 | ACAGCUUAUUUGGAAGCUGAAAUGUGAGGUUUAUAACACUC | 381 |
| | ACAAGAAUCCUGAAAAAGGAUGCCAAACUGCCCUUGUUCUU | |
| | CCGUGAA | |
| Exon #3 | ACAGCUUAUUUGGAAGCUGAAAUGUGAGGUUUAUAACACUC | 382 |
| | ACAAGAAUCCUGAAAAAGGAUGCCAAACGGCAAACCUGGAU | |
| | UAGAGUU | |
| Exon #3 | ACAGCUUAUUUGGAAGCUGAAAUGUGAGGUUUAUAACACUC | 383 |
| | ACAAGAAUCCUGAAAAAGGAUGCCAAACAUAUAUCUCUGAA | |
| | CUAAUCA | |
| Exon #3 | ACAGCUUAUUUGGAAGCUGAAAUGUGAGGUUUAUAACACUC | 384 |
| | ACAAGAAUCCUGAAAAAGGAUGCCAAACUCUCUGAACUAAU | |
| | CAUCCAG | |
| Exon #3 | ACAGCUUAUUUGGAAGCUGAAAUGUGAGGUUUAUAACACUC | 385 |
| | ACAAGAAUCCUGAAAAAGGAUGCCAAACAACUAAUCAUCCA | |
| | GGAGAUG | |
| Exon #3 | ACAGCUUAUUUGGAAGCUGAAAUGUGAGGUUUAUAACACUC | 386 |
| | ACAAGAAUCCUGAAAAAGGAUGCCAAACCCUAGACAGCGUC | |
| | GGAAGGU | |
| Exon #3 | ACAGCUUAUUUGGAAGCUGAAAUGUGAGGUUUAUAACACUC | 387 |
| | ACAAGAAUCCUGAAAAAGGAUGCCAAACGGAUCCACAGGGA | |
| | GGGGGCA | |
| Exon #3 | ACAGCUUAUUUGGAAGCUGAAAUGUGAGGUUUAUAACACUC | 388 |
| | ACAAGAAUCCUGAAAAAGGAUGCCAAACAUCCAGGUUUGCC | |
| | UAGACAG | |
| Exon #3 | ACAGCUUAUUUGGAAGCUGAAAUGUGAGGUUUAUAACACUC | 389 |
| | ACAAGAAUCCUGAAAAAGGAUGCCAAACACUCUAAUCCAGG | |
| | UUUGCCU | |
| Exon #3 | ACAGCUUAUUUGGAAGCUGAAAUGUGAGGUUUAUAACACUC | 390 |
| | ACAAGAAUCCUGAAAAAGGAUGCCAAACCCCGCUUCCUGGC | |
| | UAGACCU | |
| Exon #3 | ACAGCUUAUUUGGAAGCUGAAAUGUGAGGUUUAUAACACUC | 391 |
| | ACAAGAAUCCUGAAAAAGGAUGCCAAACUAGGAUCCACAGG | |
| | GAGGGGG | |
| Intron #2 | ACAGCUUAUUUGGAAGCUGAAAUGUGAGGUUUAUAACACUC | 392 |
| | ACAAGAAUCCUGAAAAAGGAUGCCAAACGGAGCAGCCCGGG | |
| | CAGAGCU | |
| Intron #2 | ACAGCUUAUUUGGAAGCUGAAAUGUGAGGUUUAUAACACUC | 393 |
| | ACAAGAAUCCUGAAAAAGGAUGCCAAACUCUGUCUUUGCCC | |
| | GCUUCCU | |
| Intron #2 | ACAGCUUAUUUGGAAGCUGAAAUGUGAGGUUUAUAACACUC | 394 |
| | ACAAGAAUCCUGAAAAAGGAUGCCAAACUCUUUGCCCGCUU | |
| | CCUGGCU | |
| Intron #2 | ACAGCUUAUUUGGAAGCUGAAAUGUGAGGUUUAUAACACUC | 395 |
| | ACAAGAAUCCUGAAAAAGGAUGCCAAACCGGGCAGCCGCCU | |
| | GGGCUGU | |
| Intron #2 | ACAGCUUAUUUGGAAGCUGAAAUGUGAGGUUUAUAACACUC | 396 |
| | ACAAGAAUCCUGAAAAAGGAUGCCAAACCGCCCCCGCGCCA | |
| | CCGUCGC | |
| Intron #2 | ACAGCUUAUUUGGAAGCUGAAAUGUGAGGUUUAUAACACUC | 397 |
| | ACAAGAAUCCUGAAAAAGGAUGCCAAACCCCGGGCUGCUCC | |
| | CACAGCC | |
| Intron #2 | ACAGCUUAUUUGGAAGCUGAAAUGUGAGGUUUAUAACACUC | 398 |
| | ACAAGAAUCCUGAAAAAGGAUGCCAAACCUUUUGACCGCCA | |
| | GGCGCUC | |
| Intron #2 | ACAGCUUAUUUGGAAGCUGAAAUGUGAGGUUUAUAACACUC | 399 |
| | ACAAGAAUCCUGAAAAAGGAUGCCAAACACCGCCAGGCGCU | |
| | CCGUGCU | |
| Intron #2 | ACAGCUUAUUUGGAAGCUGAAAUGUGAGGUUUAUAACACUC | 400 |
| | ACAAGAAUCCUGAAAAAGGAUGCCAAACGCCAGGAAGCGGG | |
| | CAAAGAC | |
| Downstream | ACAGCUUAUUUGGAAGCUGAAAUGUGAGGUUUAUAACACUC | 401 |
| 3′ UTR | ACAAGAAUCCUGAAAAAGGAUGCCAAACUUUCAGAACUCCA | |
| | UAGUAGA | |
| Downstream | ACAGCUUAUUUGGAAGCUGAAAUGUGAGGUUUAUAACACUC | 402 |
| 3′ UTR | ACAAGAAUCCUGAAAAAGGAUGCCAAACAUGAGUGCAGAGA | |
| | UAUGUCA | |
| Downstream | ACAGCUUAUUUGGAAGCUGAAAUGUGAGGUUUAUAACACUC | 403 |
| 3′ UTR | ACAAGAAUCCUGAAAAAGGAUGCCAAACUGAUGAGUGCAGA | |
| | GAUAUGU | |
| Downstream | ACAGCUUAUUUGGAAGCUGAAAUGUGAGGUUUAUAACACUC | 404 |
| 3′ UTR | ACAAGAAUCCUGAAAAAGGAUGCCAAACUUAAAAUUCUCGU | |
| | GUAGACA | |
| Downstream | ACAGCUUAUUUGGAAGCUGAAAUGUGAGGUUUAUAACACUC | 405 |
| 3′ UTR | ACAAGAAUCCUGAAAAAGGAUGCCAAACGAGAUAUGUUAAA | |
| | AUUCUCG | |
| Downstream | ACAGCUUAUUUGGAAGCUGAAAUGUGAGGUUUAUAACACUC | 406 |
| 3′ UTR | ACAAGAAUCCUGAAAAAGGAUGCCAAACGAUCCUAUAGAAG | |
| | AUUUGCA | |
| Downstream | ACAGCUUAUUUGGAAGCUGAAAUGUGAGGUUUAUAACACUC | 407 |
| 3′ UTR | ACAAGAAUCCUGAAAAAGGAUGCCAAACCAGAACUUCGGUG | |
| | AUCAGUG | |
| Downstream | ACAGCUUAUUUGGAAGCUGAAAUGUGAGGUUUAUAACACUC | 408 |
| 3′ UTR | ACAAGAAUCCUGAAAAAGGAUGCCAAACGAAAAAGCCUGAA | |
| | AUUGAUU | |
| Downstream | ACAGCUUAUUUGGAAGCUGAAAUGUGAGGUUUAUAACACUC | 409 |
| 3′ UTR | ACAAGAAUCCUGAAAAAGGAUGCCAAACUGUGAUGAGUGCA | |
| | GAGAUAU | |
| Downstream | ACAGCUUAUUUGGAAGCUGAAAUGUGAGGUUUAUAACACUC | 410 |
| 3′ UTR | ACAAGAAUCCUGAAAAAGGAUGCCAAACCAUCUUUUGUGUG | |
| | AUGAGUG | |
| Downstream | ACAGCUUAUUUGGAAGCUGAAAUGUGAGGUUUAUAACACUC | 411 |
| 3′ UTR | ACAAGAAUCCUGAAAAAGGAUGCCAAACGAAGAUUUGCAUC | |
| | UUUUGUG | |
| Downstream | ACAGCUUAUUUGGAAGCUGAAAUGUGAGGUUUAUAACACUC | 412 |
| 3′ UTR | ACAAGAAUCCUGAAAAAGGAUGCCAAACUCACAAUAUCCCC | |
| | UGUAGAA | |
| Downstream | ACAGCUUAUUUGGAAGCUGAAAUGUGAGGUUUAUAACACUC | 413 |
| 3′ UTR | ACAAGAAUCCUGAAAAAGGAUGCCAAACCACUGAUCACCGA | |
| | AGUUCUG | |
| Downstream | ACAGCUUAUUUGGAAGCUGAAAUGUGAGGUUUAUAACACUC | 414 |
| 3′ UTR | ACAAGAAUCCUGAAAAAGGAUGCCAAACCACUGAUCACCUA | |
| | AGUGAUG | |
| Downstream | ACAGCUUAUUUGGAAGCUGAAAUGUGAGGUUUAUAACACUC | 415 |
| 3′ UTR | ACAAGAAUCCUGAAAAAGGAUGCCAAACACAUAUCUCUACA | |
| | CUGAUCA | |
| Downstream | ACAGCUUAUUUGGAAGCUGAAAUGUGAGGUUUAUAACACUC | 416 |
| 3′ UTR | ACAAGAAUCCUGAAAAAGGAUGCCAAACACAUAUCUCUGCA | |
| | CUCAUCA | |
| Downstream | ACAGCUUAUUUGGAAGCUGAAAUGUGAGGUUUAUAACACUC | 417 |
| 3′ UTR | ACAAGAAUCCUGAAAAAGGAUGCCAAACCACUCAUCACACA | |
| | AAAGAUG | |
| Downstream | ACAGCUUAUUUGGAAGCUGAAAUGUGAGGUUUAUAACACUC | 418 |
| 3′ UTR | ACAAGAAUCCUGAAAAAGGAUGCCAAACGGUUCAGUCUACU | |
| | AUGGAGU | |
| Downstream | ACAGCUUAUUUGGAAGCUGAAAUGUGAGGUUUAUAACACUC | 419 |
| 3′ UTR | ACAAGAAUCCUGAAAAAGGAUGCCAAACAAUCAAUUUCAGG | |
| | CUUUUUC | |
| Downstream | ACAGCUUAUUUGGAAGCUGAAAUGUGAGGUUUAUAACACUC | 420 |
| 3′ UTR | ACAAGAAUCCUGAAAAAGGAUGCCAAACUAAAUCAAUUUCA | |
| | GGCUUUU | |
| Downstream | ACAGCUUAUUUGGAAGCUGAAAUGUGAGGUUUAUAACACUC | 421 |
| 3′ UTR | ACAAGAAUCCUGAAAAAGGAUGCCAAACACCAUUCUCUAGG | |
| | UUCAGUC | |
| Downstream | ACAGCUUAUUUGGAAGCUGAAAUGUGAGGUUUAUAACACUC | 422 |
| 3′ UTR | ACAAGAAUCCUGAAAAAGGAUGCCAAACCACGAGAAUUUUA | |
| | ACAUAUC | |
| Downstream | ACAGCUUAUUUGGAAGCUGAAAUGUGAGGUUUAUAACACUC | 423 |
| 3′ UTR | ACAAGAAUCCUGAAAAAGGAUGCCAAACCAGGGGAUAUUGU | |
| | GACAUAU | |
| Downstream | ACAGCUUAUUUGGAAGCUGAAAUGUGAGGUUUAUAACACUC | 424 |
| 3′ UTR | ACAAGAAUCCUGAAAAAGGAUGCCAAACAAACACAUCUGCA | |
| | CUGAUCA | |
| Downstream | ACAGCUUAUUUGGAAGCUGAAAUGUGAGGUUUAUAACACUC | 425 |
| 3′ UTR | ACAAGAAUCCUGAAAAAGGAUGCCAAACUCUACACGAGAAU | |
| | UUUAACA | |
| Downstream | ACAGCUUAUUUGGAAGCUGAAAUGUGAGGUUUAUAACACUC | 426 |
| 3′ UTR | ACAAGAAUCCUGAAAAAGGAUGCCAAACCUAUGGAGUUCUG | |
| | AAACACA | |
| Downstream | ACAGCUUAUUUGGAAGCUGAAAUGUGAGGUUUAUAACACUC | 427 |
| 3′ UTR | ACAAGAAUCCUGAAAAAGGAUGCCAAACGAGUUCUGAAACA | |
| | CAUCUGC | |
| | ACAGCUUAUUUGGAAGCUGAAAUGUGAGGUUUAUAACACUC | 462 |
| | ACAAGAAUCCUGAAAAAGGAUGCCAAACGUUCAGAGAUAUA | |
| | UCAAAAU | |
| | ACAGCUUAUUUGGAAGCUGAAAUGUGAGGUUUAUAACACUC | 463 |
| | ACAAGAAUCCUGAAAAAGGAUGCCAAACUCAAAAUGCCCCC | |
| | UCCCUGU | |
| | ACAGCUUAUUUGGAAGCUGAAAUGUGAGGUUUAUAACACUC | 464 |
| | ACAAGAAUCCUGAAAAAGGAUGCCAAACAUUAGUUCAGAGA | |
| | UAUAUCA | |
| | ACAGCUUAUUUGGAAGCUGAAAUGUGAGGUUUAUAACACUC | 465 |
| | ACAAGAAUCCUGAAAAAGGAUGCCAAACGAUGAUUAGUUCA | |
| | GAGAUAU | |
| Exon # 3 | ACAGCUUAUUUGGAAGCUGAAAUGUGAGGUUUAUAACACUC | 597 |
| | ACAAGAAUCCUGAAAAAGGAUGCCAAACUUAAAAUGCCCCC | |
| | UCCCUGU | |
| Exon # 3 | ACAGCUUAUUUGGAAGCUGAAAUGUGAGGUUUAUAACACUC | 598 |
| | ACAAGAAUCCUGAAAAAGGAUGCCAAACUAUUAAAAUGCCC | |
| | CCUCCCU | |
| Exon # 3 | ACAGCUUAUUUGGAAGCUGAAAUGUGAGGUUUAUAACACUC | 599 |
| | ACAAGAAUCCUGAAAAAGGAUGCCAAACAAAUGCCCCCUCC | |
| | CUGUGGA | |
| Exon # 3 | ACAGCUUAUUUGGAAGCUGAAAUGUGAGGUUUAUAACACUC | 600 |
| | ACAAGAAUCCUGAAAAAGGAUGCCAAACAAAAUGCCCCCUC | |
| | CCUGUGG | |
| Exon # 3 | ACAGCUUAUUUGGAAGCUGAAAUGUGAGGUUUAUAACACUC | 601 |
| | ACAAGAAUCCUGAAAAAGGAUGCCAAACAAUAUAUCUCUGA | |
| | ACUAAUC | |

In some embodiments, guide nucleic acids comprise a portion or all of a sequence as set forth in any one of TABLES 1, 3, or 4. In some embodiments, a guide nucleic acid comprises at least 9, at least 10, at least 11, at least 12 contiguous nucleotides of any one of SEQ ID NOs: 1-229, 237-242, 456, 461, 481-596 and 602-717. In some embodiments, the guide nucleic acid comprises at least 15, at least 20, at least 25, at least 30, or at least 35 contiguous nucleotides of any one of SEQ ID NOs: 1-229, 237-242, 456, 461, 481-596 and 602-717.

In some embodiments, compositions disclosed herein comprises a spacer sequence that is at least 70%, at least 75%, at least 80%, at least 85%, at least 90%, at least 95%, or 100% identical to any one of the sequences as set forth in TABLE 1, and comprising a repeat sequence that is at least 70%, at least 75%, at least 80%, at least 85%, at least 90%, at least 95%, or 100% identical to any one of the sequences of TABLE 3.

In some embodiments, compositions disclosed herein comprises a guide nucleic acid sequence that is at least 70%, at least 75%, at least 80%, at least 85%, at least 90%, at least 95%, or 100% identical to any one of the sequences as set forth in TABLE 4.

In some embodiments, guide nucleic acids comprise a portion or all of a sequence as set forth in TABLES 2, and 5 and SEQ ID NOs: 236 and 350-352. In some embodiments, guide nucleic acids comprise at least 60, at least 65, at last 70, at least 75, at least 80, at least 85, at least 86, at least 87, at least 88, or at least 89 contiguous nucleotides of a sequence selected from any of SEQ ID NOs: 236, 275-427, 457-460, 462-465, 476-480, and 597-601.

In some embodiments, compositions disclosed herein comprises a spacer sequence that is at least 70%, at least 75%, at least 80%, at least 85%, at least 90%, at least 95%, or 100% identical to any one of the sequences as set forth in TABLE 2, and comprising a repeat sequence that is at least 70%, at least 75%, at least 80%, at least 85%, at least 90%, at least 95%, or 100% identical to SEQ ID NO: 350.

In some embodiments, compositions, systems and methods described herein comprise a sequence that is at least 65%, at least 70%, at least 75%, at least 80%, at least 85%, at least 90%, at least 92%, at least 95%, at least 97%, at least 99%, or 100% identical to any one of the sequences as set forth in TABLE 5.

In some embodiments, the sequences in any one of TABLES 1, 2, 3, 4, and 5 and SEQ ID NOs: 236, and 351-352 can be modified.

In some embodiments, the modification includes at least one phosphorothioate (PS) linkage. In some embodiments, the modification includes at least one 2′-O-Methyl oligonucleotide (OMe). In some embodiments, the modification includes at least one locked nucleic acid (LNA). In some embodiments, the modification includes at least one Phosphorodiamidate morpholino oligonucleotide (PMO). In some embodiments, the modification includes at least one or more peptide nucleic acid (PNA). In some embodiments, the first 3 and last 3 amino acids are O-Me modified, and the first 3 and last 2 linkages are phosphorothioate linkages. In some embodiments, the sequence is modified mN*mN*mN*I . . . NNNmN*mN*mN where m is ‘2’ O-Me modified sugar moiety and the * denotes a PS linkage.

### Nucleic Acid Linkers

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises two or more linkers. In some embodiments, at least two or more linkers are the same. In some embodiments, at least two or more linkers are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′ (SEQ ID NO: 236).

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### IV. Effector Proteins

In some embodiments, compositions provided herein comprise one or more effector proteins. In some embodiments, compositions and systems described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature.

An effector protein may be brought into proximity of a target nucleic acid in the presence of a guide nucleic acid. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid, which may direct the modification activity of the effector protein.

In some embodiments, the effector protein is a programmable nuclease (e.g., a CRISPR-associated (Cas) protein) that modifies a target sequence in a target nucleic acid. In some embodiments, the effector protein is a programmable nuclease that modifies a region of the nucleic acid that is near, but not within, to the target sequence. Effector proteins may cleave nucleic acids, including single stranded RNA (ssRNA), double stranded DNA (dsDNA), and single-stranded DNA (ssDNA). Effector proteins may provide cis cleavage activity, trans cleavage activity, nickase activity, or a combination thereof.

An effector protein may function as a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., a dimer or a multimer). An effector protein, when functioning in a multiprotein complex, may have only one functional activity (e.g., binding to a guide nucleic acid), while other effector proteins present in the multiprotein complex are capable of another functional activity (e.g., modifying a target nucleic acid).

In some embodiments, the effector protein is a Type V Cas protein. In some embodiments, the effector protein is CasPhi.12 or a variant thereof. In some embodiments, the effector protein is CasM.265466 or a variant thereof. A CasPhi.12 is around half of the size of Cas9, and CasM.265466 is around one third of the size of Cas9. The smaller sizes of CasPhi. 12 and CasM.265466 make them ideal to be packaged together with their corresponding guide RNAs into a single AAV vector, thus overcoming the drawbacks of dual AAV vector systems.

TABLE 7 provides illustrative amino acid sequences of effector proteins. In some embodiments, the amino acid sequence of an effector protein is at least 65%, at least 70%, at least 75%, at least 80%, at least 85%, at least 90%, at least 95%, at least 97%, or at least 98%, at least 99%, or 100% identical to the sequence as set forth in TABLE 7. In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 7.

In some embodiments, compositions, systems, and methods comprise an effector protein or uses thereof, wherein the amino acid sequence of the effector protein comprises at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500, at least about 520, at least about 540, at least about 560, at least about 580, at least about 600, at least about 620, at least about 640, at least about 660, at least about 680, or at least about 700 contiguous amino acids of a sequence in TABLE 7.

In some embodiments, the effector protein may also comprise at least one additional amino acid relative to the naturally-occurring effector protein. For example, the effector protein may comprise an addition of a nuclear localization signal relative to the natural occurring effector protein. In some embodiments compositions and systems described herein may comprise a nuclear localization signal (NLS). In some embodiments, the effector protein is linked to a nuclear localization signal. In some embodiments, compositions and systems described herein may comprise a NLS sequence that is adjacent to the N terminal of the effector protein or that is adjacent to the C terminal of the effector protein, or both. In some embodiments, a nuclear localization signal can comprise a sequence of—N-MAPKKKRKVGIHGVPAA-C(SEQ ID NO: 234). In some embodiments, a nuclear localization signal can comprise a sequence of—N-KRPAATKKAGQAKKKK-C(SEQ ID NO: 235). In certain embodiments, the nucleotide sequence encoding the effector protein is codon optimized (e.g., for expression in a eukaryotic cell) relative to the naturally occurring sequence.

TABLE 6 provides exemplary nuclear localization sequences.

TABLE 6
Exemplary Nuclear Localization Sequences

| SEQ ID | | |
|---|---|---|
| NO: | Description | SEQUENCES |
| 245 | NLS | PKKKRKVGIHGVPAA |
| 246 | NLS | KRPAATKKAGQAKKKK |
| 247 | NLS | KR(K/R)R |
| 248 | NLS | (P/R)XXKR(\hat{ }DE)(K/R) |
| 249 | NLS | KRX(W/F/Y)XXAF |
| 250 | NLS | (R/P)XXKR(K/R)(\hat{ }DE) |
| 251 | NLS | LGKR(K/R)(W/F/Y) |
| 252 | NLS | KRX₁₀K(K/R)(K/R) |
| 256 | NLS | K(K/R)RK |
| 254 | NLS | KRX₁₁K(K/R)(K/R) |
| 255 | NLS | KRX₁₂K(K/R)(K/R) |
| 256 | NLS | KRX₁₀K(K/R)X(K/R) |
| 257 | NLS | KRX₁₁K(K/R)X(K/R) |
| 258 | NLS | KRX₁₂K(K/R)X(K/R) |
| 259 | NLS | APKKKRKVGIHGVPAA |
| 260 | EEP | GLFXALLXLLXSLWXLLLXA |
| 261 | EEP | GLFHALLHLLHSLWHLLLHA |

wherein X is any naturally occurring amino acid; and (\hat{ }DE) is any naturally occurring amino acid except Asp or Glu

An effector protein may function as a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., a dimer or a multimer). An effector protein, when functioning in a multiprotein complex, may have only one functional activity (e.g., binding to a guide nucleic acid), while other effector proteins present in the multiprotein complex are capable of another functional activity (e.g., modifying a target nucleic acid).

TABLE 7 provides illustrative amino acid sequences of effector proteins. In some embodiments, an effector protein is at least 65%, at least 70%, at least 75%, at least 80%, at least 85%, at least 90%, at least 95%, at least 97%, or at least 98%, at least 99%, or 100% identical to the sequence as set forth in TABLE 7.

In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to SEQ ID NO: 232, wherein the amino acid residue at position 26, is arginine (R). In some embodiments, an effector protein comprises an amino acid sequence that is at least 65%, at least 70%, at least 75%, at least 80%, at least 85%, at least 90%, at least 95%, at least 97%, or at least 98%, or at least 99%, identical to SEQ ID NO: 429 wherein the amino acid residue at position 220 is arginine (R). Bold and italicized text indicates the NLS. Underlined text indicates a 3xFLAG tag.

TABLE 7
Exemplary Effector Proteins

| Effector | | SEQ ID |
|---|---|---|
| protein | Amino Acid Sequence | NO: |
| CasPhi.12 | MIKPTVSQFLTPGFKLIRNHSRTAGLKLKNEGEEACKKFV | 230 |
| | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITA YRERYDKLESSIKLDAIKQLTSEQKIEVD | |
| | NYNNNFTPQNTKQIVCSKLNINPNDLPWDKMISGTHFISE | |
| | KAQVSNKSEIYFTSTDKGKTKDVMKSDYKWFQDYKPKLSK | |
| | EVRDALSDIEWRLRRESLEFNKLSKSREQDARQLANWISS | |
| | MCDVIGIENLVKKNNFFGGSGKREPGWDNFYKPKKENRWW | |
| | INAIHKALTELSQNKGKRVILLPAMRTSITCPKCKYCDSK | |
| | NRNGEKFNCLKCGIELNADIDVATENLATVAITAQSMPKP | |
| | TCERSGDAKKPVRARKAKAPEFHDKLAPSYTVVLREAV | |
| CasPhi.12 | PKKKRKVGIHGVPAAMIKPTVSQFLTPGFKLIRNHSRTAG | 231 |
| with NLS | LKLKNEGEEACKKFVRENEIPKDECPNFQGGPAIANIIAK | |
| | SREFTEWEIYQSSLAIQEVIFTLPKDKLPEPILKEEWRAQ | |
| | WLSEHGLDTVPYKEAAGLNLIIKNAVNTYKGVQVKVDNKN | |
| | KNNLAKINRKNEIAKLNGEQEISFEEIKAFDDKGYLLQKP | |
| | SPNKSIYCYQSVSPKPFITSKYHNVNLPEEYIGYYRKSNE | |
| | PIVSPYQFDRLRIPIGEPGYVPKWQYTFLSKKENKRRKLS | |
| | KRIKNVSPILGIICIKKDWCVFDMRGLLRTNHWKKYHKPT | |
| | DSINDLFDYFTGDPVIDTKANVVRFRYKMENGIVNYKPVR | |
| | EKKGKELLENICDQNGSCKLATVDVGQNNPVAIGLFELKK | |
| | VNGELTKTLISRHPTPIDFCNKITAYRERYDKLESSIKLD | |
| | AIKQLTSEQKIEVDNYNNNFTPQNTKQIVCSKLNINPNDL | |
| | PWDKMISGTHFISEKAQVSNKSEIYFTSTDKGKTKDVMKS | |
| | DYKWFQDYKPKLSKEVRDALSDIEWRLRRESLEFNKLSKS | |
| | REQDARQLANWISSMCDVIGIENLVKKNNFFGGSGKREPG | |
| | WDNFYKPKKENRWWINAIHKALTELSQNKGKRVILLPAMR | |
| | TSITCPKCKYCDSKNRNGEKFNCLKCGIELNADIDVATEN | |
| | LATVAITAQSMPKPTCERSGDAKKPVRARKAKAPEFHDKL | |
| | APSYTVVLREAVKRPAATKKAGQAKKKK | |
| CasM.265 | MSVLTRKVQLIPVGDKEERDRVYKYLRDGIEAQNRAMNLY | 428 |
| 466 | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| 3x Flag- | MDYKDHDGDYKDHDIDYKDDDDKMAPKKKRKVGIHGVPAA | 430 |
| SV40NLS- | MSVLTRKVQLIPVGDKEERDRVYKYLRDGIEAQNRAMNLY | |
| CasM.265 | MSGLYFAAINEASKEDRKELNQLYSRIATSSKGSAYTTDI | |
| 466- | EFPTGLASTSTLSMAVRQDFTKSLKDGLMYGRVSLPTYRK | |
| nucleoplas | DNPLFVDVRFVALRGTKQKYNGLYHEYKSHTEFLDNLYSS | |
| min NLS | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENKKRPAATKKAGQAKKKK | |

In some embodiments, compositions, systems, and methods described herein comprise an effector protein or a nucleic acid encoding the effector protein, wherein the effector protein comprises one or more amino acid alterations relative to a sequence recited in TABLE 7. In some embodiments, an amino acid alteration comprises a deletion of an amino acid. In some embodiments, an amino acid alteration comprises an insertion of an amino acid. In some embodiments, an amino acid alteration comprises a conservative amino acid substitution. In some embodiments, an amino acid alteration comprises a non-conservative amino acid substitution. In some embodiments, one or more amino acid alterations comprises a combination of one or more conservative amino acid substitutions and one or more non-conservative amino acid substitutions. When describing a conservative amino acid substitution herein, reference is made to the replacement of one amino acid for another such that the replacement takes place within a family of amino acids that are related in their side chains. Conversely, when describing a non-conservative alteration (e.g., non-conservative substitution), reference is made to the replacement of one amino acid residue for another that does not have a related side chain. It is understood that genetically encoded amino acids can be divided into four families having related side chains: (1) acidic (negatively charged): Asp (D), Glu (E); (2) basic (positively charged): Lys (K) Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl. Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

In some embodiments, effector proteins are engineered variants of CasM.265466 (SEQ ID NO: 428) and CasPhi.12 (SEQ ID NO: 230). Engineered variants of CasM.265466 (SEQ ID NO: 428) and CasPhi.12 (SEQ ID NO: 230) may comprise amino acid substitutions relative to SEQ ID NO: 428 and SEQ ID NO: 230, respectively. Exemplary amino acid substitutions are described in TABLES 8-11. The amino acid substitutions in TABLE 8 and TABLE 9 may be combined. The amino acid substitutions in TABLE 8 and TABLE 9 may be combined with other amino acid alterations described herein. The amino acid substitutions in TABLE 10 and TABLE 11 may be combined. The amino acid substitutions in TABLE 10 and TABLE 11 may be combined with other amino acid alterations described herein.

In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 98%, at least 99%, or 100% identical to any one of the sequences as set forth in TABLE 7. In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 7, wherein the amino acid residue at position 26, relative to SEQ ID NO: 232, remains unchanged. In other words, the residue of the amino acid sequence that aligns with position 26 of SEQ ID NO: 232 is an arginine when the amino acid sequence is aligned with SEQ ID NO: 230 for maximum identity. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 7. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 7, wherein the amino acid residue at position 26, relative to SEQ ID NO: 230, remains unchanged.

In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 98%, at least 99%, or 100% identical to any one of the sequences as set forth in TABLE 7. In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 7, wherein the amino acid residue at position 220, relative to SEQ ID NO: 429, remains unchanged. In other words, the residue of the amino acid sequence that aligns with position 220 of SEQ ID NO: 429 is an arginine when the amino acid sequence is aligned with SEQ ID NO: 428 for maximum identity. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 7. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 7, wherein the amino acid residue at position 220, relative to SEQ ID NO: 428, remains unchanged.

In certain embodiments, the amino acid sequence of the effector protein is based on SEQ ID NO: 230 and is modified at position 26. In some embodiments the modification at position 26 is from leucine to arginine (L26R). In some embodiments, the amino acid sequence of the effector protein is at least 95%, 96%, 97%, 98%, or 99% identical to SEQ ID NO: 232. In some embodiments, the amino acid sequence of the effector protein comprises or consists of SEQ ID NO: 232.

In certain embodiments, the amino acid sequence of the effector protein is based on SEQ ID NO: 230 and is modified at position 109. In some embodiments the modification at position 109 is from glutamic acid to arginine (E109R). In some embodiments, the amino acid sequence of the effector protein is at least 95%, 96%, 97%, 98%, or 99% identical to SEQ ID NO: 262. In some embodiments, the amino acid sequence of the effector protein comprises or consists of SEQ ID NO: 262.

In certain embodiments, the amino acid sequence of the effector protein is based on SEQ ID NO: 230 and is modified at position 208. In some embodiments the modification at position 208 is from histidine to arginine (H208R). In some embodiments, the amino acid sequence of the effector protein is at least 95%, 96%, 97%, 98%, or 99% identical to SEQ ID NO: 263. In some embodiments, the amino acid sequence of the effector protein comprises or consists of SEQ ID NO: 263.

In certain embodiments, the amino acid sequence of the effector protein is based on SEQ ID NO: 230 and is modified at position 184. In some embodiments the modification at position 184 is from lysine to arginine (K184R). In some embodiments, the amino acid sequence of the effector protein is at least 95%, 96%, 97%, 98%, or 99% identical to SEQ ID NO: 264. In some embodiments, the amino acid sequence of the effector protein comprises or consists of SEQ ID NO: 264.

In certain embodiments, the amino acid sequence of the effector protein is based on SEQ ID NO: 230 and is modified at position 38. In some embodiments the modification at position 38 is from lysine to arginine (K38R). In some embodiments, the amino acid sequence of the effector protein is at least 95%, 96%, 97%, 98%, or 99% identical to SEQ ID NO: 265. In some embodiments, the amino acid sequence of the effector protein comprises or consists of SEQ ID NO: 265.

In certain embodiments, the amino acid sequence of the effector protein is based on SEQ ID NO: 230 and is modified at position 182. In some embodiments the modification at position 182 is from leucine to arginine (L182R). In some embodiments, the amino acid sequence of the effector protein is at least 95%, 96%, 97%, 98%, or 99% identical to SEQ ID NO: 266. In some embodiments, the amino acid sequence of the effector protein comprises or consists of SEQ ID NO: 266.

In certain embodiments, the amino acid sequence of the effector protein is based on SEQ ID NO: 230 and is modified at position 183. In some embodiments the modification at position 183 is from glutamine to arginine (Q183R). In some embodiments, the amino acid sequence of the effector protein is at least 95%, 96%, 97%, 98%, or 99% identical to SEQ ID NO: 267. In some embodiments, the amino acid sequence of the effector protein comprises or consists of SEQ ID NO: 267.

In certain embodiments, the amino acid sequence of the effector protein is based on SEQ ID NO: 230 and is modified at position 108. In some embodiments the modification at position 108 is from serine to arginine (S108R). In some embodiments, the amino acid sequence of the effector protein is at least 95%, 96%, 97%, 98%, or 99% identical to SEQ ID NO: 268. In some embodiments, the amino acid sequence of the effector protein comprises or consists of SEQ ID NO: 268.

In certain embodiments, the amino acid sequence of the effector protein is based on SEQ ID NO: 230 and is modified at position 198. In some embodiments the modification at position 198 is from serine to arginine (S198R). In some embodiments, the amino acid sequence of the effector protein is at least 95%, 96%, 97%, 98%, or 99% identical to SEQ ID NO: 269. In some embodiments, the amino acid sequence of the effector protein comprises or consists of SEQ ID NO: 269.

In certain embodiments, the amino acid sequence of the effector protein is based on SEQ ID NO: 230 and is modified at position 114. In some embodiments the modification at position 114 is from threonine to arginine (T114R). In some embodiments, the amino acid sequence of the effector protein is at least 95%, 96%, 97%, 98%, or 99% identical to SEQ ID NO: 270. In some embodiments, the amino acid sequence of the effector protein comprises or consists of SEQ ID NO: 270.

In some embodiments, the effector protein is a Type V Cas protein. In some embodiments, the effector protein is CasM.265466 or a variant thereof. A CasM.265466 is around one third of the size of Cas9. The smaller size of CasM.265466 make it ideal to be packaged together with its corresponding guide RNAs into a single AAV vector, thus overcoming the drawbacks of dual AAV vector systems.

TABLE 8 provides illustrative amino acid sequences of effector proteins. In some embodiments, an effector protein is at least 65%, at least 70%, at least 75%, at least 80%, at least 85%, at least 90%, at least 95%, at least 97%, or at least 98%, at least 99%, or 100% identical to the sequence as set forth in TABLE 8.

In some embodiments, the effector protein is an engineered effector protein and comprises an amino acid sequence that is at least 90%, at least 95%, at least 97%, at least 98%, or at least 99% identical to SEQ ID NO: 428, wherein the polypeptide comprises at least one amino acid substitution relative to SEQ ID NO: 428, wherein the amino acid substitution is at a position selected from K58, 180, T84, K105, N193, C202, S209, G210, A218, D220, E225, C246, N286, M295, M298, A306, Y315, Q360, and a combination thereof. In some embodiments, the polypeptide comprises an amino acid sequence that is 100% identical to SEQ ID NO: 428, with the exception of at least one amino acid substitution relative to SEQ ID NO: 428, wherein the amino acid substitution is a position selected from K58, 180, T84, K105, N193, C202, S209, G210, A218, D220, E225, C246, N286, M295, M298, A306, Y315, Q360, and a combination thereof. In some embodiments, the amino acid substitution is selected from K58X, 180X, T84X, K105X, N193X, C202X, S209X, G210X, A218X, D220X, E225X, C246X, N286X, M295X, M298X, A306X, Y315X, and Q360X, wherein X is selected from R, K, and H.

In some embodiments, the effector protein is an engineered effector protein and comprises an amino acid sequence that is at least 90%, at least 95%, at least 97%, at least 98%, or at least 99% identical to SEQ ID NO: 428, wherein the polypeptide comprises at least one amino acid substitution relative to SEQ ID NO: 428, wherein the amino acid substitution is selected from I80R, T84R, K105R. C202R, G210R, A218R, D220R, E225R, C246R, Q360R, 180K, T84K, G210K, N193K, C202K, A218K, D220K, E225K, C246K, N286K, A306K, Q360K, 180H, T84H, K105H, G210H, C202H, A218H, D220H, E225H, C246H, Q360H, K58W, S209F, M295W, M298L, Y315M, D220R/A306K and D220R/K250N and a combination thereof. In some embodiments, the polypeptide comprises an amino acid sequence that is 100% identical to SEQ ID NO: 428, with the exception of at least one amino acid substitution relative to SEQ ID NO: 428, wherein the amino acid substitution is selected from ISOR, T84R, K105R, C202R, G210R, A218R, D220R, E225R, C246R, Q360R, 180K, T84K, G210K, N193K, C202K, A218K, D220K, E225K, C246K, N286K, A306K, Q360K, 180H, T84H, K105H, G210H, C202H, A218H, D220H, E225H, C246H, Q360H, K58W, S209F, M295W, M298L, Y315M, D220R/A306K, D220R/K250N, D220R/E335Q and a combination thereof. In some aspects, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 428, wherein the polypeptide comprises at least one amino acid substitution relative to SEQ ID NO: 428, wherein the amino acid substitution is selected from D237A, D418A, D418N, E335A, and E335Q, and a combination thereof. In some embodiments, the polypeptide comprises an amino acid sequence that is 100% identical to SEQ ID NO: 428, with the exception of at least one amino acid substitution relative to SEQ ID NO: 428, wherein the amino acid substitution is selected from D237A, D418A, D418N, E335A, and E335Q, and a combination thereof. In some aspects, these engineered effector proteins demonstrate reduced or abolished nuclease activity relative to the wild-type effector protein. TABLE 9 provides the exemplary amino acid alterations relative to SEQ ID NO: 428 useful in compositions, systems, and methods described herein.

In some embodiments, the effector protein is an engineered effector protein and comprises an amino acid sequence that is 100% identical to SEQ ID NO: 428, with the exception of two amino acid substitutions at D220 and E335 relative to SEQ ID NO: 428. In some embodiments, the amino acid substitutions are D220R and 335Q. In some embodiments, the engineered effector protein comprises or consists of SEQ ID NO: 452.

TABLE 8
Exemplary Amino Acid Sequences of
Engineered Variants of CasM.265466

| Effector | | SEQ ID |
|---|---|---|
| protein | Amino Acid Sequence | NO: |
| CasM.265 | MSVLTRKVQLIPVGDKEERDRVYKYLRDGIEAQNRAMNLY | 429 |
| 466 D220R | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMRIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 431 |
| 466 K58W | MSGLYFAAINEASKEDRWELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 432 |
| 466 | MSGLYFAAINEASKEDRKELNQLYSRIATSSKGSAYTTDI | |
| A218K | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMKMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 433 |
| 466 | MSGLYFAAINEASKEDRKELNQLYSRIATSSKGSAYTTDI | |
| M295W | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKWKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 434 |
| 466 | MSGLYFAAINEASKEDRKELNQLYSRIATSSKGSAYTTDI | |
| M298L | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPLDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 435 |
| 466 | MSGLYFAAINEASKEDRKELNQLYSRIATSSKGSAYTTDI | |
| N193K | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQKIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 436 |
| 466 | MSGLYFAAINEASKEDRKELNQLYSRIATSSKGSAYTTDI | |
| Y315M | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHMVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 437 |
| 466 S209F | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFFGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 438 |
| 466 I80K | MSGLYFAAINEASKEDRKELNQLYSRIATSSKGSAYTTDK | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 439 |
| 466 E225K | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKKIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 440 |
| 466 | MSGLYFAAINEASKEDRKELNQLYSRIATSSKGSAYTTDI | |
| N286K | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSKGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 441 |
| 466 | MSGLYFAAINEASKEDRKELNQLYSRIATSSKGSAYTTDI | |
| A306K | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEKNWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 442 |
| 466 E335Q | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLQNLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 443 |
| 466 D237A | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVALGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.265 | MSVLTRKVQLIPVGDKEERDRVYKYLRDGIEAQNRAMNLY | 444 |
| 466 D418A | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNAAFNAARNIAMSTEFQSGKKTKKQ | |
| | KKEQHENK | |
| CasM.265 | MSVLTRKVQLIPVGDKEERDRVYKYLRDGIEAQNRAMNLY | 445 |
| 466 D418N | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNANFNAARNIAMSTEFQSGKKTKKQ | |
| | KKEQHENK | |
| CasM.265 | MSVLTRKVQLIPVGDKEERDRVYKYLRDGIEAQNRAMNLY | 446 |
| 466 E335A | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMDIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLANLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasM.2654 | MSVLTRKVQLIPVGDKEERDRVYKYLRDGIEAQNRAMNLY | 452 |
| 66 D220R- | MSGLYFAAINEASKEDRKELNQLYSRIATSSKGSAYTTDI | |
| E335Q | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMRIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLQNLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |

TABLE 9
Exemplary Amino Acid Alterations Relative to SEQ ID NO: 428

| Effects | Amino Acid Alterations |
|---|---|
| | At least one substitution (i.e., with R, K or H) selected from |
| | K58, I80, T84, K105, N193, C202, S209, G210, A218, D220, |
| | E225, C246, N286, M295, M298, A306, Y315, and Q360 |
| Enhanced nuclease activity relative to | I80R, T84R, K105R, C202R, G210R, A218R, D220R, E225R, |
| the wild-type effector protein | C246R, Q360R, 180K, T84K, G210K, N193K, C202K, A218K, |
| | D220K, E225K, C246K, N286K, A306K, Q360K, I80H, |
| | T84H, K105H, G210H, C202H, A218H, D220H, E225H, |
| | C246H, Q360H, K58W, S209F, M295W, M298L, Y315M |
| | Double mutations: D220R/A306K, D220R/K250N |
| Reduced or abolished nuclease | D237A, D418A, D418N, E335A, E335Q |
| activity relative to the wild-type | |
| effector protein | |

TABLE 10 provides illustrative amino acid sequences of effector proteins. In some embodiments, an effector protein is at least 65%, at least 70%, at least 75%, at least 80%, at least 85%, at least 90%, at least 95%, at least 97%, or at least 98%, at least 99%, or 100% identical to the sequence as set forth in TABLE 10.

In some embodiments, the effector protein is an engineered effector protein and comprises an amino acid sequence that is at least 90%, at least 95%, at least 97%, at least 98%, or at least 99% identical to SEQ ID NO: 230, wherein the polypeptide comprises at least one amino acid substitution relative to SEQ ID NO: 230, wherein the amino acid substitution is at a position selected from 12, T5, K15, R18, H20, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139, N148, L149, E157, E164, E166, E170, Y180, L182, Q183, K184, S186, K189, S196, S198, K200, 1203, S205, K206, Y207, H208, N209, Y220, S223, E258, K281, K348, N355, S362, 1406, 1435, 1471, 1489, Y490, F491, D495, K496, K498, K500, D501, V502, K504, S505, D506, V521, N568, S579, Q612, S638, F701, P707, and a combination thereof. In some embodiments, the polypeptide comprises an amino acid sequence that is 100% identical to SEQ ID NO: 230, with the exception of at least one amino acid substitution relative to SEQ ID NO: 230, wherein the amino acid substitution is at a position selected from 12, T5, K15, R18, H20, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139, N148, L149, E157, E164, E166, E170, Y180, L182, Q183, K184, S186, K189, S196, S198, K200, I203, S205, K206, Y207, H208, N209, Y220, S223, E258, K281, K348, N355, S362, N406, K435, 1471, 1489, Y490, F491, D495, K496, K498, K500, D501, V502, K504, S505, D506, V521, N568, S579, Q612, S638, F701, P707, and a combination thereof. In some embodiments, the amino acid substitution is selected from 12X, TSX, K15X, R18X, H20X, S21X, L26X, N30X, E33X, E34X, A35X, K37X, K38X, R41X, N43X, Q54X, Q79RX, K92EX, K99RX, S108X, E109X, H110X, G111X, D113X, T114X, P116X, K118X, E119X, A121X, N132X, K135X, Q138X, V139X, N148X, L149X, E157X, E164X, E166X, E170X, Y180X, L182X, Q183X, K184X, S186X, K189X, S196X, S198X, K200X. I203X, S205X, K206X, Y207X, H208X, N209X, Y220X, S223X, E258X, K281X, K348X, N355X, S362X, N406X, K435X, 1471X, 1489X, Y490X, F491X, D495X, K496X, K498X, K500X, D501X, V502X, K504X, S505X, D506X, V521X, N568X, S579X, Q612X, S638X, F701X, P707X, wherein X is selected from R, K, and H.

In some embodiments, the effector protein is an engineered effector protein and comprises an amino acid sequence that is at least 90%, at least 95%, at least 97%, at least 98%, or at least 99% identical to SEQ ID NO: 230, wherein the polypeptide comprises at least one amino acid substitution relative to SEQ ID NO: 230 wherein the amino acid substitution is selected from T5R, L26R, L26K, A121Q, V139R, S198R, S223P, E258K, 1471T, S579R, F701R, P707R, K189P, S638K, Q54R, Q79R, Y220S, N406K, E119S, K92E, K435Q, N568D, and V521T, and a combination thereof. In some embodiments, the polypeptide comprises an amino acid sequence that is 100% identical to SEQ ID NO: 230, with the exception of at least one amino acid substitution relative to SEQ ID NO: 230, wherein the amino acid substitution is selected from TSR, L26R, L26K, A121Q, V139R, S198R, S223P, E258K, I471T, S579R, F701R, P707R, K189P. S638K, Q54R, Q79R, Y220S, N406K, E119S. K92E, K435Q, N568D, and V521T, and a combination thereof. In some aspects, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 230, wherein the polypeptide comprises at least one amino acid substitution relative to SEQ ID NO: 230 wherein the amino acid substitution is selected from L26K/A121Q, L26R/A121Q. K99R/L149R, K99R/N148R, L149R/H208R, S362R/L26R L26R/N148R. L26R/H208R, N30R/N148R, L26R/K99R, L26R/P707R, L26R/L149R, L26R/N30R, L26R/N355R, L26R/K281R, L26R/S108R, L26R/K348R, T5R/V139R, I2R/V139R, K99R/S186R, L26R/A673G, L26R/Q674R, S579R/L26K, F701R/E258K, T5R/L26K, L26R/K435Q, L26K/E567Q, L26R/G685R, L26R/Q674K, L26R/P699R, L26R/T70E, L26R/Q232R, L26R/T252R, L26R/P679R, L26R/E83K, L26R/B73P, L26R/K248E, L26R, T5R/S223P, S579R/S223P, L26R/S223P, T5R/A121Q, L26R/A696R, S198R/1471T, L26R/N153R, L26R/E682R, L26R/D703R, Q612R/126K, L26R/1471T, K348R/L26K, S579R/1471T, L26R/V228R, T5R/S638K, S579R/K189P, S579R/E258K, L26R/K260R, L26R/S638K, S579R/Y220S, T5R/I471T, L26R/F233R, L26R/V521T, F701R/A121Q, L26R/G361R, S198R/E258K, L26R/S472R, T5R/Y220S, L26R/A150K, L26R/S684R, L26R/E157R, L26R/K248R, F701R/L26K, S198R/N406K, S198R/Y220S, S198R/S638K, S198R/V521T, S579R/A121Q. K348R/Y220S, S198R/K189P, L26R/E242R, L26R/K678R, T5R/1406K, L26R/1158K, TSR/V521T, L26R/N259R, L26R/K257R, L26R/K256R, T5R/K189P, L26R/C405R, S579R/V521T, S579R/N406K, T5R/K92E, T5R/E258K, L26R/197R, S579R/S638K, TSR/K435Q, F701R/S638K, L26R/L236R, F701R/1471T, Q612R/S223P, F701R/S223P, S198R/E119S, S579R/K92E, L26R/E715R, Q612R/1471T, F701R/Y220S, S198R/S223P, and L26R/K266R, and a combination thereof. In some embodiments, the polypeptide comprises an amino acid sequence that is 100% identical to SEQ ID NO: 230, with the exception of at least one amino acid substitution relative to SEQ ID NO: 230, wherein the amino acid substitution is selected from L26K/A121Q, L26R/A121Q, K99R/L149R, K99R/N148R, L149R/H208R, S362R/L26R L26R/N148R, L26R/H208R, N30R/N148R, L26R/K99R, L26R/P707R, L26R/L149R, L26R/N30R, L26R/N355R, L26R/K281R, L26R/S108R, L26R/K348R, T5R/V139R, I2R/V139R, K99R/S186R, L26R/A673G, L26K/E567Q, L26R/Q674R, S579R/L26K, F701R/E258K, T5R/L26K, L26R/K435Q, L26R/G685R, L26R/Q674K, L26R/P699R, L26R/T70E, L26R/Q232R, L26R/T252R, L26R/P679R, L26R/B83K, L26R/E73P. L26R/K248E, L26R, T5R/S223P, S579R/S223P, L26R/S223P, T5R/A121Q, L26R/A696R, S198R/1471T, L26R/N153R, L26R/E682R, L26R/D703R, Q612R/L26K, L26R/1471T, K348R/126K, S579R/1471T, L26R/V228R, T5R/S638K, S579R/K189P, S579R/E258K, L26R/K260R, L26R/S638K, S579R/Y220S, T5R/1471T, L26R/F233R, L26R/V521T, F701R/A121Q, L26R/G361R, S198R/E258K, L26R/S472R, T5R/Y220S, L26R/A150K, L26R/S684R, L26R/E157R, L26R/K248R, F701R/L26K, S198R/N406K, S198R/Y220S, S198R/S638K, S198R/V521T, S579R/A121Q, K348R/Y220S, S198R/K189P, L26R/E242R, L26R/K678R, T5R/N406K, L26R/1158K, TSR/V521T, L26R/N259R, L26R/K257R, L26R/K256R, T5R/K189P, L26R/C405R, S579R/V521T, S579R/N406K, TSR/K92E, T5R/E258K, L26R/197R, S579R/S638K, T5R/K435Q, F701R/S638K, L26R/L236R, F701R/I471T, Q612R/S223P, F701R/S223P, S198R/E119S, S579R/K92E, L26R/E715R, Q612R/I47IT, F701R/Y220S, S198R/S223P, and L26R/K266R, and a combination thereof. In some aspects, these engineered effector proteins demonstrate enhanced nuclease activity relative to the wild-type effector protein.

In some embodiments, the polypeptide comprises an amino acid sequence that is 100% identical to SEQ ID NO: 230, with the exception of at least two amino acid substitutions relative to SEQ ID NO: 230, wherein the amino acid substitutions comprise L26K/E567Q. In some embodiments, the polypeptide comprises or consists of SEQ ID NO: 451.

In some embodiments, the effector protein is an engineered effector protein and comprises an amino acid sequence that is at least 90%, at least 95%, at least 97%, at least 98%, or at least 99% identical to SEQ ID NO: 230, wherein the polypeptide comprises at least one amino acid substitution relative to SEQ ID NO: 230 wherein the amino acid substitution is selected from E157A, E164A, E164L, E166A, B166I , E170A, 1489A, 1489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, and a combination thereof. In some embodiments, the polypeptide comprises an amino acid sequence that is 100% identical to SEQ ID NO: 230, with the exception of at least one amino acid substitution relative to SEQ ID NO: 230, wherein the amino acid substitution is selected from E157A, E164A, E164L, E166A, B166I, E170A, 1489A, 1489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, and a combination thereof. In some embodiments, these engineered effector proteins comprise a nickase activity.

In some embodiments, the effector protein is an engineered effector protein and comprises an amino acid sequence that is at least 90%, at least 95%, at least 97%, at least 98%, or at least 99% identical to SEQ ID NO: 230, wherein amino acids S478-S505 have been deleted. In some embodiments, the effector protein is an engineered effector protein that is at least 90%, at least 95%, at least 97%, at least 98%, or at least 99% identical to SEQ ID NO: 230, wherein amino acids S478-S505 have been deleted and replaced with SDLYIERGGDPRDVHQQVETKPKGKRKSEIRILKIR (SEQ ID NO: 447) or SDYIVDHGGDPEKVFFETKSKKDKTKRYKRR (SEQ ID NO: 448). In some embodiments, the effector protein is an engineered effector protein and comprises an amino acid sequence that is at least 90%, at least 95%, at least 97%, at least 98%, at least 99% identical, or is 100% identical to SEQ ID NO: 449. In some embodiments, the effector protein is an engineered effector protein and comprises an amino acid sequence that is at least 90%, at least 95%, at least 97%, at least 98%, at least 99% identical, or is 100% identical to SEQ ID NO: 450.

In some embodiments, the effector protein is an engineered effector protein and comprises an amino acid sequence that is at least 90%, at least 95%, at least 97%, at least 98%, or at least 99% identical to SEQ ID NO: 230, wherein the polypeptide comprises at least one amino acid substitution relative to SEQ ID NO: 230 wherein the amino acid substitution is selected from D369A, D369N, D658A, D658N, E567A, E567Q, and a combination thereof. In some embodiments, the polypeptide comprises an amino acid sequence that is 100% identical to SEQ ID NO: 230, with the exception of at least one amino acid substitution relative to SEQ ID NO: 230, wherein the amino acid substitution is selected from D369A, D369N, D658A, D658N, E567A, E567Q, and a combination thereof. In some aspects, these engineered effector proteins demonstrate reduced or abolished nuclease activity relative to the wild-type effector protein. TABLE 11 provides the exemplary amino acid alterations relative to SEQ ID NO: 230 useful in compositions, systems, and methods described herein.

TABLE 10
Exemplary Amino Acid Sequences of Engineered Variants of CasPhi.12

| Effector | | SEQ ID |
|---|---|---|
| protein | Amino Acid Sequence | NO: |
| CasPhi.12 | MIKPTVSQFLTPGFKLIRNHSRTAGRKLKNEGEEACKKFV | 232 |
| L26R | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| 3x Flag- | MDYKDHDGDYKDHDIDYKDDDDKMAPKKKRKVGIHGVPAA | 233 |
| SV40NLS- | MIKPTVSQFLTPGFKLIRNHSRTAGRKLKNEGEEACKKFV | |
| CasPhi12 | RENEIPKDECPNFQGGPAIANIIAKSREFTEWEIYQSSLA | |
| L26R-NLS | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAVKGR | |
| | RPRKRPARQKRKRNS | |
| CasPhi.12 | MIKPTVSQFLTPGFKLIRNHSRTAGLKLKNEGEEACKKFV | 243 |
| E567A | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIANLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 244 |
| E567Q | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIQNLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 262 |
| E109R | RENEIPKDECPNFQGGPAIANIIAKSREFTEWEIYQSSLA | |
| | IQEVIFTLPKDKLPEPILKEEWRAQWLSRHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 263 |
| H208R | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYRNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 264 |
| K184R | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQRPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKRFV | 265 |
| K38R | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 266 |
| L182R | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLRQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 267 |
| Q183R | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLRKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 268 |
| S108R | RENEIPKDECPNFQGGPAIANIIAKSREFTEWEIYQSSLA | |
| | IQEVIFTLPKDKLPEPILKEEWRAQWLREHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 269 |
| S198R | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVRPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVEDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 270 |
| T114R | RENEIPKDECPNFQGGPAIANIIAKSREFTEWEIYQSSLA | |
| | IQEVIFTLPKDKLPEPILKEEWRAQWLSEHGLDRVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 271 |
| D369A | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVAVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 272 |
| D369N | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVNVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 273 |
| D658A | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNAAIDVATENLATVAITAQSMPKPT | |
| | CERSGDAKKPVRARKAKAPEFHDKLAPSYTVVLREAV | |
| CasPhi.12 | MIKPTVSQFLTPGFKLIRNHSRTAGLKLKNEGEEACKKFV | 274 |
| D658N | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNANIDVATENLATVAITAQSMPKPT | |
| | CERSGDAKKPVRARKAKAPEFHDKLAPSYTVVLREAV | |
| CasPhi | MIKPTVSQFLTPGFKLIRNHSRTAGLKLKNEGEEACKKFV | 449 |
| j12_L17_1 | RENEIPKDECPNFQGGPAIANIIAKSREFTEWEIYQSSLA | |
| 8_del1 | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQGSSGDYKWFQDYKPKLSKEVRDALSDIEWRLRRESLEF | |
| | NKLSKSREQDARQLANWISSMCDVIGIENLVKKNNFFGGS | |
| | GKREPGWDNFYKPKKENRWWINAIHKALTELSQNKGKRVI | |
| | LLPAMRTSITCPKCKYCDSKNRNGEKFNCLKCGIELNADI | |
| | DVATENLATVAITAQSMPKPTCERSGDAKKPVRARKAKAP | |
| | EFHDKLAPSYTVVLREAV | |
| CasPhi | MIKPTVSQFLTPGFKLIRNHSRTAGLKLKNEGEEACKKFV | 450 |
| j12_L17_1 | RENEIPKDECPNFQGGPAIANIIAKSREFTEWEIYQSSLA | |
| 8_del2 | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEGSSGDYKWFQDYKPKLSKEVRDALSDIEWRLR | |
| | RESLEFNKLSKSREQDARQLANWISSMCDVIGIENLVKKN | |
| | NFFGGSGKREPGWDNFYKPKKENRWWINAIHKALTELSQN | |
| | KGKRVILLPAMRTSITCPKCKYCDSKNRNGEKFNCLKCGI | |
| | ELNADIDVATENLATVAITAQSMPKPTCERSGDAKKPVRA | |
| | RKAKAPEFHDKLAPSYTVVLREAV | |
| CasPhi.12- | MIKPTVSQFLTPGFKLIRNHSRTAGKKLKNEGEEACKKFV | |
| L26K- | RENEIPKDECPNFQGGPAIANIIAKSREFTEWEIYQSSLA | |
| E567Q | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIQNLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | 451 |

TABLE 11
Exemplary Amino Acid Alterations Relative to SEQ ID NO: 230

| Effects | Amino Acid Alterations |
|---|---|
| | At least one substitution (i.e., with R, K or H) selected from I2, T5, K15, R18, |
| | H20, S21, L26, N30, E33, E34, A35, K37, K38, R41, N43, Q54, Q79R, K92E, |
| | K99R, S108, E109, H110, G111, D113, T114, P116, K118, E119, A121, N132, |
| | K135, Q138, V139, N148, L149, E157, E164, E166, E170, Y180, L182, Q183, |
| | K184, S186, K189, S196, S198, K200, I203, S205, K206, Y207, H208, N209, |
| | Y220, S223, E258, K281, K348, N355, S362, N406, K435, I471, I489, Y490, |
| | F491, D495, K496, K498, K500, D501, V502, K504, S505, D506, V521, E567, |
| | N568, S579, Q612, S638, F701, and P707 |
| Enhanced nuclease | T5R, L26R, L26K, A121Q, N148R, V139R, S198R, H208R, S223P, E258K, |
| activity relative to | N355R, I471T, S579R, F701R, P707R, K189P, S638K, Q54R, Q79R, Y220S, |
| the wild-type | N406K, E119S, K92E, K435Q, N568D, and V521T |
| effector protein | Double mutations: L26K/A121Q, L26X/A121Q, K99R/L149R, K99R/N148R, |
| | L149R/H208R, S362R/L26X L26X/N148R, L26X/H208R, N30R/N148R, |
| | L26X/K99R, L26X/P707R, L26X/L149R, L26X/N30R, L26X/N355R, |
| | L26X/K281R, L26X/S108R, L26X/K348R, T5R/V139R, I2R/V139R, |
| | K99R/S186R, L26X/A673G, L26X/Q674R, S579R/L26K, F701R/E258K, |
| | T5R/L26K, L26X/K435Q, L26X/G685R, L26X/Q674K, L26X/P699R, |
| | L26X/T70E, L26X/Q232R, L26X/T252R, L26X/E567Q, L26X/P679R, |
| | L26X/E83K, L26X/E73P, L26X/K248E, L26X, T5R/S223P, S579R/S223P, |
| | L26X/S223P, T5R/A121Q, L26X/A696R, S198R/I471T, L26X/N153R, L26X/ |
| | E682R, L26X/D703R, Q612R/L26K, L26X/I471T, K348R/L26K, |
| | S579R/I471T, L26X/V228R, T5R/S638K, S579R/K189P, S579R/E258K, |
| | L26X/K260R, L26X/S638K, S579R/Y220S, T5R/I471T, L26X/F233R, |
| | L26X/V521T, F701R/A121Q, L26X/G361R, S198R/E258K, L26X/S472R, |
| | TSR/Y220S, L26X/A150K, L26X/S684R, L26X/E157R, L26X/K248R, |
| | F701R/L26K, S198R/N406K, S198R/Y220S, S198R/S638K, S198R/V521T, |
| | S579R/A121Q, K348R/Y220S, S198R/K189P, L26X/E242R, L26X/K678R, |
| | T5R/N406K, L26X/I158K, T5R/V521T, L26X/N259R, L26X/K257R, |
| | L26X/K256R, T5R/K189P, L26X/C405R, S579R/V521T, S579R/N406K, |
| | TSR/K92E, T5R/1258K, L26X/197R, S579R/S638K, TSR/K435Q, |
| | F701R/S638K, L26X/L236R, F701R/I471T, Q612R/S223P, F701R/S223P, |
| | S198R/E119S, S579R/K92E, L26X/E715R, Q612R/1471T, F701R/Y220S, |
| | S198R/S223P, and L26X/K266R, wherein X is selected from R and K. |
| Nickase activity | E157A, E164A, E164L, E166A, E166I, E170A, I489A, 1489S, Y490S, Y490A, |
| | F491A, F491S, F491G, D495G, D495R, D495K, K496A, K496S, K498A, |
| | K498S, K500A, K500S, D501R, D501G, D501K, V502A, V502S, K504A, |
| | K504S, S505R, D506A; |
| | deletion of S478-S505 of SEQ ID NO: 230; |
| | deletion of S478-S505 of SEQ ID NO: 230 and insertion of the sequence of |
| | SDLYIERGGDPRDVHQQVETKPKGKRKSEIRILKIR (SEQ ID NO: 447); |
| | deletion of S478-S505 of SEQ ID NO: 230 and insertion of the sequence of |
| | SDYIVDHGGDPEKVFFETKSKKDKTKRYKRR (SEQ ID NO: 448); |
| | an amino acid sequence that is at least 90%, at least 95%, at least 97%, at least |
| | 98%, at least 99% identical, or is 100% identical to SEQ ID NO: 449; |
| | an amino acid sequence that is at least 90%, at least 95%, at least 97%, at least |
| | 98%, at least 99% identical, or is 100% identical to SEQ ID NO: 450 |
| Reduced or | D369A, D369N, D658A, D658N, E567A, E567Q |
| abolished nuclease | |
| activity relative to | |
| the wild-type | |
| effector protein | |

In certain embodiments, compositions comprise an effector protein and an engineered guide nucleic acid, wherein the amino acid sequence of the effector protein comprises at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500, at least about 520, at least about 540, at least about 560, at least about 580, at least about 600, at least about 620, at least about 640, at least about 660, at least about 680, at least about 700, or at least about 717 contiguous amino acids or more of any one of the sequences as set forth in TABLES 7-11. In certain embodiments, compositions comprise an effector protein and an engineered guide nucleic acid, wherein the amino acid sequence of the effector protein comprises at least about 200 contiguous amino acids or more of any one of the sequences as set forth in TABLES 7-11. In certain embodiments, compositions comprise an effector protein and an engineered guide nucleic acid, wherein the amino acid sequence of the effector protein comprises at least about 300 contiguous amino acids or more of any one of the sequences as set forth in TABLES 7-11. In certain embodiments, compositions comprise an effector protein and an engineered guide nucleic acid, wherein the amino acid sequence of the effector protein comprises at least about 400 contiguous amino acids or more of any one of the sequences as set forth in TABLES 7-11.

In some embodiments, compositions, systems, and methods described herein comprise an effector protein or a nucleic acid encoding the effector protein, wherein the effector protein comprises one or more amino acid alterations relative to the sequence recited in TABLES 7-11. In some embodiments, the effector protein comprising one or more amino acid alterations is a variant of an effector protein described herein. It is understood that any reference to an effector protein herein also refers to an effector protein variant as described herein. In some embodiments, an amino acid alteration comprises a deletion of an amino acid. In some embodiments, an amino acid alteration comprises an insertion of an amino acid. In some embodiments, an amino acid alteration comprises a conservative amino acid substitution. In some embodiments, an amino acid alteration comprises a non-conservative amino acid substitution. In some embodiments, one or more amino acid alterations comprises a combination of one or more conservative amino acid substitutions and one or more non-conservative amino acid substitutions. When describing a conservative amino acid substitution herein, reference is made to the replacement of one amino acid for another such that the replacement takes place within a family of amino acids that are related in their side chains. Conversely, when describing a non-conservative alteration (e.g., non-conservative substitution), reference is made to the replacement of one amino acid residue for another that does not have a related side chain. It is understood that genetically encoded amino acids can be divided into four families having related side chains: (1) acidic (negatively charged): Asp (D), Glu (E); (2) basic (positively charged) Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl. Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 7-11, wherein the effector protein comprises 1, 2, 3, 4, 5, 6, 7, 8, 9 or 10 conservative amino acid substitutions relative to the sequence selected from TABLES 7-11. In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 7-11, wherein the effector protein comprises 1 to 10, 10 to 20, 20 to 30, or 30 to 40 conservative amino acid substitutions relative to the sequence selected from TABLES 7-11. In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 7-11, wherein the effector protein comprises not more than 1, 2, 3, 4, 5, 6, 7, 8, 9 or 10 non-conservative amino acid substitutions relative to the sequence selected from TABLES 7-11.

In certain embodiments, compositions, systems, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 98%, at least 99%, or 100% similar to any one of the sequences selected from TABLES 7-11. An amino acid sequence of the effector protein is similar to the reference amino acid sequence, when a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIq would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold ≥1.

In some cases, the effector proteins comprise a RuvC domain. In some embodiments, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein but form a RuvC domain once the protein is produced and folds. In many cases, effector proteins comprise a recognition domain with a binding affinity for a guide nucleic acid or for a guide nucleic acid-target nucleic acid heteroduplex. An effector protein may comprise a zinc finger domain.

An effector protein may be small, which may be beneficial for nucleic acid detection or editing (for example, the effector protein may be less likely to adsorb to a surface or another biological species due to its small size). The smaller nature of these effector proteins may allow for them to be more easily packaged and delivered with higher efficiency in the context of genome editing and more readily incorporated as a reagent in an assay. In some embodiments, the length of the effector protein is less than 400 linked amino acid residues. In some embodiments, the length of the effector protein is less than 425 linked amino acid residues. In some embodiments, the length of the effector protein is less than 450 linked amino acid residues. In some embodiments, the length of the effector protein is less than 475 linked amino acid residues. In some embodiments, the length of the effector protein is less than 500 linked amino acid residues. In some embodiments, the length of the effector protein is less than 550, less than 600, less than 650, less than 700, or less than 717 linked amino acid residues. In some the length of the effector protein is less than 500 linked amino acid residues. In some embodiments, the length of the effector protein is about 400 to about 717 linked amino acids. In some embodiments, the length of the effector protein is about 400 to about 700 linked amino acid residues. In some embodiments, the length of the effector protein is about 650 to about 675 linked amino acids.

### Protospacer Adjacent Motif (PAM) Sequences

Effector proteins of the present disclosure, dimers thereof, and multimeric complexes thereof may cleave or nick a target nucleic acid within or near a protospacer adjacent motif (PAM) sequence of the target nucleic acid. In some embodiments, cleavage occurs within 10, 20, 30, 40 or 50 nucleotides of a 5′ or 3′ terminus of a PAM sequence. In some embodiments, cleavage occurs within 1, 2, 3, 4, 5, 6, 7, 8, 9 or 10 nucleotides of a 5′ or 3′ terminus of a PAM sequence. A target nucleic acid may comprise a PAM sequence adjacent to a target sequence. In some embodiments, systems, compositions, and methods comprise a guide nucleic acid or use thereof, wherein the guide nucleic acid comprises a spacer sequence that is complementary to a target sequence that is adjacent to a PAM sequence. In some embodiments, guide nucleic acids comprises a spacer sequence that is complementary to a target sequence that is adjacent to a PAM sequence. A target nucleic acid may comprise a PAM sequence adjacent to a target sequence.

In some embodiments, the PAM is 5′-NTTN-3′, wherein N=any nucleic acid. Exemplary PAM sequences are disclosed in TABLE 12. In some embodiments, the effector protein recognizes a PAM sequence comprising any of the following nucleotide sequences as set forth in TABLE 12. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 7, 10 and 11.

TABLE 12
Exemplary PAM NTTN Sequences

| PAM # | PAM Sequence (5′-3′) |
|---|---|
| 1 | NTTG |
| 2 | NTTC |
| 3 | NTTT |
| 4 | NTTA |

In some embodiments, the PAM is 5′-NNTN-3′, wherein N=any nucleic acid. In some embodiments, the PAM is 5′-TNTR-3′, wherein N=any nucleic acid and wherein R=a purine nucleic acid (i.e., A or G). In some embodiments, the PAM is 5′-TNTG-3.′ Exemplary PAM sequences are disclosed in TABLE 13. In some embodiments, the effector protein recognizes a PAM sequence comprising any of the following nucleotide sequences as set forth in TABLE 13. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 7, 8, and 9.

TABLE 13
Exemplary PAM NNTN Sequences

| PAM # | PAM Sequence (5′-3′) |
|---|---|
| 1 | TTTG |
| 2 | TCTG |
| 3 | TGTG |
| 4 | TCTA |
| 5 | TATA |
| 6 | TTTA |
| 7 | TGTA |
| 8 | TATG |

### Engineered Proteins

In some embodiments, effector proteins disclosed herein are engineered proteins. Engineered proteins are not identical to a naturally-occurring protein. Engineered proteins may provide enhanced nuclease or nickase activity as compared to a naturally occurring nuclease or nickase. SEQ ID NO: 232 is a non-limiting example of an engineered protein, wherein residue 26 has been modified to an arginine from a leucine at residue 26 of SEQ ID NO: 230.

An engineered protein may comprise a modified form of a wild-type counterpart protein (e.g., an effector protein). The modified form of the wild-type counterpart may comprise an amino acid change (e.g., deletion, insertion, or substitution) that reduces the nucleic acid-cleaving activity of the effector protein relative to the wild-type counterpart. For example, a nuclease domain (e.g., RuvC domain) of an effector protein may be deleted or mutated relative to a wild-type counterpart effector protein so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart.

### Nuclease-Dead Effector Proteins

In some embodiments, the effector protein may comprise an enzymatically inactive and/or “dead” (abbreviated by “d”) effector protein in combination (e.g., fusion) with a polypeptide comprising recombinase activity. In some embodiments, nuclease-dead effector protein may also be referred to as a catalytically inactive effector protein. Although an effector protein normally has nuclease activity, in some embodiments, an effector protein does not have nuclease activity. In some embodiments, an effector protein comprising a nuclease-dead effector protein, wherein the nuclease-dead effector protein comprising an amino acid sequence that is at least 60%, at least 65%, at least 70%, at least 75%, at least 80%, at least 85%, at least 90%, at least 92%, at least 95%, at least 97%, at least 98%, at least 99%, or 100% identical to any one of the sequences recited in TABLES 7-11. In some embodiments, the effector protein comprising an amino acid sequence that is at least 60%, at least 65%, at least 70%, at least 75%, at least 80%, at least 85%, at least 90%, at least 92%, at least 95%, at least 97%, at least 98%, at least 99%, or 100% identical to any one of the sequences recited in TABLES 7-11, wherein the effector protein is modified or engineered to be a nuclease-dead effector protein.

Catalytically inactive effector proteins may comprise a modified form of a wildtype counterpart. The modified form of the wildtype counterpart may comprise an amino acid change (e.g., deletion, insertion, or substitution) that reduces the nucleic acid-cleaving activity of the effector protein. In such embodiments, the catalytically inactive effector protein may also be referred to as a catalytically reduced effector protein. For example, a nuclease domain (e.g., HEPN domain, RuvC domain) of an effector protein can be deleted or mutated so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart.

The modified form of an effector protein may have no substantial nucleic acid-cleaving activity. When an effector protein is a modified form that has no substantial nucleic acid-cleaving activity, it may be referred to as enzymatically inactive and/or dead. A dead effector polypeptide (e.g., catalytically inactive effector protein) may bind to a target nucleic acid but may not cleave the target nucleic acid. A dead effector polypeptide (e.g., catalytically inactive effector protein) may associate with a guide nucleic acid to activate or repress transcription of a target nucleic acid.

In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, or at least 99% identical to SEQ ID NO: 230, and wherein the effector protein further comprises one or more alterations selected from D369A, D369N, E567A, E567Q, D658A and D658N. In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% similar to SEQ ID NO: 230, and wherein the effector protein further comprises one or more alterations selected from D369A, D369N, E567A, E567Q, D658A and D658N.

In certain embodiments, the amino acid sequence of the dCas protein is based on SEQ ID NO: 230 and is modified at position 369. In some embodiments the modification at position 369 is from aspartic acid to alanine (D369A). In some embodiments, the amino acid sequence of the dCas protein is at least 95%, 96%, 97%, 98%, or 99% identical to SEQ ID NO: 271. In some embodiments, the amino acid sequence of the dCas protein comprises or consists of SEQ ID NO: 271.

In certain embodiments, the amino acid sequence of the dCas protein is based on SEQ ID NO: 230 and is modified at position 369. In some embodiments the modification at position 369 is from aspartic acid to asparagine (D369N). In some embodiments, the amino acid sequence of the dCas protein is at least 95%, 96%, 97%, 98%, or 99% identical to SEQ ID NO: 272. In some embodiments, the amino acid sequence of the dCas protein comprises or consists of SEQ ID NO: 272.

In certain embodiments, the amino acid sequence of the dCas protein is based on SEQ ID NO: 230 and is modified at position 658. In some embodiments the modification at position 658 is from aspartic acid to alanine (D658A). In some embodiments, the amino acid sequence of the dCas protein is at least 95%, 96%, 97%, 98%, or 99% identical to SEQ ID NO: 273. In some embodiments, the amino acid sequence of the dCas protein comprises or consists of SEQ ID NO: 273.

In certain embodiments, the amino acid sequence of the dCas protein is based on SEQ ID NO: 230 and is modified at position 658. In some embodiments the modification at position 658 is from aspartic acid to asparagine (D658N). In some embodiments, the amino acid sequence of the dCas protein is at least 95%, 96%, 97%, 98%, or 99% identical to SEQ ID NO: 274. In some embodiments, the amino acid sequence of the dCas protein comprises or consists of SEQ ID NO: 274.

In certain embodiments, the amino acid sequence of the dCas protein is based on SEQ ID NO: 230 and is modified at position 567. In some embodiments the modification at position 567 is from glutamine acid to alanine (E567A). In some embodiments, the amino acid sequence of the dCas protein is at least 95%, 96%, 97%, 98%, or 99% identical to SEQ ID NO: 243. In some embodiments, the amino acid sequence of the dCas protein comprises or consists of SEQ ID NO: 243.

In certain embodiments, the amino acid sequence of the dCas protein is based on SEQ ID NO: 230 and is modified at position 567. In some embodiments the modification at position 567 is from glutamic acid to glutamine (E567Q). In some embodiments, the amino acid sequence of the dCas protein is at least 95%, 96%, 97%, 98%, or 99% identical to SEQ ID NO: 244. In some embodiments, the amino acid sequence of the dCas protein comprises or consists of SEQ ID NO: 244.

In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, or at least 99% identical to SEQ ID NO: 428, and wherein the effector protein further comprises one or more alterations selected from D237A, D418A, D418N, E335A, and E335Q. In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% similar to SEQ ID NO: 428, and wherein the effector protein further comprises one or more alterations selected from D237A, D418A, D418N, E335A, and E335Q.

In certain embodiments, the amino acid sequence of the dCas protein is based on SEQ ID NO: 428 and is modified at position 335. In some embodiments the modification at position 335 is from glutamic acid to glutamine (E335Q). In some embodiments, the amino acid sequence of the dCas protein is at least 95%, 96%, 97%, 98%, or 99% identical to SEQ ID NO: 442. In some embodiments, the amino acid sequence of the dCas protein comprises or consists of SEQ ID NO: 442.

In certain embodiments, the amino acid sequence of the dCas protein is based on SEQ ID NO: 428 and is modified at position 237. In some embodiments the modification at position 237 is from aspartic acid to alanine (D237A). In some embodiments, the amino acid sequence of the dCas protein is at least 95%, 96%, 97%, 98%, or 99% identical to SEQ ID NO: 443. In some embodiments, the amino acid sequence of the dCas protein comprises or consists of SEQ ID NO: 443.

In certain embodiments, the amino acid sequence of the dCas protein is based on SEQ ID NO: 428 and is modified at position 418. In some embodiments the modification at position 418 is from aspartic acid to alanine (D418A). In some embodiments, the amino acid sequence of the dCas protein is at least 95%, 96%, 97%, 98%, or 99% identical to SEQ ID NO: 444. In some embodiments, the amino acid sequence of the dCas protein comprises or consists of SEQ ID NO: 444.

In certain embodiments, the amino acid sequence of the dCas protein is based on SEQ ID NO: 428 and is modified at position 418. In some embodiments the modification at position 418 is from aspartic acid to asparagine (D418N). In some embodiments, the amino acid sequence of the dCas protein is at least 95%, 96%, 97%, 98%, or 99% identical to SEQ ID NO: 445. In some embodiments, the amino acid sequence of the dCas protein comprises or consists of SEQ ID NO: 445.

In certain embodiments, the amino acid sequence of the dCas protein is based on SEQ ID NO: 428 and is modified at position 335. In some embodiments the modification at position 335 is from glutamic acid to alanine (E335A). In some embodiments, the amino acid sequence of the dCas protein is at least 95%, 96%, 97%, 98%, or 99% identical to SEQ ID NO: 446. In some embodiments, the amino acid sequence of the dCas protein comprises or consists of SEQ ID NO: 446.

### Fusion Proteins

In some embodiments, compositions, systems, and methods comprise a fusion protein, a fusion partner, or uses thereof. A fusion protein generally comprises an effector protein and a fusion partner. In some embodiments, the fusion partner comprises a polypeptide or peptide that is linked to the effector protein. In some embodiments, the fusion partner is not linked to the effector protein but is brought into proximity of the effector protein by other means. By way of non-limiting example, a fusion partner protein may comprise a peptide that binds an aptamer of a guide nucleic acid, wherein the effector protein is also capable of binding the guide nucleic acid, the guide nucleic acid thereby bringing the fusion partner into proximity of the effector protein. In some embodiments, the fusion partner is capable of binding or being bound by an effector protein. In some embodiments, the fusion partner and the effector protein are both capable of binding or being bound by an additional protein or moiety, the additional protein or moiety thereby bringing the fusion partner into proximity of the effector protein. In some embodiments, the fusion protein is a heterologous peptide or polypeptide as described herein. In some embodiments, the amino terminus of the fusion partner is linked to the carboxy terminus of the effector protein. In some embodiments, the carboxy terminus of the fusion partner protein is linked to the amino terminus of the effector protein by the linker. In some embodiments, the fusion partner is not an effector protein as described herein. In some embodiments, the fusion partner comprises a second effector protein or a multimeric form thereof. Accordingly, in some embodiments, the fusion protein comprises more than one effector protein. In such embodiments, the fusion protein can comprise at least two effector proteins that are same. In some embodiments, the fusion protein comprises at least two effector proteins that are different. In some embodiments, the multimeric form is a homomeric form. In some embodiments, the multimeric form is a heteromeric form. Unless otherwise indicated, reference to effector proteins throughout the present disclosure include fusion proteins comprising the effector protein described herein and a fusion partner.

In some embodiments, a fusion partner imparts some function or activity to a fusion protein that is not provided by an effector protein. Such activities may include but are not limited to nuclease activity, methyltransferase activity, demethylase activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity, modification of a polypeptide associated with target nucleic acid (e.g., a histone), and/or signaling activity.

In some embodiments, a fusion partner may provide signaling activity. In some embodiments, a fusion partner may inhibit or promote the formation of multimeric complex of an effector protein. In an additional example, the fusion partner may directly or indirectly edit a target nucleic acid. Edits can be of a nucleobase, nucleotide, or nucleotide sequence of a target nucleic acid. In some embodiments, the fusion partner may interact with additional proteins, or functional fragments thereof, to make modifications to a target nucleic acid. In other embodiments, the fusion partner may modify proteins associated with a target nucleic acid. In some embodiments, a fusion partner may modulate transcription (e.g., inhibits transcription, increases transcription) of a target nucleic acid. In yet another example, a fusion partner may directly or indirectly inhibit, reduce, activate or increase expression of a target nucleic acid.

In some embodiments of the above, the effector protein comprises an amino acid sequence that is at least 95% identical to any one of the sequences recited in TABLES 7, 10, and 11, and wherein the guide RNA comprises a repeat sequence that is at least 95% identical to any one of the sequences recited in TABLE 3 and a spacer sequence that is at least 95% identical to any one of the sequences recited in TABLE 1.

In some embodiments of the above, the effector protein comprises any one of the sequences recited in TABLES 7, 10, and 11, and wherein the guide RNA comprises any one of the repeat sequences recited in TABLE 3 and any one of the spacer sequences recited in TABLE 1.

In some embodiments, the effector protein comprises an amino acid sequence that is at least 90% identical to any one of the sequences of TABLES 7, 10, and 11, and wherein the guide RNA comprises a sequence that is at least 90% identical to any one of the guide RNA sequences of TABLE 4.

In some embodiments of the above, the effector protein comprises an amino acid sequence that is at least 95% identical to any one of the sequences of TABLES 7, 10, and 11, and wherein the guide RNA comprises a sequence that is at least 95% identical to any one of the guide RNA sequences of TABLE 4.

In some embodiments of the above, the effector protein amino acid sequence comprises a nuclear localization signal.

In some embodiments of the above, the composition further comprises an additional guide RNA that binds a different portion of the target nucleic acid than the guide RNA.

In some embodiments of the above, the guide RNA comprises at least one sequence that is at least 80%, at least 85%, at least 90%, at least 95% or 100% identical to a sequence selected from any one of TABLES 1, 3, and 4.

In some embodiments of the above, the effector protein comprises an amino acid sequence that is at least 95% identical to any one of the sequences recited in TABLES 7, 8, and 9, and wherein the guide RNA comprises a repeat sequence that is at least 95% identical to SEQ ID NO: 350 and a spacer sequence that is at least 95% identical to any one of the sequences recited in TABLE 2.

In some embodiments of the above, the effector protein comprises any one of the sequences recited in TABLES 7, 8, and 9, and wherein the guide RNA comprises SEQ ID NO: 350 and any one of the spacer sequences recited in TABLE 2.

In some embodiments, the effector protein comprises an amino acid sequence that is at least 90% identical to any one of the sequences of TABLES 7, 8, and 9, and wherein the guide RNA comprises a sequence that is at least 90% identical to any one of the guide RNA sequences of TABLE 5.

In some embodiments of the above, the effector protein comprises an amino acid sequence that is at least 95% identical to any one of the sequences of TABLES 7, 8, and 9, and wherein the guide RNA comprises a sequence that is at least 95% identical to any one of the guide RNA sequences of TABLE 5.

In some embodiments of the above, the effector protein amino acid sequence comprises a nuclear localization signal.

In some embodiments of the above, the composition further comprises an additional guide RNA that binds a different portion of the target nucleic acid than the guide RNA.

In some embodiments of the above, the guide RNA comprises at least one sequence that is at least 80%, at least 85%, at least 90%, at least 95% or 100% identical to a sequence selected from any one of TABLES 2 and 5 and SEQ ID NOs: 236, 350-352.

### Nucleic Acid Modification Activity

In some embodiments, fusion partners have enzymatic activity that modifies a nucleic acid, such as a target nucleic acid. In some embodiments, the target nucleic acid may comprise or consist of a ssRNA, dsRNA, ssDNA, or a dsDNA. Examples of enzymatic activity that modifies the target nucleic acid include, but are not limited to: nuclease activity, which comprises the enzymatic activity of an enzyme which allows the enzyme to cleave the phosphodiester bonds between the nucleotide subunits of nucleic acids, such as that provided by a restriction enzyme, or a nuclease (e.g., FokI nuclease); methyltransferase activity such as that provided by a methyltransferase (e.g., Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y, human immunodeficiency virus type 1 integrase (IN), Tn3 resolvase); transposase activity; recombinase activity such as that provided by a recombinase (e.g., catalytic domain of Gin recombinase); polymerase activity; ligase activity; helicase activity; photolyase activity; and glycosylase activity.

In some embodiments, fusion partners target a ssRNA, dsRNA, ssDNA, or a dsDNA. In some embodiments, fusion partners target ssRNA. Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that a fusion partner may include an entire protein, or in some embodiments, may include a fragment of the protein (e.g., a functional domain). In some embodiments, the functional domain binds or interacts with a nucleic acid, such as ssRNA, including intramolecular and/or intermolecular secondary structures thereof (e.g., hairpins, stem-loops, etc.). The functional domain may interact transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid editing, nucleic acid mutating, nucleic acid modifying, nucleic acid cleaving, protein binding or combinations thereof. The absence of the functional domain, including mutations of the functional domain, would abolish or reduce activity.

Accordingly, fusion partners may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for polyadenylation of RNA (e.g., PAP1, GLD-2, and Star-PAP); proteins and protein domains responsible for polyuridinylation of RNA (e.g., CID1 and terminal uridylate transferase); and other suitable domains that affect nucleic acid modifications.

In some embodiments, an effector protein is a fusion protein, wherein the effector protein is linked to a chromatin-modifying enzyme. In some embodiments, the fusion protein chemically modifies a target nucleic acid, for example by methylating, demethylating, or acetylating the target nucleic acid in a sequence specific or non-specific manner.

### Base Editors

In some embodiments, fusion partners edit a nucleobase of a target nucleic acid. Fusion proteins comprising such a fusion partner and an effector protein may be referred to as base editors. Such a fusion partner may be referred to as a base editing enzyme. In some embodiments, a base editor comprises a base editing enzyme variant that differs from a naturally occurring base editing enzyme, but it is understood that any reference to a base editing enzyme herein also refers to a base editing enzyme variant. In some embodiments, a base editor may be a fusion protein comprising a base editing enzyme linked to an effector protein. In some embodiments, the amino terminus of the fusion partner protein is linked to the carboxy terminus of the effector protein by the linker. In some embodiments, the carboxy terminus of the fusion partner protein is linked to the amino terminus of the effector protein by the linker. The base editor may be functional when the effector protein is coupled to a guide nucleic acid. The base editor may be functional when the effector protein is coupled to a guide nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. Additional base editors are described herein.

In some embodiments, base editors are capable of catalyzing editing (e.g., a chemical modification) of a nucleobase of a nucleic acid molecule, such as DNA or RNA (single stranded or double stranded). In some embodiments, a base editing enzyme, and therefore a base editor, is capable of converting an existing nucleobase to a different nucleobase, such as: an adenine (A) to guanine (G); cytosine (C) to thymine (T); cytosine (C) to guanine (G); uracil (U) to cytosine (C); guanine (G) to adenine (A); hydrolytic deamination of an adenine or adenosine, or methylation of cytosine (e.g., CpG, CpA, CpT or CpC). In some embodiments, base editors edit a nucleobase on a ssDNA. In some embodiments, base editors edit a nucleobase on both strands of dsDNA. In some embodiments, base editors edit a nucleobase of an RNA.

In some embodiments, a base editing enzyme itself may or may not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments, DNA bases within the R-loop are edited by the base editor having the deaminase enzyme activity. In some embodiments, base editors for improved efficiency in eukaryotic cells comprise a catalytically inactive effector protein that may generate a nick in the non-edited strand, inducing repair of the non-edited strand using the edited strand as a template.

In some embodiments, a base editing enzyme comprises a deaminase enzyme. Exemplary deaminases are described in US20210198330, WO2021041945, WO2021050571A1, and WO2020123887, all of which are incorporated herein by reference in their entirety. Exemplary deaminase domains are described WO2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3: eaao4774 (2017), and Rees et al., Nat Rev Genet. 2018 December; 19 (12): 770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editors comprise a DNA glycosylase inhibitor (e.g., an uracil glycosylase inhibitor (UGI) or uracil N-glycosylase (UNG)). In some embodiments, the fusion partner is a deaminase, e.g., ADAR1/2, ADAR-2, AID, or any function variant thereof.

In some embodiments, a base editor is a cytosine base editor (CBE). In some embodiments, the CBE may convert a cytosine to a thymine. In some embodiments, a cytosine base editing enzyme may accept ssDNA as a substrate but may not be capable of cleaving dsDNA, as linked to a catalytically inactive effector protein. In some embodiments, when bound to its cognate DNA, the catalytically inactive effector protein of the CBE may perform local denaturation of the DNA duplex to generate an R-loop in which the DNA strand not paired with a guide nucleic acid exists as a disordered single-stranded bubble. In some embodiments, the catalytically inactive effector protein generated ssDNA R-loop may enable the CBE to perform efficient and localized cytosine deamination in vitro. In some embodiments, deamination activity is exhibited in a window of about 4 to about 10 base pairs. In some embodiments, fusion to the catalytically inactive effector protein presents a target site to the cytosine base editing enzyme in high effective molarity, which may enable the CBE to deaminate cytosines located in a variety of different sequence motifs, with differing efficacies. In some embodiments, the CBE is capable of mediating RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C·G-to-G·C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U·G in DNA is initiated by a UNG, which recognizes a U·G mismatch and cleaves the glyosidic bond between a uracil and a deoxyribose backbone of DNA. In some embodiments, BER results in the reversion of the U·G intermediate created by the first CBE back to a C·G base pair. In some embodiments, the UNG may be inhibited by fusion of a UGI. In some embodiments, the CBE comprises a UGI. In some embodiments, a C-terminus of the CBE comprises the UGI. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG. In some embodiments, the CBE may mediate efficient base editing in bacterial cells and moderately efficient editing in mammalian cells, enabling conversion of a C·G base pair to a T·A base pair through a U·G intermediate. In some embodiments, the CBE is modified to increase base editing efficiency while editing more than one strand of DNA.

In some embodiments, a CBE nicks a non-edited DNA strand. In some embodiments, the non-edited DNA strand nicked by the CBE biases cellular repair of a U·G mismatch to favor a U·A outcome, elevating base editing efficiency. In some embodiments, a APOBEC1-nickase-UGI fusion efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment may be capable of excising a uracil residue from DNA by cleaving an N-glycosidic bond.

In some embodiments, the fusion protein further comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins, and Cas-CRISPR systems comprising base editing activity are described in WO2021087246, which is incorporated by reference in its entirety.

In some embodiments, a cytosine base editing enzyme, and therefore a cytosine base editor, is a cytidine deaminase. In some embodiments, the cytidine deaminase base editor is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BEI (APOBEC1-XTEN-dCas9), BE2 (APOBEC1-XTEN-dCas9-UGI), BE3 (APOBEC1-XTEN-dCas9 (A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE). A CGBE may convert a cytosine to a guanine.

In some embodiments, a base editor is an adenine base editor (ABE). An ABE may convert an adenine to a guanine. In some embodiments, an ABE converts an A·T base pair to a G·C base pair. In some embodiments, the ABE converts a target A·T base pair to G·C in vivo or in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. Non-limiting exemplary adenine base editing enzymes suitable for use with effector proteins described herein include: ABE8e, ABE8.20m, APOBEC3A, Anc APOBEC (a.k.a. AncBE4Max), and BtAPOBEC2. Non-limiting exemplary ABEs suitable for use herein include: ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, the ABE is ABE8e and comprises an amino acid sequence that is at least at least 90%, at least 95%, at least 97%, at least 98%, or at least 99% identical to SEQ ID NO: 453. In some embodiments, the ABE is ABE8e and comprises or consists of SEQ ID NO: 453.

In some embodiments, the present disclosure provides a fusion protein comprising an effector protein described herein and a base editing enzyme described herein. In some embodiments, the fusion protein comprises, from N-terminus to C-terminus, an effector protein and a base editing enzyme. In some embodiments, the fusion protein comprises, from N-terminus to C-terminus, a base editing enzyme and an effector protein. In some embodiments, the base editing enzyme is ABE8e.

In some embodiments, the fusion protein described herein comprises an effector protein comprising an amino acid sequence that is at least 90%, 91%, 92%, 93%, 94%, 95%, 96%, 97%, 98%, or 99% identical to SEQ ID NO: 428 and a base editing enzyme comprising an amino acid sequence that is at least 90%, 91%, 92%, 93%, 94%, 95%, 96%, 97%, 98%, or 99% identical to SEQ ID NO: 453. In some embodiments, the fusion protein described herein comprises an effector protein comprising or consisting of SEQ ID NO: 428 and a base editing enzyme comprising or consisting of SEQ ID NO: 453. In some embodiments, the fusion protein comprises a linker sequence comprising SEQ ID NO: 475. In some embodiments, the fusion protein comprises an amino acid sequence that is at least at least 90%, at least 95%, at least 97%, at least 98%, or at least 99% identical to SEQ ID NO: 454. In some embodiments, the ABE is ABE8e and comprises or consists of SEQ ID NO: 454.

In some embodiments, the fusion protein described herein comprises an effector protein comprising an amino acid sequence that is at least 90%, 91%, 92%, 93%, 94%, 95%, 96%, 97%, 98%, or 99% identical to SEQ ID NO: 230 and a base editing enzyme comprising an amino acid sequence that is at least 90%, 91%, 92%, 93%, 94%, 95%, 96%, 97%, 98%, or 99% identical to SEQ ID NO: 453. In some embodiments, the fusion protein described herein comprises an effector protein comprising or consisting of SEQ ID NO: 230 and a base editing enzyme comprising or consisting of SEQ ID NO: 453. In some embodiments, the fusion protein comprises a linker sequence comprising SEQ ID NO: 475. In some embodiments, the fusion protein comprises an amino acid sequence that is at least at least 90%, at least 95%, at least 97%, at least 98%, or at least 99% identical to SEQ ID NO: 455. In some embodiments, the ABE is ABE8e and comprises or consists of SEQ ID NO: 455. Exemplary fusion proteins are provided in TABLE 14.

TABLE 14
Exemplary base editing enzyme and base
editor fusion proteins

| | | SEQ |
|---|---|---|
| Protein | AA Sequence | ID |
| ABE8e | SEVEFSHEYWMRHALTLAKRARDEREVPVG | 453 |
| | AVLVLNNRVIGEGWNRAIGLHDPTAHAEIM | |
| | ALRQGGLVMQNYRLIDATLYVTFEPCVMCA | |
| | GAMIHSRIGRVVFGVRNSKRGAAGSLMNVL | |
| | NYPGMNHRVEITEGILADECAALLCDFYRM | |
| | PRQVFNAQKKAQSSIN | |
| Cas.265466- | MSVLTRKVQLIPVGDKEERDRVYKYLRDGI | 454 |
| D220R- | EAQNRAMNLYMSGLYFAAINEASKEDRKEL | |
| E335Q_ABE8e | NQLYSRIATSSKGSAYTTDIEFPTGLASTS | |
| fusion | TLSMAVRQDFTKSLKDGLMYGRVSLPTYRK | |
| | DNPLFVDVRFVALRGTKQKYNGLYHEYKSH | |
| | TEFLDNLYSSDLKVYIKFANDITFQVIFGN | |
| | PRKSSALRSEFQNIFEEYYKVCQSSIQFSG | |
| | TKIILNMAMRIPDKEIELDEDVCVGVDLGI | |
| | AIPAVCALNKNRYSRVSIGSKEDFLRVRTK | |
| | IRNQRKRLQTNLKSSNGGHGRKKKMKPMDR | |
| | FRDYEANWVQNYNHYVSRQVVDFAVKNKAK | |
| | YINLQNLEGIRDDVKNEWLLSNWSYYQLQQ | |
| | YITYKAKTYGIEVRKINPYHTSQRCSCCGY | |
| | EDAGNRPKKEKGQAYFKCLKCGEEMNADFN | |
| | AARNIAMSTEFQSGKKTKKQKKEQHENKGS | |
| | SGGSPAGSPTSTEEGTSESATPESGPGTST | |
| | EPSEGSAPGSPAGSGGGSSEVEFSHEYWMR | |
| | HALTLAKRARDEREVPVGAVLVLNNRVIGE | |
| | GWNRAIGLHDPTAHAEIMALRQGGLVMQNY | |
| | RLIDATLYVTFEPCVMCAGAMIHSRIGRVV | |
| | FGVRNSKRGAAGSLMNVLNYPGMNHRVEIT | |
| | EGILADECAALLCDFYRMPRQVFNAQKKAQ | |
| | SSIN | |
| CasPhi.12- | MIKPTVSQFLTPGFKLIRNHSRTAGKKLKN | 455 |
| L26K- | EGEEACKKFVRENEIPKDECPNFQGGPAIA | |
| E567Q_ | NIIAKSREFTEWEIYQSSLAIQEVIFTLPK | |
| ABE8e | DKLPEPILKEEWRAQWLSEHGLDTVPYKEA | |
| fusion | AGLNLIIKNAVNTYKGVQVKVDNKNKNNLA | |
| | KINRKNEIAKLNGEQEISFEEIKAFDDKGY | |
| | LLQKPSPNKSIYCYQSVSPKPFITSKYHNV | |
| | NLPEEYIGYYRKSNEPIVSPYQFDRLRIPI | |
| | GEPGYVPKWQYTFLSKKENKRRKLSKRIKN | |
| | VSPILGIICIKKDWCVFDMRGLLRTNHWKK | |
| | YHKPTDSINDLFDYFTGDPVIDTKANVVRF | |
| | RYKMENGIVNYKPVREKKGKELLENICDQN | |
| | GSCKLATVDVGQNNPVAIGLFELKKVNGEL | |
| | TKTLISRHPTPIDFCNKITAYRERYDKLES | |
| | SIKLDAIKQLTSEQKIEVDNYNNNFTPQNT | |
| | KQIVCSKLNINPNDLPWDKMISGTHFISEK | |
| | AQVSNKSEIYFTSTDKGKTKDVMKSDYKWF | |
| | QDYKPKLSKEVRDALSDIEWRLRRESLEFN | |
| | KLSKSREQDARQLANWISSMCDVIGIQNLV | |
| | KKNNFFGGSGKREPGWDNFYKPKKENRWWI | |
| | NAIHKALTELSQNKGKRVILLPAMRTSITC | |
| | PKCKYCDSKNRNGEKFNCLKCGIELNADID | |
| | VATENLATVAITAQSMPKPTCERSGDAKKP | |
| | VRARKAKAPEFHDKLAPSYTVVLREAVGSS | |
| | GGSPAGSPTSTEEGTSESATPESGPGTSTE | |
| | PSEGSAPGSPAGSGGGSSEVEFSHEYWMRH | |
| | ALTLAKRARDEREVPVGAVLVLNNRVIGEG | |
| | WNRAIGLHDPTAHAEIMALRQGGLVMQNYR | |
| | LIDATLYVTFEPCVMCAGAMIHSRIGRVVF | |
| | GVRNSKRGAAGSLMNVLNYPGMNHR VEIT | |
| | EGILADECAALLCDFYRMPRQVFNAQKKAQ | |
| | SSIN | |

In some embodiments, an adenine base editing enzyme of an ABE is an adenosine deaminase. Non-limiting exemplary adenosine base editing enzymes suitable for use herein include ABE9. In some embodiments, the ABE comprises an engineered adenosine deaminase enzyme capable of acting on ssDNA. The engineered adenosine deaminase enzyme may be an adenosine deaminase variant that differs from a naturally occurring deaminase. Relative to the naturally occurring deaminase, the adenosine deaminase variant may comprise one or more amino acid alteration, including a V82S alteration, a T166R alteration, a Y147T alteration, a Y147R alteration, a Q154S alteration, a Y123H alteration, a Q154R alteration, or a combination thereof.

In some embodiments, a base editor comprises a deaminase dimer. In some embodiments, the base editor further comprising a base editing enzyme and an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises a base editing enzyme linked to TadA by a linker (e.g., wherein the base editing enzyme is linked to TadA at N-terminus or C-terminus by a linker).

In some embodiments, a base editing enzyme is a deaminase dimer comprising an ABE. In some embodiments, the deaminase dimer comprises an adenosine deaminase. In some embodiments, the deaminase dimer comprises TadA linked to a suitable adenine base editing enzyme including an: ABE8e, ABE8.20m, APOBEC3A, Anc APOBEC (a.k.a. AncBE4Max), BtAPOBEC2, and variants thereof. In some embodiments, the adenine base editing enzyme is linked to amino-terminus or the carboxy-terminus of TadA.

In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions, systems, and methods described herein comprise a base editor and a guide nucleic acid, wherein the guide nucleic acid directs the base editor to a sequence in a target gene.

### Precision Editing Systems

In some embodiments, the fusion partner comprises a polymerase. In some embodiments, the fusion partner is an RNA-directed DNA polymerase (RDDP). In some embodiments, the RDDP is a reverse transcriptase.

In some embodiments, the RDDP that is capable of catalyzing the modification of the target nucleic acid forms a complex with an extended guide RNA. In some embodiments, the extended guide RNA comprises (not necessarily in this order): a first region (also referred to as a protein binding region or protein binding sequence) that interacts with an effector protein; a second region comprising a spacer sequence that is complementary to a target sequence of a first strand of a target dsDNA molecule; a third region comprising a template sequence that is complementary to at least a portion of the target sequence on the non-target strand of the target dsDNA molecule with the exception of at least one nucleotide; and a fourth region comprising a primer binding sequence that hybridizes to a primer sequence of the target dsDNA molecule that is formed when target nucleic acid is cleaved. The third region or template sequence may comprise a nucleotide having a different nucleobase than that of a nucleotide at the corresponding position in the target nucleic acid when the template sequence and the target sequence are aligned for maximum identity. In some embodiments, there is a linker between any one of the first, second, third and fourth regions. In some embodiments, the linker comprises a nucleotide. In some embodiments, the linker comprises multiple nucleotides.

In some embodiments, the third and fourth regions are 5′ of the first and second regions. In some embodiments, the order of the regions of the extended guide RNA from 5′ to 3′ is: third region, fourth region, first region, and second region. In some embodiments, there is a linker between any one of the first, second, third and fourth regions. In some embodiments, there is a linker between the first and fourth regions. In some embodiments, the effector protein is linked to an RDDP. In some embodiments, the RDDP comprises a reverse transcriptase.

In some embodiments, the third and fourth regions are 3′ of the first and second regions. In some embodiments, the order of the regions of the extended guide RNA from 5′ to 3′ is: first region, second region, third region, and fourth region. In some embodiments, there is a linker between the second and third regions.

### Protein Modification Activity

In some embodiments, a fusion partner provides enzymatic activity that modifies a protein associated with a target nucleic acid. The protein may be a histone, an RNA binding protein, or a DNA binding protein. Examples of such protein modification activities include: methyltransferase activity, such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SETIA, SET1B, MLL1 to 5, ASH1, SYMD2, NSD1, DOTIL, Pr-SET7/8, SUV4-20H1, EZH2, RIZ1); demethylase activity such as that provided by a histone demethylase (e.g., Lysine Demethylase 1A (KDMIA also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARIDID/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity; phosphatase activity; ubiquitin ligase activity; deubiquitinating activity; adenylation activity; deadenylation activity; SUMOylating activity; deSUMOylating activity; ribosylation activity; deribosylation activity; myristoylation activity; and demyristoylation activity.

### CRISPRa Fusions and CRISPRi Fusions

In some embodiments, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, fusion partners that increase or decrease transcription include a transcription activator domain or a transcription repressor domain, respectively.

In some embodiments, fusion partners activate or increase expression of a target nucleic acid. Such fusion proteins comprising the described fusion partners and an effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. In general, a transcriptional activator refers to a polypeptide or a fragment thereof that can activate or increase transcription of a target nucleic acid molecule. In some embodiments, the transcriptional activators may promote transcription by: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. In some embodiments, the fusion partner is a reverse transcriptase.

Non-limiting examples of fusion partners that promote or increase transcription include: transcriptional activators such as VP16, VP64, VP48, VP160, p65 subdomain (e.g., from NFKB), and activation domain of EDLL and/or TAL activation domain (e.g., for activity in plants); histone lysine methyltransferases such as SETIA, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof. Other non-limiting examples of suitable fusion partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, fusions partners inhibit or reduce expression of a target nucleic acid. Such fusion proteins comprising described fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. In some embodiments, the transcriptional repressors may inhibit transcription by: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of fusion partners that decrease or inhibit transcription include: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARIDIC/SMCX, JARIDID/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as Hhal DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable fusion partners include: proteins and protein domains responsible for repressing translation (e.g., Ago2 and Ago4); proteins and protein domains responsible for repression of RNA splicing (e.g., PTB, Sam68, and hnRNP A1); proteins and protein domains responsible for reducing the efficiency of transcription (e.g., FUS (TLS)).

In some embodiments, fusion proteins are targeted by a guide nucleic acid (e.g., guide RNA) to a specific location in a target nucleic acid and exert locus-specific regulation such as blocking RNA polymerase binding to a promoter (which selectively inhibits transcription activator function), and/or changes a local chromatin status (e.g., when a fusion sequence is used that edits the target nucleic acid or modifies a protein associated with the target nucleic acid). In some embodiments, the modifications are transient (e.g., transcription repression or activation). In some embodiments, the modifications are inheritable. For example, epigenetic modifications made to a target nucleic acid, or to proteins associated with the target nucleic acid, e.g., nucleosomal histones, in a cell, can be observed in a successive generation.

In some embodiments, fusion partner comprises an RNA splicing factor. The RNA splicing factor may be used (in whole or as fragments thereof) for modular organization, with separate sequence-specific RNA binding modules and splicing effector domains. In some embodiments, the RNA splicing factors comprise members of the Serine/Arginine-rich (SR) protein family containing N-terminal RNA recognition motifs (RRMs) that bind to exonic splicing enhancers (ESEs) in pre-mRNAs and C-terminal RS domains that promote exon inclusion. In some embodiments, a hnRNP protein hnRNP Al binds to exonic splicing silencers (ESSs) through its RRM domains and inhibits exon inclusion through a C-terminal Glycine-rich domain. In some embodiments, the RNA splicing factors may regulate alternative use of splice site (ss) by binding to regulatory sequences between two alternative sites. For example, in some embodiments, ASF/SF2 may recognize ESEs and promote the use of intron proximal sites, whereas hnRNP Al may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. Long splicing isoform Bcl-xL is a potent apoptosis inhibitor expressed in long-lived postmitotic cells and is up-regulated in many cancer cells, protecting cells against apoptotic signals. Short isoform Bcl-xS is a pro-apoptotic isoform and expressed at high levels in cells with a high turnover rate (e.g., developing lymphocytes). A ratio of the two Bcl-x splicing isoforms is regulated by multiple c\acute{ω}-elements that are located in either core exon region or exon extension region (i.e., between the two alternative 5′ splice sites). For more examples, see WO2010075303, which is hereby incorporated by reference in its entirety.

### Recombinases

In some embodiments, fusion partners comprise a recombinase. In some embodiments, effector proteins described herein are linked with the recombinase. In some embodiments, the effector proteins have reduced nuclease activity or no nuclease activity. In some embodiments, the recombinase is a site-specific recombinase.

In some embodiments, a catalytically inactive effector protein is linked with a recombinase, wherein the recombinase can be a site-specific recombinase. Such polypeptides can be used for site-directed transgene insertion. Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include: Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety. In some embodiments, the fusion protein comprises a linker that links the recombinase to the Cas-CRISPR domain of the effector protein. In some embodiments, the linker is The-Ser.

V. Exemplary systems

In some embodiments, the present disclosure provides a system comprising a guide RNA or a polynucleotide encoding the same and an effector protein or fusion protein thereof or a polynucleotide encoding the same.

In some embodiments, the system comprises an effector protein comprising an amino acid sequence that is at least 90%, at least 95%, or 100% identical to any one of the sequences recited in TABLES 7, 10 or 11, and the guide RNA comprises a repeat sequence that is at least 90%, at least 95%, or 100% identical to any one of SEQ ID NOs: 115 or 237-242 and a spacer sequence that is at least 90%, at least 95%, or 100% identical to any one of SEQ ID NOs: 1-114, 456, or 481-596. In some embodiments, the system comprises an effector protein comprising an amino acid sequence that is at least 90%, at least 95%, or 100% identical to any one of the sequences recited in TABLES 7, 10 or 11, and the guide RNA comprises a sequence that is at least 90% identical to any one of SEQ ID NOs: 116-229, 461, or 602-717.

In some embodiments, the effector protein comprises an amino acid substitution relative to SEQ ID NO: 230 selected from the group consisting of L26R, E109R, H208R, K184R, K38R, L182R, Q183R, S108R, S198R, and T114R. In some embodiments, the effector protein is a dCas protein. In some embodiments, the dCas protein comprises an amino acid substation of D369A, D369N, D658A, D658N, E567A, and E567Q relative to SEQ ID NO: 230.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 230 and a guide RNA comprising a spacer sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 1-114, 456, and 481-596. In some embodiments, the system comprises an effector protein comprising SEQ ID NO: 230 and a guide RNA comprising a spacer sequence selected from SEQ ID NOs: 1-114, 456, and 481-596. In some embodiments, the system comprises an effector protein consisting of SEQ ID NO: 230 and a guide RNA comprising a spacer sequence consisting of a sequence selected from SEQ ID NOs: 1-114, 456, and 481-596.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 230 and a guide RNA comprising a sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 116-229, 461, and 602-717. In some embodiments, the system comprises an effector protein comprising SEQ ID NO: 230 and a guide RNA selected from SEQ ID NOs: 116-229, 461, and 602-717. In some embodiments, the system comprises an effector protein consisting of SEQ ID NO: 230 and a guide RNA consisting of a sequence selected from SEQ ID NOs: 116-229, 461, and 602-717.

In some embodiments, the system comprises an effector protein comprising an amino acid sequence that is at least 90%, at least 95%, or 100% identical to any one of the sequences recited in TABLE 7, 8, or 9, and the guide RNA comprises a repeat sequence that is at least 90%, at least 95%, or 100% identical to SEQ ID NO: 350 and a spacer sequence that is at least 90%, at least 95%, or 100% identical to any one of SEQ ID NOs: 275-349, 457-460, and 476-480. In some embodiments, the guide RNA further comprises an intermediary sequence that is at least 90%, at least 95%, or 100% identical to SEQ ID NO: 351. In some embodiments, the guide RNA further comprises a handle sequence that is at least 90%, at least 95%, or 100% identical to SEQ ID NO: 352. In some embodiments, the system comprises an effector protein comprising an amino acid sequence that is at least 90%, at least 95%, or 100% identical to any one of the sequences recited in TABLE 7, 8, or 9, and the guide RNA comprises a sequence that is at least 90% identical to any one of SEQ ID NO: 353-427, 462-465, or 597-601. In some embodiments, the effector protein comprises an amino acid substitution relative to SEQ ID NO: 428 selected from the group consisting of D220R, N286K, E225K, I80K, S209F, Y315M, N193K, M298L, M295W, A306K, A218K, and K58W. In some embodiments, the effector protein is a dCas protein. In some embodiments, the dCas protein comprises an amino acid substation of E335Q, D237A D418A, D418N, and E335 relative to SEQ ID NO: 428.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 428 and a guide RNA comprising a sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 353-427, 462-465, and 597-601. In some embodiments, the system comprises an effector protein comprising SEQ ID NO: 428 and a guide RNA selected from SEQ ID NOs: 353-427, 462-465, and 597-601. In some embodiments, the system comprises an effector protein consisting of SEQ ID NO: 428 and a guide RNA consisting of a sequence selected from SEQ ID NOs: 353-427, 462-465, and 597-601.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 428 and a guide RNA comprising a spacer sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 275-349, 457-460, and 476-480. In some embodiments, the system comprises an effector protein comprising SEQ ID NO: 428 and a guide RNA comprising a spacer sequence selected from SEQ ID NOs: 275-349, 457-460, and 476-480. In some embodiments, the system comprises an effector protein consisting of SEQ ID NO: 428 and a guide RNA comprising a spacer sequence consisting of a sequence selected from SEQ ID NOs: 275-349, 457-460, and 476-480.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 230, wherein the effector protein is fused to a base editing enzyme and a guide RNA comprising a spacer sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 481-485. In some embodiments, the system comprises an effector protein comprising SEQ ID NO: 230, wherein the effector protein is fused to a base editing enzyme and a guide RNA comprising a spacer sequence comprising a sequence selected from SEQ ID NOs: 481-485. In some embodiments, the system comprises an effector protein consisting of SEQ ID NO: 230, wherein the effector protein is fused to a base editing enzyme and a guide RNA comprising a spacer sequence consisting of a sequence selected from SEQ ID NOs: 481-485.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 428, wherein the effector protein is fused to a base editing enzyme and a guide RNA comprising a spacer sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 476-480. In some embodiments, the system comprises an effector protein comprising SEQ ID NO: 428, wherein the effector protein is fused to a base editing enzyme and a guide RNA comprising a spacer sequence comprising a sequence selected from SEQ ID NOs: 476-480. In some embodiments, the system comprises an effector protein consisting of SEQ ID NO: 428, wherein the effector protein is fused to a base editing enzyme and a guide RNA comprising a spacer sequence consisting of a sequence selected from SEQ ID NOs: 476-480.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 230, wherein the effector protein is fused to a KRAB domain, a methyltransferase, or a combination thereof and a guide RNA comprising a spacer sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 456 and 486-596. In some embodiments, the system comprises an effector protein comprising SEQ ID NO: 230, wherein the effector protein is fused to a KRAB domain, a methyltransferase, or a combination thereof and a guide RNA comprising a spacer sequence comprising a sequence selected from SEQ ID NOs: 456 and 486-596. In some embodiments, the system comprises an effector protein consisting of SEQ ID NO: 230, wherein the effector protein is fused to a KRAB domain, a methyltransferase, or a combination thereof and a guide RNA comprising a spacer sequence consisting of a sequence selected from SEQ ID NOs: 456 and 486-596.

### VI. Target Nucleic Acids

Disclosed herein are compositions, systems and methods for detecting and/or editing a target nucleic acid (e.g., the DUX4 gene). In general, the target nucleic acid is the DUX4 gene or a portion thereof. In general, guide nucleic acids described herein comprise a sequence that is complementary to and/or hybridizes to a target sequence in the DUX4 gene. Exemplary reference sequences for the DUX4 gene are provided in TABLE 15. The target sequence of the DUX4 gene may be a portion of the DUX4 gene that encodes the DUX4 protein. Exemplary reference sequences for the DUX4 protein are listed in TABLE 16.

TABLE 15
Exemplary reference DUX4 gene

HGNC: 50800; NCBI Entrez Gene: 100288687; Ensembl: ENSG00000260596; OMIM: 606009;
RefSeq:NC_000004.12; RefSeq NC_060928.1; NCBI Reference Sequence: NG_034189.3

TABLE 16
Exemplary reference DUX4 proteins

NCBI Reference Sequence: NP_001292997.1; NP_001280727.1;
Protein Accession: Q9UBX2; Protein Accessions: E2JJS1

### Certain Samples

Systems, compositions, and methods described herein may be useful for detecting a mutated DUX4 gene in a sample. In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some embodiments, an environmental sample is taken as a swab from a surface of interest or taken directly from the surface of interest.

### VII. Vectors

Compositions, systems, and methods described herein comprise a vector or a use thereof. A vector can comprise a nucleic acid of interest (e.g., a DUX4-targeting guide nucleic acid or polynucleotide encoding the same). In some embodiments, the nucleic acid of interest comprises one or more components of a composition or system described herein (e.g., a DUX4-targeting guide nucleic acid or polynucleotide encoding the same). In some embodiments, the nucleic acid of interest comprises a nucleotide sequence that encodes one or more components of the composition or system described herein. In some embodiments, one or more components comprises a polypeptide(s), guide nucleic acid(s), target nucleic acid(s), and donor nucleic acid(s). In some embodiments, the component comprises a nucleic acid encoding an effector protein and a guide nucleic acid or a nucleic acid encoding the guide nucleic acid. The vector may be part of a vector system, wherein a vector system comprises a library of vectors each encoding one or more component of a composition or system described herein. In some embodiments, components described herein (e.g., an effector protein, a guide nucleic acid, and/or a target nucleic acid) are encoded by the same vector. In some embodiments, components described herein (e.g., an effector protein, a guide nucleic acid, and/or a target nucleic acid) are each encoded by different vectors of the system.

In some embodiments, a vector comprises a nucleotide sequence encoding one or more effector proteins as described herein. In some embodiments, the one or more effector proteins comprise at least two effector proteins. In some embodiments, the at least two effector protein are the same. In some embodiments, the at least two effector proteins are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more effector proteins.

In some embodiments, a vector may encode one or more of any system components, including but not limited to effector proteins, guide nucleic acids, donor nucleic acids, and target nucleic acids as described herein. In some embodiments, a system component encoding sequence is operably linked to a promoter that is operable in a target cell, such as a eukaryotic cell. In some embodiments, a vector may encode 1, 2, 3, 4 or more of any system components. For example, a vector may encode two or more guide nucleic acids, wherein each guide nucleic acid comprises a different sequence. A vector may comprise the nucleic acid encoding an effector protein and a guide nucleic acid. A vector may encode an effector protein, a guide nucleic acid, and a donor nucleic acid.

In some embodiments, a vector comprises one or more guide nucleic acids, or a nucleotide sequence encoding the one or more guide nucleic acids as described herein (e.g., a DUX4-targeting guide nucleic acid or polynucleotide encoding the same). In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter-a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or the effector protein.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, the length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin promoter, CAG, TRE, UAS, Ac5, Polyhedrin, CaMKIIa, GAL1-10, H1, TEF1, GDS, ADH1, HSV TK, Ubi, U6, MNDU3, MSCV, MND and CAG. In some embodiments, the promoter allows for expression in a muscle cell. Non-limiting examples of such promoters are Ck8e, Spc5-12, Mb, and Desmin.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. In some embodiments, the promoter for expressing effector protein is a muscle-specific promoter. In some embodiments, the muscle-specific promoter comprises Ck8e, SPC5-12, Mb, or Desmin promoter sequence. In some embodiments, the promoter for expressing effector protein is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA.

In some embodiments, the expression vector comprises the DNA molecule encoding a guide nucleic acid. In some embodiments, the expression vector further comprises the nucleic acid encoding an effector protein. In some embodiments, the expression vector further comprises or encodes a donor nucleic acid. In some embodiments, the expression vector encoding a guide nucleic acid, wherein the guide nucleic acid comprises a first region comprising a repeat; and a second region comprising a spacer sequence that is complementary to a target sequence of a DUX4 gene. In some embodiments, wherein the first region is located 5′ of the second region.

In some embodiments, the expression vector further comprises an effector protein that binds the repeat sequence or a nucleic acid encoding the effector protein. In some embodiments, the spacer comprises a nucleotide sequence that is at least 70%, at least 75%, at least 80%, at least 85%, at least 90%, at least 95%, or 100% identical to a sequence selected from SEQ ID NOs: 1-114, 456, and 481-596; the repeat sequence comprises a nucleotide sequence that is at least 70%, at least 75%, at least 80%, at least 85%, at least 90%, at least 95%, or 100% identical to a sequence selected from SEQ ID NOs: 115 and 237-242; the effector protein comprises an amino acid sequence that is at least 65%, at least 70%, at least 75%, at least 80%, at least 85%, at least 90%, at least 95%, at least 97%, at least 98%, at least 99%, or 100% identical to a sequence selected from SEQ ID NOs: 230-233, 243-244, and 262-274; or a combination thereof. In some embodiments, the spacer comprises a nucleotide sequence that is at least 70%, at least 75%, at least 80%, at least 85%, at least 90%, at least 95%, or 100% identical to a sequence selected from SEQ ID NOs: 275-349, 457-460, and 476-480; the repeat sequence comprises a nucleotide sequence that is at least 70%, at least 75%, at least 80%, at least 85%, at least 90%, at least 95%, or 100% identical to SEQ ID NO: 350; the effector protein comprises an amino acid sequence that is at least 65%, at least 70%, at least 75%, at least 80%, at least 85%, at least 90%, at least 95%, at least 97%, at least 98%, at least 99%, or 100% identical to a sequence selected from SEQ ID NOs: 428-446; or a combination thereof.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I.

In some embodiments, disclosed herein comprise one or more nucleic acids encoding an effector protein, fusion effector protein, fusion partner, a guide nucleic acid, or a combination thereof. The effector protein, fusion effector protein, fusion partner protein, or combination thereof may be any one of those described herein. In some embodiments, of the above, the nucleic acid expression vector comprises a polynucleotide encoding an effector protein that is at least 80%, at least 85%, at least 90%, at least 95%, at least 99%, or 100% identical to any one of the sequences recited in TABLES 7-11.

The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein. In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more effector proteins, one or more donor nucleic acids, and one or more guide nucleic acids or a nucleic acid encoding the one or more guide nucleic acid, are provided in the single expression vector. In some embodiments, components, such as a guide nucleic acid and an effector protein, are encoded by the same vector. In some embodiments, an effector protein (or a nucleic acid encoding same) and/or an engineered guide nucleic acid (or a nucleic acid that, when transcribed, produces same) are not co-administered with donor nucleic acid in a single vehicle. In some embodiments, an effector protein (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector may be part of a vector system. In some embodiments, the vector system comprises a library of vectors each encoding one or more components of a composition or system described herein. In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more effector proteins, one or more donor nucleic acids, and one or more guide nucleic acids or a nucleic acid encoding the one or more guide nucleic acids, or a variant thereof is provided in a different vector. In some embodiments, the nucleic acid encoding the effector protein, and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28 (3): 146-157). In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino) propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEG), derivatives, analogs, or variants thereof. In some embodiments, the LNP has a negative net overall charge prior to complexation with one or more of a guide nucleic acid, a nucleic acid encoding the one or more guide nucleic acid, a nucleic acid encoding the effector protein, and/or a donor nucleic acid. In some embodiments, the inner core is a hydrophobic core. In some embodiments, the one or more of a guide nucleic acid, the nucleic acid encoding the one or more guide nucleic acid, the nucleic acid encoding the effector protein, and/or the donor nucleic acid forms a complex with one or more of the cationic lipids and the ionizable lipids. In some embodiments, the nucleic acid encoding the effector protein or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

### Delivery of Viral Vectors

In some embodiments, a vector described herein comprises a viral vector. In some embodiments, the viral vector comprises a nucleic acid to be delivered into a host cell by a recombinantly produced virus or viral particle. The nucleic acid may be single-stranded or double stranded, linear or circular, segmented or non-segmented. The nucleic acid may comprise DNA, RNA, or a combination thereof. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein may be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector provided herein comprises a sequence encoding genome editing tools. In some embodiments, the genome editing tools comprise a nucleic acid encoding one or more effector proteins, a nucleic acid encoding one or more fusion proteins (e.g., a nuclear localization signal (NLS), polyA tail), one or more guide nucleic acids, a nucleic acid encoding the one or more guide nucleic acids, respective promoter(s), one or more donor nucleic acid, or any combinations thereof. In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

### Producing AAV Delivery Vectors

In some embodiments, methods of producing AAV delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging an effector encoding nucleic acid into the AAV particle, thereby generating an AAV delivery vector. In some embodiments, promoters, stuffer sequences, and any combination thereof may be packaged in the AAV vector. In some examples, the AAV vector may package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some embodiments, the AAV vector comprises a recombinant AAV expression cassette comprising sequences encoding: a) a first inverted terminal repeat (ITR) and a first promoter; b) an effector protein disclosed herein; c) optionally a second promoter; d) a second polynucleotide encoding a guide nucleic acid disclosed here; and e) a second ITR. In some embodiments, the AAV expression cassette is a self-complementary AAV vector.

### Producing AAV Particles

In some embodiments, AAV particles described herein are recombinant AAV (rAAV). In some embodiments, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31 (4): 317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3 (12): 2156-65; Urabe et al., (2002), Hum. Gene. Ther., 1; 13 (16): 1935-43; and Benskey et al., (2019), Methods Mol Biol., 1937:3-26, each of which is incorporated by reference in its entirety.

### VIII. Pharmaceutical Compositions and Modes of Administration

Disclosed herein are compositions comprising one or more effector proteins described herein or nucleic acids encoding the one or more effector proteins, one or more guide nucleic acids described herein or nucleic acids encoding the one or more guide nucleic acids described herein (e.g., DUX4-targeting guide nucleic acids or polynucleotides encoding the same), or combinations thereof. In some embodiments, a repeat sequence of the one or more guide nucleic acids are capable of interacting with the one or more of the effector proteins. In some embodiments, spacer sequences of the one or more guide nucleic acids hybridizes with a target sequence of a target nucleic acid. In some embodiments, the compositions are capable of editing a target nucleic acid in a cell or a subject. In some embodiments, the compositions are capable of editing a target nucleic acid or the expression thereof in a cell, in a tissue, in an organ, in vitro, in vivo, or ex vivo. In some embodiments, the compositions are capable of editing a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

In some embodiments, compositions described herein are pharmaceutical compositions. In some embodiments, the pharmaceutical compositions comprise compositions described herein and a pharmaceutically acceptable carrier or diluent. Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

Pharmaceutical compositions described herein comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺ SO₄²⁻.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

Disclosed herein, in some embodiments, are pharmaceutical compositions for modifying a target nucleic acid in a cell or a subject, comprising any one of the effector proteins, engineered effector proteins, fusion effector proteins, or guide nucleic acids as described herein and any combination thereof. Also disclosed herein, in some aspects, are pharmaceutical compositions comprising a nucleic acid encoding any one of the effector proteins, engineered effector proteins, fusion effector proteins, or guide nucleic acids as described herein and any combination thereof. Also disclosed herein, are pharmaceutical compositions comprising the nucleic acid expression vector, the cell, or the population of cells disclosed herein. In some embodiments, pharmaceutical compositions comprise a plurality of guide nucleic acids. In some embodiments, the pharmaceutical composition disclosed herein also comprise a pharmaceutical acceptable carrier. Pharmaceutical compositions may be used to modify a target nucleic acid or the expression thereof in a cell in vitro, in vivo or ex vivo. In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein.

### IX. Methods of Detecting a Target Nucleic Acid

Provided herein are methods of detecting target nucleic acids. Methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some embodiments, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid.

### X. Methods of Nucleic Acid Modification

Provided herein are methods of editing and modifying the expression of target nucleic acids (e.g., a target nucleic acid in the DUX4 gene). In general, editing refers to modifying the nucleobase sequence of a target nucleic acid. However, compositions and systems disclosed herein may also be capable of making epigenetic modifications of target nucleic acids. Effector proteins, multimeric complexes thereof and systems described herein may be used for editing or modifying a target nucleic acid. Editing a target nucleic acid may comprise one or more of: cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid.

Methods of editing may comprise contacting a target nucleic acid with an effector protein described herein and a guide nucleic acid, wherein the effector protein comprises an amino acid sequence that is at least 70%, at least 75%, at least 80%, at least 85%, at least 90%, at least 95%, at least 97%, or at least 98%, at least 99%, or 100% identical to any one of the sequences set forth in TABLES 7, 10, and 11. In some embodiments, the effector protein comprises an amino acid sequence that is at least 70%, at least 75%, at least 80%, at least 85%, at least 90%, at least 95%, at least 97%, at least 98%, at least 99%, or 100% identical to any one of the sequences set forth in TABLES 7, 10, and 11, wherein the amino acid residue at position 26, relative to SEQ ID NO:230, remains unchanged. In some embodiments, the effector protein comprises an amino acid substitution relative to SEQ ID NO: 230 selected from the group consisting of L26R, E109R, H208R, K184R, K38R, L182R, Q183R, S108R, S198R, and T114R. In some embodiments, the effector protein is a dCas protein. In some embodiments, the dCas protein comprises an amino acid substation D369A, D369N, D658A, D658N, E567A, and E567Q relative to SEQ ID NO: 230. In some embodiments, the guide nucleic acid comprises a nucleotide sequence that is at least 70%, at least 75%, at least 80%, at least 85%, at least 90%, at least 92%, at least 95%, or 100% identical to any one of the sequences set forth in TABLE 4. In some embodiments, the guide nucleic acid comprises a spacer sequence that is at least 70%, at least 75%, at least 80%, at least 85%, at least 90%, at least 92%, at least 95%, or 100% identical to any one of the sequences set forth in TABLE 1 and a repeat sequence that is at least 70%, at least 75%, at least 80%, at least 85%, at least 90%, at least 92%, at least 95%, or 100% identical to the sequence set forth in TABLE 3.

Methods of editing may comprise contacting a target nucleic acid with an effector protein described herein and a guide nucleic acid, wherein the effector protein comprises an amino acid sequence that is at least 70%, at least 75%, at least 80%, at least 85%, at least 90%, at least 95%, at least 97%, or at least 98%, at least 99%, or 100% identical to any one of the sequences set forth in TABLES 7, 8, and 9. In some embodiments, the effector protein comprises an amino acid sequence that is at least 70%, at least 75%, at least 80%, at least 85%, at least 90%, at least 95%, at least 97%, at least 98%, at least 99%, or 100% identical to any one of the sequences set forth in TABLES 7, 8, and 9, wherein the amino acid residue at position 220, relative to SEQ ID NO: 428, remains unchanged. In some embodiments, the effector protein comprises an amino acid substitution relative to SEQ ID NO: 428 selected from the group consisting of D220R, N286K, E225K, 180K, S209F, Y315M, N193K, M298L, M295W, A306K, A218K, and K58W. In some embodiments, the effector protein is a dCas protein. In some embodiments, the dCas protein comprises an amino acid substation of E335Q, D237A D418A, D418N, and E335A relative to SEQ ID NO: 428. In some embodiments, the guide nucleic acid comprises a nucleotide sequence that is at least 70%, at least 75%, at least 80%, at least 85%, at least 90%, at least 92%, at least 95%, or 100% identical to any one of the sequences set forth in TABLE 5. In some embodiments, the guide nucleic acid comprises a spacer sequence that is at least 70%, at least 75%, at least 80%, at least 85%, at least 90%, at least 92%, at least 95%, or 100% identical to any one of the sequences set forth in TABLE 2 and a repeat sequence that is at least 70%, at least 75%, at least 80%, at least 85%, at least 90%, at least 92%, at least 95%, or 100% identical to SEQ ID NO: 350. In some embodiments the guide nucleic acid comprises a handle sequence that is at least 70%, at least 75%, at least 80%, at least 85%, at least 90%, at least 92%, at least 95%, or 100% identical to SEQ ID NO: 352. In some embodiments the guide nucleic acid comprises an intermediary sequence that is at least 70%, at least 75%, at least 80%, at least 85%, at least 90%, at least 92%, at least 95%, or 100% identical to SEQ ID NO: 351.

Editing may introduce a mutation (e.g., point mutations, deletions) in a target nucleic acid relative to a corresponding wildtype nucleobase sequence. Editing may remove or correct a disease-causing mutation in a nucleic acid sequence to produce a corresponding wildtype nucleobase sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer region. In some embodiments, the target nucleic acid, and the resulting cleaved nucleic acid is contacted with a nucleic acid for homologous recombination (e.g., homology directed repair (HDR)) or non-homologous end joining (NHEJ). In some cases, a double-stranded break in the target nucleic acid may be repaired (e.g., by NHEJ or HDR) without insertion of a donor template, such that the repair results in an indel in the target nucleic acid at or near the site of the double-stranded break.

In some embodiments, an indel, sometimes referred to as an insertion-deletion or indel mutation, is a type of genetic mutation that results from the insertion and/or deletion of nucleotides in a target nucleic acid. An indel can vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation.

In some embodiments, wherein the compositions, systems, and methods of the present disclosure comprise an additional guide nucleic acid or a use thereof, the dual-guided compositions, systems, and methods described herein can modify the target nucleic acid in two locations. In some cases, dual-guided editing can comprise cleavage of the target nucleic acid in the two locations targeted by the guide RNAs. In certain embodiments, upon removal of the sequence between the guide nucleic acids, the wild-type reading frame is restored. A wild-type reading frame can be a reading frame that produces at least a partially, or fully, functional protein. A non-wild-type reading frame can be a reading frame that produces a non-functional or partially non-functional protein.

Accordingly, in some embodiments, compositions, systems, and methods described herein can edit 1 to 1,000 nucleotides or any integer in between, in a target nucleic acid. In certain embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, can be edited by the compositions, systems, and methods described herein. In some embodiments, 1, 2, 3, 4, 5, 6, 7, 8, 9, 10, or more nucleotides can be edited by the compositions, systems, and methods described herein. In some embodiments, 10, 20, 30, 40, 50, 60, 70, 80 90, 100 or more nucleotides, or any integer in between, can be edited by the compositions, systems, and methods described herein. In some embodiments, 100, 200, 300, 400, 500, 600, 700, 800, 900 or more nucleotides, or any integer in between, can be edited by the compositions, systems, and methods described herein.

In some cases, methods comprise editing a target nucleic acid with two or more effector proteins. Editing a target nucleic acid may comprise introducing a two or more single-stranded breaks in a target nucleic acid. In some embodiments, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with donor nucleic acid), thereby modifying the target nucleic acid.

Methods, systems and compositions described herein can edit or modify a target nucleic acid wherein such editing or modification can be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity can be detected by next generation sequencing of one or more target loci of a target nucleic acid where indel percentage is calculated as the fraction of sequencing reads containing insertions or deletions relative to an unedited reference sequence. In certain embodiments, methods, systems, and compositions comprising an effector protein and guide nucleic acid described herein can exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein can exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In certain embodiments, sequence deletion is a modification where one or more sequences in a target nucleic acid are deleted relative to a target nucleic acid without the sequence deletion. In certain embodiments, a sequence deletion can result in or effect a splicing disruption or a frameshift mutation. In certain embodiments, a sequence deletion result in or effect a splicing disruption.

In certain embodiments, a modification is a deletion of an entire exon. In some embodiments, the exon is associated with a disease. In some embodiments, compositions, systems, and methods described herein comprise a combination of a first gRNA, a second gRNA, a first effector protein, and a second effector protein, wherein the combination can be used for deleting the entire exon or a portion thereof. In some embodiments, the first effector protein and the second effector protein are the same. In some embodiments, the first effector protein and the second effector protein are not the same.

In certain embodiments, sequence skipping is a modification where one or more sequences in a target nucleic acid are skipped upon transcription or translation of the target nucleic acid relative to a target nucleic acid without the sequence skipping. In certain embodiments, sequence skipping can result in or effect a splicing disruption or a frameshift mutation. In certain embodiments, sequence skipping can result in or effect a splicing disruption.

In certain embodiments, sequence reframing is a modification where one or more bases in a target are modified so that the reading frame of the sequence is reframed relative to a target nucleic acid without the sequence reframing. In certain embodiments, sequence reframing can result in or effect a splicing disruption or a frameshift mutation. In certain embodiments, sequence reframing can result in or effect a frameshift mutation.

In certain embodiments, sequence knock-in is a modification where one or more sequences is inserted into a target nucleic acid relative to a target nucleic acid without the sequence knock-in. In certain embodiments, sequence knock-in can result in or effect a splicing disruption or a frameshift mutation. In certain embodiments, sequence knock-in can result in or effect a splicing disruption.

In certain embodiments, editing or modification of a target nucleic acid can be locus specific, wherein compositions, systems, and methods described herein can edit or modify a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing or modification of a specific locus can affect any one of a splicing disruption, frameshift (e.g., 1+ or 2+ frameshift), sequence deletion, sequence skipping, sequence reframing, sequence knock-in, or any combination thereof. In certain embodiments, editing or modification of a target nucleic acid can be locus specific, modification specific, or both. In certain embodiments, editing or modification of a target nucleic acid can be locus specific, modification specific, or both, wherein compositions, systems, and methods described herein comprise an effector protein described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. Methods of editing a target nucleic acid or modulating the expression of a target nucleic acid may be performed ex vivo. Editing methods include, but are not limited to, introduction of double stranded breaks (DSB), which can result in deleting some nucleotides and disrupting the translation of a functional protein, base editing, and splice acceptor disruption (SA).

In some embodiments the method of editing by the effector proteins can be promotor silencing, frameshift mutation, base editing, or splice disruption.

In some embodiments the editing by the effector protein targets exon 1. In some embodiments the editing by the effector protein targets exon 3. In some embodiments the editing by the effector protein targets intron 2. In some embodiments the editing by the effector protein targets the 3′ UTR. In some embodiments the editing by the effector protein targets the poly-A tail. In some embodiments the editing by the effector protein decreases transcription of the DNA sequence. In some embodiments the editing by the effector protein decreases translation of the RNA sequence.

In some embodiments the gene regulation is regulated by effector protein repressing a promoter. In some embodiments the repression is temporary or transient. In some embodiments the repression is permanent. In some embodiments the effector protein is linked to a KRAB sequence. In some embodiments the effector protein is linked to an acetylase sequence. In some embodiments the effector protein is linked to a methyltransferase. In some embodiments the effector protein is linked to a Ezh2 sequence.

In some embodiments the effector protein causes a frameshift mutation. In some embodiments the effector protein causes the addition of one or more nucleotides causing a shift in the reading frame. In some embodiments the effector protein causes a deletion of one or more nucleotides causing a shift in the reading frame. In some embodiments the effector protein causes the deletion or addition of 1, 2, or 4 nucleotides. In some embodiments the effector protein causes an alternation in the amino acid sequence at protein translation. In some embodiments the alteration is a missense mutation. In some embodiments the alteration is a premature stop codon. In some embodiments the effector protein causes a change in the ribosome reading frame and cause premature termination of translation at a new nonsense or chain termination codon (TAA, TAG, and TGA).

In some embodiments the effector protein causes a nucleobase to be edited. In some embodiments the effector protein is linked to an adenine base editing enzyme (e.g., an ABE). In some embodiments, the effector protein is linked to a cytosine base editing enzyme (e.g., a CBE). In some embodiments, the fusion protein causes a cytodine to thymidine transition. In some embodiments, the fusion protein causes a cytodine to uracil transition. In some embodiments, the fusion protein causes a thymidine to cytodine transition. In some embodiments, the fusion protein causes an adenosine to guanosine transition. In some embodiments, the fusion protein causes a guanosine to adenosine conversion. In some embodiments, the alteration results in a missense mutation. In some embodiments, the alteration is a premature stop codon. In some embodiments, the fusion protein causes a premature termination of translation at a new nonsense or chain termination codon (TAA, TAG, and TGA).

### XI. Method of Treating a Disorder

Described herein are methods for treating or preventing a disease in a subject by modifying a target nucleic acid associated with a gene (e.g., DUX4) or expression of a gene related to the disease. In some embodiments, the disease or disorder comprises a mutation in the DUX4 gene. In some embodiments the disease or disorder is a muscular dystrophy. In some embodiments the muscular dystrophy is facioscapulohumeral muscular dystrophy (FSHD). In some embodiments the muscular dystrophy is FSHD type 1 (FSHD1). In some embodiments the muscular dystrophy is FSHD type 2 (FSHD2). In some embodiments the disease or disorder is a cancer. In some embodiments the cancer is a B-cell cancer. In some embodiments the cancer is a sarcoma.

In some embodiments, the method for treating a disease comprises modifying the at least one gene associated with the disease or modifying expression of the at least one gene such that the disease is treated. In some embodiments, the gene encodes a DUX4 protein. In some embodiments, the disease is any one of the diseases or disorders listed above and the gene is the gene set forth in TABLE 15.

In some embodiments, methods comprise administering a guide RNA comprising one or more sequences selected from the sequences in TABLES 1-5 and SEQ ID NOs: 236 and 351-352, or a nucleic acid encoding the same. In some embodiments, methods comprise administering a Cas protein or a nucleic acid encoding the same. In some embodiments, the Cas protein comprises an amino acid sequence that is at least 90% or 95% identical to any one of the sequences in TABLES 7-11. The Cas protein or nucleic acid encoding the same, and the guide RNA or nucleic acid encoding the same may be administered in a single composition. The Cas protein or nucleic acid encoding the same, and the guide RNA or nucleic acid encoding the same may be administered separately (formulaically or chronologically). In some embodiments, methods comprise administering: a Cas protein or a messenger RNA encoding a Cas protein and a lipid nanoparticle; and a viral vector encoding a guide RNA. In some embodiments, methods comprise administering a viral vector encoding the Cas protein and the guide RNA. In some embodiments, methods comprise administering a Cas protein and a lipid nanoparticle. In some embodiments, methods comprise administering a messenger RNA encoding a Cas protein.

### Further Numbered Embodiments

The present invention is also described, for example and without limitation, in the following numbered embodiments which are not to be construed as limiting the scope thereof in any manner.

Embodiment 1: A guide ribonucleic acid (RNA) or a polynucleotide encoding the same, wherein the guide RNA comprises: (a) a first region comprising a protein binding sequence, and (b) a second region comprising a targeting sequence that is complementary to a target sequence that is within a DUX4 gene; wherein the protein binding sequence is capable of being bound by a clustered regularly interspaced short palindromic repeats (CRISPR) Cas protein other than a Cas9 protein.

Embodiment 2: The guide RNA of embodiment 1, wherein the protein binding sequence comprises a repeat sequence.

Embodiment 3: The guide RNA of any one of embodiments 1-2, wherein the targeting sequence comprises a spacer sequence.

Embodiment 4: The guide RNA of any one of embodiments 1-3, wherein the target sequence comprises at least a portion of a DUX4 promoter, a DUX4 exon 1, a DUX4 exon 2, a DUX4 intron 2-exon 3 junction, a DUX4 intron 2, a 3′ untranslated region (UTR) region of the DUX4 gene, a DUX4 exon 3, or a combination thereof.

Embodiment 5: The guide RNA of any one of embodiments 1-4, wherein the target sequence is within the exon 1 region of the DUX4 gene.

Embodiment 6: The guide RNA of embodiment 5, wherein the targeting sequence is at least 80%, at least 85%, at least 90%, at least 95%, or 100% identical to a sequence selected from SEQ ID NOs: 1-55 and 508-596.

Embodiment 7: The guide RNA of any one of embodiments 1-4, wherein the target sequence is within the exon 2 region of the DUX4 gene.

Embodiment 8: The guide RNA of embodiment 7, wherein the targeting sequence is at least 80%, at least 85%, at least 90%, at least 95%, or 100% identical to a sequence selected from SEQ ID NOs: 56-75.

Embodiment 9: The guide RNA of any one of embodiments 1-4, wherein the target sequence is within the intron 2 region of the DUX4 gene.

Embodiment 10: The guide RNA of embodiment 9, wherein the targeting sequence is at least 80%, at least 85%, at least 90%, at least 95%, or 100% identical to a sequence selected from SEQ ID NOs: 76-85.

Embodiment 11: The guide RNA of any one of embodiments 1-4, wherein the target sequence straddles intron 2 and exon 3 regions of the DUX4 gene.

Embodiment 12: The guide RNA of embodiment 11, wherein the targeting sequence is at least 80%, at least 85%, at least 90%, at least 95% or 100% identical to a sequence selected from SEQ ID NO: 86.

Embodiment 13: The guide RNA of any one of embodiments 1-4, wherein the target sequence within the 3′UTR of the DUX4 gene.

Embodiment 14: The guide RNA of embodiment 13, wherein the targeting sequence is at least 80%, at least 85%, at least 90%, at least 95%, or 100% identical to a sequence selected from SEQ ID NOs: 87-114.

Embodiment 15: The guide RNA of any one of embodiments 1-4, wherein the target sequence within exon 3 of the DUX4 gene.

Embodiment 16: The guide RNA of embodiment 15, wherein the targeting sequence is at least 80%, at least 85%, at least 90%, at least 95%, or 100% identical to a sequence selected from SEQ ID NOs: 481-485.

Embodiment 17: The guide RNA of any one of embodiments 1-4, wherein the targeting sequence is at least 80%, at least 85%, at least 90%, at least 95%, or 100% identical to a sequence selected from SEQ ID NOs: 456 and 486-507.

Embodiment 18: The guide RNA of any one of embodiments 1-17, wherein the protein binding sequence is at least 80%, at least 85%, at least 90%, at least 95% or 100% identical to a sequence selected from TABLE 3.

Embodiment 19: The guide RNA of any one of embodiments 1-18, wherein the Cas protein is at least 80%, at least 85%, at least 90%, at least 95%, or 100% identical to a sequence selected from TABLES 7, 10 and 11.

Embodiment 20: The guide RNA of any one of embodiments 1-4, wherein the targeting sequence is at least 80%, at least 85%, at least 90%, at least 95%, or 100% identical to a sequence selected from SEQ ID NOs: 481-485, wherein the Cas protein is at least 80%, at least 85%, at least 90%, at least 95%, or 100% identical to SEQ ID NO: 230 and wherein the Cas protein is fused to a base editing enzyme.

Embodiment 21: The guide RNA of any one of embodiments 1-4, wherein the targeting sequence is at least 80%, at least 85%, at least 90%, at least 95%, or 100% identical to a sequence selected from SEQ ID NOs: 476-480, wherein the Cas protein is at least 80%, at least 85%, at least 90%, at least 95%, or 100% identical to SEQ ID NO: 428 and wherein the Cas protein is fused to a base editing enzyme.

Embodiment 22: The guide RNA of any one of embodiments 1-4, wherein the targeting sequence is at least 80%, at least 85%, at least 90%, at least 95%, or 100% identical to a sequence selected from SEQ ID NOs: 456 and 486-596, wherein the Cas protein is at least 80%, at least 85%, at least 90%, at least 95%, or 100% identical to SEQ ID NO: 230 and wherein the Cas protein is fused to a KRAB domain, a methyltransferase, or a combination thereof.

Embodiment 23: The guide RNA of any one of embodiments 1-4, wherein the target sequence is within the exon 1 region of the DUX4 gene.

Embodiment 24: The guide RNA of embodiments 24, wherein the targeting sequence is at least 80%, at least 85%, at least 90%, at least 95% of 100% identical to a sequence selected from SEQ ID NOs: 275-299.

Embodiment 25: The guide RNA of any one of embodiments 1-4, wherein the target sequence is within the exon 3 region of the DUX4 gene.

Embodiment 26: The guide RNA of embodiment 25, wherein the targeting sequence is at least 80%, at least 85%, at least 90%, at least 95% of 100% identical to a sequence selected from SEQ ID NOs: 300-313 and 476-480.

Embodiment 27: The guide RNA of any one of embodiments 1-4, wherein the target sequence is within the intron 2 region of the DUX4 gene.

Embodiment 28: The guide RNA of embodiment 27, wherein the targeting sequence is at least 80%, at least 85%, at least 90%, at least 95% of 100% identical to a sequence selected from SEQ ID NOs: 314-322.

Embodiment 29: The guide RNA of any one of embodiments 1-4, wherein the target sequence within the 3′UTR of the DUX4 gene.

Embodiment 30: The guide RNA of embodiment 29, wherein the targeting sequence is at least 80%, at least 85%, at least 90%, at least 95% of 100% identical to a sequence selected from SEQ ID NOs: 323-349.

Embodiment 31: The guide RNA of embodiment 30, wherein the targeting sequence is at least 80%, at least 85%, at least 90%, at least 95%, or 100% identical to a sequence selected from SEQ ID NOs: 457-460.

Embodiment 32: The guide RNA of any one of embodiments 25-31, wherein the protein binding sequence is at least 80%, at least 85%, at least 90%, at least 95% or 100% identical to SEQ ID NO: 350.

Embodiment 33: The guide RNA of any one of embodiments 25-32, wherein the Cas protein is at least 80%, at least 85%, at least 90%, at least 95%, or 100% identical to a sequence selected from TABLES 7, 8, and 9.

Embodiment 34: A system comprising the guide RNA of any one of embodiments 1-33, or the polynucleotide encoding the same.

Embodiment 35: The system of embodiment 34, comprising a Cas protein or a polynucleotide encoding the same.

Embodiment 36: The system of embodiment 35, wherein the polynucleotide is an mRNA polynucleotide.

Embodiment 37: The system of any of embodiments 34-36, wherein the polynucleotide is a DNA expression vector.

Embodiment 38: The system of embodiment 37, wherein the DNA expression vector is an adeno-associated viral (AAV) vector.

Embodiment 39: The system of embodiment 38, comprising a recombinant adeno-associated virus (AAV) expression cassette comprising sequences encoding (a) a first inverted terminal repeat (ITR) and a first promoter; (b) the Cas protein; (c) optionally a second promoter; (d) a second polynucleotide encoding the guide RNA of any one of embodiments 1-32; and (e) a second ITR; wherein the AAV expression cassette is a self-complementary AAV vector.

Embodiment 40: The system of any one of embodiments 34-39, comprising a lipid or lipid nanoparticle.

Embodiment 41: The system of any one of embodiments 34-40, wherein the Cas protein recognizes a protospacer motif (PAM) of 5′-TTN-3′.

Embodiment 42: The system of embodiment 41, wherein the Cas protein recognizes the PAM sequence selected from the group consisting of 5′-TTG-3′, 5′-TTC-3′, 5′-TTT-3′, and 5′-TTA-3′.

Embodiment 43: The system of any one of embodiments 34-42, wherein the Cas protein comprises a sequence that is at least 80%, at least 85%, at least 90%, at least 95%, at least 97%, at least 98%, at least 99%, or 100% identical to SEQ ID NO: 230.

Embodiment 44: The system of embodiment 43, wherein the Cas protein has a positively charged amino acid at position 26 of SEQ ID NO: 230.

Embodiment 45: The system of embodiment 44, wherein the positively charged amino acid is selected from arginine, histidine and lysine.

Embodiment 46: The system of embodiment 45, wherein the positively charged amino acid is arginine.

Embodiment 47: The system of any one of embodiments 34-40, wherein the Cas protein recognizes a protospacer motif (PAM) of 5′-TNTR-3′.

Embodiment 48: The system of embodiment 47, wherein the Cas protein recognizes the PAM sequence selected from the group consisting of 5′-TTTG-3′, 5′-TCTG-3′, 5′-TGTG-3′, 5′-TCTA-3′, 5′-TATA-3′, 5′-TTTA-3′, 5′-TGTA-3′, and 5′-TATG-3′.

Embodiment 49: The system of any one of embodiments 34-40 and 47-48, wherein the Cas protein comprises a sequence that is at least 80%, at least 85%, at least 90%, at least 95%, at least 97%, at least 98%, at least 99%, or 100% identical to SEQ ID NO: 428.

Embodiment 50: The system of embodiment 49, wherein the Cas protein has a positively charged amino acid at position 220 of SEQ ID NO: 428.

Embodiment 51: The system of embodiment 50, wherein the positively charged amino acid is selected from arginine, histidine and lysine.

Embodiment 52: The system of embodiment 51, wherein the positively charged amino acid is arginine.

Embodiment 53: The system of any one of embodiments 34-52, wherein the Cas protein amino acid sequence comprises a nuclear localization signal.

Embodiment 54: The system of any one of embodiments 34-53, wherein the Cas protein amino acid sequence is at least 80%, at least 85%, at least 90%, at least 95%, at least 97%, at least 98%, at least 99%, or 100% identical to a sequence from Tables 7-11.

Embodiment 55: The system of any one of embodiments 34-54, wherein the system further comprises an additional guide RNA that binds a different portion of the target nucleic acid than the guide RNA.

Embodiment 56: The system of any one of embodiments 34-55, wherein the Cas protein reduces expression of the DUX4 gene.

Embodiment 57: The system of any one of embodiments 34-56, wherein the Cas protein is linked to a heterologous protein.

Embodiment 58: The system of embodiment 57, wherein the heterologous protein is linked to the N terminus or C terminus of the Cas protein

Embodiment 59: The system of any of any one of embodiments 55-58, wherein the Cas protein is linked to a KRAB domain, acetylase domain, or a base editing enzyme.

Embodiment 60: The system of embodiment 59, wherein the base editing enzyme is a cytosine base editing enzyme (CBE), adenine base editing enzymes (ABE), or a C-to-G base editing enzymes (CGBE).

Embodiment 61: The system of any one of embodiments 55-60, wherein the expression of the DUX4 gene is reduced by promoter inhibition, a frameshift mutation, base editing, and/or 3′ UTR disruption.

Embodiment 62: The system of any of embodiments 55-61, wherein the reduced expression of the DUX4 gene is transient or permanent.

Embodiment 63: A pharmaceutical composition comprising the guide RNA of any one of embodiments 1-33 or the system of any one of embodiments 34-62, and a pharmaceutical acceptable carrier.

Embodiment 64: A cell, or population of cells, comprising or modified by the guide RNA of any one of embodiments 1-33 or the system of any one of embodiments 34-62.

Embodiment 65: A method of modifying a DUX4 gene, comprising contacting the DUX4 gene with the guide RNA of any one of embodiments 1-33, system of any one of embodiments 34-62, or pharmaceutical composition of embodiment 63.

Embodiment 66: The method of embodiment 65, wherein modifying of the DUX4 gene comprises inserting, deleting, or substituting one or more nucleotides in the DUX4 gene.

Embodiment 67: The method of embodiment 66, wherein the modifying of the DUX4 gene reduces the expression of the DUX4 gene.

Embodiment 68: The method of embodiment 67, wherein the reduced expression of the DUX4 gene is transient.

Embodiment 69: The method of embodiment 67, wherein the reduced expression of the DUX4 gene is permanent.

Embodiment 70: The method of any one of embodiments 65-69, wherein the DUX4 gene expression is reduced in a muscle cell.

Embodiment 71: The method of embodiment 70, wherein the cell is in vivo.

Embodiment 72: The method of any one of embodiments 70-71, wherein the muscle cell is a skeletal cell, a myoblast, or a myotube muscle cell.

Embodiment 73: The method of any of embodiments 70-72, wherein the muscle cell is within a subject having facioscapulohumeral muscular dystrophy (FSHD).

Embodiment 74: A nucleic acid expression vector that encodes a guide RNA, wherein the guide RNA comprises at least one sequence that is at least 80%, at least 85%, at least 90%, at least 95% or 100% identical to a sequence selected from any one of TABLES 1-5 and SEQ ID NOs: 236 and 351-352.

Embodiment 75: The nucleic acid expression vector of embodiment 74, wherein the nucleic acid expression vector is an adenoviral associated viral (AAV) vector.

Embodiment 76: The nucleic acid expression vector of embodiments 74 or 75, wherein the nucleic acid expression vector further comprises a polynucleotide encoding an effector protein that is at least 80%, at least 85%, at least 90%, at least 95%, at least 99%, or 100% identical to any one of the sequences recited in and one of TABLES 7-11.

Embodiment 77: A pharmaceutical composition, comprising the nucleic acid expression vector of any one of embodiments 74-76; and a pharmaceutically acceptable excipient.

Embodiment 78: A system comprising the nucleic acid expression vector of any one of embodiments 74-76.

Embodiment 79: The system of embodiment 78, comprising at least one detection reagent for detecting a target nucleic acid.

Embodiment 80: A method of modifying a DUX4 gene, the method comprising contacting the DUX4 gene genome with the nucleic acid expression vector of any one of embodiments 74-76, the pharmaceutical composition of embodiment 77, or the system of any one of embodiments 78-79, thereby modifying the DUX4 gene.

Embodiment 81: The method of embodiment 80, wherein the modifying of the DUX4 gene comprises cleaving the DUX4 gene, deleting a nucleotide of the DUX4 gene, inserting a nucleotide into the DUX4 gene, substituting a nucleotide of the DUX4 gene with an alternative nucleotide, or editing a nucleotide, more than one of the foregoing, or any combination thereof.

Embodiment 82: The method of embodiments 80 or 81, wherein the composition further comprises an additional guide RNA that binds a different portion of the DUX4 gene than the guide RNA.

Embodiment 83: The method of embodiment 82, wherein the composition removes the sequence between the guide RNA and the additional guide RNA.

Embodiment 84: The method of any one of embodiments 80-83, further comprising contacting the DUX4 gene with a donor nucleic acid.

Embodiment 85: The method of any one of embodiments 80-84, wherein the method is performed in a cell.

Embodiment 86: The method of embodiment 85, wherein the method is performed in vivo.

Embodiment 87: An expression cassette comprising, from 5′ to 3′: (a) a first inverted terminal repeat (ITR); (b) a first promoter sequence operably linked to a nucleic acid sequence encoding a guide RNA wherein the guide RNA comprises: (i) a first region comprising a protein binding sequence; and (ii) a second region comprising a spacer sequence that is complementary to a target sequence of a DUX4 gene, wherein the spacer sequence is selected from SEQ ID NOs: 1-114, 275-349, 456-460, and 476-596; (c) a second promoter sequence operably linked to a nucleic acid sequence encoding an effector protein; (d) a poly(A) signal; and (e) a second ITR.

Embodiment 88: The expression cassette of embodiment 87, wherein the expression cassette further comprises a WPRE sequence located between the nucleic acid sequence encoding an effector protein and the poly(A) signal.

Embodiment 89: The expression cassette of embodiments 87 or 88, wherein the first promoter is a U6 promoter.

Embodiment 90: The expression cassette of any one of embodiments 87-89, wherein the second promoter is a CK8E promoter or a SPC5 promoter.

Embodiment 91: The expression cassette of any one of embodiments 87-90, wherein the poly(A) signal is a bGH or an hGH poly(A) signal.

Embodiment 92: The expression cassette of any one of embodiments 87-91, wherein the effector protein comprises an amino acid sequence that is at least 90% identical to SEQ ID NO:230.

Embodiment 93: The expression cassette of embodiment 92, wherein the effector protein comprises the amino acid substitution of L26R relative to SEQ ID NO: 230.

Embodiment 94: The expression cassette of embodiment 93, wherein the effector protein comprises SEQ ID NO: 232.

Embodiment 95: The expression cassette of any one of embodiments 87-91, wherein the effector protein comprises an amino acid sequence that is at least 90% identical to SEQ ID NO:428.

Embodiment 96: The expression cassette of embodiment 95, wherein the effector protein comprises the amino acid substitution of D220R relative to SEQ ID NO: 428.

Embodiment 97: The expression cassette of embodiment 96, wherein the effector protein comprises SEQ ID NO: 429.

Embodiment 98: An adeno-associated virus (AAV) vector comprising the expression cassette of any one of embodiments 87-97.

Embodiment 99: A cell comprising the nucleic acid expression vector of any one of embodiments 74-76 or 87-97, or the AAV vector of embodiment 98.

Embodiment 100: A cell that comprises a target nucleic acid modified by the nucleic acid expression vector of any one of embodiments 74-76 or 87-97.

Embodiment 101: The cell of any one of embodiments 99 or 100, wherein the cell is a eukaryotic cell.

Embodiment 102: The cell of any one of embodiments 99-101, wherein the cell is a mammalian cell.

Embodiment 103: The cell of any one of embodiments 99-102, wherein the cell is a human cell.

Embodiment 104: A population of cells that comprises at least one cell of any one of embodiments 100-103.

Embodiment 105: A method of treating a disease caused by a misexpression of the DUX4 gene, the method comprising contacting a cell that has the misexpression of the DUX4 gene, comprising contacting the DUX4 gene with the guide RNA of any of embodiments 1-33, the system of any one of embodiments 34-62, or the composition of embodiment 63.

Embodiment 106: The method of embodiment 105, comprising modifying the DUX4 gene.

Embodiment 107: The method of embodiment 106, wherein modifying the DUX4 gene comprises inserting, deleting, or substituting one or more nucleotides in the DUX4 gene.

Embodiment 108: The method of any one of embodiments 105-107, wherein the disease is a muscular dystrophy.

Embodiment 109: The method of embodiment 108, wherein the muscular dystrophy is Facioscapulohumeral muscular dystrophy (FSHD).

Embodiment 110: A fusion protein comprising an effector protein sequence and a base editing enzyme, wherein (a) the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 428; and (b) the base editing enzyme comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 453.

Embodiment 111: The fusion protein of embodiment 110, wherein the effector protein comprises the amino acid substitutions of D220R and E335Q relative to SEQ ID NO: 428.

Embodiment 112: The fusion protein of any one of embodiments 110 or 111, wherein the fusion protein comprises an amino acid sequence that is at least 90% or at least 95% identical to SEQ ID NO: 454.

Embodiment 113: The fusion protein of any one of embodiments 110 or 111, wherein the fusion protein comprises or consists of SEQ ID NO: 454.

Embodiment 114: A system comprising (a) a guide nucleic acid or a DNA molecule encoding the guide nucleic acid, wherein the guide nucleic acid comprises: (i) a first region comprising a protein binding sequence; and (ii) a second region comprising a targeting sequence that is complementary to a target sequence of an DUX4 gene and is selected from SEQ ID NOs: 457-460; wherein the first region is located 5′ of the second region; (b) a fusion protein comprising an effector protein and a base editing enzyme, or a nucleic acid encoding the fusion protein.

Embodiment 115: A system comprising (a) a guide nucleic acid or a DNA molecule encoding the guide nucleic acid, wherein the guide nucleic acid comprises a nucleotide sequence that is at least 70%, at least 75%, at least 80%, at least 85%, at least 90%, at least 95%, or 100% identical to a sequence selected from SEQ ID NOs: 462-465; and (b) a fusion protein comprising an effector protein and a base editing enzyme, or a nucleic acid encoding the fusion protein, wherein the fusion protein comprises an amino acid sequence that is at least 90% or at least 95% identical to SEQ ID NO: 454.

Embodiment 116: A fusion protein comprising an effector protein sequence and a base editing enzyme sequence, wherein (a) the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 230; and (b) the base editing enzyme comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 453.

Embodiment 117: The fusion protein of embodiment 116, wherein the effector protein comprises the amino acid substitutions of L26K and E567Q relative to SEQ ID NO: 230.

Embodiment 118: The fusion protein of any one of embodiments 116 or 117, wherein the fusion protein comprises an amino acid sequence that is at least 90% or at least 95% identical to SEQ ID NO: 455.

Embodiment 119: The fusion protein of any one of embodiments 116 or 117, wherein the fusion protein comprises or consists of SEQ ID NO: 455.

Embodiment 120: A system comprising (a) a guide nucleic acid or a DNA molecule encoding the guide nucleic acid, wherein the guide nucleic acid comprises: (i) a first region comprising a protein binding sequence; and (ii) a second region comprising a targeting sequence that is complementary to a target sequence of an DUX4 gene and is SEQ ID NO: 456; wherein the first region is located 5′ of the second region; (b) a fusion protein comprising an effector protein and a base editing enzyme, or a nucleic acid encoding the fusion protein.

Embodiment 121: A system comprising (a) a guide nucleic acid or a DNA molecule encoding the guide nucleic acid, wherein the guide nucleic acid comprises a nucleotide sequence that is at least 70%, at least 75%, at least 80%, at least 85%, at least 90%, at least 95%, or 100% identical to SEQ ID NO: 461; and (b) a fusion protein comprising an effector protein and a base editing enzyme, or a nucleic acid encoding the fusion protein, wherein the fusion protein comprises an amino acid sequence that is at least 90% or at least 95% identical to SEQ ID NO: 455.

### EXAMPLES

The following examples are included for illustrative purposes only and are not intended to limit the scope of the invention.

### Example 1: Activity of CasPhi.12 L26R and DUX4 gRNAs in FSHD Donor Myoblasts

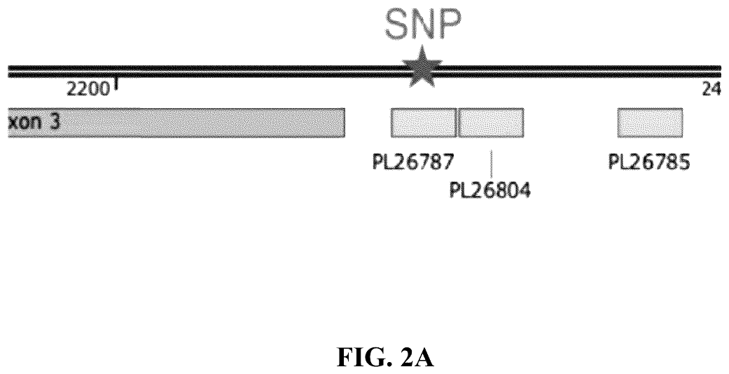

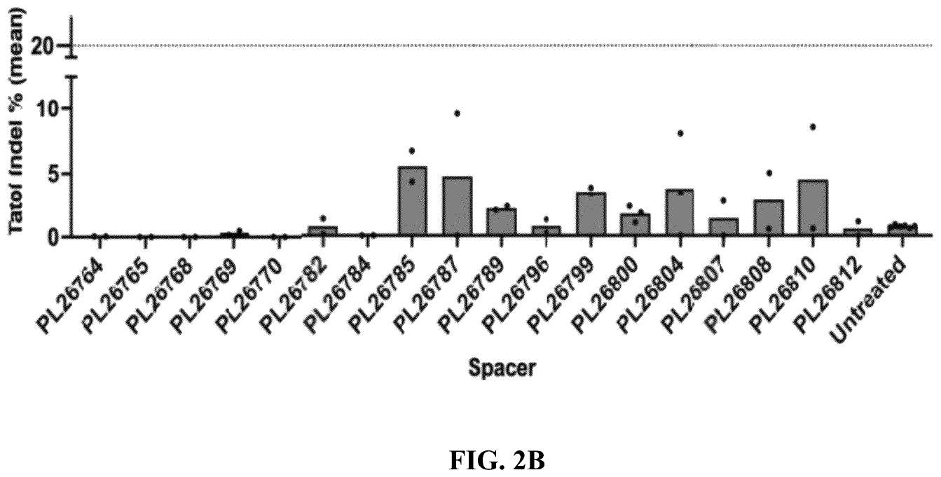

CasPhi.12 L26R (SEQ ID NO: 232) and guide RNAs were screened for their ability to modify a portion of the DUX4 promoter, DUX4 exon 1, DUX4 exon 3, DUX4 intron 2-exon 3 junction, DUX4 intron 2, or the 3′ untranslated region (UTR) region within the DUX4 gene in iPSC derived myoblasts from FSHD patients (FSHD iPSC-d myoblasts). Locations of exemplary guides within the DUX4 gene are illustrated in FIG. 2A.

Plasmids (1 μg) co-expressing CasPhi12 L26R (SEQ ID NO: 232) and gRNA were delivered via lipofection to FSHD iPSC-d myoblasts. gRNA spacer sequences that were screened in this experiment are provided in TABLE 17. Cells were selected with blasticidin for 48h and allowed to expand for 1 week prior to gDNA isolation and NGS. gRNAs tiling the entire exon 3 plus intron-exon boundary regions of DUX4 were included in this screen. Results are represented in FIG. 2B.

TABLE 17
gRNA spacer sequences

| Internal Ref: | Spacer sequence | SEQ ID: |
|---|---|---|
| PL26764 | AGAGAUAUAUUAAAAUGCCC | 57 |
| PL26765 | CGUGAAAUUCUGGCUGAAUG | 58 |
| PL26768 | UUCUUCCGUGAAAUUCUGGC | 61 |
| PL26769 | UGGCUGAAUGUCUCCCCCCA | 62 |
| PL26770 | CAUCUCCUGGAUGAUUAGUU | 63 |
| PL26782 | CCCGCUUCCUGGCUAGACCU | 73 |
| PL26784 | CUGGCUAGACCUGCGCGCAG | 74 |
| PL26785 | GGUGAUCAGUGCAGAUGUGU | 87 |
| PL26787 | UGUGUGAUGAGUGCAGAGAU | 89 |
| PL26789 | UGUGAUGAGUGCAGAGAUAU | 91 |
| PL26796 | GGUGAUCAGUGUAGAGAUAU | 98 |
| PL26799 | UGAAACACAUCUGCACUGAU | 101 |
| PL26800 | UUCUACAGGGGAUAUUGUGA | 102 |
| PL26804 | CUACAGGGGAUAUUGUGACA | 106 |
| PL26807 | UGACAUAUCUCUGCACUCAU | 109 |
| PL26808 | AACAUAUCUCUACACUGAUC | 110 |
| PL26810 | UACAGGGGAUAUUGUGACAU | 112 |
| PL26812 | AGGCUUUUUCUACAGGGGAU | 114 |

### Example 2: Activity of CasM.265466 and DUX4 gRNAs in FSHD Donor Myoblasts

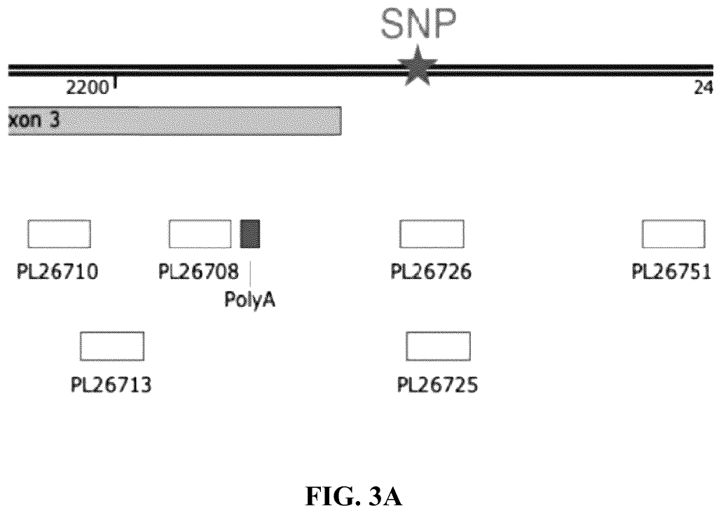

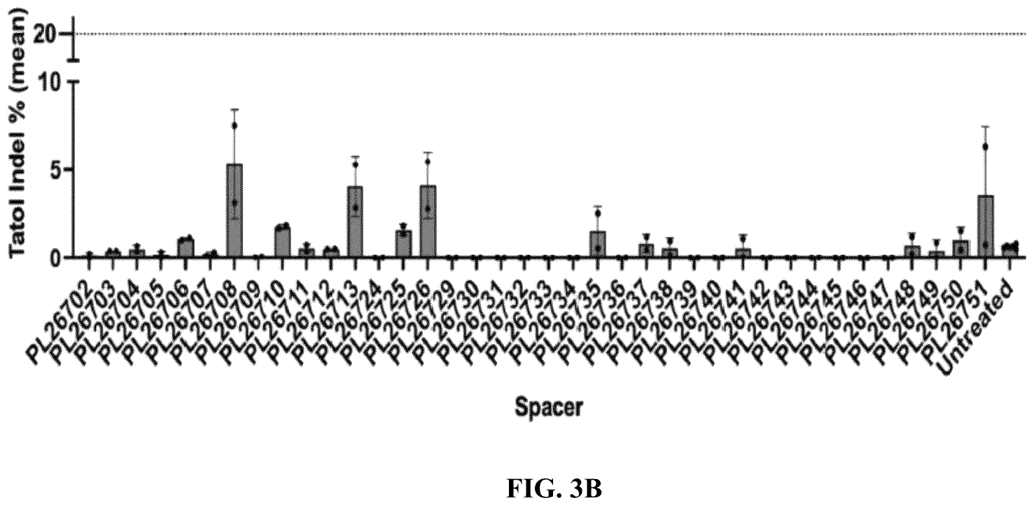

CasM.265466 (SEQ ID NO:428) and guide RNAs were screened for their ability to modify a portion of the DUX4 promoter, DUX4 exon 1, DUX4 exon 3, DUX4 intron 2-exon 3 junction, DUX4 intron 2, or the 3′ untranslated region (UTR) region within the DUX4 gene in iPSC derived myoblasts from FSHD patients (FSHD iPSC-d myoblasts). Locations of exemplary guides within the DUX4 gene are illustrated in FIG. 3A.

Plasmids (1 μg) co-expressing CasM.265466 (SEQ ID NO: 428) and gRNA were delivered via lipofection to FSHD iPSC-d myoblasts. gRNA spacer sequences that were screened in this experiment are provided in Table 18. Cells were selected with blasticidin for 48h and allowed to expand for 1 week prior to gDNA isolation and NGS. gRNAs tiling the entire Exon 3 plus intron-exon boundary regions were included in this screen. Results are represented in FIG. 3B.

TABLE 18
gRNA spacer sequences

| Internal Ref: | Spacer sequence | SEQ ID NO: |
|---|---|---|
| PL26702 | CCCUUGUUCUUCCGUGAAAU | 300 |
| PL26703 | UUAAAAUGCCCCCUCCCUGU | 301 |
| PL26704 | GCUGAAUGUCUCCCCCCACC | 302 |
| PL26705 | UGCCCUUGUUCUUCCGUGAA | 303 |
| PL26706 | GGCAAACCUGGAUUAGAGUU | 304 |
| PL26707 | AUAUAUCUCUGAACUAAUCA | 305 |
| PL26708 | UCUCUGAACUAAUCAUCCAG | 306 |
| PL26709 | AACUAAUCAUCCAGGAGAUG | 307 |
| PL26710 | CCUAGACAGCGUCGGAAGGU | 308 |
| PL26711 | GGAUCCACAGGGAGGGGGCA | 309 |
| PL26712 | AUCCAGGUUUGCCUAGACAG | 310 |
| PL26713 | ACUCUAAUCCAGGUUUGCCU | 311 |
| PL26724 | UUUCAGAACUCCAUAGUAGA | 323 |
| PL26725 | AUGAGUGCAGAGAUAUGUCA | 324 |
| PL26726 | UGAUGAGUGCAGAGAUAUGU | 325 |
| PL26729 | GAUCCUAUAGAAGAUUUGCA | 328 |
| PL26730 | CAGAACUUCGGUGAUCAGUG | 329 |
| PL26731 | GAAAAAGCCUGAAAUUGAUU | 330 |
| PL26732 | UGUGAUGAGUGCAGAGAUAU | 331 |
| PL26733 | CAUCUUUUGUGUGAUGAGUG | 332 |
| PL26734 | GAAGAUUUGCAUCUUUUGUG | 333 |
| PL26735 | UCACAAUAUCCCCUGUAGAA | 334 |
| PL26736 | CACUGAUCACCGAAGUUCUG | 335 |
| PL26737 | CACUGAUCACCUAAGUGAUG | 336 |
| PL26738 | ACAUAUCUCUACACUGAUCA | 337 |
| PL26739 | ACAUAUCUCUGCACUCAUCA | 338 |
| PL26740 | CACUCAUCACACAAAAGAUG | 339 |
| PL26741 | GGUUCAGUCUACUAUGGAGU | 340 |
| PL26742 | AAUCAAUUUCAGGCUUUUUC | 341 |
| PL26743 | UAAAUCAAUUUCAGGCUUUU | 342 |
| PL26744 | ACCAUUCUCUAGGUUCAGUC | 343 |
| PL26745 | CACGAGAAUUUUAACAUAUC | 344 |
| PL26746 | CAGGGGAUAUUGUGACAUAU | 345 |
| PL26747 | AAACACAUCUGCACUGAUCA | 346 |
| PL26748 | UAGGAUCCACAGGGAGGGGG | 313 |
| PL26749 | UCUACACGAGAAUUUUAACA | 347 |
| PL26750 | CUAUGGAGUUCUGAAACACA | 348 |
| PL26751 | GAGUUCUGAAACACAUCUGC | 349 |

The initial screen resulted in the observance of active CasM.265466 guide RNAs. CasM.265466 spacers targeting the exon 3 region of DUX4 achieved editing rates of up to ˜10% (FIG. 3B).

### Example 3: Target Sequence Editing by Base Editor Fusions

Briefly, HEK293T cells will be transfected with plasmids encoding a base editor fusion protein and guide nucleic acids.

The following Effector-base editor fusion proteins are tested: (a) CasM.265466 D220R/E335Q-ABE8e (SEQ ID NO: 454); and (b) CasPhi.12 L26K/E567Q-ABE8e (SEQ ID NO: 455).

TABLE 19 and TABLE 20 show the spacers and guide nucleic acids that are tested, respectively.

TABLE 19
Spacer RNAs to be tested

| Internal | | Effector | SEQ ID |
|---|---|---|---|
| Ref: | Spacer sequence | protein | NO: |
| PL34721 | AGAGAUAUAUCAAAAUGCCC | CasPhi.12 | 456 |
| PL34685 | GUUCAGAGAUAUAUCAAAAU | CasM.265466 | 457 |
| PL34686 | UCAAAAUGCCCCCUCCCUGU | CasM.265466 | 458 |
| PL34687 | AUUAGUUCAGAGAUAUAUCA | CasM.265466 | 459 |
| PL34688 | GAUGAUUAGUUCAGAGAUAU | CasM.265466 | 460 |

TABLE 20
Full guide RNAs to be tested

| | | | SEQ |
|---|---|---|---|
| Internal | | Effector | ID |
| Ref: | Full Guide Sequence | protein | NO: |
| PL34721 | AUUGCUCCUUACGAGGAGAC | CasPhi.12 | 461 |
| | AGAGAUAUAUCAAAAUGCCC | | |
| PL34685 | ACAGCUUAUUUGGAAGCUGA | CasM.265466 | 462 |
| | AAUGUGAGGUUUAUAACACU | | |
| | CACAAGAAUCCUGAAAAAGG | | |
| | AUGCCAAACGUUCAGAGAUA | | |
| | UAUCAAAAU | | |
| PL34686 | ACAGCUUAUUUGGAAGCUGA | CasM.265466 | 463 |
| | AAUGUGAGGUUUAUAACACU | | |
| | CACAAGAAUCCUGAAAAAGG | | |
| | AUGCCAAACUCAAAAUGCCC | | |
| | CCUCCCUGU | | |
| PL34687 | ACAGCUUAUUUGGAAGCUGA | CasM.265466 | 464 |
| | AAUGUGAGGUUUAUAACACU | | |
| | CACAAGAAUCCUGAAAAAGG | | |
| | AUGCCAAACAUUAGUUCAGA | | |
| | GAUAUAUCA | | |
| PL34688 | ACAGCUUAUUUGGAAGCUGA | CasM.265466 | 465 |
| | AAUGUGAGGUUUAUAACACU | | |
| | CACAAGAAUCCUGAAAAAGG | | |
| | AUGCCAAACGAUGAUUAGUU | | |
| | CAGAGAUAU | | |

Cells are harvested 48-72 hours after transfection. Base editing is analyzed by NGS.

### Example 4-In Vivo Muscle Targeting Via AAV9-4A Delivery of CasPhi.12 and CasM.265466 Variants

The purpose of this study was to assess the capability of two effector protein variants, CasPhi.12 L26R (a variant of CasPhi.12) and CasM.265466 D220R (a variant of CasM.265466), to edit nucleic acid sequences within muscle tissues in vivo. The study focused on PCSK9 as an exemplary gene target.

In this study, an AAV9-4A vector was employed as the delivery vehicle for introducing the effector protein and gRNA into the specific target tissues. The DNA encoding the effector protein (e.g., SaCas9, CasPhi.12 L26R, or CasM.265466 D220R) and its corresponding promoter (e.g., ck8e or spc5), along with the DNA encoding the gRNA containing the targeting spacer sequence specific to PCSK9 (referred to as PCSK9 in the plasmid) and its u6 promoter were cloned into the AAV9-4A plasmid between the AAV inverted terminal repeats (ITRs), creating AAV9 constructs as follows:
1) pssAAV-ITR-u6-PCSK9-ck8e-saCas9-bGHpolya-ITR (PL26295)
2) pssAAV-ITR-u6-PCSK9-ck8e-L26R-wpre-hGHpolya-ITR (PL26297)
3) pssAAV-ITR-u6-PCSK9-spc5-12-L26R-wpre-hGHpolya-ITR (PL31718)
4) pssAAV-ITR-u6-PCSK9-ck8e-D220R-wpre-hGHpolya-ITR (PL31719)
5) pssAAV-ITR-u6-PCSK9-spc5-12-D220R-wpre-hGHpolya-ITR (PL31720)
6) pscAAV-ITR-u6-PCSK9-Spc5-12-D220R-HSVpolyA-ITR (PL31721)

The sequences of the gRNA designed for use in conjunction with SaCas9, CasPhi.12 L26R, or CasM.265466 D220R for targeting the mouse PCSK9 gene are provided in TABLE 21.

TABLE 21
Exemplary gRNA sequences

| Target | | | PAM |
|---|---|---|---|
| Locus | Nuclease | Spacer sequence | sequence |
| PCSK9 | SaCas9 | CACCGCAGCCACGCA | NNGRRT |
| | | GAGCA | (GTGGGT) |
| | | (SEQ ID NO: 466) | |
| PCSK9 | CasPhi.12 | GAGCAACGGCGGAAGGU | TTN |
| | L26R | (SEQ ID NO: 467) | (TTG) |
| PCSK9 | CasM. | UAGAACCUUGAUGACA | TNTR |
| | 265466 | UAGC | (TATG) |
| | D220R | (SEQ ID NO: 468) | |

The gRNA for CasPhi.12 comprises a repeat sequence of AUUGCUCCUUACGAGGAGAC (SEQ ID NO: 242)
The gRNA for CasM.265466 D220R comprises a handle sequence of ACAGCUUAUUUGGAAGCUGAAAUGUGAGGUUUAUAACACUCACAAGAAUCCUGAAAAAGGAUGCCAAAC (SEQ ID NO: 352)

The sequences of the other AAV components are provided in TABLE 22.

TABLE 22
The sequences of AAV components

| AAV | | SEQ ID |
|---|---|---|
| Components | Sequences | NO: |
| Ck8e | cgctccggtgcccgtTGCCCATGTAAGGAGGCAAGGCCTG | 469 |
| promoter | GGGACACCCGAGATGCCTGGTTATAATTAACCCAGACATG | |
| | TGGCTGCCCCCCCCCCCCCAACACCTGCTGCCTCTAAAAA | |
| | TAACCCTGCATGCCATGTTCCCGGCGAAGGGCCAGCTGTC | |
| | CCCCGCCAGCTAGACTCAGCACTTAGTTTAGGAACCAGTG | |
| | AGCAAGTCAGCCCTTGGGGCAGCCCATACAAGGCCATGGG | |
| | GCTGGGCAAGCTGCACGCCTGGGTCCGGGGTGGGCACGGT | |
| | GCCCGGGCAACGAGCTGAAAGCTCATCTGCTCTCAGGGGC | |
| | CCCTCCCTGGGGACAGCCCCTCCTGGCTAGTCACACCCTG | |
| | TAGGCTCCTCTATATAACCCAGGGGCACAGGGGCTGCCCT | |
| | CATTCTACCACCACCTCCACAGCACAGACAGACACTCAGG | |
| | AGCCAGCCAGCa | |
| Spc5-12 | ggtaccgctccggtgcccgtCGAGCTCCACCGCGGTGGCG | 470 |
| promoter | GCCGTCCGCCCTCGGCACCATCCTCACGACACCCAAATAT | |
| | GGCGACGGGTGAGGAATGGTGGGGAGTTATTTTTAGAGCG | |
| | GTGAGGAAGGTGGGCAGGCAGCAGGTGTTGGCGCTCTAAA | |
| | AATAACTCCCGGGAGTTATTTTTAGAGCGGAGGAATGGTG | |
| | GACACCCAAATATGGCGACGGTTCCTCACCCGTCGCCATA | |
| | TTTGGGTGTCCGCCCTCGGCCGGGGCCGCATTCCTGGGGG | |
| | CCGGGCGGTGCTCCCGCCCGCCTCGATAAAAGGCTCCGGG | |
| | GCCGGCGGCGGCCCACGAGCTACCCGGAGGAGCGGGAGGC | |
| | GCCAAGCTCTAGAACTAGTGGATCCCCCGGGCTGCAGGAA | |
| | TTCaccggt | |
| U6 promoter | GAGGGCCTATTTCCCATGATTCCTTCATATTTGCATATAC | 471 |
| | GATACAAGGCTGTTAGAGAGATAATTGGAATTAATTTGAC | |
| | TGTAAACACAAAGATATTAGTACAAAATACGTGACGTAGA | |
| | AAGTAATAATTTCTTGGGTAGTTTGCAGTTTTAAAATTAT | |
| | GTTTTAAAATGGACTATCATATGCTTACCGTAACTTGAAA | |
| | GTATTTCGATTTCTTGGCTTTATATATCTTGTGGAAAGGA | |
| | CGAAACACC | |
| CasM.265466 | MSVLTRKVQLIPVGDKEERDRVYKYLRDGIEAQNRAMNLY | 429 |
| D220R | MSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYK | |
| | VCQSSIQFSGTKIILNMAMRIPDKEIELDEDVCVGVDLGI | |
| | AIPAVCALNKNRYSRVSIGSKEDFLRVRTKIRNQRKRLQT | |
| | NLKSSNGGHGRKKKMKPMDRFRDYEANWVQNYNHYVSRQV | |
| | VDFAVKNKAKYINLENLEGIRDDVKNEWLLSNWSYYQLQQ | |
| | YITYKAKTYGIEVRKINPYHTSQRCSCCGYEDAGNRPKKE | |
| | KGQAYFKCLKCGEEMNADFNAARNIAMSTEFQSGKKTKKQ | |
| | KKEQHENK | |
| CasPhi.12 | MIKPTVSQFLTPGFKLIRNHSRTAGRKLKNEGEEACKKFV | 232 |
| L26R | RENEIPKDECPNFQGGPAIANIIAKSREFTEWEIYQSSLA | |
| | IQEVIFTLPKDKLPEPILKEEWRAQWLSEHGLDTVPYKEA | |
| | AGLNLIIKNAVNTYKGVQVKVDNKNKNNLAKINRKNEIAK | |
| | LNGEQEISFEEIKAFDDKGYLLQKPSPNKSIYCYQSVSPK | |
| | PFITSKYHNVNLPEEYIGYYRKSNEPIVSPYQFDRLRIPI | |
| | GEPGYVPKWQYTFLSKKENKRRKLSKRIKNVSPILGIICI | |
| | KKDWCVFDMRGLLRTNHWKKYHKPTDSINDLFDYFTGDPV | |
| | IDTKANVVRFRYKMENGIVNYKPVREKKGKELLENICDQN | |
| | GSCKLATVDVGQNNPVAIGLFELKKVNGELTKTLISRHPT | |
| | PIDFCNKITAYRERYDKLESSIKLDAIKQLTSEQKIEVDN | |
| | YNNNFTPQNTKQIVCSKLNINPNDLPWDKMISGTHFISEK | |
| | AQVSNKSEIYFTSTDKGKTKDVMKSDYKWFQDYKPKLSKE | |
| | VRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKN | |
| | RNGEKFNCLKCGIELNADIDVATENLATVAITAQSMPKPT | |
| | CERSGDAKKPVRARKAKAPEFHDKLAPSYTVVLREAV | |
| WPRE | AATCAACCTCTGGATTACAAAATTTGTGAAAGATTGACTG | 472 |
| | GTATTCTTAACTATGTTGCTCCTTTTACGCTATGTGGATA | |
| | CGCTGCTTTAATGCCTTTGTATCATGCTATTGCTTCCCGT | |
| | ATGGCTTTCATTTTCTCCTCCTTGTATAAATCCTGGTTGC | |
| | TGTCTCTTTATGAGGAGTTGTGGCCCGTTGTCAGGCAACG | |
| | TGGCGTGGTGTGCACTGTGTTTGCTGACGCAACCCCCACT | |
| | GGTTGGGGCATTGCCACCACCTGTCAGCTCCTTTCCGGGA | |
| | CTTTCGCTTTCCCCCTCCCTATTGCCACGGCGGAACTCAT | |
| | CGCCGCCTGCCTTGCCCGCTGCTGGACAGGGGCTCGGCTG | |
| | TTGGGCACTGACAATTCCGTGGTGTTGTCGGGGAAGCTGA | |
| | CGTCCTTTCCATGGCTGCTCGCCTGTGTTGCCACCTGGAT | |
| | TCTGCGCGGGACGTCCTTCTGCTACGTCCCTTCGGCCCTC | |
| | AATCCAGCGGACCTTCCTTCCCGCGGCCTGCTGCCGGCTC | |
| | TGCGGCCTCTTCCGCGTCTTgGCCTTCGCCCTCAGACGAG | |
| | TCGGATCTCCCTTTGGGCCGCCTCCCCGC | |
| bGH PolyA | CTGTGCCTTCTAGTTGCCAGCCATCTGTTGTTTGCCCCTC | 473 |
| | CCCCGTGCCTTCCTTGACCCTGGAAGGTGCCACTCCCACT | |
| | GTCCTTTCCTAATAAAATGAGGAAATTGCATCGCATTGTC | |
| | TGAGTAGGTGTCATTCTATTCTGGGGGGTGGGGTGGGGCA | |
| | GGACAGCAAGGGGGAGGATTGGGAAGAGAATAGCAGGCAT | |
| | GCTGGGGA | |
| hGH PolyA | ACGGGTGGCATCCCTGTGACCCCTCCCCAGTGCCTCTCCTG | 474 |
| | GCCCTGGAAGTTGCCACTCCAGTGCCCACCAGCCTTGTCC | |
| | TAATAAAATTAAGTTGCATCATTTTGTCTGACTAGGTGTC | |
| | CTTCTATAATATTATGGGGTGGAGGGGGGTGGTATGGAGC | |
| | AAGGGGCAAGTTGGGAAcACAACCTGTAGGGCCTGCGGGG | |
| | TCTATTGGGAACCAAGCTGGAGTGCAGTGGCACAATCTTG | |
| | GCTCACTGCAATCTCCGCCTCCTGGGTTCAAGCGATTCTC | |
| | CTGCCTCAGCCTCCCGAGTTGTTGGGATTCCAGGCATGCA | |
| | TGACCAGGCTCAGCTAATTTTTGTTTTTTTGGTAGAGACG | |
| | GGGTTTCACCATATTGGCCAGGCTGGTCTCCAACTCCTAA | |
| | TCTCAGGTGATCTACCCACCTTGGCCTCCCAAATTGCTGG | |
| | GATTACAGGCGTGAACCACTGCTCCCTTCCCTGTCCTTCT | |
| | GATTTTGTAGGTAACCACGTGCGGACCGA | |

6-week old male C57BL/6J mice were given a single intravenous (IV) bolus through the tail vein using a 1.0 cc syringe with a 27G-⅝″ needle or a single intramuscular (IM) bolus into the gastrocnemius. The study design is provided in TABLE 23.

TABLE 23
Study design

| | | Size | | |
|---|---|---|---|---|
| Groups | Construct design (PL#) | (Kb) | Route, dose | N |
| 1 | Vehicle (PBS) | — | — | 15 |
| 2, 3 | pssAAV-ITR-u6-PCSK9-ck8e-saCas9- | 4.7 | 14 vg/kg IV and 12 vg | 20 |
| | hGHpolya-ITR (PL26295) | | IM | |
| 4, 5 | pssAAV-ITR-u6-PCSK9-ck8e-L26R- | 4.6 | 14 vg/kg IV and 12 vg | 20 |
| | wpre-hGHpolya-ITR (PL26297) | | IM | |
| 6 | pssAAV-ITR-u6-PCSK9-spc5-12-L26R- | 4.5 | 14 vg/kg IV | 10 |
| | wpre-hGHpolya-ITR (PL31718) | | | |
| 7 | pssAAV-ITR-u6-PCSK9-ck8e-D220R- | 3.8 | 14 vg/kg IV | 10 |
| | wpre-hGHpolya-ITR (PL31719) | | | |
| 8 | pssAAV-ITR-u6-PCSK9-spc5-12- | 3.79 | 14 vg/kg IV | 10 |
| | D220R-wpre-hGHpolya-ITR (PL31720) | | | |
| 9 | pssAAV-ITR-U6-PCSK9-Spc5-12- | 2.48 | 14 vg/kg IV | 15 |
| | D220R-HSVpolyA-ITR (PL31721) | | | |

4 weeks after the treatment, the mice were euthanized for assessment. Before collecting the tissues, the mice underwent whole-body perfusion via the left ventricle using 5.0-10.0 mL of PBS to remove any remaining blood from the tissues. Tissue samples weighing 100 mg were then dissected from the liver, heart, gastrocnemius, diaphragm, pectoral, and masseter, respectively, and placed on a plate for subsequent Next-Generation Sequencing (NGS) analysis. For the intramuscular (IM) groups, both the left and right gastrocnemius were weighed and harvested. For all other groups, only the left gastrocnemius was weighed and harvested.

The genomic DNA isolated from the muscle tissues was subject to NGS and aligned to a reference DNA sequence for the analysis of insertions or deletions (indels).

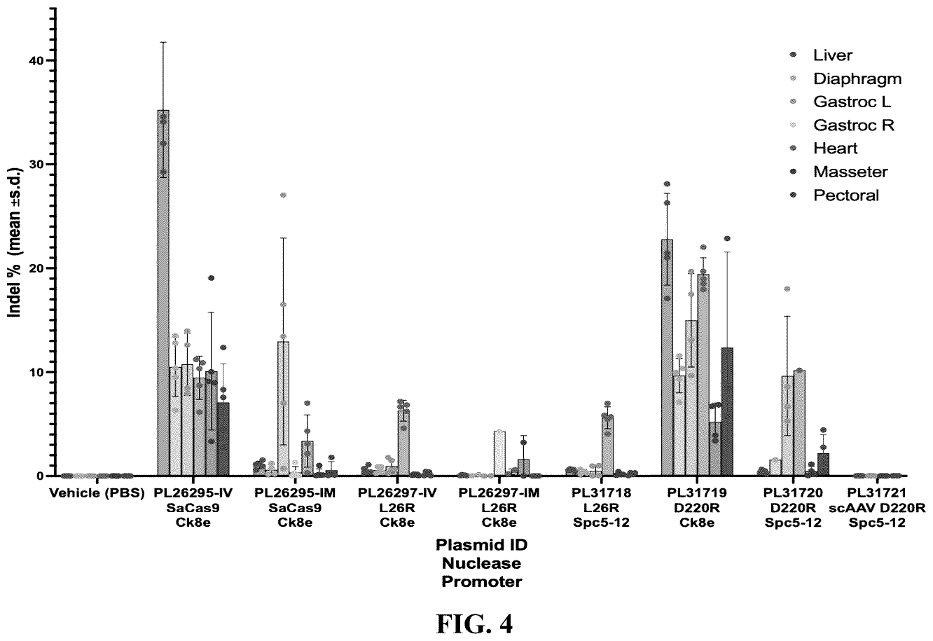

In FIG. 4, the data reveals that CasPhi.12 L26R, delivered in the PL26297 vector via IV, resulted in an about 8% indel rate in the PCSK9 gene in the heart. Likewise, CasPhi.12 L26R delivered in the PL26297 vector via IM administration generated about 5% indel rate in the right gastrocnemius and about 3% indel rate in masseter. Additionally, CasPhi.12 L26R delivered in the PL31718 vector via IV administration was able to generate about 8% indel rate in the PCSK9 gene in the heart.

As shown in FIG. 4, CasM.265466 D220R delivered in the PL31719 vector via IV administration resulted in an about 25% indel rate in the liver, about 10% in the diaphragm, about 15% in the left gastrocnemius, about 20% in the heart, about 5% in the masseter, and about 13% in the pectoral. Remarkably, CasM.265466 D220R demonstrated an approximately 2-fold greater indel rate in the heart compared to SaCas9 delivered in the PL26295 vector via IV administration. Additionally, CasM.265466 D220R delivered in the PL31719 vector via IV administration generated about 10% in the left gastrocnemius, about 11% in the heart, and about 3% in the pectoral.

The results demonstrate that both CasPhi.12 variant L26R and CasM.265466 variant D220R are highly efficient in inducing indels in vivo in different muscle tissues. This experiment is repeated with various combinations of: (1) an effector protein described herein; and (2) a guide nucleic acid described herein that comprises a spacer sequence complementary to DUX4. In some instances, the effector protein is CasPhi.12 or a variant thereof described herein. In some instances, the effector protein is CasM.265466 or a variant thereof described herein.

1. A composition or system comprising a guide ribonucleic acid (RNA) or a polynucleotide encoding the same, wherein the guide RNA comprises:
a. a first region comprising a protein binding sequence, and
b. a second region comprising a targeting sequence that is complementary to a target sequence that is within a DUX4 gene,
wherein the target sequence is adjacent to a protospacer adjacent motif (PAM) selected from 5′-NTTN-3′ and 5′-NNTN-3′.

2. The composition or system of claim 1, wherein the targeting sequence comprises a nucleotide sequence that is at least 70%, at least 75%, at least 80%, at least 85%, at least 90%, at least 95% or 100% identical to any one of SEQ ID NOs: 1-114, 275-349, 456-460, and 476-596.

3. The composition or system of claim 1 or 2, wherein the PAM is 5′-NTTN-3′ and wherein
a. the targeting sequence comprises a nucleotide sequence that is at least 70%, at least 75%, at least 80%, at least 85%, at least 90%, at least 95% or 100% identical to any one of SEQ ID NOs: 1-114, 456, and 481-596, and
b. the protein binding sequence comprises a nucleotide sequence that is at least 70%, at least 75%, at least 80%, at least 85%, at least 90%, at least 95% or 100% identical to any one of SEQ ID NOs: 115, and 237-242.

4. The composition or system of claim 3, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NO: 230.

5. The composition or system of any one of claims 1-4, wherein the guide RNA comprises a nucleotide sequence that is at least 70%, at least 75%, at least 80%, at least 85%, at least 90%, at least 95% or 100% identical to any one of SEQ ID NOs: 116-229, 461, and 602-717.

6. The composition or system of claim 1 or 2, wherein the PAM is 5′-NNTN-3′, and wherein
a. the targeting sequence comprises a nucleotide sequence that is at least 70%, at least 75%, at least 80%, at least 85%, at least 90%, at least 95% or 100% identical to any one of SEQ ID NOs: 275-349, 457-460, and 476-480, and
b. the protein binding sequence comprises a nucleotide sequence that is at least 70%, at least 75%, at least 80%, at least 85%, at least 90%, at least 95% or 100% identical to SEQ ID NO: 350.

7. The composition or system of claim 6, wherein the protein binding sequence further comprises a nucleotide sequence that is at least 70%, at least 75%, at least 80%, at least 85%, at least 90%, at least 95% or 100% identical to SEQ ID NOs: 351 or 352.

8. The composition or system of claim 6 or claim 7, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NO: 428.

9. The composition or system of any one of claims 1, 2, and 6-8, wherein the guide RNA comprises a nucleotide sequence that is at least 70%, at least 75%, at least 80%, at least 85%, at least 90%, at least 95% or 100% identical to any one of SEQ ID NOs: 353-427, 462-465, and 597-601.

10. The composition or system of claim 4, 5, 8, or 9, wherein the effector protein is fused to an effector partner protein, optionally wherein the effector partner protein is selected from a deaminase, a reverse transcriptase, a recombinase, and a methyltransferase.

11. The composition or system of claim 4, wherein the targeting sequence is at least 80%, at least 85%, at least 90%, at least 95%, or 100% identical to a sequence selected from SEQ ID NOs: 481-485, and wherein the effector protein is at least 80%, at least 85%, at least 90%, at least 95%, or 100% identical to SEQ ID NO: 230 and wherein the effector protein is fused to a base editing enzyme.

12. The composition or system of claim 8, wherein the targeting sequence is at least 80%, at least 85%, at least 90%, at least 95%, or 100% identical to a sequence selected from SEQ ID NOs: 476-480, wherein the effector protein is at least 80%, at least 85%, at least 90%, at least 95%, or 100% identical to SEQ ID NO: 428 and wherein the effector protein is fused to a base editing enzyme.

13. The composition or system of claim 4, wherein the targeting sequence is at least 80%, at least 85%, at least 90%, at least 95%, or 100% identical to a sequence selected from SEQ ID NOs: 486-596, wherein the effector protein is at least 80%, at least 85%, at least 90%, at least 95%, or 100% identical to SEQ ID NO: 230 and wherein the effector protein is fused to a KRAB domain, a methyltransferase, or a combination thereof.

14. An expression cassette comprising, from 5′ to 3′:
a. a first inverted terminal repeat (ITR);
b. a first promoter sequence operably linked to a nucleic acid sequence encoding a guide RNA wherein the guide RNA comprises:
i. a first region comprising a protein binding sequence; and
ii. a second region comprising a spacer sequence that is complementary to a target sequence of a DUX4 gene, wherein the spacer sequence is at least 70%, at least 75%, at least 80%, at least 85%, at least 90%, at least 95% or 100% identical to any one of SEQ ID NOs: 1-114, 275-349, 456-460, and 476-596;
c. a second promoter sequence operably linked to a nucleic acid sequence encoding an effector protein;
d. a poly(A) signal; and
e. a second ITR.

15. The expression cassette of claim 14, wherein the expression cassette further comprises a WPRE sequence located between the nucleic acid sequence encoding an effector protein and the poly(A) signal.

16. The expression cassette of claim 14 or 15, wherein the first promoter is a U6 promoter, the second promoter is a CK8E promoter or a SPC5 promoter or a combination thereof.

17. The expression cassette of any one of claims 14-16, wherein the poly(A) signal is a bGH or an hGH poly(A) signal.

18. The expression cassette of any one of claims 14-17, wherein
a. the targeting sequence comprises a nucleotide sequence that is at least 70%, at least 75%, at least 80%, at least 85%, at least 90%, at least 95% or 100% identical to any one of SEQ ID NOs: 1-114, 456, and 481-596, and
b. the effector protein comprises an amino acid sequence that is at least 70%, at least 75%, at least 80%, at least 85%, at least 90%, at least 95% or 100% identical to SEQ ID NO: 230,
c. optionally wherein the protein binding sequence comprises a nucleotide sequence that is at least 70%, at least 75%, at least 80%, at least 85%, at least 90%, at least 95% or 100% identical to any one of SEQ ID NOs: 115 and 237-242.

19. The expression cassette of claim 18, wherein the guide RNA comprises a nucleotide sequence that is at least 70%, at least 75%, at least 80%, at least 85%, at least 90%, at least 95% or 100% identical to any one of SEQ ID NOs: 116-229, 461, and 602-717.

20. The expression cassette of any one of claims 14-17, wherein
a. the targeting sequence comprises a nucleotide sequence that is at least 70%, at least 75%, at least 80%, at least 85%, at least 90%, at least 95% or 100% identical to any one of SEQ ID NOs: 275-349, 457-460, and 476-480, and
b. the effector protein comprises an amino acid sequence that is at least 70%, at least 75%, at least 80%, at least 85%, at least 90%, at least 95% or 100% identical to SEQ ID NO: 428,
c. optionally wherein the protein binding sequence comprises a nucleotide sequence that is at least 70%, at least 75%, at least 80%, at least 85%, at least 90%, at least 95% or 100% identical to SEQ ID NOs: 350 or 351, or a combination thereof.

21. The expression cassette of claim 20, wherein the guide RNA comprises a nucleotide sequence that is at least 70%, at least 75%, at least 80%, at least 85%, at least 90%, at least 95% or 100% identical to any one of SEQ ID NOs: 353-427, 462-465, and 597-601.

22. An adeno-associated virus (AAV) vector comprising the expression cassette of any one of claims 14-21.

23. A pharmaceutical composition comprising the composition, system, expression cassette, or AAV vector of any one of claims 1-22.

24. A cell, or population of cells, comprising or modified by the composition, system, expression cassette, or AAV vector of any one of claims 1-22.

25. A method of modifying a DUX4 gene, comprising contacting the DUX4 gene with the composition, system, expression cassette, or AAV vector of any one of claims 1-22.

26. The method of claim 25, wherein modifying of the DUX4 gene comprises inserting, deleting, or substituting one or more nucleotides in the DUX4 gene.

27. The method of claim 26, wherein the modifying of the DUX4 gene reduces the expression of the DUX4 gene.

28. The method of claim 27, wherein the reduced expression of the DUX4 gene is transient.

29. The method of claim 27, wherein the reduced expression of the DUX4 gene is permanent.

30. The method of any one of claims 25-29, comprising modifying the DUX4 gene in a muscle cell, optionally wherein the muscle cell is selected from a skeletal muscle cell, a myoblast, and a myotube muscle cell.

31. The method of claim 30, wherein the muscle cell is in vivo.

32. The method of any of claim 30 or 31, wherein the muscle cell is within a subject having facioscapulohumeral muscular dystrophy (FSHD).

33. A cell modified by the composition, system, expression cassette, AAV vector, or method of any one of claims 1-32.


SEQUENCE LISTING (717 sequences)

SEQ ID NO: 1 - RNA, 20 nt, synthetic construct
  source 1..20: mol_type = other RNA
agcggaacccgtacccgggc

SEQ ID NO: 2 - RNA, 20 nt, synthetic construct
  source 1..20: mol_type = other RNA
agaaggatcgctttccaggc

SEQ ID NO: 3 - RNA, 20 nt, synthetic construct
  source 1..20: mol_type = other RNA
gcctacgccgccccggctcc

SEQ ID NO: 4 - RNA, 20 nt, synthetic construct
  source 1..20: mol_type = other RNA
agatctggtttcagaatcga

SEQ ID NO: 5 - RNA, 20 nt, synthetic construct
  source 1..20: mol_type = other RNA
cgcctacgccgccccggctc

SEQ ID NO: 6 - RNA, 20 nt, synthetic construct
  source 1..20: mol_type = other RNA
cgtgagccaggcagcgaggg

SEQ ID NO: 7 - RNA, 20 nt, synthetic construct
  source 1..20: mol_type = other RNA
caggcatcgccgcccgggag

SEQ ID NO: 8 - RNA, 20 nt, synthetic construct
  source 1..20: mol_type = other RNA
gaccccgagccaaagcgagg

SEQ ID NO: 9 - RNA, 20 nt, synthetic construct
  source 1..20: mol_type = other RNA
cagaatgagaggtcacgcca

SEQ ID NO: 10 - RNA, 20 nt, synthetic construct
  source 1..20: mol_type = other RNA
gtgagccaggcagcgagggc

SEQ ID NO: 11 - RNA, 20 nt, synthetic construct
  source 1..20: mol_type = other RNA
ccaggcatcgccgcccggga

SEQ ID NO: 12 - RNA, 20 nt, synthetic construct
  source 1..20: mol_type = other RNA
gagcggaacccgtacccggg

SEQ ID NO: 13 - RNA, 20 nt, synthetic construct
  source 1..20: mol_type = other RNA
ggaccccgagccaaagcgag

SEQ ID NO: 14 - RNA, 20 nt, synthetic construct
  source 1..20: mol_type = other RNA
ggtttcagaatgagaggtca

SEQ ID NO: 15 - RNA, 20 nt, synthetic construct
  source 1..20: mol_type = other RNA
tgcagcaggcgcaacctctc

SEQ ID NO: 16 - RNA, 20 nt, synthetic construct
  source 1..20: mol_type = other RNA
gtttcagaatgagaggtcac

SEQ ID NO: 17 - RNA, 20 nt, synthetic construct
  source 1..20: mol_type = other RNA
agaatgagaggtcacgccag

SEQ ID NO: 18 - RNA, 20 nt, synthetic construct
  source 1..20: mol_type = other RNA
ccgcaccccacgtgccctgc

SEQ ID NO: 19 - RNA, 20 nt, synthetic construct
  source 1..20: mol_type = other RNA
cggagcccagggtccagatt

SEQ ID NO: 20 - RNA, 20 nt, synthetic construct
  source 1..20: mol_type = other RNA
agaatcgaagggccaggcac

SEQ ID NO: 21 - RNA, 20 nt, synthetic construct
  source 1..20: mol_type = other RNA
gcccacaccggcgcgtgggg

SEQ ID NO: 22 - RNA, 20 nt, synthetic construct
  source 1..20: mol_type = other RNA
cagaatcgaagggccaggca

SEQ ID NO: 23 - RNA, 20 nt, synthetic construct
  source 1..20: mol_type = other RNA
ctgcagcaggcgcaacctct

SEQ ID NO: 24 - RNA, 20 nt, synthetic construct
  source 1..20: mol_type = other RNA
cgccacccacgtcccagggg

SEQ ID NO: 25 - RNA, 20 nt, synthetic construct
  source 1..20: mol_type = other RNA
gagaaggatcgctttccagg

SEQ ID NO: 26 - RNA, 20 nt, synthetic construct
  source 1..20: mol_type = other RNA
tgaaaccagatctgaatcct

SEQ ID NO: 27 - RNA, 20 nt, synthetic construct
  source 1..20: mol_type = other RNA
gcccgggtgcggaggccacc

SEQ ID NO: 28 - RNA, 20 nt, synthetic construct
  source 1..20: mol_type = other RNA
tcaaaggctcggaggagcag

SEQ ID NO: 29 - RNA, 20 nt, synthetic construct
  source 1..20: mol_type = other RNA
cgcggggagggtgctgtccg

SEQ ID NO: 30 - RNA, 20 nt, synthetic construct
  source 1..20: mol_type = other RNA
ccgccggtgctgcctcagct

SEQ ID NO: 31 - RNA, 20 nt, synthetic construct
  source 1..20: mol_type = other RNA
cccgggtgcggaggccaccg

SEQ ID NO: 32 - RNA, 20 nt, synthetic construct
  source 1..20: mol_type = other RNA
catctgcccctgccgcgcgg

SEQ ID NO: 33 - RNA, 20 nt, synthetic construct
  source 1..20: mol_type = other RNA
cgcctgctgcagaaactccg

SEQ ID NO: 34 - RNA, 20 nt, synthetic construct
  source 1..20: mol_type = other RNA
ccacgccgccccggcgacct

SEQ ID NO: 35 - RNA, 20 nt, synthetic construct
  source 1..20: mol_type = other RNA
gccctgcggccccgcttgag

SEQ ID NO: 36 - RNA, 20 nt, synthetic construct
  source 1..20: mol_type = other RNA
ctaggagaggttgcgcctgc

SEQ ID NO: 37 - RNA, 20 nt, synthetic construct
  source 1..20: mol_type = other RNA
agcgggcccaggctgtgcca

SEQ ID NO: 38 - RNA, 20 nt, synthetic construct
  source 1..20: mol_type = other RNA
ctcgctgaggggtgcttcca

SEQ ID NO: 39 - RNA, 20 nt, synthetic construct
  source 1..20: mol_type = other RNA
cgctcaaagcaggctcgcag

SEQ ID NO: 40 - RNA, 20 nt, synthetic construct
  source 1..20: mol_type = other RNA
ggctcggggtccaaacgagt

SEQ ID NO: 41 - RNA, 20 nt, synthetic construct
  source 1..20: mol_type = other RNA
gattctgaaaccagatctga

SEQ ID NO: 42 - RNA, 20 nt, synthetic construct
  source 1..20: mol_type = other RNA
tgcccgggtgcggaggccac

SEQ ID NO: 43 - RNA, 20 nt, synthetic construct
  source 1..20: mol_type = other RNA
tgaaaccaaatctggaccct

SEQ ID NO: 44 - RNA, 20 nt, synthetic construct
  source 1..20: mol_type = other RNA
cgaggcctccagctcccccg

SEQ ID NO: 45 - RNA, 20 nt, synthetic construct
  source 1..20: mol_type = other RNA
tctggtggcgatgcccgggt

SEQ ID NO: 46 - RNA, 20 nt, synthetic construct
  source 1..20: mol_type = other RNA
ggagatcccctctgccggcg

SEQ ID NO: 47 - RNA, 20 nt, synthetic construct
  source 1..20: mol_type = other RNA
taggagaggttgcgcctgct

SEQ ID NO: 48 - RNA, 20 nt, synthetic construct
  source 1..20: mol_type = other RNA
gggttcccacgccgccccgg

SEQ ID NO: 49 - RNA, 20 nt, synthetic construct
  source 1..20: mol_type = other RNA
cgccggccttctggcgggcc

SEQ ID NO: 50 - RNA, 20 nt, synthetic construct
  source 1..20: mol_type = other RNA
cccacgcgccggtgtgggcg

SEQ ID NO: 51 - RNA, 20 nt, synthetic construct
  source 1..20: mol_type = other RNA
cagcgaggcggcctcttccg

SEQ ID NO: 52 - RNA, 20 nt, synthetic construct
  source 1..20: mol_type = other RNA
ttcctcgctgaggggtgctt

SEQ ID NO: 53 - RNA, 20 nt, synthetic construct
  source 1..20: mol_type = other RNA
gctcggggtccaaacgagtc

SEQ ID NO: 54 - RNA, 20 nt, synthetic construct
  source 1..20: mol_type = other RNA
tggcgggccgcgtctcccgg

SEQ ID NO: 55 - RNA, 20 nt, synthetic construct
  source 1..20: mol_type = other RNA
gccggccttctggcgggccg

SEQ ID NO: 56 - RNA, 20 nt, synthetic construct
  source 1..20: mol_type = other RNA
cgacgctgtctaggcaaacc

SEQ ID NO: 57 - RNA, 20 nt, synthetic construct
  source 1..20: mol_type = other RNA
agagatatattaaaatgccc

SEQ ID NO: 58 - RNA, 20 nt, synthetic construct
  source 1..20: mol_type = other RNA
cgtgaaattctggctgaatg

SEQ ID NO: 59 - RNA, 20 nt, synthetic construct
  source 1..20: mol_type = other RNA
ttccgtgaaattctggctga

SEQ ID NO: 60 - RNA, 20 nt, synthetic construct
  source 1..20: mol_type = other RNA
gagttacatctcctggatga

SEQ ID NO: 61 - RNA, 20 nt, synthetic construct
  source 1..20: mol_type = other RNA
ttcttccgtgaaattctggc

SEQ ID NO: 62 - RNA, 20 nt, synthetic construct
  source 1..20: mol_type = other RNA
tggctgaatgtctcccccca

SEQ ID NO: 63 - RNA, 20 nt, synthetic construct
  source 1..20: mol_type = other RNA
catctcctggatgattagtt

SEQ ID NO: 64 - RNA, 20 nt, synthetic construct
  source 1..20: mol_type = other RNA
aaatgccccctccctgtgga

SEQ ID NO: 65 - RNA, 20 nt, synthetic construct
  source 1..20: mol_type = other RNA
cacgtcagccggggtgcgca

SEQ ID NO: 66 - RNA, 20 nt, synthetic construct
  source 1..20: mol_type = other RNA
atatatctctgaactaatca

SEQ ID NO: 67 - RNA, 20 nt, synthetic construct
  source 1..20: mol_type = other RNA
acggaagaacaagggcacag

SEQ ID NO: 68 - RNA, 20 nt, synthetic construct
  source 1..20: mol_type = other RNA
cctagacagcgtcggaaggt

SEQ ID NO: 69 - RNA, 20 nt, synthetic construct
  source 1..20: mol_type = other RNA
cacggaagaacaagggcaca

SEQ ID NO: 70 - RNA, 20 nt, synthetic construct
  source 1..20: mol_type = other RNA
gcctagacagcgtcggaagg

SEQ ID NO: 71 - RNA, 20 nt, synthetic construct
  source 1..20: mol_type = other RNA
aatatatctctgaactaatc

SEQ ID NO: 72 - RNA, 20 nt, synthetic construct
  source 1..20: mol_type = other RNA
agccagaatttcacggaaga

SEQ ID NO: 73 - RNA, 20 nt, synthetic construct
  source 1..20: mol_type = other RNA
cccgcttcctggctagacct

SEQ ID NO: 74 - RNA, 20 nt, synthetic construct
  source 1..20: mol_type = other RNA
ctggctagacctgcgcgcag

SEQ ID NO: 75 - RNA, 20 nt, synthetic construct
  source 1..20: mol_type = other RNA
tataggatccacagggaggg

SEQ ID NO: 76 - RNA, 20 nt, synthetic construct
  source 1..20: mol_type = other RNA
cgggcagccgcctgggctgt

SEQ ID NO: 77 - RNA, 20 nt, synthetic construct
  source 1..20: mol_type = other RNA
gcgggcagccgcctgggctg

SEQ ID NO: 78 - RNA, 20 nt, synthetic construct
  source 1..20: mol_type = other RNA
cgggggtggggggtgggggt

SEQ ID NO: 79 - RNA, 20 nt, synthetic construct
  source 1..20: mol_type = other RNA
gcgggacgggggtctccacc

SEQ ID NO: 80 - RNA, 20 nt, synthetic construct
  source 1..20: mol_type = other RNA
gaccgccaggcgctccgtgc

SEQ ID NO: 81 - RNA, 20 nt, synthetic construct
  source 1..20: mol_type = other RNA
tccgggggtggggggtgggg

SEQ ID NO: 82 - RNA, 20 nt, synthetic construct
  source 1..20: mol_type = other RNA
ccgggggtggggggtggggg

SEQ ID NO: 83 - RNA, 20 nt, synthetic construct
  source 1..20: mol_type = other RNA
cgcgggacgggggtctccac

SEQ ID NO: 84 - RNA, 20 nt, synthetic construct
  source 1..20: mol_type = other RNA
tgaccgccaggcgctccgtg

SEQ ID NO: 85 - RNA, 20 nt, synthetic construct
  source 1..20: mol_type = other RNA
accgccaggcgctccgtgct

SEQ ID NO: 86 - RNA, 20 nt, synthetic construct
  source 1..20: mol_type = other RNA
gcccgcttcctggctagacc

SEQ ID NO: 87 - RNA, 20 nt, synthetic construct
  source 1..20: mol_type = other RNA
ggtgatcagtgcagatgtgt

SEQ ID NO: 88 - RNA, 20 nt, synthetic construct
  source 1..20: mol_type = other RNA
cagaactccatagtagactg

SEQ ID NO: 89 - RNA, 20 nt, synthetic construct
  source 1..20: mol_type = other RNA
tgtgtgatgagtgcagagat

SEQ ID NO: 90 - RNA, 20 nt, synthetic construct
  source 1..20: mol_type = other RNA
catcttttgtgtgatgagtg

SEQ ID NO: 91 - RNA, 20 nt, synthetic construct
  source 1..20: mol_type = other RNA
tgtgatgagtgcagagatat

SEQ ID NO: 92 - RNA, 20 nt, synthetic construct
  source 1..20: mol_type = other RNA
acagaacttcggtgatcagt

SEQ ID NO: 93 - RNA, 20 nt, synthetic construct
  source 1..20: mol_type = other RNA
gcatcttttgtgtgatgagt

SEQ ID NO: 94 - RNA, 20 nt, synthetic construct
  source 1..20: mol_type = other RNA
cagaacttcggtgatcagtg

SEQ ID NO: 95 - RNA, 20 nt, synthetic construct
  source 1..20: mol_type = other RNA
agaactccatagtagactga

SEQ ID NO: 96 - RNA, 20 nt, synthetic construct
  source 1..20: mol_type = other RNA
gtgtgatgagtgcagagata

SEQ ID NO: 97 - RNA, 20 nt, synthetic construct
  source 1..20: mol_type = other RNA
catcacttaggtgatcagtg

SEQ ID NO: 98 - RNA, 20 nt, synthetic construct
  source 1..20: mol_type = other RNA
ggtgatcagtgtagagatat

SEQ ID NO: 99 - RNA, 20 nt, synthetic construct
  source 1..20: mol_type = other RNA
aaattctcgtgtagacagag

SEQ ID NO: 100 - RNA, 20 nt, synthetic construct
  source 1..20: mol_type = other RNA
atttacagaacttcggtgat

SEQ ID NO: 101 - RNA, 20 nt, synthetic construct
  source 1..20: mol_type = other RNA
tgaaacacatctgcactgat

SEQ ID NO: 102 - RNA, 20 nt, synthetic construct
  source 1..20: mol_type = other RNA
ttctacaggggatattgtga

SEQ ID NO: 103 - RNA, 20 nt, synthetic construct
  source 1..20: mol_type = other RNA
agtctactatggagttctga

SEQ ID NO: 104 - RNA, 20 nt, synthetic construct
  source 1..20: mol_type = other RNA
caggctttttctacagggga

SEQ ID NO: 105 - RNA, 20 nt, synthetic construct
  source 1..20: mol_type = other RNA
tctacaggggatattgtgac

SEQ ID NO: 106 - RNA, 20 nt, synthetic construct
  source 1..20: mol_type = other RNA
ctacaggggatattgtgaca

SEQ ID NO: 107 - RNA, 20 nt, synthetic construct
  source 1..20: mol_type = other RNA
taacatatctctacactgat

SEQ ID NO: 108 - RNA, 20 nt, synthetic construct
  source 1..20: mol_type = other RNA
acatatctctacactgatca

SEQ ID NO: 109 - RNA, 20 nt, synthetic construct
  source 1..20: mol_type = other RNA
tgacatatctctgcactcat

SEQ ID NO: 110 - RNA, 20 nt, synthetic construct
  source 1..20: mol_type = other RNA
aacatatctctacactgatc

SEQ ID NO: 111 - RNA, 20 nt, synthetic construct
  source 1..20: mol_type = other RNA
tgtaaatcaatttcaggctt

SEQ ID NO: 112 - RNA, 20 nt, synthetic construct
  source 1..20: mol_type = other RNA
tacaggggatattgtgacat

SEQ ID NO: 113 - RNA, 20 nt, synthetic construct
  source 1..20: mol_type = other RNA
tctaggttcagtctactatg

SEQ ID NO: 114 - RNA, 20 nt, synthetic construct
  source 1..20: mol_type = other RNA
aggctttttctacaggggat

SEQ ID NO: 115 - RNA, 25 nt, synthetic construct
  source 1..25: mol_type = other RNA
gatagattgctccttacgaggagac

SEQ ID NO: 116 - RNA, 43 nt, synthetic construct
  source 1..43: mol_type = other RNA
tagattgctccttacgaggagacagcggaacccgtacccgggc

SEQ ID NO: 117 - RNA, 44 nt, synthetic construct
  source 1..44: mol_type = other RNA
atagattgctccttacgaggagacagaaggatcgctttccaggc

SEQ ID NO: 118 - RNA, 44 nt, synthetic construct
  source 1..44: mol_type = other RNA
atagattgctccttacgaggagacgcctacgccgccccggctcc

SEQ ID NO: 119 - RNA, 44 nt, synthetic construct
  source 1..44: mol_type = other RNA
atagattgctccttacgaggagacagatctggtttcagaatcga

SEQ ID NO: 120 - RNA, 44 nt, synthetic construct
  source 1..44: mol_type = other RNA
atagattgctccttacgaggagaccgcctacgccgccccggctc

SEQ ID NO: 121 - RNA, 44 nt, synthetic construct
  source 1..44: mol_type = other RNA
atagattgctccttacgaggagaccgtgagccaggcagcgaggg

SEQ ID NO: 122 - RNA, 44 nt, synthetic construct
  source 1..44: mol_type = other RNA
atagattgctccttacgaggagaccaggcatcgccgcccgggag

SEQ ID NO: 123 - RNA, 44 nt, synthetic construct
  source 1..44: mol_type = other RNA
atagattgctccttacgaggagacgaccccgagccaaagcgagg

SEQ ID NO: 124 - RNA, 44 nt, synthetic construct
  source 1..44: mol_type = other RNA
atagattgctccttacgaggagaccagaatgagaggtcacgcca

SEQ ID NO: 125 - RNA, 44 nt, synthetic construct
  source 1..44: mol_type = other RNA
atagattgctccttacgaggagacgtgagccaggcagcgagggc

SEQ ID NO: 126 - RNA, 44 nt, synthetic construct
  source 1..44: mol_type = other RNA
atagattgctccttacgaggagacccaggcatcgccgcccggga

SEQ ID NO: 127 - RNA, 44 nt, synthetic construct
  source 1..44: mol_type = other RNA
atagattgctccttacgaggagacgagcggaacccgtacccggg

SEQ ID NO: 128 - RNA, 44 nt, synthetic construct
  source 1..44: mol_type = other RNA
atagattgctccttacgaggagacggaccccgagccaaagcgag

SEQ ID NO: 129 - RNA, 44 nt, synthetic construct
  source 1..44: mol_type = other RNA
atagattgctccttacgaggagacggtttcagaatgagaggtca

SEQ ID NO: 130 - RNA, 44 nt, synthetic construct
  source 1..44: mol_type = other RNA
atagattgctccttacgaggagactgcagcaggcgcaacctctc

SEQ ID NO: 131 - RNA, 44 nt, synthetic construct
  source 1..44: mol_type = other RNA
atagattgctccttacgaggagacgtttcagaatgagaggtcac

SEQ ID NO: 132 - RNA, 44 nt, synthetic construct
  source 1..44: mol_type = other RNA
atagattgctccttacgaggagacagaatgagaggtcacgccag

SEQ ID NO: 133 - RNA, 44 nt, synthetic construct
  source 1..44: mol_type = other RNA
atagattgctccttacgaggagacccgcaccccacgtgccctgc

SEQ ID NO: 134 - RNA, 44 nt, synthetic construct
  source 1..44: mol_type = other RNA
atagattgctccttacgaggagaccggagcccagggtccagatt

SEQ ID NO: 135 - RNA, 44 nt, synthetic construct
  source 1..44: mol_type = other RNA
atagattgctccttacgaggagacagaatcgaagggccaggcac

SEQ ID NO: 136 - RNA, 44 nt, synthetic construct
  source 1..44: mol_type = other RNA
atagattgctccttacgaggagacgcccacaccggcgcgtgggg

SEQ ID NO: 137 - RNA, 44 nt, synthetic construct
  source 1..44: mol_type = other RNA
atagattgctccttacgaggagaccagaatcgaagggccaggca

SEQ ID NO: 138 - RNA, 44 nt, synthetic construct
  source 1..44: mol_type = other RNA
atagattgctccttacgaggagacctgcagcaggcgcaacctct

SEQ ID NO: 139 - RNA, 44 nt, synthetic construct
  source 1..44: mol_type = other RNA
atagattgctccttacgaggagaccgccacccacgtcccagggg

SEQ ID NO: 140 - RNA, 44 nt, synthetic construct
  source 1..44: mol_type = other RNA
atagattgctccttacgaggagacgagaaggatcgctttccagg

SEQ ID NO: 141 - RNA, 44 nt, synthetic construct
  source 1..44: mol_type = other RNA
atagattgctccttacgaggagactgaaaccagatctgaatcct

SEQ ID NO: 142 - RNA, 44 nt, synthetic construct
  source 1..44: mol_type = other RNA
atagattgctccttacgaggagacgcccgggtgcggaggccacc

SEQ ID NO: 143 - RNA, 44 nt, synthetic construct
  source 1..44: mol_type = other RNA
atagattgctccttacgaggagactcaaaggctcggaggagcag

SEQ ID NO: 144 - RNA, 44 nt, synthetic construct
  source 1..44: mol_type = other RNA
atagattgctccttacgaggagaccgcggggagggtgctgtccg

SEQ ID NO: 145 - RNA, 44 nt, synthetic construct
  source 1..44: mol_type = other RNA
atagattgctccttacgaggagacccgccggtgctgcctcagct

SEQ ID NO: 146 - RNA, 44 nt, synthetic construct
  source 1..44: mol_type = other RNA
atagattgctccttacgaggagaccccgggtgcggaggccaccg

SEQ ID NO: 147 - RNA, 44 nt, synthetic construct
  source 1..44: mol_type = other RNA
atagattgctccttacgaggagaccatctgcccctgccgcgcgg

SEQ ID NO: 148 - RNA, 44 nt, synthetic construct
  source 1..44: mol_type = other RNA
atagattgctccttacgaggagaccgcctgctgcagaaactccg

SEQ ID NO: 149 - RNA, 44 nt, synthetic construct
  source 1..44: mol_type = other RNA
atagattgctccttacgaggagacccacgccgccccggcgacct

SEQ ID NO: 150 - RNA, 44 nt, synthetic construct
  source 1..44: mol_type = other RNA
atagattgctccttacgaggagacgccctgcggccccgcttgag

SEQ ID NO: 151 - RNA, 44 nt, synthetic construct
  source 1..44: mol_type = other RNA
atagattgctccttacgaggagacctaggagaggttgcgcctgc

SEQ ID NO: 152 - RNA, 44 nt, synthetic construct
  source 1..44: mol_type = other RNA
atagattgctccttacgaggagacagcgggcccaggctgtgcca

SEQ ID NO: 153 - RNA, 44 nt, synthetic construct
  source 1..44: mol_type = other RNA
atagattgctccttacgaggagacctcgctgaggggtgcttcca

SEQ ID NO: 154 - RNA, 44 nt, synthetic construct
  source 1..44: mol_type = other RNA
atagattgctccttacgaggagaccgctcaaagcaggctcgcag

SEQ ID NO: 155 - RNA, 44 nt, synthetic construct
  source 1..44: mol_type = other RNA
atagattgctccttacgaggagacggctcggggtccaaacgagt

SEQ ID NO: 156 - RNA, 44 nt, synthetic construct
  source 1..44: mol_type = other RNA
atagattgctccttacgaggagacgattctgaaaccagatctga

SEQ ID NO: 157 - RNA, 44 nt, synthetic construct
  source 1..44: mol_type = other RNA
atagattgctccttacgaggagactgcccgggtgcggaggccac

SEQ ID NO: 158 - RNA, 44 nt, synthetic construct
  source 1..44: mol_type = other RNA
atagattgctccttacgaggagactgaaaccaaatctggaccct

SEQ ID NO: 159 - RNA, 44 nt, synthetic construct
  source 1..44: mol_type = other RNA
atagattgctccttacgaggagaccgaggcctccagctcccccg

SEQ ID NO: 160 - RNA, 44 nt, synthetic construct
  source 1..44: mol_type = other RNA
atagattgctccttacgaggagactctggtggcgatgcccgggt

SEQ ID NO: 161 - RNA, 44 nt, synthetic construct
  source 1..44: mol_type = other RNA
atagattgctccttacgaggagacggagatcccctctgccggcg

SEQ ID NO: 162 - RNA, 44 nt, synthetic construct
  source 1..44: mol_type = other RNA
atagattgctccttacgaggagactaggagaggttgcgcctgct

SEQ ID NO: 163 - RNA, 44 nt, synthetic construct
  source 1..44: mol_type = other RNA
atagattgctccttacgaggagacgggttcccacgccgccccgg

SEQ ID NO: 164 - RNA, 44 nt, synthetic construct
  source 1..44: mol_type = other RNA
atagattgctccttacgaggagaccgccggccttctggcgggcc

SEQ ID NO: 165 - RNA, 44 nt, synthetic construct
  source 1..44: mol_type = other RNA
atagattgctccttacgaggagaccccacgcgccggtgtgggcg

SEQ ID NO: 166 - RNA, 44 nt, synthetic construct
  source 1..44: mol_type = other RNA
atagattgctccttacgaggagaccagcgaggcggcctcttccg

SEQ ID NO: 167 - RNA, 44 nt, synthetic construct
  source 1..44: mol_type = other RNA
atagattgctccttacgaggagacttcctcgctgaggggtgctt

SEQ ID NO: 168 - RNA, 44 nt, synthetic construct
  source 1..44: mol_type = other RNA
atagattgctccttacgaggagacgctcggggtccaaacgagtc

SEQ ID NO: 169 - RNA, 44 nt, synthetic construct
  source 1..44: mol_type = other RNA
atagattgctccttacgaggagactggcgggccgcgtctcccgg

SEQ ID NO: 170 - RNA, 44 nt, synthetic construct
  source 1..44: mol_type = other RNA
atagattgctccttacgaggagacgccggccttctggcgggccg

SEQ ID NO: 171 - RNA, 44 nt, synthetic construct
  source 1..44: mol_type = other RNA
atagattgctccttacgaggagaccgacgctgtctaggcaaacc

SEQ ID NO: 172 - RNA, 44 nt, synthetic construct
  source 1..44: mol_type = other RNA
atagattgctccttacgaggagacagagatatattaaaatgccc

SEQ ID NO: 173 - RNA, 44 nt, synthetic construct
  source 1..44: mol_type = other RNA
atagattgctccttacgaggagaccgtgaaattctggctgaatg

SEQ ID NO: 174 - RNA, 44 nt, synthetic construct
  source 1..44: mol_type = other RNA
atagattgctccttacgaggagacttccgtgaaattctggctga

SEQ ID NO: 175 - RNA, 44 nt, synthetic construct
  source 1..44: mol_type = other RNA
atagattgctccttacgaggagacgagttacatctcctggatga

SEQ ID NO: 176 - RNA, 44 nt, synthetic construct
  source 1..44: mol_type = other RNA
atagattgctccttacgaggagacttcttccgtgaaattctggc

SEQ ID NO: 177 - RNA, 44 nt, synthetic construct
  source 1..44: mol_type = other RNA
atagattgctccttacgaggagactggctgaatgtctcccccca

SEQ ID NO: 178 - RNA, 44 nt, synthetic construct
  source 1..44: mol_type = other RNA
atagattgctccttacgaggagaccatctcctggatgattagtt

SEQ ID NO: 179 - RNA, 44 nt, synthetic construct
  source 1..44: mol_type = other RNA
atagattgctccttacgaggagacaaatgccccctccctgtgga

SEQ ID NO: 180 - RNA, 44 nt, synthetic construct
  source 1..44: mol_type = other RNA
atagattgctccttacgaggagaccacgtcagccggggtgcgca

SEQ ID NO: 181 - RNA, 44 nt, synthetic construct
  source 1..44: mol_type = other RNA
atagattgctccttacgaggagacatatatctctgaactaatca

SEQ ID NO: 182 - RNA, 44 nt, synthetic construct
  source 1..44: mol_type = other RNA
atagattgctccttacgaggagacacggaagaacaagggcacag

SEQ ID NO: 183 - RNA, 44 nt, synthetic construct
  source 1..44: mol_type = other RNA
atagattgctccttacgaggagaccctagacagcgtcggaaggt

SEQ ID NO: 184 - RNA, 44 nt, synthetic construct
  source 1..44: mol_type = other RNA
atagattgctccttacgaggagaccacggaagaacaagggcaca

SEQ ID NO: 185 - RNA, 44 nt, synthetic construct
  source 1..44: mol_type = other RNA
atagattgctccttacgaggagacgcctagacagcgtcggaagg

SEQ ID NO: 186 - RNA, 44 nt, synthetic construct
  source 1..44: mol_type = other RNA
atagattgctccttacgaggagacaatatatctctgaactaatc

SEQ ID NO: 187 - RNA, 44 nt, synthetic construct
  source 1..44: mol_type = other RNA
atagattgctccttacgaggagacagccagaatttcacggaaga

SEQ ID NO: 188 - RNA, 44 nt, synthetic construct
  source 1..44: mol_type = other RNA
atagattgctccttacgaggagaccccgcttcctggctagacct

SEQ ID NO: 189 - RNA, 44 nt, synthetic construct
  source 1..44: mol_type = other RNA
atagattgctccttacgaggagacctggctagacctgcgcgcag

SEQ ID NO: 190 - RNA, 44 nt, synthetic construct
  source 1..44: mol_type = other RNA
atagattgctccttacgaggagactataggatccacagggaggg

SEQ ID NO: 191 - RNA, 44 nt, synthetic construct
  source 1..44: mol_type = other RNA
atagattgctccttacgaggagaccgggcagccgcctgggctgt

SEQ ID NO: 192 - RNA, 44 nt, synthetic construct
  source 1..44: mol_type = other RNA
atagattgctccttacgaggagacgcgggcagccgcctgggctg

SEQ ID NO: 193 - RNA, 44 nt, synthetic construct
  source 1..44: mol_type = other RNA
atagattgctccttacgaggagaccgggggtggggggtgggggt

SEQ ID NO: 194 - RNA, 44 nt, synthetic construct
  source 1..44: mol_type = other RNA
atagattgctccttacgaggagacgcgggacgggggtctccacc

SEQ ID NO: 195 - RNA, 44 nt, synthetic construct
  source 1..44: mol_type = other RNA
atagattgctccttacgaggagacgaccgccaggcgctccgtgc

SEQ ID NO: 196 - RNA, 44 nt, synthetic construct
  source 1..44: mol_type = other RNA
atagattgctccttacgaggagactccgggggtggggggtgggg

SEQ ID NO: 197 - RNA, 44 nt, synthetic construct
  source 1..44: mol_type = other RNA
atagattgctccttacgaggagacccgggggtggggggtggggg

SEQ ID NO: 198 - RNA, 44 nt, synthetic construct
  source 1..44: mol_type = other RNA
atagattgctccttacgaggagaccgcgggacgggggtctccac

SEQ ID NO: 199 - RNA, 44 nt, synthetic construct
  source 1..44: mol_type = other RNA
atagattgctccttacgaggagactgaccgccaggcgctccgtg

SEQ ID NO: 200 - RNA, 44 nt, synthetic construct
  source 1..44: mol_type = other RNA
atagattgctccttacgaggagacaccgccaggcgctccgtgct

SEQ ID NO: 201 - RNA, 44 nt, synthetic construct
  source 1..44: mol_type = other RNA
atagattgctccttacgaggagacgcccgcttcctggctagacc

SEQ ID NO: 202 - RNA, 44 nt, synthetic construct
  source 1..44: mol_type = other RNA
atagattgctccttacgaggagacggtgatcagtgcagatgtgt

SEQ ID NO: 203 - RNA, 44 nt, synthetic construct
  source 1..44: mol_type = other RNA
atagattgctccttacgaggagaccagaactccatagtagactg

SEQ ID NO: 204 - RNA, 44 nt, synthetic construct
  source 1..44: mol_type = other RNA
atagattgctccttacgaggagactgtgtgatgagtgcagagat

SEQ ID NO: 205 - RNA, 44 nt, synthetic construct
  source 1..44: mol_type = other RNA
atagattgctccttacgaggagaccatcttttgtgtgatgagtg

SEQ ID NO: 206 - RNA, 44 nt, synthetic construct
  source 1..44: mol_type = other RNA
atagattgctccttacgaggagactgtgatgagtgcagagatat

SEQ ID NO: 207 - RNA, 44 nt, synthetic construct
  source 1..44: mol_type = other RNA
atagattgctccttacgaggagacacagaacttcggtgatcagt

SEQ ID NO: 208 - RNA, 44 nt, synthetic construct
  source 1..44: mol_type = other RNA
atagattgctccttacgaggagacgcatcttttgtgtgatgagt

SEQ ID NO: 209 - RNA, 44 nt, synthetic construct
  source 1..44: mol_type = other RNA
atagattgctccttacgaggagaccagaacttcggtgatcagtg

SEQ ID NO: 210 - RNA, 44 nt, synthetic construct
  source 1..44: mol_type = other RNA
atagattgctccttacgaggagacagaactccatagtagactga

SEQ ID NO: 211 - RNA, 44 nt, synthetic construct
  source 1..44: mol_type = other RNA
atagattgctccttacgaggagacgtgtgatgagtgcagagata

SEQ ID NO: 212 - RNA, 44 nt, synthetic construct
  source 1..44: mol_type = other RNA
atagattgctccttacgaggagaccatcacttaggtgatcagtg

SEQ ID NO: 213 - RNA, 44 nt, synthetic construct
  source 1..44: mol_type = other RNA
atagattgctccttacgaggagacggtgatcagtgtagagatat

SEQ ID NO: 214 - RNA, 44 nt, synthetic construct
  source 1..44: mol_type = other RNA
atagattgctccttacgaggagacaaattctcgtgtagacagag

SEQ ID NO: 215 - RNA, 44 nt, synthetic construct
  source 1..44: mol_type = other RNA
atagattgctccttacgaggagacatttacagaacttcggtgat

SEQ ID NO: 216 - RNA, 44 nt, synthetic construct
  source 1..44: mol_type = other RNA
atagattgctccttacgaggagactgaaacacatctgcactgat

SEQ ID NO: 217 - RNA, 44 nt, synthetic construct
  source 1..44: mol_type = other RNA
atagattgctccttacgaggagacttctacaggggatattgtga

SEQ ID NO: 218 - RNA, 44 nt, synthetic construct
  source 1..44: mol_type = other RNA
atagattgctccttacgaggagacagtctactatggagttctga

SEQ ID NO: 219 - RNA, 44 nt, synthetic construct
  source 1..44: mol_type = other RNA
atagattgctccttacgaggagaccaggctttttctacagggga

SEQ ID NO: 220 - RNA, 44 nt, synthetic construct
  source 1..44: mol_type = other RNA
atagattgctccttacgaggagactctacaggggatattgtgac

SEQ ID NO: 221 - RNA, 44 nt, synthetic construct
  source 1..44: mol_type = other RNA
atagattgctccttacgaggagacctacaggggatattgtgaca

SEQ ID NO: 222 - RNA, 44 nt, synthetic construct
  source 1..44: mol_type = other RNA
atagattgctccttacgaggagactaacatatctctacactgat

SEQ ID NO: 223 - RNA, 44 nt, synthetic construct
  source 1..44: mol_type = other RNA
atagattgctccttacgaggagacacatatctctacactgatca

SEQ ID NO: 224 - RNA, 44 nt, synthetic construct
  source 1..44: mol_type = other RNA
atagattgctccttacgaggagactgacatatctctgcactcat

SEQ ID NO: 225 - RNA, 44 nt, synthetic construct
  source 1..44: mol_type = other RNA
atagattgctccttacgaggagacaacatatctctacactgatc

SEQ ID NO: 226 - RNA, 44 nt, synthetic construct
  source 1..44: mol_type = other RNA
atagattgctccttacgaggagactgtaaatcaatttcaggctt

SEQ ID NO: 227 - RNA, 44 nt, synthetic construct
  source 1..44: mol_type = other RNA
atagattgctccttacgaggagactacaggggatattgtgacat

SEQ ID NO: 228 - RNA, 44 nt, synthetic construct
  source 1..44: mol_type = other RNA
atagattgctccttacgaggagactctaggttcagtctactatg

SEQ ID NO: 229 - RNA, 44 nt, synthetic construct
  source 1..44: mol_type = other RNA
atagattgctccttacgaggagacaggctttttctacaggggat

SEQ ID NO: 230 - protein, 717 aa, unidentified
  REGION 1..717: note = CasPhi.12
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 231 - protein, 748 aa, synthetic construct
  REGION 1..748: note = CasPhi.12 with NLS
PKKKRKVGIHGVPAAMIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKK

SEQ ID NO: 232 - protein, 717 aa, unidentified
  REGION 1..717: note = CasPhi.12 L26R
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 233 - protein, 775 aa, synthetic construct
  REGION 1..775: note = 3x Flag-SV40NLS -CasPhi12 L26R-NLS
MDYKDHDGDYKDHDIDYKDDDDKMAPKKKRKVGIHGVPAAMIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKGRRPRKRPARQKRKRNS

SEQ ID NO: 234 - protein, 17 aa, synthetic construct
  REGION 1..17: note = Nuclear localization signal 1 SV40NLS
MAPKKKRKVGIHGVPAA

SEQ ID NO: 235 - protein, 16 aa, synthetic construct
  REGION 1..16: note = Nuclear localization signal 2
KRPAATKKAGQAKKKK

SEQ ID NO: 236 - protein, 4 aa, synthetic construct
  REGION 1..4: note = linker
GAAA

SEQ ID NO: 237 - RNA, 36 nt, synthetic construct
  source 1..36: mol_type = other RNA
ctttcaagactaatagattgctccttacgaggagac

SEQ ID NO: 238 - RNA, 24 nt, synthetic construct
  source 1..24: mol_type = other RNA
atagattgctccttacgaggagac

SEQ ID NO: 239 - RNA, 23 nt, synthetic construct
  source 1..23: mol_type = other RNA
tagattgctccttacgaggagac

SEQ ID NO: 240 - RNA, 22 nt, synthetic construct
  source 1..22: mol_type = other RNA
agattgctccttacgaggagac

SEQ ID NO: 241 - RNA, 21 nt, synthetic construct
  source 1..21: mol_type = other RNA
gattgctccttacgaggagac

SEQ ID NO: 242 - RNA, 20 nt, synthetic construct
  source 1..20: mol_type = other RNA
attgctccttacgaggagac

SEQ ID NO: 243 - protein, 717 aa, unidentified
  REGION 1..717: note = CasPhi.12 E567A
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIANLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 244 - protein, 717 aa, unidentified
  REGION 1..717: note = CasPhi.12 E567Q
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 245 - protein, 15 aa, synthetic construct
  REGION 1..15: note = nuclear localization signal
PKKKRKVGIHGVPAA

SEQ ID NO: 246 - protein, 16 aa, synthetic construct
  REGION 1..16: note = nuclear localization signal
KRPAATKKAGQAKKKK

SEQ ID NO: 247-248 - skipped (2 entries)

SEQ ID NO: 249 - protein, 8 aa, synthetic construct
  REGION 1..8: note = nuclear localization signal
  VARIANT 3: note = Xaa is any naturally occuring amino acid
  VARIANT 4: note = Tryptophan, Phenyalanine or Tyrosine
  VARIANT 5..6: note = Xaa is any naturally occuring amino acid
KRXXXXAF

SEQ ID NO: 250 - skipped

SEQ ID NO: 251 - protein, 6 aa, synthetic construct
  REGION 1..6: note = nuclear localization signal
  VARIANT 5: note = Lysing or Arginine
  VARIANT 6: note = Tryptophan, Phenylalanine or Tyrosine
LGKRXX

SEQ ID NO: 252-258 - skipped (7 entries)

SEQ ID NO: 259 - protein, 16 aa, synthetic construct
  REGION 1..16: note = nuclear localization signal
APKKKRKVGIHGVPAA

SEQ ID NO: 260 - protein, 20 aa, synthetic construct
  VARIANT 4: note = Xaa is any naturally occurring amino acid
  VARIANT 8: note = Xaa is any naturally occurring amino acid
  VARIANT 11: note = Xaa is any naturally occurring amino acid
  VARIANT 15: note = Xaa is any naturally occurring amino acid
  VARIANT 19: note = Xaa is any naturally occurring amino acid
GLFXALLXLLXSLWXLLLXA

SEQ ID NO: 261 - protein, 20 aa, synthetic construct
GLFHALLHLLHSLWHLLLHA

SEQ ID NO: 262 - protein, 717 aa, unidentified
  REGION 1..717: note = CasPhi.12 E109R
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSRHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 263 - protein, 717 aa, unidentified
  REGION 1..717: note = CasPhi.12 H208R
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYRNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 264 - protein, 717 aa, unidentified
  REGION 1..717: note = CasPhi.12 K184R
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQRPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 265 - protein, 717 aa, unidentified
  REGION 1..717: note = CasPhi.12 K38R
MIKPTVSQFLTPGFKLIRNHSRTAGLKLKNEGEEACKRFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 266 - protein, 717 aa, unidentified
  REGION 1..717: note = CasPhi.12 L182R
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLRQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 267 - protein, 717 aa, unidentified
  REGION 1..717: note = CasPhi.12 Q183R
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLRKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 268 - protein, 717 aa, unidentified
  REGION 1..717: note = CasPhi.12 S108R
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLREHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 269 - protein, 717 aa, unidentified
  REGION 1..717: note = CasPhi.12 S198R
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVRPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 270 - protein, 717 aa, unidentified
  REGION 1..717: note = CasPhi.12 T114R
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDRVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 271 - protein, 717 aa, unidentified
  REGION 1..717: note = CasPhi.12 D369A
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVAVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 272 - protein, 717 aa, unidentified
  REGION 1..717: note = CasPhi.12 D369N
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVNVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 273 - protein, 717 aa, unidentified
  REGION 1..717: note = CasPhi.12 D658A
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNAAIDVATENLATVAITAQSMPKPTCERSGDAKKPVRARKAKAPEFHDKLAPSYTVVLREAV

SEQ ID NO: 274 - protein, 717 aa, unidentified
  REGION 1..717: note = CasPhi.12 D658N
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNANIDVATENLATVAITAQSMPKPTCERSGDAKKPVRARKAKAPEFHDKLAPSYTVVLREAV

SEQ ID NO: 275 - RNA, 20 nt, synthetic construct
  source 1..20: mol_type = other RNA
gaccccgagccaaagcgagg

SEQ ID NO: 276 - RNA, 20 nt, synthetic construct
  source 1..20: mol_type = other RNA
agcggaacccgtacccgggc

SEQ ID NO: 277 - RNA, 20 nt, synthetic construct
  source 1..20: mol_type = other RNA
ctggaggagctttaggacgc

SEQ ID NO: 278 - RNA, 20 nt, synthetic construct
  source 1..20: mol_type = other RNA
agaaggatcgctttccaggc

SEQ ID NO: 279 - RNA, 20 nt, synthetic construct
  source 1..20: mol_type = other RNA
cagcgcggcccccggcgggg

SEQ ID NO: 280 - RNA, 20 nt, synthetic construct
  source 1..20: mol_type = other RNA
cagcaggcgcaacctctcct

SEQ ID NO: 281 - RNA, 20 nt, synthetic construct
  source 1..20: mol_type = other RNA
gtttcagaatgagaggtcac

SEQ ID NO: 282 - RNA, 20 nt, synthetic construct
  source 1..20: mol_type = other RNA
cactcccctgcggcctgctg

SEQ ID NO: 283 - RNA, 20 nt, synthetic construct
  source 1..20: mol_type = other RNA
gtttcagaatcgaagggcca

SEQ ID NO: 284 - RNA, 20 nt, synthetic construct
  source 1..20: mol_type = other RNA
ggatccggtgacggcggtcc

SEQ ID NO: 285 - RNA, 20 nt, synthetic construct
  source 1..20: mol_type = other RNA
cccctgccgcgcggaggcgg

SEQ ID NO: 286 - RNA, 20 nt, synthetic construct
  source 1..20: mol_type = other RNA
ccggcgcggcctggctgggc

SEQ ID NO: 287 - RNA, 20 nt, synthetic construct
  source 1..20: mol_type = other RNA
ggagaggttgcgcctgctgc

SEQ ID NO: 288 - RNA, 20 nt, synthetic construct
  source 1..20: mol_type = other RNA
ggcgaaggcgacccacgagg

SEQ ID NO: 289 - RNA, 20 nt, synthetic construct
  source 1..20: mol_type = other RNA
aatcctggactccgggaggc

SEQ ID NO: 290 - RNA, 20 nt, synthetic construct
  source 1..20: mol_type = other RNA
gccagctcctcccgggcggc

SEQ ID NO: 291 - RNA, 20 nt, synthetic construct
  source 1..20: mol_type = other RNA
aaaccaaatctggaccctgg

SEQ ID NO: 292 - RNA, 20 nt, synthetic construct
  source 1..20: mol_type = other RNA
cccgggtgcggaggccaccg

SEQ ID NO: 293 - RNA, 20 nt, synthetic construct
  source 1..20: mol_type = other RNA
gcgggccgcgtctcccgggc

SEQ ID NO: 294 - RNA, 20 nt, synthetic construct
  source 1..20: mol_type = other RNA
gctcggggtccaaacgagtc

SEQ ID NO: 295 - RNA, 20 nt, synthetic construct
  source 1..20: mol_type = other RNA
aaaccagatctgaatcctgg

SEQ ID NO: 296 - RNA, 20 nt, synthetic construct
  source 1..20: mol_type = other RNA
gtggcgatgcccgggtacgg

SEQ ID NO: 297 - RNA, 20 nt, synthetic construct
  source 1..20: mol_type = other RNA
ggagagccccaggcgcgcag

SEQ ID NO: 298 - RNA, 20 nt, synthetic construct
  source 1..20: mol_type = other RNA
ccaccgcgcaggggcccggc

SEQ ID NO: 299 - RNA, 20 nt, synthetic construct
  source 1..20: mol_type = other RNA
gaccctgggctccggaatgc

SEQ ID NO: 300 - RNA, 20 nt, synthetic construct
  source 1..20: mol_type = other RNA
cccttgttcttccgtgaaat

SEQ ID NO: 301 - RNA, 20 nt, synthetic construct
  source 1..20: mol_type = other RNA
ttaaaatgccccctccctgt

SEQ ID NO: 302 - RNA, 20 nt, synthetic construct
  source 1..20: mol_type = other RNA
gctgaatgtctccccccacc

SEQ ID NO: 303 - RNA, 20 nt, synthetic construct
  source 1..20: mol_type = other RNA
tgcccttgttcttccgtgaa

SEQ ID NO: 304 - RNA, 20 nt, synthetic construct
  source 1..20: mol_type = other RNA
ggcaaacctggattagagtt

SEQ ID NO: 305 - RNA, 20 nt, synthetic construct
  source 1..20: mol_type = other RNA
atatatctctgaactaatca

SEQ ID NO: 306 - RNA, 20 nt, synthetic construct
  source 1..20: mol_type = other RNA
tctctgaactaatcatccag

SEQ ID NO: 307 - RNA, 20 nt, synthetic construct
  source 1..20: mol_type = other RNA
aactaatcatccaggagatg

SEQ ID NO: 308 - RNA, 20 nt, synthetic construct
  source 1..20: mol_type = other RNA
cctagacagcgtcggaaggt

SEQ ID NO: 309 - RNA, 20 nt, synthetic construct
  source 1..20: mol_type = other RNA
ggatccacagggagggggca

SEQ ID NO: 310 - RNA, 20 nt, synthetic construct
  source 1..20: mol_type = other RNA
atccaggtttgcctagacag

SEQ ID NO: 311 - RNA, 20 nt, synthetic construct
  source 1..20: mol_type = other RNA
actctaatccaggtttgcct

SEQ ID NO: 312 - RNA, 20 nt, synthetic construct
  source 1..20: mol_type = other RNA
cccgcttcctggctagacct

SEQ ID NO: 313 - RNA, 20 nt, synthetic construct
  source 1..20: mol_type = other RNA
taggatccacagggaggggg

SEQ ID NO: 314 - RNA, 20 nt, synthetic construct
  source 1..20: mol_type = other RNA
ggagcagcccgggcagagct

SEQ ID NO: 315 - RNA, 20 nt, synthetic construct
  source 1..20: mol_type = other RNA
tctgtctttgcccgcttcct

SEQ ID NO: 316 - RNA, 20 nt, synthetic construct
  source 1..20: mol_type = other RNA
tctttgcccgcttcctggct

SEQ ID NO: 317 - RNA, 20 nt, synthetic construct
  source 1..20: mol_type = other RNA
cgggcagccgcctgggctgt

SEQ ID NO: 318 - RNA, 20 nt, synthetic construct
  source 1..20: mol_type = other RNA
cgcccccgcgccaccgtcgc

SEQ ID NO: 319 - RNA, 20 nt, synthetic construct
  source 1..20: mol_type = other RNA
cccgggctgctcccacagcc

SEQ ID NO: 320 - RNA, 20 nt, synthetic construct
  source 1..20: mol_type = other RNA
cttttgaccgccaggcgctc

SEQ ID NO: 321 - RNA, 20 nt, synthetic construct
  source 1..20: mol_type = other RNA
accgccaggcgctccgtgct

SEQ ID NO: 322 - RNA, 20 nt, synthetic construct
  source 1..20: mol_type = other RNA
gccaggaagcgggcaaagac

SEQ ID NO: 323 - RNA, 20 nt, synthetic construct
  source 1..20: mol_type = other RNA
tttcagaactccatagtaga

SEQ ID NO: 324 - RNA, 20 nt, synthetic construct
  source 1..20: mol_type = other RNA
atgagtgcagagatatgtca

SEQ ID NO: 325 - RNA, 20 nt, synthetic construct
  source 1..20: mol_type = other RNA
tgatgagtgcagagatatgt

SEQ ID NO: 326 - RNA, 20 nt, synthetic construct
  source 1..20: mol_type = other RNA
ttaaaattctcgtgtagaca

SEQ ID NO: 327 - RNA, 20 nt, synthetic construct
  source 1..20: mol_type = other RNA
gagatatgttaaaattctcg

SEQ ID NO: 328 - RNA, 20 nt, synthetic construct
  source 1..20: mol_type = other RNA
gatcctatagaagatttgca

SEQ ID NO: 329 - RNA, 20 nt, synthetic construct
  source 1..20: mol_type = other RNA
cagaacttcggtgatcagtg

SEQ ID NO: 330 - RNA, 20 nt, synthetic construct
  source 1..20: mol_type = other RNA
gaaaaagcctgaaattgatt

SEQ ID NO: 331 - RNA, 20 nt, synthetic construct
  source 1..20: mol_type = other RNA
tgtgatgagtgcagagatat

SEQ ID NO: 332 - RNA, 20 nt, synthetic construct
  source 1..20: mol_type = other RNA
catcttttgtgtgatgagtg

SEQ ID NO: 333 - RNA, 20 nt, synthetic construct
  source 1..20: mol_type = other RNA
gaagatttgcatcttttgtg

SEQ ID NO: 334 - RNA, 20 nt, synthetic construct
  source 1..20: mol_type = other RNA
tcacaatatcccctgtagaa

SEQ ID NO: 335 - RNA, 20 nt, synthetic construct
  source 1..20: mol_type = other RNA
cactgatcaccgaagttctg

SEQ ID NO: 336 - RNA, 20 nt, synthetic construct
  source 1..20: mol_type = other RNA
cactgatcacctaagtgatg

SEQ ID NO: 337 - RNA, 20 nt, synthetic construct
  source 1..20: mol_type = other RNA
acatatctctacactgatca

SEQ ID NO: 338 - RNA, 20 nt, synthetic construct
  source 1..20: mol_type = other RNA
acatatctctgcactcatca

SEQ ID NO: 339 - RNA, 20 nt, synthetic construct
  source 1..20: mol_type = other RNA
cactcatcacacaaaagatg

SEQ ID NO: 340 - RNA, 20 nt, synthetic construct
  source 1..20: mol_type = other RNA
ggttcagtctactatggagt

SEQ ID NO: 341 - RNA, 20 nt, synthetic construct
  source 1..20: mol_type = other RNA
aatcaatttcaggctttttc

SEQ ID NO: 342 - RNA, 20 nt, synthetic construct
  source 1..20: mol_type = other RNA
taaatcaatttcaggctttt

SEQ ID NO: 343 - RNA, 20 nt, synthetic construct
  source 1..20: mol_type = other RNA
accattctctaggttcagtc

SEQ ID NO: 344 - RNA, 20 nt, synthetic construct
  source 1..20: mol_type = other RNA
cacgagaattttaacatatc

SEQ ID NO: 345 - RNA, 20 nt, synthetic construct
  source 1..20: mol_type = other RNA
caggggatattgtgacatat

SEQ ID NO: 346 - RNA, 20 nt, synthetic construct
  source 1..20: mol_type = other RNA
aaacacatctgcactgatca

SEQ ID NO: 347 - RNA, 20 nt, synthetic construct
  source 1..20: mol_type = other RNA
tctacacgagaattttaaca

SEQ ID NO: 348 - RNA, 20 nt, synthetic construct
  source 1..20: mol_type = other RNA
ctatggagttctgaaacaca

SEQ ID NO: 349 - RNA, 20 nt, synthetic construct
  source 1..20: mol_type = other RNA
gagttctgaaacacatctgc

SEQ ID NO: 350 - RNA, 13 nt, synthetic construct
  source 1..13: mol_type = other RNA
aaggatgccaaac

SEQ ID NO: 351 - RNA, 52 nt, synthetic construct
  source 1..52: mol_type = other RNA
acagcttatttggaagctgaaatgtgaggtttataacactcacaagaatcct

SEQ ID NO: 352 - RNA, 69 nt, synthetic construct
  source 1..69: mol_type = other RNA
acagcttatttggaagctgaaatgtgaggtttataacactcacaagaatcctgaaaaaggatgccaaac

SEQ ID NO: 353 - RNA, 89 nt, synthetic construct
  source 1..89: mol_type = other RNA
acagcttatttggaagctgaaatgtgaggtttataacactcacaagaatcctgaaaaaggatgccaaacgaccccgagccaaagcgagg

SEQ ID NO: 354 - RNA, 89 nt, synthetic construct
  source 1..89: mol_type = other RNA
acagcttatttggaagctgaaatgtgaggtttataacactcacaagaatcctgaaaaaggatgccaaacagcggaacccgtacccgggc

SEQ ID NO: 355 - RNA, 89 nt, synthetic construct
  source 1..89: mol_type = other RNA
acagcttatttggaagctgaaatgtgaggtttataacactcacaagaatcctgaaaaaggatgccaaacctggaggagctttaggacgc

SEQ ID NO: 356 - RNA, 89 nt, synthetic construct
  source 1..89: mol_type = other RNA
acagcttatttggaagctgaaatgtgaggtttataacactcacaagaatcctgaaaaaggatgccaaacagaaggatcgctttccaggc

SEQ ID NO: 357 - RNA, 89 nt, synthetic construct
  source 1..89: mol_type = other RNA
acagcttatttggaagctgaaatgtgaggtttataacactcacaagaatcctgaaaaaggatgccaaaccagcgcggcccccggcgggg

SEQ ID NO: 358 - RNA, 89 nt, synthetic construct
  source 1..89: mol_type = other RNA
acagcttatttggaagctgaaatgtgaggtttataacactcacaagaatcctgaaaaaggatgccaaaccagcaggcgcaacctctcct

SEQ ID NO: 359 - RNA, 89 nt, synthetic construct
  source 1..89: mol_type = other RNA
acagcttatttggaagctgaaatgtgaggtttataacactcacaagaatcctgaaaaaggatgccaaacgtttcagaatgagaggtcac

SEQ ID NO: 360 - RNA, 89 nt, synthetic construct
  source 1..89: mol_type = other RNA
acagcttatttggaagctgaaatgtgaggtttataacactcacaagaatcctgaaaaaggatgccaaaccactcccctgcggcctgctg

SEQ ID NO: 361 - RNA, 89 nt, synthetic construct
  source 1..89: mol_type = other RNA
acagcttatttggaagctgaaatgtgaggtttataacactcacaagaatcctgaaaaaggatgccaaacgtttcagaatcgaagggcca

SEQ ID NO: 362 - RNA, 89 nt, synthetic construct
  source 1..89: mol_type = other RNA
acagcttatttggaagctgaaatgtgaggtttataacactcacaagaatcctgaaaaaggatgccaaacggatccggtgacggcggtcc

SEQ ID NO: 363 - RNA, 89 nt, synthetic construct
  source 1..89: mol_type = other RNA
acagcttatttggaagctgaaatgtgaggtttataacactcacaagaatcctgaaaaaggatgccaaaccccctgccgcgcggaggcgg

SEQ ID NO: 364 - RNA, 89 nt, synthetic construct
  source 1..89: mol_type = other RNA
acagcttatttggaagctgaaatgtgaggtttataacactcacaagaatcctgaaaaaggatgccaaacccggcgcggcctggctgggc

SEQ ID NO: 365 - RNA, 89 nt, synthetic construct
  source 1..89: mol_type = other RNA
acagcttatttggaagctgaaatgtgaggtttataacactcacaagaatcctgaaaaaggatgccaaacggagaggttgcgcctgctgc

SEQ ID NO: 366 - RNA, 89 nt, synthetic construct
  source 1..89: mol_type = other RNA
acagcttatttggaagctgaaatgtgaggtttataacactcacaagaatcctgaaaaaggatgccaaacggcgaaggcgacccacgagg

SEQ ID NO: 367 - RNA, 89 nt, synthetic construct
  source 1..89: mol_type = other RNA
acagcttatttggaagctgaaatgtgaggtttataacactcacaagaatcctgaaaaaggatgccaaacaatcctggactccgggaggc

SEQ ID NO: 368 - RNA, 89 nt, synthetic construct
  source 1..89: mol_type = other RNA
acagcttatttggaagctgaaatgtgaggtttataacactcacaagaatcctgaaaaaggatgccaaacgccagctcctcccgggcggc

SEQ ID NO: 369 - RNA, 89 nt, synthetic construct
  source 1..89: mol_type = other RNA
acagcttatttggaagctgaaatgtgaggtttataacactcacaagaatcctgaaaaaggatgccaaacaaaccaaatctggaccctgg

SEQ ID NO: 370 - RNA, 89 nt, synthetic construct
  source 1..89: mol_type = other RNA
acagcttatttggaagctgaaatgtgaggtttataacactcacaagaatcctgaaaaaggatgccaaaccccgggtgcggaggccaccg

SEQ ID NO: 371 - RNA, 89 nt, synthetic construct
  source 1..89: mol_type = other RNA
acagcttatttggaagctgaaatgtgaggtttataacactcacaagaatcctgaaaaaggatgccaaacgcgggccgcgtctcccgggc

SEQ ID NO: 372 - RNA, 89 nt, synthetic construct
  source 1..89: mol_type = other RNA
acagcttatttggaagctgaaatgtgaggtttataacactcacaagaatcctgaaaaaggatgccaaacgctcggggtccaaacgagtc

SEQ ID NO: 373 - RNA, 89 nt, synthetic construct
  source 1..89: mol_type = other RNA
acagcttatttggaagctgaaatgtgaggtttataacactcacaagaatcctgaaaaaggatgccaaacaaaccagatctgaatcctgg

SEQ ID NO: 374 - RNA, 89 nt, synthetic construct
  source 1..89: mol_type = other RNA
acagcttatttggaagctgaaatgtgaggtttataacactcacaagaatcctgaaaaaggatgccaaacgtggcgatgcccgggtacgg

SEQ ID NO: 375 - RNA, 89 nt, synthetic construct
  source 1..89: mol_type = other RNA
acagcttatttggaagctgaaatgtgaggtttataacactcacaagaatcctgaaaaaggatgccaaacggagagccccaggcgcgcag

SEQ ID NO: 376 - RNA, 89 nt, synthetic construct
  source 1..89: mol_type = other RNA
acagcttatttggaagctgaaatgtgaggtttataacactcacaagaatcctgaaaaaggatgccaaacccaccgcgcaggggcccggc

SEQ ID NO: 377 - RNA, 89 nt, synthetic construct
  source 1..89: mol_type = other RNA
acagcttatttggaagctgaaatgtgaggtttataacactcacaagaatcctgaaaaaggatgccaaacgaccctgggctccggaatgc

SEQ ID NO: 378 - RNA, 89 nt, synthetic construct
  source 1..89: mol_type = other RNA
acagcttatttggaagctgaaatgtgaggtttataacactcacaagaatcctgaaaaaggatgccaaaccccttgttcttccgtgaaat

SEQ ID NO: 379 - RNA, 89 nt, synthetic construct
  source 1..89: mol_type = other RNA
acagcttatttggaagctgaaatgtgaggtttataacactcacaagaatcctgaaaaaggatgccaaacttaaaatgccccctccctgt

SEQ ID NO: 380 - RNA, 89 nt, synthetic construct
  source 1..89: mol_type = other RNA
acagcttatttggaagctgaaatgtgaggtttataacactcacaagaatcctgaaaaaggatgccaaacgctgaatgtctccccccacc

SEQ ID NO: 381 - RNA, 89 nt, synthetic construct
  source 1..89: mol_type = other RNA
acagcttatttggaagctgaaatgtgaggtttataacactcacaagaatcctgaaaaaggatgccaaactgcccttgttcttccgtgaa

SEQ ID NO: 382 - RNA, 89 nt, synthetic construct
  source 1..89: mol_type = other RNA
acagcttatttggaagctgaaatgtgaggtttataacactcacaagaatcctgaaaaaggatgccaaacggcaaacctggattagagtt

SEQ ID NO: 383 - RNA, 89 nt, synthetic construct
  source 1..89: mol_type = other RNA
acagcttatttggaagctgaaatgtgaggtttataacactcacaagaatcctgaaaaaggatgccaaacatatatctctgaactaatca

SEQ ID NO: 384 - RNA, 89 nt, synthetic construct
  source 1..89: mol_type = other RNA
acagcttatttggaagctgaaatgtgaggtttataacactcacaagaatcctgaaaaaggatgccaaactctctgaactaatcatccag

SEQ ID NO: 385 - RNA, 89 nt, synthetic construct
  source 1..89: mol_type = other RNA
acagcttatttggaagctgaaatgtgaggtttataacactcacaagaatcctgaaaaaggatgccaaacaactaatcatccaggagatg

SEQ ID NO: 386 - RNA, 89 nt, synthetic construct
  source 1..89: mol_type = other RNA
acagcttatttggaagctgaaatgtgaggtttataacactcacaagaatcctgaaaaaggatgccaaaccctagacagcgtcggaaggt

SEQ ID NO: 387 - RNA, 89 nt, synthetic construct
  source 1..89: mol_type = other RNA
acagcttatttggaagctgaaatgtgaggtttataacactcacaagaatcctgaaaaaggatgccaaacggatccacagggagggggca

SEQ ID NO: 388 - RNA, 89 nt, synthetic construct
  source 1..89: mol_type = other RNA
acagcttatttggaagctgaaatgtgaggtttataacactcacaagaatcctgaaaaaggatgccaaacatccaggtttgcctagacag

SEQ ID NO: 389 - RNA, 89 nt, synthetic construct
  source 1..89: mol_type = other RNA
acagcttatttggaagctgaaatgtgaggtttataacactcacaagaatcctgaaaaaggatgccaaacactctaatccaggtttgcct

SEQ ID NO: 390 - RNA, 89 nt, synthetic construct
  source 1..89: mol_type = other RNA
acagcttatttggaagctgaaatgtgaggtttataacactcacaagaatcctgaaaaaggatgccaaaccccgcttcctggctagacct

SEQ ID NO: 391 - RNA, 89 nt, synthetic construct
  source 1..89: mol_type = other RNA
acagcttatttggaagctgaaatgtgaggtttataacactcacaagaatcctgaaaaaggatgccaaactaggatccacagggaggggg

SEQ ID NO: 392 - RNA, 89 nt, synthetic construct
  source 1..89: mol_type = other RNA
acagcttatttggaagctgaaatgtgaggtttataacactcacaagaatcctgaaaaaggatgccaaacggagcagcccgggcagagct

SEQ ID NO: 393 - RNA, 89 nt, synthetic construct
  source 1..89: mol_type = other RNA
acagcttatttggaagctgaaatgtgaggtttataacactcacaagaatcctgaaaaaggatgccaaactctgtctttgcccgcttcct

SEQ ID NO: 394 - RNA, 89 nt, synthetic construct
  source 1..89: mol_type = other RNA
acagcttatttggaagctgaaatgtgaggtttataacactcacaagaatcctgaaaaaggatgccaaactctttgcccgcttcctggct

SEQ ID NO: 395 - RNA, 89 nt, synthetic construct
  source 1..89: mol_type = other RNA
acagcttatttggaagctgaaatgtgaggtttataacactcacaagaatcctgaaaaaggatgccaaaccgggcagccgcctgggctgt

SEQ ID NO: 396 - RNA, 89 nt, synthetic construct
  source 1..89: mol_type = other RNA
acagcttatttggaagctgaaatgtgaggtttataacactcacaagaatcctgaaaaaggatgccaaaccgcccccgcgccaccgtcgc

SEQ ID NO: 397 - RNA, 89 nt, synthetic construct
  source 1..89: mol_type = other RNA
acagcttatttggaagctgaaatgtgaggtttataacactcacaagaatcctgaaaaaggatgccaaaccccgggctgctcccacagcc

SEQ ID NO: 398 - RNA, 89 nt, synthetic construct
  source 1..89: mol_type = other RNA
acagcttatttggaagctgaaatgtgaggtttataacactcacaagaatcctgaaaaaggatgccaaaccttttgaccgccaggcgctc

SEQ ID NO: 399 - RNA, 89 nt, synthetic construct
  source 1..89: mol_type = other RNA
acagcttatttggaagctgaaatgtgaggtttataacactcacaagaatcctgaaaaaggatgccaaacaccgccaggcgctccgtgct

SEQ ID NO: 400 - RNA, 89 nt, synthetic construct
  source 1..89: mol_type = other RNA
acagcttatttggaagctgaaatgtgaggtttataacactcacaagaatcctgaaaaaggatgccaaacgccaggaagcgggcaaagac

SEQ ID NO: 401 - RNA, 89 nt, synthetic construct
  source 1..89: mol_type = other RNA
acagcttatttggaagctgaaatgtgaggtttataacactcacaagaatcctgaaaaaggatgccaaactttcagaactccatagtaga

SEQ ID NO: 402 - RNA, 89 nt, synthetic construct
  source 1..89: mol_type = other RNA
acagcttatttggaagctgaaatgtgaggtttataacactcacaagaatcctgaaaaaggatgccaaacatgagtgcagagatatgtca

SEQ ID NO: 403 - RNA, 89 nt, synthetic construct
  source 1..89: mol_type = other RNA
acagcttatttggaagctgaaatgtgaggtttataacactcacaagaatcctgaaaaaggatgccaaactgatgagtgcagagatatgt

SEQ ID NO: 404 - RNA, 89 nt, synthetic construct
  source 1..89: mol_type = other RNA
acagcttatttggaagctgaaatgtgaggtttataacactcacaagaatcctgaaaaaggatgccaaacttaaaattctcgtgtagaca

SEQ ID NO: 405 - RNA, 89 nt, synthetic construct
  source 1..89: mol_type = other RNA
acagcttatttggaagctgaaatgtgaggtttataacactcacaagaatcctgaaaaaggatgccaaacgagatatgttaaaattctcg

SEQ ID NO: 406 - RNA, 89 nt, synthetic construct
  source 1..89: mol_type = other RNA
acagcttatttggaagctgaaatgtgaggtttataacactcacaagaatcctgaaaaaggatgccaaacgatcctatagaagatttgca

SEQ ID NO: 407 - RNA, 89 nt, synthetic construct
  source 1..89: mol_type = other RNA
acagcttatttggaagctgaaatgtgaggtttataacactcacaagaatcctgaaaaaggatgccaaaccagaacttcggtgatcagtg

SEQ ID NO: 408 - RNA, 89 nt, synthetic construct
  source 1..89: mol_type = other RNA
acagcttatttggaagctgaaatgtgaggtttataacactcacaagaatcctgaaaaaggatgccaaacgaaaaagcctgaaattgatt

SEQ ID NO: 409 - RNA, 89 nt, synthetic construct
  source 1..89: mol_type = other RNA
acagcttatttggaagctgaaatgtgaggtttataacactcacaagaatcctgaaaaaggatgccaaactgtgatgagtgcagagatat

SEQ ID NO: 410 - RNA, 89 nt, synthetic construct
  source 1..89: mol_type = other RNA
acagcttatttggaagctgaaatgtgaggtttataacactcacaagaatcctgaaaaaggatgccaaaccatcttttgtgtgatgagtg

SEQ ID NO: 411 - RNA, 89 nt, synthetic construct
  source 1..89: mol_type = other RNA
acagcttatttggaagctgaaatgtgaggtttataacactcacaagaatcctgaaaaaggatgccaaacgaagatttgcatcttttgtg

SEQ ID NO: 412 - RNA, 89 nt, synthetic construct
  source 1..89: mol_type = other RNA
acagcttatttggaagctgaaatgtgaggtttataacactcacaagaatcctgaaaaaggatgccaaactcacaatatcccctgtagaa

SEQ ID NO: 413 - RNA, 89 nt, synthetic construct
  source 1..89: mol_type = other RNA
acagcttatttggaagctgaaatgtgaggtttataacactcacaagaatcctgaaaaaggatgccaaaccactgatcaccgaagttctg

SEQ ID NO: 414 - RNA, 89 nt, synthetic construct
  source 1..89: mol_type = other RNA
acagcttatttggaagctgaaatgtgaggtttataacactcacaagaatcctgaaaaaggatgccaaaccactgatcacctaagtgatg

SEQ ID NO: 415 - RNA, 89 nt, synthetic construct
  source 1..89: mol_type = other RNA
acagcttatttggaagctgaaatgtgaggtttataacactcacaagaatcctgaaaaaggatgccaaacacatatctctacactgatca

SEQ ID NO: 416 - RNA, 89 nt, synthetic construct
  source 1..89: mol_type = other RNA
acagcttatttggaagctgaaatgtgaggtttataacactcacaagaatcctgaaaaaggatgccaaacacatatctctgcactcatca

SEQ ID NO: 417 - RNA, 89 nt, synthetic construct
  source 1..89: mol_type = other RNA
acagcttatttggaagctgaaatgtgaggtttataacactcacaagaatcctgaaaaaggatgccaaaccactcatcacacaaaagatg

SEQ ID NO: 418 - RNA, 89 nt, synthetic construct
  source 1..89: mol_type = other RNA
acagcttatttggaagctgaaatgtgaggtttataacactcacaagaatcctgaaaaaggatgccaaacggttcagtctactatggagt

SEQ ID NO: 419 - RNA, 89 nt, synthetic construct
  source 1..89: mol_type = other RNA
acagcttatttggaagctgaaatgtgaggtttataacactcacaagaatcctgaaaaaggatgccaaacaatcaatttcaggctttttc

SEQ ID NO: 420 - RNA, 89 nt, synthetic construct
  source 1..89: mol_type = other RNA
acagcttatttggaagctgaaatgtgaggtttataacactcacaagaatcctgaaaaaggatgccaaactaaatcaatttcaggctttt

SEQ ID NO: 421 - RNA, 89 nt, synthetic construct
  source 1..89: mol_type = other RNA
acagcttatttggaagctgaaatgtgaggtttataacactcacaagaatcctgaaaaaggatgccaaacaccattctctaggttcagtc

SEQ ID NO: 422 - RNA, 89 nt, synthetic construct
  source 1..89: mol_type = other RNA
acagcttatttggaagctgaaatgtgaggtttataacactcacaagaatcctgaaaaaggatgccaaaccacgagaattttaacatatc

SEQ ID NO: 423 - RNA, 89 nt, synthetic construct
  source 1..89: mol_type = other RNA
acagcttatttggaagctgaaatgtgaggtttataacactcacaagaatcctgaaaaaggatgccaaaccaggggatattgtgacatat

SEQ ID NO: 424 - RNA, 89 nt, synthetic construct
  source 1..89: mol_type = other RNA
acagcttatttggaagctgaaatgtgaggtttataacactcacaagaatcctgaaaaaggatgccaaacaaacacatctgcactgatca

SEQ ID NO: 425 - RNA, 89 nt, synthetic construct
  source 1..89: mol_type = other RNA
acagcttatttggaagctgaaatgtgaggtttataacactcacaagaatcctgaaaaaggatgccaaactctacacgagaattttaaca

SEQ ID NO: 426 - RNA, 89 nt, synthetic construct
  source 1..89: mol_type = other RNA
acagcttatttggaagctgaaatgtgaggtttataacactcacaagaatcctgaaaaaggatgccaaacctatggagttctgaaacaca

SEQ ID NO: 427 - RNA, 89 nt, synthetic construct
  source 1..89: mol_type = other RNA
acagcttatttggaagctgaaatgtgaggtttataacactcacaagaatcctgaaaaaggatgccaaacgagttctgaaacacatctgc

SEQ ID NO: 428 - protein, 448 aa, unidentified
  REGION 1..448: note = CasM.265.466
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 429 - protein, 448 aa, synthetic construct
  REGION 1..448: note = CasM.265 466 D220R
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 430 - protein, 504 aa, synthetic construct
  REGION 1..504: note = 3x Flag-SV40NLS-CasM.265466-nucleoplasmin NLS
MDYKDHDGDYKDHDIDYKDDDDKMAPKKKRKVGIHGVPAAMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKKRPAATKKAGQAKKKK

SEQ ID NO: 431 - protein, 448 aa, synthetic construct
  REGION 1..448: note = CasM.265466 K58W
MSVLTRKVQLIPVGDKEERDRVYKYLRDGIEAQNRAMNLYMSGLYFAAINEASKEDRWELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 432 - protein, 448 aa, synthetic construct
  REGION 1..448: note = CasM.265466 A218K
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMKMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 433 - protein, 448 aa, synthetic construct
  REGION 1..448: note = CasM.265466 M295W
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKWKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 434 - protein, 448 aa, synthetic construct
  REGION 1..448: note = CasM.265466 M298L
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPLDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 435 - protein, 448 aa, synthetic construct
  REGION 1..448: note = CasM.265466 N193K
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQKIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 436 - protein, 448 aa, synthetic construct
  REGION 1..448: note = CasM.265466 Y315M
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHMVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 437 - protein, 448 aa, synthetic construct
  REGION 1..448: note = CasM.265466 S209F
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFFGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 438 - protein, 448 aa, synthetic construct
  REGION 1..448: note = CasM.265466 I80K
MSVLTRKVQLIPVGDKEERDRVYKYLRDGIEAQNRAMNLYMSGLYFAAINEASKEDRKELNQLYSRIATSSKGSAYTTDKEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 439 - protein, 448 aa, synthetic construct
  REGION 1..448: note = CasM.265466 E225K
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKKIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 440 - protein, 448 aa, synthetic construct
  REGION 1..448: note = CasM.265466 N286K
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSKGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 441 - protein, 448 aa, synthetic construct
  REGION 1..448: note = CasM.265466 A306K
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEKNWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 442 - protein, 448 aa, synthetic construct
  REGION 1..448: note = CasM.265466 E335Q
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLQNLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 443 - protein, 448 aa, synthetic construct
  REGION 1..448: note = CasM.265466 D237A
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVALGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 444 - protein, 448 aa, synthetic construct
  REGION 1..448: note = CasM.265466 D418A
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNAAFNAARNIAMSTEFQSGKKTKKQKKEQHENK

SEQ ID NO: 445 - protein, 448 aa, synthetic construct
  REGION 1..448: note = CasM.265466 D418N
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNANFNAARNIAMSTEFQSGKKTKKQKKEQHENK

SEQ ID NO: 446 - protein, 448 aa, synthetic construct
  REGION 1..448: note = CasM.265466 E335A
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLANLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 447 - protein, 36 aa, synthetic construct
SDLYIERGGDPRDVHQQVETKPKGKRKSEIRILKIR

SEQ ID NO: 448 - protein, 31 aa, synthetic construct
SDYIVDHGGDPEKVFFETKSKKDKTKRYKRR

SEQ ID NO: 449 - protein, 698 aa, synthetic construct
  REGION 1..698: note = CasPhi j12_L17_18_del1
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 450 - protein, 704 aa, synthetic construct
  REGION 1..704: note = CasPhi j12_L17_18_del2
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 451 - protein, 717 aa, synthetic construct
  REGION 1..717: note = CasPhi.12-L26K-E567Q
MIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 452 - protein, 448 aa, synthetic construct
  REGION 1..448: note = CasM D220R-E335Q
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLQNLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 453 - protein, 166 aa, Escherichia coli
  REGION 1..166: note = ABE8e
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 454 - protein, 664 aa, synthetic construct
  REGION 1..664: note = Cas.265466-D220R-E335Q_ABE8e fusion
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLQNLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKGSSGGSPAGSPTSTEEGTSESATPESGPGTSTEPSEGSAPGSPAGSGGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 455 - protein, 933 aa, synthetic construct
  REGION 1..933: note = CasPhi.12-L26K-E567Q_ABE8e fusion
MIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVGSSGGSPAGSPTSTEEGTSESATPESGPGTSTEPSEGSAPGSPAGSGGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 456 - RNA, 20 nt, synthetic construct
  source 1..20: mol_type = other RNA
agagatatatcaaaatgccc

SEQ ID NO: 457 - RNA, 20 nt, synthetic construct
  source 1..20: mol_type = other RNA
gttcagagatatatcaaaat

SEQ ID NO: 458 - RNA, 20 nt, synthetic construct
  source 1..20: mol_type = other RNA
tcaaaatgccccctccctgt

SEQ ID NO: 459 - RNA, 20 nt, synthetic construct
  source 1..20: mol_type = other RNA
attagttcagagatatatca

SEQ ID NO: 460 - RNA, 20 nt, synthetic construct
  source 1..20: mol_type = other RNA
gatgattagttcagagatat

SEQ ID NO: 461 - RNA, 40 nt, synthetic construct
  source 1..40: mol_type = other RNA
attgctccttacgaggagacagagatatatcaaaatgccc

SEQ ID NO: 462 - RNA, 89 nt, synthetic construct
  source 1..89: mol_type = other RNA
acagcttatttggaagctgaaatgtgaggtttataacactcacaagaatcctgaaaaaggatgccaaacgttcagagatatatcaaaat

SEQ ID NO: 463 - RNA, 89 nt, synthetic construct
  source 1..89: mol_type = other RNA
acagcttatttggaagctgaaatgtgaggtttataacactcacaagaatcctgaaaaaggatgccaaactcaaaatgccccctccctgt

SEQ ID NO: 464 - RNA, 89 nt, synthetic construct
  source 1..89: mol_type = other RNA
acagcttatttggaagctgaaatgtgaggtttataacactcacaagaatcctgaaaaaggatgccaaacattagttcagagatatatca

SEQ ID NO: 465 - RNA, 89 nt, synthetic construct
  source 1..89: mol_type = other RNA
acagcttatttggaagctgaaatgtgaggtttataacactcacaagaatcctgaaaaaggatgccaaacgatgattagttcagagatat

SEQ ID NO: 466 - RNA, 20 nt, synthetic construct
  source 1..20: mol_type = other RNA
caccgcagccacgcagagca

SEQ ID NO: 467 - RNA, 17 nt, synthetic construct
  source 1..17: mol_type = other RNA
gagcaacggcggaaggt

SEQ ID NO: 468 - RNA, 20 nt, synthetic construct
  source 1..20: mol_type = other RNA
tagaaccttgatgacatagc

SEQ ID NO: 469 - DNA, 452 bp, synthetic construct
  source 1..452: mol_type = other DNA
  misc_feature 1..452: note = AAV component Ck8e promoter
cgctccggtgcccgttgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagca

SEQ ID NO: 470 - DNA, 409 bp, synthetic construct
  source 1..409: mol_type = other DNA
  misc_feature 1..409: note = AAV component Spc5-12 promoter
ggtaccgctccggtgcccgtcgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcaccggt

SEQ ID NO: 471 - DNA, 249 bp, synthetic construct
  source 1..249: mol_type = other DNA
  misc_feature 1..249: note = AAV component U6 promoter
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 472 - DNA, 589 bp, synthetic construct
  source 1..589: mol_type = other DNA
  misc_feature 1..589: note = AAV component WPRE
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttggccttcgccctcagacgagtcggatctccctttgggccgcctccccgc

SEQ ID NO: 473 - DNA, 208 bp, synthetic construct
  source 1..208: mol_type = other DNA
  misc_feature 1..208: note = AAV component bGH PolyA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 474 - DNA, 510 bp, synthetic construct
  source 1..510: mol_type = other DNA
  misc_feature 1..510: note = AAV component hGH PolyA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaacacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 475 - protein, 50 aa, synthetic construct
GSSGGSPAGSPTSTEEGTSESATPESGPGTSTEPSEGSAPGSPAGSGGGS

SEQ ID NO: 476 - RNA, 20 nt, synthetic construct
  source 1..20: mol_type = other RNA
ttaaaatgccccctccctgt

SEQ ID NO: 477 - RNA, 20 nt, synthetic construct
  source 1..20: mol_type = other RNA
tattaaaatgccccctccct

SEQ ID NO: 478 - RNA, 20 nt, synthetic construct
  source 1..20: mol_type = other RNA
aaatgccccctccctgtgga

SEQ ID NO: 479 - RNA, 20 nt, synthetic construct
  source 1..20: mol_type = other RNA
aaaatgccccctccctgtgg

SEQ ID NO: 480 - RNA, 20 nt, synthetic construct
  source 1..20: mol_type = other RNA
aatatatctctgaactaatc

SEQ ID NO: 481 - RNA, 20 nt, synthetic construct
  source 1..20: mol_type = other RNA
agagatatattaaaatgccc

SEQ ID NO: 482 - RNA, 20 nt, synthetic construct
  source 1..20: mol_type = other RNA
aaatgccccctccctgtgga

SEQ ID NO: 483 - RNA, 20 nt, synthetic construct
  source 1..20: mol_type = other RNA
atatatctctgaactaatca

SEQ ID NO: 484 - RNA, 20 nt, synthetic construct
  source 1..20: mol_type = other RNA
aatatatctctgaactaatc

SEQ ID NO: 485 - RNA, 17 nt, synthetic construct
  source 1..17: mol_type = other RNA
aaatgccccctccctgt

SEQ ID NO: 486 - RNA, 17 nt, synthetic construct
  source 1..17: mol_type = other RNA
ctcttcgtctctccggc

SEQ ID NO: 487 - RNA, 17 nt, synthetic construct
  source 1..17: mol_type = other RNA
caagggcggctggctgg

SEQ ID NO: 488 - RNA, 17 nt, synthetic construct
  source 1..17: mol_type = other RNA
cggggtggggcgggctg

SEQ ID NO: 489 - RNA, 17 nt, synthetic construct
  source 1..17: mol_type = other RNA
gtctctccggccccacc

SEQ ID NO: 490 - RNA, 17 nt, synthetic construct
  source 1..17: mol_type = other RNA
cacactcccctccaccc

SEQ ID NO: 491 - RNA, 17 nt, synthetic construct
  source 1..17: mol_type = other RNA
ccgttcccgcgggatcc

SEQ ID NO: 492 - RNA, 17 nt, synthetic construct
  source 1..17: mol_type = other RNA
agttccacactcccctc

SEQ ID NO: 493 - RNA, 17 nt, synthetic construct
  source 1..17: mol_type = other RNA
acggagagagggcctgg

SEQ ID NO: 494 - RNA, 17 nt, synthetic construct
  source 1..17: mol_type = other RNA
tccctgctgccgacgcg

SEQ ID NO: 495 - RNA, 17 nt, synthetic construct
  source 1..17: mol_type = other RNA
ccgcgggatccctggag

SEQ ID NO: 496 - RNA, 17 nt, synthetic construct
  source 1..17: mol_type = other RNA
atgaaggggtggagcct

SEQ ID NO: 497 - RNA, 17 nt, synthetic construct
  source 1..17: mol_type = other RNA
acaagggcggctggctg

SEQ ID NO: 498 - RNA, 17 nt, synthetic construct
  source 1..17: mol_type = other RNA
cctccttcacggagaga

SEQ ID NO: 499 - RNA, 17 nt, synthetic construct
  source 1..17: mol_type = other RNA
cggccggggctcaccgc

SEQ ID NO: 500 - RNA, 17 nt, synthetic construct
  source 1..17: mol_type = other RNA
ctccctgctgccgacgc

SEQ ID NO: 501 - RNA, 17 nt, synthetic construct
  source 1..17: mol_type = other RNA
cgggggccggctctccg

SEQ ID NO: 502 - RNA, 17 nt, synthetic construct
  source 1..17: mol_type = other RNA
gggggccggctctccgg

SEQ ID NO: 503 - RNA, 17 nt, synthetic construct
  source 1..17: mol_type = other RNA
agtgtgccaggccctct

SEQ ID NO: 504 - RNA, 17 nt, synthetic construct
  source 1..17: mol_type = other RNA
atgaatggcggtgagcc

SEQ ID NO: 505 - RNA, 17 nt, synthetic construct
  source 1..17: mol_type = other RNA
cacggacggacgcgggc

SEQ ID NO: 506 - RNA, 17 nt, synthetic construct
  source 1..17: mol_type = other RNA
acggacggacgcgggca

SEQ ID NO: 507 - RNA, 17 nt, synthetic construct
  source 1..17: mol_type = other RNA
taaaggcccacaggcag

SEQ ID NO: 508 - RNA, 20 nt, synthetic construct
  source 1..20: mol_type = other RNA
gagcggaacccgtacccggg

SEQ ID NO: 509 - RNA, 20 nt, synthetic construct
  source 1..20: mol_type = other RNA
agaatcgaagggccaggcac

SEQ ID NO: 510 - RNA, 20 nt, synthetic construct
  source 1..20: mol_type = other RNA
ccgcaccccacgtgccctgc

SEQ ID NO: 511 - RNA, 20 nt, synthetic construct
  source 1..20: mol_type = other RNA
gcccacaccggcgcgtgggg

SEQ ID NO: 512 - RNA, 20 nt, synthetic construct
  source 1..20: mol_type = other RNA
gagaaggatcgctttccagg

SEQ ID NO: 513 - RNA, 20 nt, synthetic construct
  source 1..20: mol_type = other RNA
agaatgagaggtcacgccag

SEQ ID NO: 514 - RNA, 20 nt, synthetic construct
  source 1..20: mol_type = other RNA
agaaggatcgctttccaggc

SEQ ID NO: 515 - RNA, 20 nt, synthetic construct
  source 1..20: mol_type = other RNA
ggtttcagaatgagaggtca

SEQ ID NO: 516 - RNA, 20 nt, synthetic construct
  source 1..20: mol_type = other RNA
agcggaacccgtacccgggc

SEQ ID NO: 517 - RNA, 20 nt, synthetic construct
  source 1..20: mol_type = other RNA
ccaggcatcgccgcccggga

SEQ ID NO: 518 - RNA, 20 nt, synthetic construct
  source 1..20: mol_type = other RNA
cggagcccagggtccagatt

SEQ ID NO: 519 - RNA, 20 nt, synthetic construct
  source 1..20: mol_type = other RNA
agatctggtttcagaatcga

SEQ ID NO: 520 - RNA, 20 nt, synthetic construct
  source 1..20: mol_type = other RNA
gtttcagaatgagaggtcac

SEQ ID NO: 521 - RNA, 20 nt, synthetic construct
  source 1..20: mol_type = other RNA
caggcatcgccgcccgggag

SEQ ID NO: 522 - RNA, 20 nt, synthetic construct
  source 1..20: mol_type = other RNA
cagaatcgaagggccaggca

SEQ ID NO: 523 - RNA, 20 nt, synthetic construct
  source 1..20: mol_type = other RNA
cagaatgagaggtcacgcca

SEQ ID NO: 524 - RNA, 20 nt, synthetic construct
  source 1..20: mol_type = other RNA
gtgagccaggcagcgagggc

SEQ ID NO: 525 - RNA, 20 nt, synthetic construct
  source 1..20: mol_type = other RNA
ggaccccgagccaaagcgag

SEQ ID NO: 526 - RNA, 20 nt, synthetic construct
  source 1..20: mol_type = other RNA
gaccccgagccaaagcgagg

SEQ ID NO: 527 - RNA, 20 nt, synthetic construct
  source 1..20: mol_type = other RNA
cgtgagccaggcagcgaggg

SEQ ID NO: 528 - RNA, 20 nt, synthetic construct
  source 1..20: mol_type = other RNA
cgcggggagggtgctgtccg

SEQ ID NO: 529 - RNA, 20 nt, synthetic construct
  source 1..20: mol_type = other RNA
ggctcggggtccaaacgagt

SEQ ID NO: 530 - RNA, 20 nt, synthetic construct
  source 1..20: mol_type = other RNA
ccgccggtgctgcctcagct

SEQ ID NO: 531 - RNA, 20 nt, synthetic construct
  source 1..20: mol_type = other RNA
cgccggccttctggcgggcc

SEQ ID NO: 532 - RNA, 20 nt, synthetic construct
  source 1..20: mol_type = other RNA
tgaaaccagatctgaatcct

SEQ ID NO: 533 - RNA, 20 nt, synthetic construct
  source 1..20: mol_type = other RNA
cgctcaaagcaggctcgcag

SEQ ID NO: 534 - RNA, 20 nt, synthetic construct
  source 1..20: mol_type = other RNA
tggcgggccgcgtctcccgg

SEQ ID NO: 535 - RNA, 20 nt, synthetic construct
  source 1..20: mol_type = other RNA
gccggccttctggcgggccg

SEQ ID NO: 536 - RNA, 20 nt, synthetic construct
  source 1..20: mol_type = other RNA
gctcggggtccaaacgagtc

SEQ ID NO: 537 - RNA, 20 nt, synthetic construct
  source 1..20: mol_type = other RNA
tcaaaggctcggaggagcag

SEQ ID NO: 538 - RNA, 20 nt, synthetic construct
  source 1..20: mol_type = other RNA
cccacgcgccggtgtgggcg

SEQ ID NO: 539 - RNA, 20 nt, synthetic construct
  source 1..20: mol_type = other RNA
tctggtggcgatgcccgggt

SEQ ID NO: 540 - RNA, 20 nt, synthetic construct
  source 1..20: mol_type = other RNA
tgaaaccaaatctggaccct

SEQ ID NO: 541 - RNA, 20 nt, synthetic construct
  source 1..20: mol_type = other RNA
gattctgaaaccagatctga

SEQ ID NO: 542 - RNA, 17 nt, synthetic construct
  source 1..17: mol_type = other RNA
gaccccgagccaaagcg

SEQ ID NO: 543 - RNA, 17 nt, synthetic construct
  source 1..17: mol_type = other RNA
cgccacccacgtcccag

SEQ ID NO: 544 - RNA, 17 nt, synthetic construct
  source 1..17: mol_type = other RNA
gagaaggatcgctttcc

SEQ ID NO: 545 - RNA, 17 nt, synthetic construct
  source 1..17: mol_type = other RNA
cagaatcgaagggccag

SEQ ID NO: 546 - RNA, 17 nt, synthetic construct
  source 1..17: mol_type = other RNA
ccgcaccccacgtgccc

SEQ ID NO: 547 - RNA, 17 nt, synthetic construct
  source 1..17: mol_type = other RNA
cggagcccagggtccag

SEQ ID NO: 548 - RNA, 17 nt, synthetic construct
  source 1..17: mol_type = other RNA
ccaggcatcgccgcccg

SEQ ID NO: 549 - RNA, 17 nt, synthetic construct
  source 1..17: mol_type = other RNA
cagaatgagaggtcacg

SEQ ID NO: 550 - RNA, 17 nt, synthetic construct
  source 1..17: mol_type = other RNA
agaaggatcgctttcca

SEQ ID NO: 551 - RNA, 17 nt, synthetic construct
  source 1..17: mol_type = other RNA
gagcggaacccgtaccc

SEQ ID NO: 552 - RNA, 17 nt, synthetic construct
  source 1..17: mol_type = other RNA
gtttcagaatgagaggt

SEQ ID NO: 553 - RNA, 17 nt, synthetic construct
  source 1..17: mol_type = other RNA
gcctacgccgccccggc

SEQ ID NO: 554 - RNA, 17 nt, synthetic construct
  source 1..17: mol_type = other RNA
agaatgagaggtcacgc

SEQ ID NO: 555 - RNA, 17 nt, synthetic construct
  source 1..17: mol_type = other RNA
agcggaacccgtacccg

SEQ ID NO: 556 - RNA, 17 nt, synthetic construct
  source 1..17: mol_type = other RNA
cgtgagccaggcagcga

SEQ ID NO: 557 - RNA, 17 nt, synthetic construct
  source 1..17: mol_type = other RNA
agatctggtttcagaat

SEQ ID NO: 558 - RNA, 17 nt, synthetic construct
  source 1..17: mol_type = other RNA
agaatcgaagggccagg

SEQ ID NO: 559 - RNA, 17 nt, synthetic construct
  source 1..17: mol_type = other RNA
cgcctacgccgccccgg

SEQ ID NO: 560 - RNA, 17 nt, synthetic construct
  source 1..17: mol_type = other RNA
ctgcagcaggcgcaacc

SEQ ID NO: 561 - RNA, 17 nt, synthetic construct
  source 1..17: mol_type = other RNA
tgcagcaggcgcaacct

SEQ ID NO: 562 - RNA, 17 nt, synthetic construct
  source 1..17: mol_type = other RNA
ggaccccgagccaaagc

SEQ ID NO: 563 - RNA, 17 nt, synthetic construct
  source 1..17: mol_type = other RNA
caggcatcgccgcccgg

SEQ ID NO: 564 - RNA, 17 nt, synthetic construct
  source 1..17: mol_type = other RNA
gtgagccaggcagcgag

SEQ ID NO: 565 - RNA, 17 nt, synthetic construct
  source 1..17: mol_type = other RNA
gcccacaccggcgcgtg

SEQ ID NO: 566 - RNA, 17 nt, synthetic construct
  source 1..17: mol_type = other RNA
ggtttcagaatgagagg

SEQ ID NO: 567 - RNA, 17 nt, synthetic construct
  source 1..17: mol_type = other RNA
ttcctcgctgaggggtg

SEQ ID NO: 568 - RNA, 17 nt, synthetic construct
  source 1..17: mol_type = other RNA
cgcctgctgcagaaact

SEQ ID NO: 569 - RNA, 17 nt, synthetic construct
  source 1..17: mol_type = other RNA
tgcccgggtgcggaggc

SEQ ID NO: 570 - RNA, 17 nt, synthetic construct
  source 1..17: mol_type = other RNA
cagcgaggcggcctctt

SEQ ID NO: 571 - RNA, 17 nt, synthetic construct
  source 1..17: mol_type = other RNA
tgaaaccaaatctggac

SEQ ID NO: 572 - RNA, 17 nt, synthetic construct
  source 1..17: mol_type = other RNA
cgaggcctccagctccc

SEQ ID NO: 573 - RNA, 17 nt, synthetic construct
  source 1..17: mol_type = other RNA
taggagaggttgcgcct

SEQ ID NO: 574 - RNA, 17 nt, synthetic construct
  source 1..17: mol_type = other RNA
agcgggcccaggctgtg

SEQ ID NO: 575 - RNA, 17 nt, synthetic construct
  source 1..17: mol_type = other RNA
gggttcccacgccgccc

SEQ ID NO: 576 - RNA, 17 nt, synthetic construct
  source 1..17: mol_type = other RNA
tgaaaccagatctgaat

SEQ ID NO: 577 - RNA, 17 nt, synthetic construct
  source 1..17: mol_type = other RNA
gctcggggtccaaacga

SEQ ID NO: 578 - RNA, 17 nt, synthetic construct
  source 1..17: mol_type = other RNA
ggagatcccctctgccg

SEQ ID NO: 579 - RNA, 17 nt, synthetic construct
  source 1..17: mol_type = other RNA
cccgggtgcggaggcca

SEQ ID NO: 580 - RNA, 17 nt, synthetic construct
  source 1..17: mol_type = other RNA
ccgccggtgctgcctca

SEQ ID NO: 581 - RNA, 17 nt, synthetic construct
  source 1..17: mol_type = other RNA
ccacgccgccccggcga

SEQ ID NO: 582 - RNA, 17 nt, synthetic construct
  source 1..17: mol_type = other RNA
gccctgcggccccgctt

SEQ ID NO: 583 - RNA, 17 nt, synthetic construct
  source 1..17: mol_type = other RNA
cgctcaaagcaggctcg

SEQ ID NO: 584 - RNA, 17 nt, synthetic construct
  source 1..17: mol_type = other RNA
ctaggagaggttgcgcc

SEQ ID NO: 585 - RNA, 17 nt, synthetic construct
  source 1..17: mol_type = other RNA
ctcgctgaggggtgctt

SEQ ID NO: 586 - RNA, 17 nt, synthetic construct
  source 1..17: mol_type = other RNA
catctgcccctgccgcg

SEQ ID NO: 587 - RNA, 17 nt, synthetic construct
  source 1..17: mol_type = other RNA
gcccgggtgcggaggcc

SEQ ID NO: 588 - RNA, 17 nt, synthetic construct
  source 1..17: mol_type = other RNA
cgcggggagggtgctgt

SEQ ID NO: 589 - RNA, 17 nt, synthetic construct
  source 1..17: mol_type = other RNA
cgccggccttctggcgg

SEQ ID NO: 590 - RNA, 17 nt, synthetic construct
  source 1..17: mol_type = other RNA
ggctcggggtccaaacg

SEQ ID NO: 591 - RNA, 17 nt, synthetic construct
  source 1..17: mol_type = other RNA
tcaaaggctcggaggag

SEQ ID NO: 592 - RNA, 17 nt, synthetic construct
  source 1..17: mol_type = other RNA
gccggccttctggcggg

SEQ ID NO: 593 - RNA, 17 nt, synthetic construct
  source 1..17: mol_type = other RNA
gattctgaaaccagatc

SEQ ID NO: 594 - RNA, 17 nt, synthetic construct
  source 1..17: mol_type = other RNA
cccacgcgccggtgtgg

SEQ ID NO: 595 - RNA, 17 nt, synthetic construct
  source 1..17: mol_type = other RNA
tctggtggcgatgcccg

SEQ ID NO: 596 - RNA, 17 nt, synthetic construct
  source 1..17: mol_type = other RNA
tggcgggccgcgtctcc

SEQ ID NO: 597 - RNA, 89 nt, synthetic construct; identical to SEQ ID NO: 379
  source 1..89: mol_type = other RNA

SEQ ID NO: 598 - RNA, 89 nt, synthetic construct
  source 1..89: mol_type = other RNA
acagcttatttggaagctgaaatgtgaggtttataacactcacaagaatcctgaaaaaggatgccaaactattaaaatgccccctccct

SEQ ID NO: 599 - RNA, 89 nt, synthetic construct
  source 1..89: mol_type = other RNA
acagcttatttggaagctgaaatgtgaggtttataacactcacaagaatcctgaaaaaggatgccaaacaaatgccccctccctgtgga

SEQ ID NO: 600 - RNA, 89 nt, synthetic construct
  source 1..89: mol_type = other RNA
acagcttatttggaagctgaaatgtgaggtttataacactcacaagaatcctgaaaaaggatgccaaacaaaatgccccctccctgtgg

SEQ ID NO: 601 - RNA, 89 nt, synthetic construct
  source 1..89: mol_type = other RNA
acagcttatttggaagctgaaatgtgaggtttataacactcacaagaatcctgaaaaaggatgccaaacaatatatctctgaactaatc

SEQ ID NO: 602 - RNA, 40 nt, synthetic construct
  source 1..40: mol_type = other RNA
attgctccttacgaggagacagagatatattaaaatgccc

SEQ ID NO: 603 - RNA, 40 nt, synthetic construct
  source 1..40: mol_type = other RNA
attgctccttacgaggagacaaatgccccctccctgtgga

SEQ ID NO: 604 - RNA, 40 nt, synthetic construct
  source 1..40: mol_type = other RNA
attgctccttacgaggagacatatatctctgaactaatca

SEQ ID NO: 605 - RNA, 40 nt, synthetic construct
  source 1..40: mol_type = other RNA
attgctccttacgaggagacaatatatctctgaactaatc

SEQ ID NO: 606 - RNA, 37 nt, synthetic construct
  source 1..37: mol_type = other RNA
attgctccttacgaggagacaaatgccccctccctgt

SEQ ID NO: 607 - RNA, 37 nt, synthetic construct
  source 1..37: mol_type = other RNA
attgctccttacgaggagacctcttcgtctctccggc

SEQ ID NO: 608 - RNA, 37 nt, synthetic construct
  source 1..37: mol_type = other RNA
attgctccttacgaggagaccaagggcggctggctgg

SEQ ID NO: 609 - RNA, 37 nt, synthetic construct
  source 1..37: mol_type = other RNA
attgctccttacgaggagaccggggtggggcgggctg

SEQ ID NO: 610 - RNA, 37 nt, synthetic construct
  source 1..37: mol_type = other RNA
attgctccttacgaggagacgtctctccggccccacc

SEQ ID NO: 611 - RNA, 37 nt, synthetic construct
  source 1..37: mol_type = other RNA
attgctccttacgaggagaccacactcccctccaccc

SEQ ID NO: 612 - RNA, 37 nt, synthetic construct
  source 1..37: mol_type = other RNA
attgctccttacgaggagacccgttcccgcgggatcc

SEQ ID NO: 613 - RNA, 37 nt, synthetic construct
  source 1..37: mol_type = other RNA
attgctccttacgaggagacagttccacactcccctc

SEQ ID NO: 614 - RNA, 37 nt, synthetic construct
  source 1..37: mol_type = other RNA
attgctccttacgaggagacacggagagagggcctgg

SEQ ID NO: 615 - RNA, 37 nt, synthetic construct
  source 1..37: mol_type = other RNA
attgctccttacgaggagactccctgctgccgacgcg

SEQ ID NO: 616 - RNA, 37 nt, synthetic construct
  source 1..37: mol_type = other RNA
attgctccttacgaggagacccgcgggatccctggag

SEQ ID NO: 617 - RNA, 37 nt, synthetic construct
  source 1..37: mol_type = other RNA
attgctccttacgaggagacatgaaggggtggagcct

SEQ ID NO: 618 - RNA, 37 nt, synthetic construct
  source 1..37: mol_type = other RNA
attgctccttacgaggagacacaagggcggctggctg

SEQ ID NO: 619 - RNA, 37 nt, synthetic construct
  source 1..37: mol_type = other RNA
attgctccttacgaggagaccctccttcacggagaga

SEQ ID NO: 620 - RNA, 37 nt, synthetic construct
  source 1..37: mol_type = other RNA
attgctccttacgaggagaccggccggggctcaccgc

SEQ ID NO: 621 - RNA, 37 nt, synthetic construct
  source 1..37: mol_type = other RNA
attgctccttacgaggagacctccctgctgccgacgc

SEQ ID NO: 622 - RNA, 37 nt, synthetic construct
  source 1..37: mol_type = other RNA
attgctccttacgaggagaccgggggccggctctccg

SEQ ID NO: 623 - RNA, 37 nt, synthetic construct
  source 1..37: mol_type = other RNA
attgctccttacgaggagacgggggccggctctccgg

SEQ ID NO: 624 - RNA, 37 nt, synthetic construct
  source 1..37: mol_type = other RNA
attgctccttacgaggagacagtgtgccaggccctct

SEQ ID NO: 625 - RNA, 37 nt, synthetic construct
  source 1..37: mol_type = other RNA
attgctccttacgaggagacatgaatggcggtgagcc

SEQ ID NO: 626 - RNA, 37 nt, synthetic construct
  source 1..37: mol_type = other RNA
attgctccttacgaggagaccacggacggacgcgggc

SEQ ID NO: 627 - RNA, 37 nt, synthetic construct
  source 1..37: mol_type = other RNA
attgctccttacgaggagacacggacggacgcgggca

SEQ ID NO: 628 - RNA, 37 nt, synthetic construct
  source 1..37: mol_type = other RNA
attgctccttacgaggagactaaaggcccacaggcag

SEQ ID NO: 629 - RNA, 40 nt, synthetic construct
  source 1..40: mol_type = other RNA
attgctccttacgaggagacgagcggaacccgtacccggg

SEQ ID NO: 630 - RNA, 40 nt, synthetic construct
  source 1..40: mol_type = other RNA
attgctccttacgaggagacagaatcgaagggccaggcac

SEQ ID NO: 631 - RNA, 40 nt, synthetic construct
  source 1..40: mol_type = other RNA
attgctccttacgaggagacccgcaccccacgtgccctgc

SEQ ID NO: 632 - RNA, 40 nt, synthetic construct
  source 1..40: mol_type = other RNA
attgctccttacgaggagacgcccacaccggcgcgtgggg

SEQ ID NO: 633 - RNA, 40 nt, synthetic construct
  source 1..40: mol_type = other RNA
attgctccttacgaggagacgagaaggatcgctttccagg

SEQ ID NO: 634 - RNA, 40 nt, synthetic construct
  source 1..40: mol_type = other RNA
attgctccttacgaggagacagaatgagaggtcacgccag

SEQ ID NO: 635 - RNA, 40 nt, synthetic construct
  source 1..40: mol_type = other RNA
attgctccttacgaggagacagaaggatcgctttccaggc

SEQ ID NO: 636 - RNA, 40 nt, synthetic construct
  source 1..40: mol_type = other RNA
attgctccttacgaggagacggtttcagaatgagaggtca

SEQ ID NO: 637 - RNA, 40 nt, synthetic construct
  source 1..40: mol_type = other RNA
attgctccttacgaggagacagcggaacccgtacccgggc

SEQ ID NO: 638 - RNA, 40 nt, synthetic construct
  source 1..40: mol_type = other RNA
attgctccttacgaggagacccaggcatcgccgcccggga

SEQ ID NO: 639 - RNA, 40 nt, synthetic construct
  source 1..40: mol_type = other RNA
attgctccttacgaggagaccggagcccagggtccagatt

SEQ ID NO: 640 - RNA, 40 nt, synthetic construct
  source 1..40: mol_type = other RNA
attgctccttacgaggagacagatctggtttcagaatcga

SEQ ID NO: 641 - RNA, 40 nt, synthetic construct
  source 1..40: mol_type = other RNA
attgctccttacgaggagacgtttcagaatgagaggtcac

SEQ ID NO: 642 - RNA, 40 nt, synthetic construct
  source 1..40: mol_type = other RNA
attgctccttacgaggagaccaggcatcgccgcccgggag

SEQ ID NO: 643 - RNA, 40 nt, synthetic construct
  source 1..40: mol_type = other RNA
attgctccttacgaggagaccagaatcgaagggccaggca

SEQ ID NO: 644 - RNA, 40 nt, synthetic construct
  source 1..40: mol_type = other RNA
attgctccttacgaggagaccagaatgagaggtcacgcca

SEQ ID NO: 645 - RNA, 40 nt, synthetic construct
  source 1..40: mol_type = other RNA
attgctccttacgaggagacgtgagccaggcagcgagggc

SEQ ID NO: 646 - RNA, 40 nt, synthetic construct
  source 1..40: mol_type = other RNA
attgctccttacgaggagacggaccccgagccaaagcgag

SEQ ID NO: 647 - RNA, 40 nt, synthetic construct
  source 1..40: mol_type = other RNA
attgctccttacgaggagacgaccccgagccaaagcgagg

SEQ ID NO: 648 - RNA, 40 nt, synthetic construct
  source 1..40: mol_type = other RNA
attgctccttacgaggagaccgtgagccaggcagcgaggg

SEQ ID NO: 649 - RNA, 40 nt, synthetic construct
  source 1..40: mol_type = other RNA
attgctccttacgaggagaccgcggggagggtgctgtccg

SEQ ID NO: 650 - RNA, 40 nt, synthetic construct
  source 1..40: mol_type = other RNA
attgctccttacgaggagacggctcggggtccaaacgagt

SEQ ID NO: 651 - RNA, 40 nt, synthetic construct
  source 1..40: mol_type = other RNA
attgctccttacgaggagacccgccggtgctgcctcagct

SEQ ID NO: 652 - RNA, 40 nt, synthetic construct
  source 1..40: mol_type = other RNA
attgctccttacgaggagaccgccggccttctggcgggcc

SEQ ID NO: 653 - RNA, 40 nt, synthetic construct
  source 1..40: mol_type = other RNA
attgctccttacgaggagactgaaaccagatctgaatcct

SEQ ID NO: 654 - RNA, 40 nt, synthetic construct
  source 1..40: mol_type = other RNA
attgctccttacgaggagaccgctcaaagcaggctcgcag

SEQ ID NO: 655 - RNA, 40 nt, synthetic construct
  source 1..40: mol_type = other RNA
attgctccttacgaggagactggcgggccgcgtctcccgg

SEQ ID NO: 656 - RNA, 40 nt, synthetic construct
  source 1..40: mol_type = other RNA
attgctccttacgaggagacgccggccttctggcgggccg

SEQ ID NO: 657 - RNA, 40 nt, synthetic construct
  source 1..40: mol_type = other RNA
attgctccttacgaggagacgctcggggtccaaacgagtc

SEQ ID NO: 658 - RNA, 40 nt, synthetic construct
  source 1..40: mol_type = other RNA
attgctccttacgaggagactcaaaggctcggaggagcag

SEQ ID NO: 659 - RNA, 40 nt, synthetic construct
  source 1..40: mol_type = other RNA
attgctccttacgaggagaccccacgcgccggtgtgggcg

SEQ ID NO: 660 - RNA, 40 nt, synthetic construct
  source 1..40: mol_type = other RNA
attgctccttacgaggagactctggtggcgatgcccgggt

SEQ ID NO: 661 - RNA, 40 nt, synthetic construct
  source 1..40: mol_type = other RNA
attgctccttacgaggagactgaaaccaaatctggaccct

SEQ ID NO: 662 - RNA, 40 nt, synthetic construct
  source 1..40: mol_type = other RNA
attgctccttacgaggagacgattctgaaaccagatctga

SEQ ID NO: 663 - RNA, 37 nt, synthetic construct
  source 1..37: mol_type = other RNA
attgctccttacgaggagacgaccccgagccaaagcg

SEQ ID NO: 664 - RNA, 37 nt, synthetic construct
  source 1..37: mol_type = other RNA
attgctccttacgaggagaccgccacccacgtcccag

SEQ ID NO: 665 - RNA, 37 nt, synthetic construct
  source 1..37: mol_type = other RNA
attgctccttacgaggagacgagaaggatcgctttcc

SEQ ID NO: 666 - RNA, 37 nt, synthetic construct
  source 1..37: mol_type = other RNA
attgctccttacgaggagaccagaatcgaagggccag

SEQ ID NO: 667 - RNA, 37 nt, synthetic construct
  source 1..37: mol_type = other RNA
attgctccttacgaggagacccgcaccccacgtgccc

SEQ ID NO: 668 - RNA, 37 nt, synthetic construct
  source 1..37: mol_type = other RNA
attgctccttacgaggagaccggagcccagggtccag

SEQ ID NO: 669 - RNA, 37 nt, synthetic construct
  source 1..37: mol_type = other RNA
attgctccttacgaggagacccaggcatcgccgcccg

SEQ ID NO: 670 - RNA, 37 nt, synthetic construct
  source 1..37: mol_type = other RNA
attgctccttacgaggagaccagaatgagaggtcacg

SEQ ID NO: 671 - RNA, 37 nt, synthetic construct
  source 1..37: mol_type = other RNA
attgctccttacgaggagacagaaggatcgctttcca

SEQ ID NO: 672 - RNA, 37 nt, synthetic construct
  source 1..37: mol_type = other RNA
attgctccttacgaggagacgagcggaacccgtaccc

SEQ ID NO: 673 - RNA, 37 nt, synthetic construct
  source 1..37: mol_type = other RNA
attgctccttacgaggagacgtttcagaatgagaggt

SEQ ID NO: 674 - RNA, 37 nt, synthetic construct
  source 1..37: mol_type = other RNA
attgctccttacgaggagacgcctacgccgccccggc

SEQ ID NO: 675 - RNA, 37 nt, synthetic construct
  source 1..37: mol_type = other RNA
attgctccttacgaggagacagaatgagaggtcacgc

SEQ ID NO: 676 - RNA, 37 nt, synthetic construct
  source 1..37: mol_type = other RNA
attgctccttacgaggagacagcggaacccgtacccg

SEQ ID NO: 677 - RNA, 37 nt, synthetic construct
  source 1..37: mol_type = other RNA
attgctccttacgaggagaccgtgagccaggcagcga

SEQ ID NO: 678 - RNA, 37 nt, synthetic construct
  source 1..37: mol_type = other RNA
attgctccttacgaggagacagatctggtttcagaat

SEQ ID NO: 679 - RNA, 37 nt, synthetic construct
  source 1..37: mol_type = other RNA
attgctccttacgaggagacagaatcgaagggccagg

SEQ ID NO: 680 - RNA, 37 nt, synthetic construct
  source 1..37: mol_type = other RNA
attgctccttacgaggagaccgcctacgccgccccgg

SEQ ID NO: 681 - RNA, 37 nt, synthetic construct
  source 1..37: mol_type = other RNA
attgctccttacgaggagacctgcagcaggcgcaacc

SEQ ID NO: 682 - RNA, 37 nt, synthetic construct
  source 1..37: mol_type = other RNA
attgctccttacgaggagactgcagcaggcgcaacct

SEQ ID NO: 683 - RNA, 37 nt, synthetic construct
  source 1..37: mol_type = other RNA
attgctccttacgaggagacggaccccgagccaaagc

SEQ ID NO: 684 - RNA, 37 nt, synthetic construct
  source 1..37: mol_type = other RNA
attgctccttacgaggagaccaggcatcgccgcccgg

SEQ ID NO: 685 - RNA, 37 nt, synthetic construct
  source 1..37: mol_type = other RNA
attgctccttacgaggagacgtgagccaggcagcgag

SEQ ID NO: 686 - RNA, 37 nt, synthetic construct
  source 1..37: mol_type = other RNA
attgctccttacgaggagacgcccacaccggcgcgtg

SEQ ID NO: 687 - RNA, 37 nt, synthetic construct
  source 1..37: mol_type = other RNA
attgctccttacgaggagacggtttcagaatgagagg

SEQ ID NO: 688 - RNA, 37 nt, synthetic construct
  source 1..37: mol_type = other RNA
attgctccttacgaggagacttcctcgctgaggggtg

SEQ ID NO: 689 - RNA, 37 nt, synthetic construct
  source 1..37: mol_type = other RNA
attgctccttacgaggagaccgcctgctgcagaaact

SEQ ID NO: 690 - RNA, 37 nt, synthetic construct
  source 1..37: mol_type = other RNA
attgctccttacgaggagactgcccgggtgcggaggc

SEQ ID NO: 691 - RNA, 37 nt, synthetic construct
  source 1..37: mol_type = other RNA
attgctccttacgaggagaccagcgaggcggcctctt

SEQ ID NO: 692 - RNA, 37 nt, synthetic construct
  source 1..37: mol_type = other RNA
attgctccttacgaggagactgaaaccaaatctggac

SEQ ID NO: 693 - RNA, 37 nt, synthetic construct
  source 1..37: mol_type = other RNA
attgctccttacgaggagaccgaggcctccagctccc

SEQ ID NO: 694 - RNA, 37 nt, synthetic construct
  source 1..37: mol_type = other RNA
attgctccttacgaggagactaggagaggttgcgcct

SEQ ID NO: 695 - RNA, 37 nt, synthetic construct
  source 1..37: mol_type = other RNA
attgctccttacgaggagacagcgggcccaggctgtg

SEQ ID NO: 696 - RNA, 37 nt, synthetic construct
  source 1..37: mol_type = other RNA
attgctccttacgaggagacgggttcccacgccgccc

SEQ ID NO: 697 - RNA, 37 nt, synthetic construct
  source 1..37: mol_type = other RNA
attgctccttacgaggagactgaaaccagatctgaat

SEQ ID NO: 698 - RNA, 37 nt, synthetic construct
  source 1..37: mol_type = other RNA
attgctccttacgaggagacgctcggggtccaaacga

SEQ ID NO: 699 - RNA, 37 nt, synthetic construct
  source 1..37: mol_type = other RNA
attgctccttacgaggagacggagatcccctctgccg

SEQ ID NO: 700 - RNA, 37 nt, synthetic construct
  source 1..37: mol_type = other RNA
attgctccttacgaggagaccccgggtgcggaggcca

SEQ ID NO: 701 - RNA, 37 nt, synthetic construct
  source 1..37: mol_type = other RNA
attgctccttacgaggagacccgccggtgctgcctca

SEQ ID NO: 702 - RNA, 37 nt, synthetic construct
  source 1..37: mol_type = other RNA
attgctccttacgaggagacccacgccgccccggcga

SEQ ID NO: 703 - RNA, 37 nt, synthetic construct
  source 1..37: mol_type = other RNA
attgctccttacgaggagacgccctgcggccccgctt

SEQ ID NO: 704 - RNA, 37 nt, synthetic construct
  source 1..37: mol_type = other RNA
attgctccttacgaggagaccgctcaaagcaggctcg

SEQ ID NO: 705 - RNA, 37 nt, synthetic construct
  source 1..37: mol_type = other RNA
attgctccttacgaggagacctaggagaggttgcgcc

SEQ ID NO: 706 - RNA, 37 nt, synthetic construct
  source 1..37: mol_type = other RNA
attgctccttacgaggagacctcgctgaggggtgctt

SEQ ID NO: 707 - RNA, 37 nt, synthetic construct
  source 1..37: mol_type = other RNA
attgctccttacgaggagaccatctgcccctgccgcg

SEQ ID NO: 708 - RNA, 37 nt, synthetic construct
  source 1..37: mol_type = other RNA
attgctccttacgaggagacgcccgggtgcggaggcc

SEQ ID NO: 709 - RNA, 37 nt, synthetic construct
  source 1..37: mol_type = other RNA
attgctccttacgaggagaccgcggggagggtgctgt

SEQ ID NO: 710 - RNA, 37 nt, synthetic construct
  source 1..37: mol_type = other RNA
attgctccttacgaggagaccgccggccttctggcgg

SEQ ID NO: 711 - RNA, 37 nt, synthetic construct
  source 1..37: mol_type = other RNA
attgctccttacgaggagacggctcggggtccaaacg

SEQ ID NO: 712 - RNA, 37 nt, synthetic construct
  source 1..37: mol_type = other RNA
attgctccttacgaggagactcaaaggctcggaggag

SEQ ID NO: 713 - RNA, 37 nt, synthetic construct
  source 1..37: mol_type = other RNA
attgctccttacgaggagacgccggccttctggcggg

SEQ ID NO: 714 - RNA, 37 nt, synthetic construct
  source 1..37: mol_type = other RNA
attgctccttacgaggagacgattctgaaaccagatc

SEQ ID NO: 715 - RNA, 37 nt, synthetic construct
  source 1..37: mol_type = other RNA
attgctccttacgaggagaccccacgcgccggtgtgg

SEQ ID NO: 716 - RNA, 37 nt, synthetic construct
  source 1..37: mol_type = other RNA
attgctccttacgaggagactctggtggcgatgcccg

SEQ ID NO: 717 - RNA, 37 nt, synthetic construct
  source 1..37: mol_type = other RNA
attgctccttacgaggagactggcgggccgcgtctcc